(19) United States
(12) Patent Application Publication
(10) Pub. No.: US 2025/0282887 A1
(43) Pub. Date: Sep. 11, 2025

(54) MAGEB2 BINDING CONSTRUCTS

(71) Applicant: Amgen Inc., Thousand Oaks, CA (US)

(72) Inventors: Agnieszka KIELCZEWSKA, Vancouver (CA); Christine Elaine TINBERG, San Francisco, CA (US)

(73) Assignee: Amgen Inc., Thousand Oaks, CA (US)

(21) Appl. No.: 18/285,125

(22) PCT Filed: Apr. 1, 2022
(86) PCT No.: PCT/US2022/023034

§ 371 Date: Sep. 29, 2023

Related U.S. Application Data
(60) Provisional application No. 63/170,050, filed on Apr. 2, 2021.

(51) Int. Cl. C07K 16/30; A61K 39/00; A61P 35/00; G01N 33/574; G01N 33/68

(52) U.S. Cl. CPC C07K 16/30; A61K 39/0011; A61P 35/00; G01N 33/57496; G01N 33/68; C07K 2317/33; C07K 2317/565

(57) ABSTRACT

The present invention relates to binding constructs comprising a domain which binds to MAGEB2. Moreover, the invention provides polynucleotides encoding the binding constructs, a vector comprising said polynucleotides and a host cell transformed or transfected with said polynucleotides or vectors. Furthermore, the invention provides processes for producing the binding constructs, methods of treatment using the binding constructs, diagnostic uses of the binding constructs, and kits comprising the binding constructs.

### SEQUENCE LISTING

The instant application contains a Sequence Listing which has been submitted electronically in ASCII format and is hereby incorporated by reference in its entirety. Said ASCII copy, created on May 26, 2022, is named A-2784-WO01-SEC_SL.txt and is 298,117 bytes in size.

### FIELD OF THE INVENTION

The field of this invention relates to compositions and methods related to cancer treatments and diagnostics, including binding constructs that bind to MAGEB2.

### BACKGROUND OF THE INVENTION

The MAGE (melanoma antigen genes) family contains about 60 genes that are categorized into several subfamilies. The MAGE-A, -B, and -C subfamilies are expressed mainly in the testis and are aberrantly expressed in various cancer types. The MAGE-D, -E, -F, -G, -H, -L, and -N subfamilies are expressed in a wide variety of tissues. See, e.g., Lee and Potts, J. Mol. Biol., 2018. One of the MAGE family members, MAGEB2, is typically only expressed in normal testis. MAGEB2, which may function to enhance ubiquitin ligase activity of RING-type zinc finger containing E3 ubiquitin protein ligases, has been found to be aberrantly expressed in a variety of human tumors such as lung carcinoma, breast carcinoma, melanoma, and others. Given this aberrant expression, MAGEB2 is a potential target for new therapeutic agents.

Early detection and classification of cancer is a crucial factor in successful treatment of the disease. A sensitive and precise diagnostic assay that allows detection and quantification of tumor antigens, e.g., MAGEB2, would aid in earlier detection and classification of cancer in patients and could also predict clinical response and outcome for appropriate cancer therapeutics.

The ability to reliably detect these tumor antigens may provide early indication of the disease and/or the disease progression. Immunological diagnostic assays are an important tool for detecting a variety of disease conditions, including cancer. However, such assays may not always be sensitive and/or specific enough to reliably detect particular tumor antigens located on tumor cells, particularly if they are expressed in low levels and/or are not expressed on the surface of tumor cells.

In some instances, a molecular diagnostic assay may be desired and may provide the required specificity and sensitivity, and therefore be the best option to detect a particular tumor antigen. However, in other instances it may also be desired to confirm proper expression of the tumor antigen within the tumor tissue sample and thus an immunohistochemical assay may be better suited.

Accordingly, there remains a need for sensitive and precise diagnostic assays to detect cancer antigens that are useful for detecting malignant cells and/or to help predict efficacy and to improve safety of the relevant therapeutic.

### SUMMARY OF THE INVENTION

In one embodiment, the invention provides an isolated antigen binding construct that binds MAGEB2, wherein the binding construct binds to an epitope comprising a sequence selected from SEQ ID NOs: 2, 3, 4, 562, or 388-554.

In another embodiment, the invention provides an isolated antigen binding construct, wherein the antigen binding construct comprises a CDRL1, a CDRL2, a CDRL3, a CDRH1, a CDRH2, and a CDRH3, wherein the CDRL1 comprises a sequence set forth in SEQ ID NO: 85; the CDRL2 comprises a sequence set forth in SEQ ID NO: 86; the CDRL3 comprises a sequence set forth in SEQ ID NO: 87; the CDRH1 comprises a sequence set forth in SEQ ID NO: 229; the CDRH2 comprises a sequence set forth in SEQ ID NO: 230; and the CDRH3 comprises a sequence set forth in SEQ ID NO: 231.

In another embodiment, the invention provides an antigen binding construct, wherein the antigen binding construct comprises a CDRL1, a CDRL2, a CDRL3, a CDRH1, a CDRH2, and a CDRH3, wherein the CDRL1 comprises a sequence set forth in SEQ ID NO: 73; the CDRL2 comprises a sequence set forth in SEQ ID NO: 74; the CDRL3 comprises a sequence set forth in SEQ ID NO: 75; the CDRH1 comprises a sequence set forth in SEQ ID NO: 217; the CDRH2 comprises a sequence set forth in SEQ ID NO: 218; and the CDRH3 comprises a sequence set forth in SEQ ID NO: 219.

In another embodiment, the invention provides an isolated antigen binding construct, wherein the antigen binding construct comprises a CDRL1, a CDRL2, a CDRL3, a CDRH1, a CDRH2, and a CDRH3, wherein the CDRL1 comprises a sequence set forth in SEQ ID NO: 91; the CDRL2 comprises a sequence set forth in SEQ ID NO: 92; the CDRL3 comprises a sequence set forth in SEQ ID NO: 93; the CDRH1 comprises a sequence set forth in SEQ ID NO: 235; the CDRH2 comprises a sequence set forth in SEQ ID NO: 236; and the CDRH3 comprises a sequence set forth in SEQ ID NO: 237.

In a further embodiment, the invention provides an isolated antigen binding construct, wherein the antigen binding construct comprises a light chain variable region comprising a sequence set forth in SEQ ID NO: 346 and a heavy chain variable region comprising a sequence set forth in SEQ ID NO: 347. In yet a further embodiment, the invention provides an isolated antigen binding construct, wherein the antigen binding construct comprises a light chain variable region comprising a sequence set forth in SEQ ID NO: 338 and a heavy chain variable region comprising a sequence set forth in SEQ ID NO: 339.

In another embodiment, the invention provides an antigen binding construct, wherein the antigen binding construct comprises a light chain variable region comprising a sequence set forth in SEQ ID NO: 350 and a heavy chain variable region comprising a sequence set forth in SEQ ID NO: 351.

In another embodiment, the invention provides a method of making an antibody that binds to MAGEB2 comprising immunizing an animal with a peptide comprising a sequence selected from SEQ ID NO: 2, 3, 4, or 562, and isolating from said animal antibodies that bind to MAGEB2.

In another embodiment, the invention provides a method for treating a tumor in a subject, said method comprising: determining the subject as responsive to treatment with an anti-MAGEB2 therapeutic by obtaining a sample from the subject, wherein the sample comprises a cell from the tumor, measuring the level of MAGEB2 in the sample using an antigen binding construct provided herein, and determining the subject as responsive to treatment with an anti-MAGEB2 therapeutic, and administering to the subject an effective amount of the anti-MAGEB2 therapeutic.

In another embodiment, the invention provides a method of identifying a subject as needing an anti-MAGEB2 therapeutic comprising: a) determining the level of MAGEB2 in a sample obtained from the subject using an antigen binding construct provided herein; and b) identifying the subject as needing the anti-MAGEB2 therapeutic when the level of MAGEB2 is increased relative to a control.

In another embodiment, the invention provides a method of determining treatment for a subject with a MAGEB2 positive tumor comprising: determining the level of MAGEB2 in a sample obtained from the subject using an antigen binding construct provided herein; and determining the treatment as comprising an anti-MAGEB2 therapeutic when the level of MAGEB2 is increased, relative to a control. In another embodiment, the invention provides a method of determining efficacy of treatment with an anti-MAGEB2 therapeutic in a subject comprising: determining the level of MAGEB2 in a sample obtained from the subject using an antigen binding construct provided herein before treatment with an anti-MAGEB2 therapeutic and after treatment with an anti-MAGEB2 therapeutic; and determining the treatment as effective when the level of MAGEB2 positive tumor cells is decreased after treatment with the anti-MAGEB2 therapeutic.

In another embodiment, the invention provides a method of diagnosing a subject with a tumor, comprising: a) determining the level of MAGEB2 in a sample obtained from the subject using an antigen binding construct provided herein; and b) diagnosing the subject as having a MAGEB2 positive tumor when the level of MAGEB2 is increased relative to a control.

In another embodiment, the invention provides a method of identifying a subject having a MAGEB2 positive tumor comprising: a) determining the level of MAGEB2 in a sample obtained from the subject using an antigen binding construct provided herein; and b) identifying the subject as having a MAGEB2 positive tumor when the level of MAGEB2 is increased relative to a control.

In another embodiment, the invention provides a method of identifying a subject as needing an anti-MAGEB2 therapeutic comprising: a) determining the level of MAGEB2 in a sample obtained from the subject using an antigen binding construct provided herein; and b) identifying the subject as needing the anti-MAGEB2 therapeutic when the level of MAGEB2 is increased relative to a control.

In another embodiment, the invention provides a method of determining treatment for a subject with a MAGEB2 positive tumor comprising: determining the level of MAGEB2 in a sample obtained from the subject using an antigen binding construct provided herein; and determining the treatment as comprising an anti-MAGEB2 therapeutic when the level of MAGEB2 is increased, relative to a control.

### BRIEF DESCRIPTION OF THE DRAWINGS

FIG. 1. This figure shows the gating hierarchy used in the enrichment of MAGEB2 N-Term Helix, N-Term Peptide, and Middle Loop Region peptide binders as described in Example 2 herein.

FIG. 2. This figure shows two plots that show binding by various anti-MAGEB2 antibodies binding to immunogen peptide and full length MAGEB2 protein. The MAGEB2 panel was run in a limited antigen style assay (only highest concentration shown) to identify highest affinity binders to full length MAGEB2 protein.

FIG. 3. This figure shows immunohistochemical results for a MAGEB2 IHC assay that measured immunoreactivity in MAGEB2 transfected cells. Intense MAGEB2 IHC staining was observed in CHO-MAGE-B2+ cells with the four anti-MAGEB2 antibodies 4G17, 1J15, 1C3, 1I14, but not with IgG control.

FIG. 4. This shows immunohistochemical results for a MAGEB2 IHC assay that measured immunoreactivity in MAGEB5 transfected cells. No MAGEB2 IHC staining was observed in CHO-MAGEB5+ cells with the four anti-MAGEB2 antibodies 4G17, 1J15, 1C3, 1I14, and with IgG control.

FIG. 5. This figure shows immunohistochemical results for a MAGEB2 IHC assay that measured immunoreactivity in control testis tissues. Intense MAGEB2 IHC staining in spermatogonia cells was observed in testis with the four anti-MAGEB2 antibodies 4G17, 1J15, 1C3, 1I14, but not IgG control at 2 μg/ml. Some nuclear stain was seen with antibodies 4G17, 1J15, 1C3; Leydig cell stain was seen with antibody 1J15.

FIG. 6. This figure shows immunohistochemical results for a MAGEB2 IHC Assay that measured immunoreactivity in normal human tissue. Non-specific particulate staining in hepatocytes was seen in liver tissue core (in normal human tissue with anti-MAGEB2 antibodies 4G17, 1J15, 1C3, but not with anti-MAGEB2 antibody 1I14, and IgG control at 5 μg/ml.

FIG. 7. This figure shows immunohistochemical results for a MAGEB2 IHC assay that measured immunoreactivity in testis and liver tissue, with titration down of the 1C3 antibody. At 2 μg/ml Ab concentration, anti-MAGEB2 antibody 1C3 clone has weak to mild intensity specific staining in appropriate proportions of spermatogonia cells as previously observed. There is rare nuclear staining and the intensity is slightly less than that observed at higher concentrations. However, at this concentration there is no non-specific background staining in liver.

### DETAILED DESCRIPTION

MAGEB2 is aberrantly expressed in a variety of human cancer types. MAGEB2 is not expressed on the surface of cells. MAGEB2 peptides, however, are displayed on the surface of tumor cells by the MHC class I molecule. In particular, the MAGEB2 peptide GVYDGEEHSV (SEQ ID NO: 561) is displayed on the surface of tumor cells by the MHC class I molecule as a peptide-MHC complex. See, for example, U.S. Patent Appl. Publ. No. US2016/0250307A1 (U.S. patent application Ser. No. 14/975,952) and US2017/0080070A1 (U.S. patent application Ser. No. 15/357,757). Although the MAGEB2 peptide-MHC complex is displayed on the surface of cells and would potentially be a target for a diagnostic agent, the number of copies on the cell surface of this peptide-MHC complex is far too low to be detected. Accordingly, binding constructs (e.g., antibodies) that are able to bind and detect MAGEB2 that is intracellularly expressed are described herein.

### Binding Constructs

The present invention provides binding constructs comprising domains which bind to a MAGEB2 protein. These binding constructs are alternatively referred to as antigen binding constructs.

The term binding construct refers to a construct that is capable of binding to its specific target or antigen and comprises the variable heavy chain (VH) and/or variable light chain (VL) domains of an antibody or fragment thereof. Typically, a binding domain according to the present invention comprises the minimum structural requirements of an antibody which allow for the target binding. This minimum requirement may e.g. be defined by the presence of at least the three light chain CDRs (i.e. CDR1, CDR2 and CDR3 of the VL region) and/or the three heavy chain CDRs (i.e. CDR1, CDR2 and CDR3 of the VH region), preferably of all six CDRs. An alternative approach to define the minimal structure requirements of an antibody is the definition of the epitope the antibody binds within the structure of a specific target, respectively, the protein domain of the target protein composing the epitope region (epitope cluster) or by reference to a specific antibody competing with the epitope of the defined antibody. Alternatively, the minimal structure requirements may be defined by the paratope sequences within the binding domain of the antibody.

Binding constructs of the present invention comprise at least one binding domain. The term “binding domain” characterizes in connection with the present invention a domain which specifically binds to, interacts with, or recognizes a given target epitope or a given target region on the target molecule, e.g., MAGEB2 or a specific region within MAGEB2. The structure and function of the binding domain is based on the structure and/or function of an antibody, e.g. of a full-length or whole immunoglobulin molecule, and is from the variable heavy chain (VH) and/or variable light chain (VL) domains of an antibody or fragment thereof. In certain embodiments, the binding domain comprises the presence of three light chain CDRs (i.e. CDR1, CDR2 and CDR3 of the VL region) and/or three heavy chain CDRs (i.e. CDR1, CDR2 and CDR3 of the VH region). In certain embodiments, the binding domain is produced by or obtainable by phage-display or library screening methods rather than by grafting CDR sequences from a pre-existing (monoclonal) antibody into a scaffold.

The binding domain of a binding construct according to the invention may e.g. comprise the above referred groups of CDRs. Preferably, those CDRs are comprised in the framework of an antibody light chain variable region (VL) and an antibody heavy chain variable region (VH); however, it does not have to comprise both. Fd fragments, for example, have two VH regions and often retain some antigen-binding function of the intact antigen-binding domain. Additional examples for the format of antibody fragments, antibody variants or binding domains include (1) a Fab fragment, a monovalent fragment having the VL, VH, CL and CH1 domains; (2) a F(ab′)₂ fragment, a bivalent fragment having two Fab fragments linked by a disulfide bridge at the hinge region; (3) an Fd fragment having the two VH and CH1 domains; (4) an Fv fragment having the VL and VH domains of a single arm of an antibody, (5) a dAb fragment (Ward et al., (1989) Nature 341:544-546), which has a VH domain; (6) an isolated complementarity determining region (CDR), and (7) a single chain Fv (scFv), the latter being preferred (for example, derived from an scFV-library). Examples for embodiments of binding constructs according to the invention are e.g. described in WO 00/006605, WO 2005/040220, WO 2008/119567, WO 2010/037838, WO 2013/026837, WO 2013/026833, US 2014/0308285, US 2014/0302037, WO 2014/144722, WO 2014/151910, and WO 2015/048272.

In a specific embodiment of the invention, the binding construct is a full-length antibody, as described herein below.

Also within the definition of “binding domain” or “domain which binds” are fragments of full-length antibodies, such as VH, VHH, VL, (s)dAb, Fv, light chain (VL-CL), Fd (VH-CH1), heavy chain, Fab, Fab′, F(ab′)₂ or “r IgG” (“half antibody” consisting of a heavy chain and a light chain). Binding constructs according to the invention may also comprise modified fragments of antibodies, also called antibody variants or antibody derivatives. Examples include, but are not limited to, scFv, di-scFv or bi(s)-scFv, scFv-Fc, scFv-zipper, scFab, Fab₂, Fab₃, diabodies, single chain diabodies, tandem diabodies (Tandab's), tandem di-scFv, tandem tri-scFv, “minibodies” exemplified by a structure which is as follows: (VH-VL-CH3)₂, (scFv-CH3)₂, ((scFv)₂-CH3+CH3), ((scFv)₂-CH3) or (scFv-CH3-scFv)₂, multibodies such as triabodies or tetrabodies, and single domain antibodies such as nanobodies or single variable domain antibodies comprising merely one variable region, which might be VHH, VH or VL, that specifically binds to an antigen or target independently of other variable regions or domains. Further possible formats of the binding constructs according to the invention are cross bodies, maxi bodies, hetero Fc constructs, mono Fc constructs and scFc constructs. Examples for those formats will be described herein below.

According to the present invention, binding domains are in the form of one or more polypeptides. Such polypeptides may include proteinaceous parts and non-proteinaceous parts (e.g. chemical linkers or chemical cross-linking agents such as glutaraldehyde). Proteins (including fragments thereof, preferably biologically active fragments, and peptides, usually having less than 30 amino acids) comprise two or more amino acids coupled to each other via a covalent peptide bond (resulting in a chain of amino acids).

The term “polypeptide” as used herein describes a group of molecules, which usually consist of more than 30 amino acids. Polypeptides may further form multimers such as dimers, trimers and higher oligomers, i.e., consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a heteromultimer is an antibody molecule, which, in its naturally occurring form, consists of two identical light polypeptide chains and two identical heavy polypeptide chains. The terms “peptide”, “polypeptide” and “protein” also refer to naturally modified peptides/polypeptides/proteins wherein the modification is effected e.g. by post-translational modifications like glycosylation, acetylation, phosphorylation and the like. A “peptide”, “polypeptide” or “protein” when referred to herein may also be chemically modified such as pegylated. Such modifications are well known in the art and described herein below.

The definition of “antibody” according to the invention comprises full-length antibodies, also including camelid antibodies and other immunoglobulins generated by biotechnological or protein engineering methods or processes. These full-length antibodies may be for example monoclonal, recombinant, chimeric, deimmunized, humanized and human antibodies, as well as antibodies from other species such as mouse, hamster, rabbit, rat, goat, or non-human primates.

For the antibodies provided herein, the variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain/light chain pair mentioned above typically are aligned by the framework regions to form a structure that binds specifically to the target epitope. From N-terminal to C-terminal, naturally-occurring light and heavy chain variable regions both typically conform with the following order of these elements: FR1, CDR1, FR2, CDR2, FR3, CDR3 and FR4. A numbering system has been devised for assigning numbers to amino acids that occupy positions in each of these domains.

This numbering system is defined in Kabat Sequences of Proteins of Immunological Interest (1987 and 1991, NIH, Bethesda, Md.), or Chothia & Lesk, 1987, J. Mol. Biol. 196:901-917; Chothia et al., 1989, Nature 342:878-883.

The term “variable” refers to the portions of the antibody or immunoglobulin domains that exhibit variability in their sequence and that are involved in determining the specificity and binding affinity of a particular antibody (i.e., the “variable domain(s)”). The pairing of a variable heavy chain (VH) and a variable light chain (VL) together forms an antigen-binding domain.

Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM or FR) and provide a scaffold for the six CDRs in three-dimensional space to form an antigen-binding surface. The variable domains of naturally occurring heavy and light chains each comprise four framework (FRM) regions (FR1, FR2, FR3, and FR4), largely adopting a β-sheet configuration, connected by three hypervariable regions, which form loops connecting, and in some cases forming part of, the β-sheet structure. The hypervariable regions in each chain are held together in close proximity by the framework regions and, with the hypervariable regions from the other chain, contribute to the formation of the antigen-binding site.

The terms “CDR”, and its plural “CDRs”, refer to the complementarity determining region of which three make up the binding character of a light chain variable region (CDR-L1, CDR-L2 and CDR-L3) and three make up the binding character of a heavy chain variable region (CDR-H1, CDR-H2 and CDR-H3). CDRs contain most of the residues responsible for specific interactions of the antibody with the antigen and hence contribute to the functional activity of an antibody molecule, i.e., they are the main determinants of binding specificity to a particular target.

The exact definitional CDR boundaries and lengths are subject to different classification and numbering systems. CDRs may therefore be referred to by Kabat, Chothia, contact or any other boundary definitions, including the numbering system described herein. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat (an approach based on cross-species sequence variability), Chothia (an approach based on crystallographic studies of antigen-antibody complexes), and/or MacCallum (Kabat et al., Sequences of Proteins of Immunological Interest, 5th Ed. Public Health Service, National Institutes of Health, Bethesda, Md., 1991; Chothia et al., J. Mol. Biol, 1987, 196: 901-917; and MacCallum et al., J. Mol. Biol, 1996, 262: 732). Still another standard for characterizing the antigen binding side is the AbM definition used by Oxford Molecular's AbM antibody modeling software. See, e.g., Protein Sequence and Structure Analysis of Antibody Variable Domains, Antibody Engineering Lab Manual (Ed.: Duebel, S. and Kontermann, R., Springer-Verlag, Heidelberg). To the extent that two residue identification techniques define regions of overlapping, but not identical regions, they can be combined to define a hybrid CDR. However, the numbering in accordance with the Kabat system is preferred.

Typically, CDRs form a loop structure that can be classified as a canonical structure. The term “canonical structure” refers to the main chain conformation that is adopted by the antigen binding (CDR) loops. Each canonical structure can be characterized by the torsion angles of the polypeptide backbone. Correspondent loops between antibodies may, therefore, have very similar three dimensional structures, despite high amino acid sequence variability in most parts of the loops (Chothia and Lesk, J. Mol. Biol., 1987, 196: 901; Chothia et al., Nature, 1989, 342: 877; Martin and Thornton, J. Mol. Biol, 1996, 263: 800). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

The term “canonical structure” may also include considerations as to the linear sequence of the antibody, for example, as catalogued by Kabat (Kabat et al.). The Kabat numbering scheme (system) is a widely adopted standard for numbering the amino acid residues of an antibody variable domain in a consistent manner and is the preferred scheme applied in the present invention as also mentioned elsewhere herein. Additional structural considerations can also be used to determine the canonical structure of an antibody. For example, those differences not fully reflected by Kabat numbering can be described by the numbering system of Chothia et al. and/or revealed by other techniques, for example, crystallography and two- or three-dimensional computational modeling. Accordingly, a given antibody sequence may be placed into a canonical class which allows for, among other things, identifying appropriate chassis sequences (e.g., based on a desire to include a variety of canonical structures in a library). Kabat numbering of antibody amino acid sequences and structural considerations as described by Chothia et al., loc. cit. and their implications for construing canonical aspects of antibody structure, are described in the literature. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988.

As used herein, the terms “single-chain Fv,” “single-chain antibodies” or “scFv” refer to single polypeptide chain antibody fragments that comprise the variable regions from both the heavy and light chains, but lack the constant regions. Generally, a single-chain antibody further comprises a polypeptide linker between the VH and VL domains which enables it to form the desired structure which would allow for antigen binding. Single chain antibodies are discussed in detail by Pluckthun in The Pharmacology of Monoclonal Antibodies, vol. 113, Rosenburg and Moore eds. Springer-Verlag, New York, pp. 269-315 (1994). Various methods of generating single chain antibodies are known, including those described in U.S. Pat. Nos. 4,694,778 and 5,260,203; International Patent Application Publication No. WO 88/01649; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041. In specific embodiments, single-chain antibodies can also be bispecific, multispecific, human, and/or humanized and/or synthetic.

In some embodiments, the binding constructs of the present invention are “in vitro generated binding constructs”. This term refers to a binding construct according to the above definition where all or part of the variable region (e.g., at least one CDR) is generated in a non-immune cell selection, e.g., an in vitro phage display, protein chip or any other method in which candidate sequences can be tested for their ability to bind to an antigen. In other embodiments, the binding construct sequences are generated by genomic rearrangement in an immune cell in an animal. This term thus preferably excludes sequences generated solely by genomic rearrangement in an immune cell in an animal. It is envisaged that the first and/or second domain of the binding construct is produced by or obtainable by phage display or library screening methods rather than by grafting CDR sequences from a pre-existing (monoclonal) antibody into a scaffold.

A “recombinant antibody” is an antibody made through the use of recombinant DNA technology or genetic engineering.

The term “monoclonal antibody” (mAb) or monoclonal antibody construct as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site or epitope on the antigen, in contrast to conventional (polyclonal) antibody preparations which typically include different antibodies directed against different antigenic sites or epitopes. In addition to their specificity, the monoclonal antibodies are advantageous in that they are synthesized by clonal cell culture and are uncontaminated by other immunoglobulins. The modifier “monoclonal” indicates the character of the antibody as being obtained from a substantially homogeneous population of antibodies, and is not to be construed as requiring production of the antibody by any particular method.

For the preparation of monoclonal antibodies, any technique providing antibodies produced by continuous cell line cultures can be used. For example, monoclonal antibodies to be used may be made by the hybridoma method first described by Koehler et al., Nature, 256: 495 (1975), or may be made by recombinant DNA methods (see, e.g., U.S. Pat. No. 4,816,567). Examples for further techniques to produce human monoclonal antibodies include the trioma technique, the human B-cell hybridoma technique (Kozbor, Immunology Today 4 (1983), 72) and the EBV-hybridoma technique (Cole et al., Monoclonal Antibodies and Cancer Therapy, Alan R. Liss, Inc. (1985), 77-96).

Hybridomas can then be screened using standard methods, such as enzyme-linked immunosorbent assay (ELISA) and surface plasmon resonance analysis, e.g. Biacore™ to identify one or more hybridomas that produce an antibody that specifically binds with a specified antigen. Any form of the relevant antigen may be used as the immunogen, e.g., recombinant antigen, naturally occurring forms, any variants or fragments thereof, as well as an antigenic peptide thereof. Surface plasmon resonance as employed in the Biacore system can be used to increase the efficiency of phage antibodies which bind to an epitope of a target cell surface antigen (Schier, Human Antibodies Hybridomas 7 (1996), 97-105; Malmborg, J. Immunol. Methods 183 (1995), 7-13).

Another exemplary method of making monoclonal antibodies includes screening protein expression libraries, e.g., phage display or ribosome display libraries. Phage display is described, for example, in Ladner et al., U.S. Pat. No. 5,223,409; Smith (1985) Science 228:1315-1317, Clackson et al., Nature, 352: 624-628 (1991) and Marks et al., J. Mol. Biol., 222: 581-597 (1991).

In addition to the use of display libraries, the relevant antigen can be used to immunize a non-human animal, e.g., a rodent (such as a mouse, hamster, rabbit or rat). In one embodiment, the non-human animal includes at least a part of a human immunoglobulin gene. For example, it is possible to engineer mouse strains deficient in mouse antibody production with large fragments of the human Ig (immunoglobulin) loci. Using the hybridoma technology, antigen-specific monoclonal antibodies derived from the genes with the desired specificity may be produced and selected. See, e.g., XENOMOUSE™, Green et al. (1994) Nature Genetics 7:13-21, US 2003-0070185, WO 96/34096, and WO 96/33735.

A monoclonal antibody can also be obtained from a non-human animal, and then modified, e.g., humanized, deimmunized, rendered chimeric etc., using recombinant DNA techniques known in the art. Examples of modified binding constructs include humanized variants of non-human antibodies, “affinity matured” antibodies (see, e.g. Hawkins et al. J. Mol. Biol. 254, 889-896 (1992) and Lowman et al., Biochemistry 30, 10832-10837 (1991)) and antibody mutants with altered effector function(s) (see, e.g., U.S. Pat. No. 5,648,260, Kontermann and Dubel (2010), loc. cit. and Little (2009), loc. cit.).

In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. The in vitro affinity maturation has successfully been used to optimize binding constructs, e.g., antibodies or antibody fragments. Random mutations inside the CDRs are introduced using radiation, chemical mutagens or error-prone PCR. In addition, the genetic diversity can be increased by chain shuffling. Two or three rounds of mutation and selection using display methods like phage display usually results in antibody fragments with affinities in the low nanomolar range.

A preferred type of an amino acid substitutional variation of the binding constructs involves substituting one or more hypervariable region residues of a parent antibody (e. g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sides (e. g. 6-7 sides) are mutated to generate all possible amino acid substitutions at each side. The antibody variants thus generated are displayed in a monovalent fashion from filamentous phage particles as fusions to the gene III product of M13 packaged within each particle. The phage-displayed variants are then screened for their biological activity (e. g. binding affinity) as herein disclosed. In order to identify candidate hypervariable region sides for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the binding domain and, e.g., the MAGEB2. Such contact residues and neighboring residues are candidates for substitution according to the techniques elaborated herein. Once such variants are generated, the panel of variants is subjected to screening as described herein and antibodies with superior properties in one or more relevant assays may be selected for further development.

The antibodies of the present invention specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81: 6851-6855 (1984)). Chimeric antibodies of interest herein include “primitized” antibodies comprising variable domain antigen-binding sequences derived from a non-human primate (e.g., Old World Monkey, Ape etc.) and human constant region sequences. A variety of approaches for making chimeric antibodies have been described. See e.g., Morrison et al., Proc. Natl. Acad. Sci. U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., EP 0171496; EP 0173494; and GB 2177096.

“Humanized” antibodies, variants or fragments thereof (such as Fv, Fab, Fab′, F(ab′)2 or other antigen-binding subsequences of antibodies) are antibodies or immunoglobulins of mostly human sequences, which contain (a) minimal sequence(s) derived from non-human immunoglobulin. For the most part, humanized antibodies are human immunoglobulins (recipient antibody) in which residues from a hypervariable region (also CDR) of the recipient are replaced by residues from a hypervariable region of a non-human (e.g., rodent) species (donor antibody) such as mouse, rat, hamster or rabbit having the desired specificity, affinity, and capacity. In some instances, Fv framework region (FR) residues of the human immunoglobulin are replaced by corresponding non-human residues. Furthermore, “humanized antibodies” as used herein may also comprise residues which are found neither in the recipient antibody nor the donor antibody. These modifications are made to further refine and optimize antibody performance. The humanized antibody may also comprise at least a portion of an immunoglobulin constant region (Fc), typically that of a human immunoglobulin. For further details, see Jones et al., Nature, 321: 522-525 (1986); Reichmann et al., Nature, 332: 323-329 (1988); and Presta, Curr. Op. Struct. Biol., 2: 593-596 (1992).

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by Morrison (1985) Science 229:1202-1207; by Oi et al. (1986) BioTechniques 4:214; and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

Humanized antibodies may also be produced using transgenic animals such as mice that express human heavy and light chain genes, but are incapable of expressing the endogenous mouse immunoglobulin heavy and light chain genes. Winter describes an exemplary CDR grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

A humanized antibody can be optimized by the introduction of conservative substitutions, consensus sequence substitutions, germline substitutions and/or back mutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982, and EP 239 400).

The term “human antibody”, “human antibody construct” and “human binding domain” includes antibodies, antibody fragments, and binding domains having antibody regions such as variable and constant regions or domains which correspond substantially to human germline immunoglobulin sequences known in the art, including, for example, those described by Kabat et al. (1991) (loc. cit.). The human antibodies, antibody fragments or binding domains of the invention may include amino acid residues not encoded by human germline immunoglobulin sequences (e.g., mutations introduced by random or side-specific mutagenesis in vitro or by somatic mutation in vivo), for example in the CDRs, and in particular, in CDR3. The human antibodies, antibody fragments or binding domains can have at least one, two, three, four, five, or more positions replaced with an amino acid residue that is not encoded by the human germline immunoglobulin sequence. The definition of human antibodies, antibody fragments and binding domains as used herein also contemplates fully human antibodies, which include only non-artificially and/or genetically altered human sequences of antibodies as those can be derived by using technologies or systems such as the Xenomouse. Preferably, a “fully human antibody” does not include amino acid residues not encoded by human germline immunoglobulin sequences.

The ability to clone and reconstruct megabase-sized human loci in yeast artificial chromosomes YACs and to introduce them into the mouse germline provides a powerful approach to elucidating the functional components of very large or crudely mapped loci as well as generating useful models of human disease. Furthermore, the use of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (mAbs)—an important milestone towards fulfilling the promise of antibody therapy in human disease. Fully human antibodies are expected to minimize the immunogenic and allergic responses intrinsic to mouse or mouse-derivatized mAbs and thus to increase the efficacy and safety of the administered antibodies. The use of fully human antibodies can be expected to provide a substantial advantage in the treatment of chronic and recurring human diseases, such as inflammation, autoimmunity, and cancer, which require repeated compound administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human mAbs with the desired specificity could be readily produced and selected. This general strategy was demonstrated in connection with the generation of the first XenoMouse mouse strains (see Green et al. Nature Genetics 7:13-21 (1994)). The XenoMouse strains were engineered with YACs containing 245 kb and 190 kb-sized germline configuration fragments of the human heavy chain locus and kappa light chain locus, respectively, which contained core variable and constant region sequences. The human Ig containing YACs proved to be compatible with the mouse system for both rearrangement and expression of antibodies and were capable of substituting for the inactivated mouse Ig genes. This was demonstrated by their ability to induce B cell development, to produce an adult-like human repertoire of fully human antibodies, and to generate antigen-specific human mAbs. These results also suggested that introduction of larger portions of the human Ig loci containing greater numbers of V genes, additional regulatory elements, and human Ig constant regions may recapitulate substantially the full repertoire that is characteristic of the human humoral response to infection and immunization. The work of Green et al. was recently extended to the introduction of greater than approximately 80% of the human antibody repertoire through introduction of megabase sized, germline configuration YAC fragments of the human heavy chain loci and kappa light chain loci, respectively. See Mendez et al. Nature Genetics 15:146-156 (1997) and U.S. patent application Ser. No. 08/759,620. The production of the XenoMouse® animals is further discussed and delineated in U.S. patent application Ser. No. 07/466,008, Ser. No. 07/610,515, Ser. No. 07/919,297, Ser. No. 07/922,649, Ser. No. 08/031,801, Ser. No. 08/112,848, Ser. No. 08/234,145, Ser. No. 08/376,279, Ser. No. 08/430,938, Ser. No. 08/464,584, Ser. No. 08/464,582, Ser. No. 08/463,191, Ser. No. 08/462,837, Ser. No. 08/486,853, Ser. No. 08/486,857, Ser. No. 08/486,859, Ser. No. 08/462,513, Ser. No. 08/724,752, and Ser. No. 08/759,620; and U.S. Pat. Nos. 6,162,963; 6,150,584; 6,114,598; 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998), EP 0 463 151 B1, WO 94/02602, WO 96/34096, WO 98/24893, WO 00/76310, and WO 03/47336.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806; 5,625,825; 5,625,126; 5,633,425; 5,661,016; 5,770,429; 5,789,650; 5,814,318; 5,877,397; 5,874,299; and 6,255,458 each to Lonberg and Kay, U.S. Pat. Nos. 5,591,669 and 6,023,010 to Krimpenfort and Berns, U.S. Pat. Nos. 5,612,205; 5,721,367; and U.S. Pat. No. 5,789,215 to Berns et al., and U.S. Pat. No. 5,643,763 to Choi and Dunn, and GenPharm International U.S. patent application Ser. No. 07/574,748, Ser. No. 07/575,962, Ser. No. 07/810,279, Ser. No. 07/853,408, Ser. No. 07/904,068, Ser. No. 07/990,860, Ser. No. 08/053,131, Ser. No. 08/096,762, Ser. No. 08/155,301, Ser. No. 08/161,739, Ser. No. 08/165,699, Ser. No. 08/209,741. See also EP 0 546 073 B1, WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175. See further Taylor et al. (1992), Chen et al. (1993), Tuaillon et al. (1993), Choi et al. (1993), Lonberg et al. (1994), Taylor et al. (1994), and Tuaillon et al. (1995), Fishwild et al. (1996).

Kirin has also demonstrated the generation of human antibodies from mice in which, through microcell fusion, large pieces of chromosomes, or entire chromosomes, have been introduced. See European Patent Application Nos. 773 288 and 843 961. Xenerex Biosciences is developing a technology for the potential generation of human antibodies. In this technology, SCID mice are reconstituted with human lymphatic cells, e.g., B and/or T cells. Mice are then immunized with an antigen and can generate an immune response against the antigen. See U.S. Pat. Nos. 5,476,996; 5,698,767; and 5,958,765.

In some embodiments, the binding constructs of the invention are “isolated” or “substantially pure” binding constructs. “Isolated” or “substantially pure”, when used to describe the binding constructs disclosed herein, means a binding construct that has been identified, separated and/or recovered from a component of its production environment. Preferably, the binding construct is free or substantially free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. It is understood that the isolated protein may constitute a wide range of percent concentration, e.g., from 5% to 99.9% by weight of the total protein content, depending on the circumstances. The polypeptide may be made at a significantly higher concentration through the use of an inducible promoter or high expression promoter, such that it is made at increased concentration levels. The definition includes the production of a binding construct in a wide variety of organisms and/or host cells that are known in the art. In preferred embodiments, the binding construct will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated binding construct will be prepared by at least one purification step.

Peptides are short chains of amino acid monomers linked by covalent peptide (amide) bonds. Hence, peptides fall under the broad chemical classes of biological oligomers and polymers. Amino acids that are part of a peptide or polypeptide chain are termed “residues” and can be consecutively numbered. All peptides except cyclic peptides have an N-terminal residue at one end and a C-terminal residue at the other end of the peptide. An oligopeptide consists of only a few amino acids (usually between two and twenty). A polypeptide is a longer, continuous, and unbranched peptide chain. Peptides are distinguished from proteins on the basis of size, and as an arbitrary benchmark can be understood to contain approximately 50 or fewer amino acids. Proteins consist of one or more polypeptides, usually arranged in a biologically functional way. While aspects of the lab techniques applied to peptides versus polypeptides and proteins differ (e.g., the specifics of electrophoresis, chromatography, etc.), the size boundaries that distinguish peptides from polypeptides and proteins are not absolute. Therefore, in the context of the present invention, the terms “peptide”, “polypeptide” and “protein” may be used interchangeably, and the term “polypeptide” is often preferred.

Polypeptides may further form multimers such as dimers, trimers and higher oligomers, which consist of more than one polypeptide molecule. Polypeptide molecules forming such dimers, trimers etc. may be identical or non-identical. The corresponding structures of higher order of such multimers are, consequently, termed homo- or heterodimers, homo- or heterotrimers etc. An example for a hereteromultimer is a full-length antibody or immunoglobulin molecule, which, in its naturally occurring form, consists of two identical light polypeptide chains and two identical heavy polypeptide chains. The terms “peptide”, “polypeptide” and “protein” also refer to naturally modified peptides/polypeptides/proteins wherein the modification is accomplished e.g. by post-translational modifications like glycosylation, acetylation, phosphorylation and the like. A “peptide”, “polypeptide” or “protein” when referred to herein may also be chemically modified such as pegylated. Such modifications are well known in the art and described herein below.

A binding construct is said to “specifically bind” or “immunospecifically bind” to its antigen when the binding construct binds its antigen with a dissociation constant (KD) is ≤10-7 M as measured via a surface plasma resonance technique (e.g., BIACore, GE-Healthcare Uppsala, Sweden) or Kinetic Exclusion Assay (KinExA, Sapidyne, Boise, Idaho). In accordance with this invention a binding construct specifically binds or immunospecifically binds to MAGEB2.

Because of the sequence similarity between homologous proteins in different species, a binding construct or a binding domain that specifically binds to its target (such as a human target) may, however, cross-react with homologous target molecules from different species (such as, from non-human primates). The term “specific/immunospecific binding” can hence include the binding of a binding construct or binding domain to epitopes or structurally related epitopes in more than one species.

The term “epitope” refers to a region on an antigen, or specific amino acid residues, to which a binding domain, such as an antibody or immunoglobulin, or a derivative, fragment or variant of an antibody or an immunoglobulin, specifically binds. An “epitope” is antigenic and thus the term epitope is sometimes also referred to herein as “antigenic structure” or “antigenic determinant”. Thus, the binding domain is an “antigen interaction site”. Said binding/interaction is also understood to define a “specific recognition”.

“Epitopes” can be formed both by contiguous amino acids or non-contiguous amino acids juxtaposed by tertiary folding of a protein. A “linear epitope” is an epitope where a contiguous amino acid primary sequence comprises the recognized epitope. A linear epitope typically includes at least 3 or at least 4, and more typically, at least 5 or at least 6 or at least 7, and frequently, about 8 to about 10 amino acids in a unique sequence, or even longer than 10 amino acids.

A “conformational epitope”, in contrast to a linear epitope, is an epitope wherein the primary sequence of the amino acids comprising the epitope is not the sole defining component of the epitope recognized (e.g., an epitope wherein the primary sequence of amino acids is not necessarily recognized by the binding domain). Typically, a conformational epitope comprises an increased number of amino acids relative to a linear epitope, and comprises noncontiguous amino acid sequences. With regard to recognition of conformational epitopes, the binding domain paratope recognizes a three-dimensional structure of the antigen, preferably a peptide or protein or fragment thereof (in the context of the present invention, the antigenic structure for one of the binding domains is comprised within the target cell surface antigen protein). For example, when a protein molecule folds to form a three-dimensional structure, certain amino acids and/or the polypeptide backbone forming the conformational epitope become juxtaposed enabling the antibody to recognize the epitope. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy.

Various methods for identifying proteins, regions of proteins, or peptides that are useful as immunogens or for screening assays are known in the art. In one embodiment, and as described herein in the Examples, a Shannon entropy analysis of the MAGEB2 protein amino acid sequence can identify particular peptide sequences that can be used as immunogens.

Exemplary MAGEB2 peptides that were identified to be used as immunogens included: MAGEB2 peptide a.a.43-76: SSVSGGAASSSPAAGIPQEPQRAPTTAAAAAAGV (N-terminal region peptide) (SEQ ID NO: 2), MAGEB2 peptide a.a.95-125: SSSQASTSTKSPSEDPLTRKSGSLVQFLLYK (MHD N-terminal helix peptide) (SEQ ID NO: 3), MAGEB2 peptide a.a.185-200: DLTDEESLLSSWDFPR (MHD middle loop peptide) (SEQ ID NO: 562).

Accordingly, in one embodiment of the present invention, provided is a method of immunizing an animal comprising administration to the animal any of the peptides of SEQ ID NOs: 2, 3, 4, or 562.

In another embodiment, provided is a method of generating an isolated antibody comprising immunizing an animal with any of the peptides of SEQ ID NOs: 2, 3, 4, or 562 and isolating said antibody. In further embodiments, provided is a method of generating an isolated monoclonal antibody comprising immunizing an animal with any of the peptides of SEQ ID NOs: 2, 3, 4 or 562 and further generating a monoclonal antibody using art recognized steps described herein.

In yet another embodiment, provided is an isolated antibody generated by a process comprising immunizing an animal with any of the peptides of SEQ ID NOs: 2, 3, 4, or 562.

In yet other embodiments, fragments of the peptides of SEQ ID NOs: 2, 3, 4 or 562 can be used as the immunogen. Accordingly, in one embodiment of the present invention, the peptide immunogen comprises a fragment of the MAGEB2 peptide a.a.43-76: SSVSGGAASSSPAAGIPQEPQRAPTTAAAAAAGV (N-terminal region peptide) (SEQ ID NO: 2) that is 10, 11, 12, 13, 14, 15, 16, 17, 18, 19, 20, 21, 22, 23, 24, 25, 26, 27, 28, 29, 30, 31, 32, or 33 amino acids long.

In another embodiment of the present invention, the peptide immunogen comprises a fragment of the MAGEB2 peptide a.a.95-125: SSSQASTSTKSPSEDPLTRKSGSLVQFLLYK (MHD N-terminal helix peptide) (SEQ ID NO: 3) that is 10, 11, 12, 13, 14, 15, 16, 17, 18, 19, 20, 21, 22, 23, 24, 25, 26, 27, 28, 29, or 30, amino acids long.

In another embodiment of the present invention, the peptide immunogen comprises a fragment of the MAGEB2 peptide a.a.185-200: DLTDEESLLSSWDFPR (MHD middle loop peptide) (SEQ ID NO: 562) that is 10, 11, 12, 13, 14, or 15 amino acids long.

In further embodiments of the present invention, provided are binding constructs that compete for binding with any of the binding constructs described in the present invention. Competition assays are well known in the art and exemplary assays are described further herein.

In other embodiments, alternative peptides that can be used as immunogens for screening are provided in Table 1 below. Accordingly, the invention provides

TABLE 1

| MAGEB2 Peptide Amino Acid Sequence | SEQ ID NO: |
|---|---|
| SVSGGAASSSPAAGIPQEPQRAPTTAAAAAAGV | 388 |
| VSGGAASSSPAAGIPQEPQRAPTTAAAAAAGV | 389 |
| SGGAASSSPAAGIPQEPQRAPTTAAAAAAGV | 390 |
| SGGAASSSPAAGIPQEPQRAPTTAAAAAAGV | 391 |
| GGAASSSPAAGIPQEPQRAPTTAAAAAAGV | 392 |
| GAASSSPAAGIPQEPQRAPTTAAAAAAGV | 393 |
| AASSSPAAGIPQEPQRAPTTAAAAAAGV | 394 |
| ASSSPAAGIPQEPQRAPTTAAAAAAGV | 395 |
| SSSPAAGIPQEPQRAPTTAAAAAAGV | 396 |
| SSPAAGIPQEPQRAPTTAAAAAAGV | 397 |
| SPAAGIPQEPQRAPTTAAAAAAGV | 398 |
| PAAGIPQEPQRAPTTAAAAAAGV | 399 |
| AAGIPQEPQRAPTTAAAAAAGV | 400 |
| AGIPQEPQRAPTTAAAAAAGV | 401 |
| GIPQEPQRAPTTAAAAAAGV | 402 |
| IPQEPQRAPTTAAAAAAGV | 403 |
| PQEPQRAPTTAAAAAAGV | 404 |
| QEPQRAPTTAAAAAAGV | 405 |
| EPQRAPTTAAAAAAGV | 406 |
| PQRAPTTAAAAAAGV | 407 |
| QRAPTTAAAAAAGV | 408 |
| RAPTTAAAAAAGV | 409 |
| APTTAAAAAAGV | 410 |
| PTTAAAAAAGV | 411 |
| TTAAAAAAGV | 412 |
| TAAAAAAGV | 413 |
| AAAAAAGV | 414 |
| AAAAAGV | 415 |
| AAAAGV | 416 |
| AAAGV | 417 |
| SSVSGGAASSSPAAGIPQEPQRAPTTAAAAAAG | 418 |
| SSVSGGAASSSPAAGIPQEPQRAPTTAAAAAA | 419 |
| SSVSGGAASSSPAAGIPQEPQRAPTTAAAAA | 420 |
| SSVSGGAASSSPAAGIPQEPQRAPTTAAAA | 421 |
| SSVSGGAASSSPAAGIPQEPQRAPTTAAA | 422 |
| SSVSGGAASSSPAAGIPQEPQRAPTTAA | 423 |
| SSVSGGAASSSPAAGIPQEPQRAPTTA | 424 |
| SSVSGGAASSSPAAGIPQEPQRAPTT | 425 |
| SSVSGGAASSSPAAGIPQEPQRAPT | 426 |
| SSVSGGAASSSPAAGIPQEPQRAP | 427 |
| SSVSGGAASSSPAAGIPQEPQRA | 428 |
| SSVSGGAASSSPAAGIPQEPQR | 429 |
| SSVSGGAASSSPAAGIPQEPQ | 430 |
| SSVSGGAASSSPAAGIPQEP | 431 |
| SSVSGGAASSSPAAGIPQE | 432 |
| SSVSGGAASSSPAAGIPQ | 433 |
| SSVSGGAASSSPAAGIP | 434 |
| SSVSGGAASSSPAAGI | 435 |
| SSVSGGAASSSPAAG | 436 |
| SSVSGGAASSSPAA | 437 |
| SSVSGGAASSSPA | 438 |
| SSVSGGAASSSP | 439 |
| SSVSGGAASSS | 440 |
| SSVSGGAASS | 441 |
| SSVSGGAAS | 442 |
| SSVSGGAA | 443 |
| SSVSGGA | 444 |
| SSVSGG | 445 |
| SSVSG | 446 |
| SVSGGAASSSPAAGIPQEPQRAPTTAAAAAAG | 447 |
| VSGGAASSSPAAGIPQEPQRAPTTAAAAAA | 448 |
| SGGAASSSPAAGIPQEPQRAPTTAAAAA | 449 |
| GGAASSSPAAGIPQEPQRAPTTAAAA | 450 |
| GAASSSPAAGIPQEPQRAPTTAAA | 451 |
| AASSSPAAGIPQEPQRAPTTAA | 452 |
| ASSSPAAGIPQEPQRAPTTA | 453 |
| SSSPAAGIPQEPQRAPTT | 454 |
| SSPAAGIPQEPQRAPT | 455 |
| SPAAGIPQEPQRAP | 456 |
| PAAGIPQEPQRA | 457 |
| AAGIPQEPQR | 458 |
| AGIPQEPQ | 459 |
| GIPQEP | 460 |
| IPQE | 461 |
| SSQASTSTKSPSEDPLTRKSGSLVQFLLYK | 462 |
| SQASTSTKSPSEDPLTRKSGSLVQFLLYK | 463 |
| QASTSTKSPSEDPLTRKSGSLVQFLLYK | 464 |
| ASTSTKSPSEDPLTRKSGSLVQFLLYK | 465 |
| STSTKSPSEDPLTRKSGSLVQFLLYK | 466 |
| TSTKSPSEDPLTRKSGSLVQFLLYK | 467 |
| STKSPSEDPLTRKSGSLVQFLLYK | 468 |
| TKSPSEDPLTRKSGSLVQFLLYK | 469 |
| KSPSEDPLTRKSGSLVQFLLYK | 470 |
| SPSEDPLTRKSGSLVQFLLYK | 471 |
| PSEDPLTRKSGSLVQFLLYK | 472 |
| SEDPLTRKSGSLVQFLLYK | 473 |
| EDPLTRKSGSLVQFLLYK | 474 |
| DPLTRKSGSLVQFLLYK | 475 |
| PLTRKSGSLVQFLLYK | 476 |
| LTRKSGSLVQFLLYK | 477 |
| TRKSGSLVQFLLYK | 478 |
| RKSGSLVQFLLYK | 479 |
| KSGSLVQFLLYK | 480 |
| SGSLVQFLLYK | 481 |
| GSLVQFLLYK | 482 |
| SLVQFLLYK | 483 |
| LVQFLLYK | 484 |
| VQFLLYK | 485 |
| QFLLYK | 486 |
| FLLYK | 487 |
| SSSQASTSTKSPSEDPLTRKSGSLVQFLLY | 488 |
| SSSQASTSTKSPSEDPLTRKSGSLVQFLL | 489 |
| SSSQASTSTKSPSEDPLTRKSGSLVQFL | 490 |
| SSSQASTSTKSPSEDPLTRKSGSLVQF | 491 |
| SSSQASTSTKSPSEDPLTRKSGSLVQ | 492 |
| SSSQASTSTKSPSEDPLTRKSGSLV | 493 |
| SSSQASTSTKSPSEDPLTRKSGSL | 494 |
| SSSQASTSTKSPSEDPLTRKSGS | 495 |
| SSSQASTSTKSPSEDPLTRKSG | 496 |
| SSSQASTSTKSPSEDPLTRKS | 497 |
| SSSQASTSTKSPSEDPLTRK | 498 |
| SSSQASTSTKSPSEDPLTR | 499 |
| SSSQASTSTKSPSEDPLT | 500 |
| SSSQASTSTKSPSEDPL | 501 |
| SSSQASTSTKSPSEDP | 502 |
| SSSQASTSTKSPSED | 503 |
| SSSQASTSTKSPSE | 504 |
| SSSQASTSTKSPS | 505 |
| SSSQASTSTKSP | 506 |
| SSSQASTSTKS | 507 |
| SSSQASTSTK | 508 |
| SSSQASTST | 509 |
| SSSQASTS | 510 |
| SSSQAST | 511 |
| SSSQAS | 512 |
| SSSQA | 513 |
| SSQASTSTKSPSEDPLTRKSGSLVQFLLY | 514 |
| SQASTSTKSPSEDPLTRKSGSLVQFLL | 515 |
| QASTSTKSPSEDPLTRKSGSLVQFL | 516 |
| ASTSTKSPSEDPLTRKSGSLVQF | 517 |
| STSTKSPSEDPLTRKSGSLVQ | 518 |
| TSTKSPSEDPLTRKSGSLV | 519 |
| STKSPSEDPLTRKSGSL | 520 |
| TKSPSEDPLTRKSGS | 521 |
| KSPSEDPLTRKSG | 522 |
| SPSEDPLTRKS | 523 |
| PSEDPLTRK | 524 |
| SEDPLTR | 525 |
| EDPLT | 526 |
| LTDEESLLSSWDFPR | 527 |
| TDEESLLSSWDFPR | 528 |
| DEESLLSSWDFPR | 529 |
| EESLLSSWDFPR | 530 |
| ESLLSSWDFPR | 531 |
| SLLSSWDFPR | 532 |
| LLSSWDFPR | 533 |
| LSSWDFPR | 534 |
| SSWDFPR | 535 |
| SWDFPR | 536 |
| WDFPR | 537 |
| DLTDEESLLSSWDFP | 538 |
| DLTDEESLLSSWDF | 539 |
| DLTDEESLLSSWD | 540 |
| DLTDEESLLSSW | 541 |
| DLTDEESLLSS | 542 |
| DLTDEESLLS | 543 |
| DLTDEESLL | 544 |
| DLTDEESL | 545 |
| DLTDEES | 546 |
| DLTDEE | 547 |
| DLTDE | 548 |
| LTDEESLLSSWDFP | 549 |
| TDEESLLSSWDF | 550 |
| DEESLLSSWD | 551 |
| EESLLSSW | 552 |
| ESLLSS | 553 |
| SLLS | 554 |

### Affinity

The interaction between the binding domain and the epitope or the region comprising the epitope implies that a binding domain exhibits appreciable affinity for the epitope and/or the region comprising the epitope on a particular protein or antigen, and unless otherwise specified, does not exhibit significant binding or reactivity with proteins or antigens other than MAGEB2. This affinity can be measured by various techniques known to one skilled in the art, such as in a surface plasmon resonance assay, such as a Biacore assay, or in a cell based assay.

“Appreciable affinity” includes binding with an affinity of about 106 M (KD) or stronger. Preferably, binding is considered specific when the binding affinity is about 10⁻¹² to 10⁻⁸ M, 10⁻¹² to 10⁻⁹ M, 10⁻¹² to 10⁻¹⁰ M, 10⁻¹¹ to 10⁻⁸ M, preferably of about 10⁻¹¹ to 10⁻⁹ M. Whether a binding domain specifically reacts with or binds to a target can be tested readily by, inter alia, comparing the reaction of said binding domain with a target protein or antigen with the reaction of said binding domain with proteins or antigens other than the MAGEB2. Preferably, a binding domain of the invention does not essentially or substantially bind to proteins or antigens other than MAGEB2.

### Specificity

The term “does not significantly bind” means that a binding construct or binding domain of the present invention does not bind to a protein or antigen other than MAGEB2. For example, a binding construct or binding domain that exhibits binding to proteins with similar amino acid sequences, e.g., MAGEA4 or MAGEA8, would not be a desirable binding construct.

In the present invention, the MAGEB2 binding constructs possess surprising levels of specificity and selectivity to their target, as evidenced by its lack of binding to target negative cells. See, e.g., Examples 2 and 3 herein.

This specificity and selectivity are highly desired, yet difficult to achieve, properties for a binding construct in a diagnostic assay as it limits, reduces, or eliminates off-target binding and any potential false readouts.

Specific binding is believed to be effected by specific motifs in the amino acid sequence of the binding domain and the antigen. Thus, binding is achieved as a result of their primary, secondary and/or tertiary structure as well as the result of secondary modifications of said structures. The specific interaction of the antigen-interaction-side with its specific antigen may result in a simple binding of said side to the antigen. Moreover, the specific interaction of the antigen-interaction-side with its specific antigen may alternatively or additionally result in the initiation of a signal, e.g. due to the induction of a change of the conformation of the antigen, an oligomerization of the antigen, etc.

### MAGEB2 Binding Constructs

The invention provides binding constructs comprising a domain which binds to MAGEB2, and in specific embodiments, to the MAGEB2 peptides SSVSGGAASSSPAAGIPQEPQRAPTTAAAAAAGV (N-terminal region peptide) (SEQ ID NO: 2), a.a.95-125: SSSQASTSTKSPSEDPLTRKSGSLVQFLLYK (MHD N-terminal helix peptide) (SEQ ID NO: 3), or a.a.185-200: DLTDEESLLSSWDFPR (SEQ ID NO: 562).

Table 2 below provides amino acid sequences of exemplary MAGEB2 binding constructs VH-CDRs and VL-CDRs. Table 3 below provides amino acid sequences of exemplary MAGEB2 binding construct VH and VL domains.

TABLE 2
Exemplary Light Chain CDR and Heavy Chain CDR Sequences

| Molecule | CDR-L1 | CDR-L2 | CDR-L3 | CDR-H1 | CDR-H2 | CDR-H3 |
|---|---|---|---|---|---|---|
| 1I14 | QSSQSVYDNNAL | GASTLAS | QCTYYVSSYQND | SYAMS | SIGGGGSAVYASW | GFYSIDL |
| | A | (SEQ ID | (SEQ ID NO: 87) | (SEQ ID | AKG | (SEQ ID NO: 231) |
| | (SEQ ID NO: 85) | NO: 86) | | NO: 229) | (SEQ ID NO: 230) | |
| 1C3 | QASQNISSYLA | RASTLAS | QSYDDSRSSNFFY | NYYIC | CIDNANGRTYYAS | SLATPL |
| | (SEQ ID NO: 73) | (SEQ ID | A | (SEQ ID | WAKG | (SEQ ID NO: 219) |
| | | NO: 74) | (SEQ ID NO: 75) | NO: 217) | (SEQ ID NO: 218) | |
| 1H17 | QSSKSVYNKNWL | GASTLAS | AGGYSSSSDTFA | SGQLMC | CIGSGSNAISTFYAS | VGSDDYGDSDVFDP |
| | S | (SEQ ID | (SEQ ID NO: 93) | (SEQ ID | WAQG | (SEQ ID NO: 237) |
| | (SEQ ID NO: 91) | NO: 92) | | NO: 235) | (SEQ ID NO: 236) | |

TABLE 3
Exemplary Variable Light Chain and Variable Heavy Chain Sequences

| Molecule | VL | VH |
|---|---|---|
| 1I14 | DVVMTQTPASVEATVGGTVTIKCQSSQSVYDNNALA | QSVEESGGRLVTPGTPLTLTCTISGFSLSSYAMSWVRQAPGKGL |
| | WYQQNAGQRPRLLIYGASTLASGVPSRFSASGSGTEF | EWIGSIGGGGSAVYASWAKGRFTISKTSTTVDLRITSPTTEDTAM |
| | TLTISDLECADAATYYCQCTYYVSSYQNDFGGGTEVVV | YFCGRGFYSIDLWGPGTLVTVSS |
| | K | (SEQ ID NO: 347) |
| | (SEQ ID NO: 346) | |
| 1C3 | DIVMTQTPSSVEAAVGGTVTIKCQASQNISSYLAWYQ | QSLEESGGGLVQPEGSLTLTCTAFGVTLTNYYICWVRQAPGKGL |
| | QKPGQPPKLLIYRASTLASGVPSRFKGSGSGTQFTLTIS | EWVGCIDNANGRTYYASWAKGRFTISKTSSTTGTLQMTSLTAA |
| | DLECADAATYYCQSYDDSRSSNFFYAFGGGTEVVVK | DTATYFCARSLATPLWGPGTLVTVSS |
| | (SEQ ID NO: 338) | (SEQ ID NO: 339) |
| 1H17 | AAVLTQTPSPVSAAVGGTVSASCQSSKSVYNKNWLS | QEQLVESGGGLVKPGASLTLTCKASGFSFSSGQLMCWVRQAPG |
| | WFQQKPGQPPKLLIYGASTLASGVPSRFKGSGSGTQF | KGLEWIACIGSGSNAISTFYASWAQGRFTISKSSSTTVTLQLTSLT |
| | TLTISDVQCDDAATYYCAGGYSSSSDTFAFGGGTEVVV | AADTATYFCARVGSDDYGDSDVFDPWGPGTLVTVSS |
| | K | (SEQ ID NO: 351) |
| | (SEQ ID NO: 350) | |

In one embodiment, the invention provides an isolated binding domain that binds to MAGEB2, wherein the binding domain comprises:
a) a VH region comprising CDR-H1 as depicted in SEQ ID NO: 229, CDR-H2 as depicted in SEQ ID NO: 230, and CDR-H3 as depicted in SEQ ID NO: 231, and a VL region comprising CDR-L1 as depicted in SEQ ID NO: 85, CDR-L2 as depicted in SEQ ID NO: 86 and CDR-L3 as depicted in SEQ ID NO: 87; or
b) a VH region comprising CDR-H1 as depicted in SEQ ID NO: 217, CDR-H2 as depicted in SEQ ID NO: 218, and CDR-H3 as depicted in SEQ ID NO: 219, and a VL region comprising CDR-L1 as depicted in SEQ ID NO: 73, CDR-L2 as depicted in SEQ ID NO: 74 and CDR-L3 as depicted in SEQ ID NO: 75; or
c) a VH region comprising CDR-H1 as depicted in SEQ ID NO: 235, CDR-H2 as depicted in SEQ ID NO: 236, and CDR-H3 as depicted in SEQ ID NO: 237, and a VL region comprising CDR-L1 as depicted in SEQ ID NO: 91, CDR-L2 as depicted in SEQ ID NO: 92 and CDR-L3 as depicted in SEQ ID NO: 93.

In one embodiment, the invention provides an isolated binding construct comprising a binding domain that binds to MAGEB2, wherein the binding domain comprises:
a) a VH region comprising CDR-H1 as depicted in SEQ ID NO: 229, CDR-H2 as depicted in SEQ ID NO: 230, and CDR-H3 as depicted in SEQ ID NO: 231, and a VL region comprising CDR-L1 as depicted in SEQ ID NO: 85, CDR-L2 as depicted in SEQ ID NO: 86 and CDR-L3 as depicted in SEQ ID NO: 87; or
b) a VH region comprising CDR-H1 as depicted in SEQ ID NO: 217, CDR-H2 as depicted in SEQ ID NO: 218, and CDR-H3 as depicted in SEQ ID NO: 219, and a VL region comprising CDR-L1 as depicted in SEQ ID NO: 73, CDR-L2 as depicted in SEQ ID NO: 74 and CDR-L3 as depicted in SEQ ID NO: 75; or
c) a VH region comprising CDR-H1 as depicted in SEQ ID NO: 235, CDR-H2 as depicted in SEQ ID NO: 236, and CDR-H3 as depicted in SEQ ID NO: 237, and a VL region comprising CDR-L1 as depicted in SEQ ID NO: 91, CDR-L2 as depicted in SEQ ID NO: 92 and CDR-L3 as depicted in SEQ ID NO: 93.

In one embodiment, the invention provides an isolated binding domain that binds to MAGEB2, wherein the binding domain binds to the same epitope as an antibody or binding construct that comprises:
a) a VH region comprising CDR-H1 as depicted in SEQ ID NO: 229, CDR-H2 as depicted in SEQ ID NO: 230, and CDR-H3 as depicted in SEQ ID NO: 231, and a VL region comprising CDR-L1 as depicted in SEQ ID NO: 85, CDR-L2 as depicted in SEQ ID NO: 86 and CDR-L3 as depicted in SEQ ID NO: 87; or
b) a VH region comprising CDR-H1 as depicted in SEQ ID NO: 217, CDR-H2 as depicted in SEQ ID NO: 218, and CDR-H3 as depicted in SEQ ID NO: 219, and a VL region comprising CDR-L1 as depicted in SEQ ID NO: 73, CDR-L2 as depicted in SEQ ID NO: 74 and CDR-L3 as depicted in SEQ ID NO: 75; or
c) a VH region comprising CDR-H1 as depicted in SEQ ID NO: 235, CDR-H2 as depicted in SEQ ID NO: 236, and CDR-H3 as depicted in SEQ ID NO: 237, and a VL region comprising CDR-L1 as depicted in SEQ ID NO: 91, CDR-L2 as depicted in SEQ ID NO: 92 and CDR-L3 as depicted in SEQ ID NO: 93.

In one embodiment, the invention provides an isolated binding construct comprising a binding domain that binds to MAGEB2, wherein the binding domain binds to the same epitope as an antibody or binding construct that comprises:
a) a VH region comprising CDR-H1 as depicted in SEQ ID NO: 229, CDR-H2 as depicted in SEQ ID NO: 230, and CDR-H3 as depicted in SEQ ID NO: 231, and a VL region comprising CDR-L1 as depicted in SEQ ID NO: 85, CDR-L2 as depicted in SEQ ID NO: 86 and CDR-L3 as depicted in SEQ ID NO: 87; or
b) a VH region comprising CDR-H1 as depicted in SEQ ID NO: 217, CDR-H2 as depicted in SEQ ID NO: 218, and CDR-H3 as depicted in SEQ ID NO: 219, and a VL region comprising CDR-L1 as depicted in SEQ ID NO: 73, CDR-L2 as depicted in SEQ ID NO: 74 and CDR-L3 as depicted in SEQ ID NO: 75; or
c) a VH region comprising CDR-H1 as depicted in SEQ ID NO: 235, CDR-H2 as depicted in SEQ ID NO: 236, and CDR-H3 as depicted in SEQ ID NO: 237, and a VL region comprising CDR-L1 as depicted in SEQ ID NO: 91, CDR-L2 as depicted in SEQ ID NO: 92 and CDR-L3 as depicted in SEQ ID NO: 93.

In one embodiment, the invention provides an isolated binding domain that binds to MAGEB2, wherein the binding domain comprises:
a) a VH region comprising SEQ ID NO: 347 and a VL region comprising SEQ ID NO: 346; or
b) a VH region comprising SEQ ID NO: 339 and a VL region comprising SEQ ID NO: 338; or
c) a VH region comprising SEQ ID NO: 351 and a VL region comprising SEQ ID NO: 350.

In one embodiment, the invention provides an isolated binding construct comprising a binding domain that binds to MAGEB2, wherein the binding domain comprises:
a) a VH region comprising SEQ ID NO: 347 and a VL region comprising SEQ ID NO: 346; or
b) a VH region comprising SEQ ID NO: 339 and a VL region comprising SEQ ID NO: 338; or
c) a VH region comprising SEQ ID NO: 351 and a VL region comprising SEQ ID NO: 350.

In one embodiment, the invention provides an isolated binding domain that binds to MAGEB2, wherein the binding domain binds to the same epitope as an antibody or binding construct that comprises:
a) a VH region comprising SEQ ID NO: 347 and a VL region comprising SEQ ID NO: 346; or
b) a VH region comprising SEQ ID NO: 339 and a VL region comprising SEQ ID NO: 338; or
c) a VH region comprising SEQ ID NO: 351 and a VL region comprising SEQ ID NO: 350.

In one embodiment, the invention provides an isolated binding construct comprising a binding domain that binds to MAGEB2, wherein the binding domain binds to the same epitope as an antibody or binding construct that comprises:
a) a VH region comprising SEQ ID NO: 347 and a VL region comprising SEQ ID NO: 346; or
b) a VH region comprising SEQ ID NO: 339 and a VL region comprising SEQ ID NO: 338; or
c) a VH region comprising SEQ ID NO: 351 and a VL region comprising SEQ ID NO: 350.

In another embodiment, the invention provides an isolated binding construct comprising a binding domain that binds to MAGEB2, wherein the binding domain comprises a VH region selected from the group consisting of any VH region and a corresponding VL region as depicted in any of the sequences in Tables 21-25 herein.

In another embodiment, the invention provides an isolated binding construct comprising a binding domain that binds to MAGEB2, wherein the binding domain binds to the same epitope as an antibody or binding construct that comprises a VH region selected from the group consisting of any VH region and a corresponding VL region as depicted in any of the sequences in Table 23 herein.

In another embodiment, the invention provides an isolated binding construct comprising a binding domain that binds to MAGEB2, wherein the binding domain comprises a CDR-H1, CDR-H2, CDR-H3 from a VH region selected from the group consisting of any VH region as depicted in any of the sequences in Table 22 herein and further comprises a corresponding CDR-L1, CDR-L2, CDR-L3 from a VL region selected from the group consisting of any VL region as depicted in any of the sequences in Table 21 herein.

In another embodiment, the invention provides an isolated binding construct comprising a binding domain that binds to MAGEB2, wherein the binding domain binds to the same epitope as an antibody or binding construct that comprises a CDR-H1, CDR-H2, CDR-H3 from a VH region selected from the group consisting of any VH region as depicted in any of the sequences in Table 22 herein and further comprises a corresponding CDR-L1, CDR-L2, CDR-L3 from a VL region selected from the group consisting of any VL region as depicted in any of the sequences in Table 21 herein.

In another embodiment, the invention provides an isolated binding construct comprising a binding domain that binds to MAGEB2, wherein the binding domain comprises a VH and a VL region consensus sequence selected from any of the sequences in Table 25 herein.

In another embodiment, the invention provides an isolated binding construct comprising a binding domain that binds to MAGEB2, wherein the binding domain comprises a CDR-H1, CDR-H2, CDR-H3 from a VH region consensus sequence selected from the group consisting of any VH region as depicted in any of the sequences in Table 25 herein and further comprises a corresponding CDR-L1, CDR-L2, CDR-L3 from a VL region consensus sequence selected from the group consisting of any VL region as depicted in any of the sequences in Table 25 herein.

Exemplary binding construct full sequences are presented below:

1C3_LC
(SEQ ID NO: 555)
MDMRVPAQLLGLLLLWLRGARCDIVMTQTPSSVEAAVGGTVTIKCQASQN
ISSYLAWYQQKPGQPPKLLIYRASTLASGVPSRFKGSGSGTQFTLTISDL
ECADAATYYCQSYDDSRSSNFFYAFGGGTEVVVKGDPVAPTVLLFPPSSD
EVATGTVTIVCVANKYFPDVTVTWEVDGTTQTTGIENSKTPQNSADCTYN
LSSTLTLTSTQYNSHKEYTCKVTQGTTSVVQSFSRKNC
1C3_HC
(SEQ ID NO: 556)
MDMRVPAQLLGLLLLWLRGARCQSLEESGGGLVQPEGSLTLTCTAFGVTL
TNYYICWVRQAPGKGLEWVGCIDNANGRTYYASWAKGRFTISKTSSTTGT
LQMTSLTAADTATYFCARSLATPLWGPGTLVTVSSGQPKAPSVFPLAPCC
GDTPSSTVTLGCLVKGYLPEPVTVTWNSGTLTNGVRTFPSVRQSSGLYSL
SSVVSVTSSSQPVTCNVAHPATNTKVDKTVAPSTCSKPTCPPPELLGGPS
VFIFPPKPKDTLMISRTPEVTCVVVDVSQDDPEVQFTWYINNEQVRTARP
PLREQQFNSTIRVVSTLPIAHQDWLRGKEFKCKVHNKALPAPIEKTISKA
RGQPLEPKVYTMGPPREELSSRSVSLTCMINGFYPSDISVEWEKNGKAED
NYKTTPAVLDSDGSYFLYSKLSVPTSEWQRGDVFTCSVMHEALHNHYTQK
SISRSPGK
1I14_LC
(SEQ ID NO: 557)
MDMRVPAQLLGLLLLWLRGARCDVVMTQTPASVEATVGGTVTIKCQSSQS
VYDNNALAWYQQNAGQRPRLLIYGASTLASGVPSRFSASGSGTEFTLTIS
DLECADAATYYCQCTYYVSSYQNDFGGGTEVVVKGDPVAPTVLLFPPSSD
EVATGTVTIVCVANKYFPDVTVTWEVDGTTQTTGIENSKTPQNSADCTYN
LSSTLTLTSTQYNSHKEYTCKVTQGTTSVVQSFSRKNC
1I14_HC
(SEQ ID NO: 558)
MDMRVPAQLLGLLLLWLRGARCQSVEESGGRLVTPGTPLTLTCTISGFSL
SSYAMSWVRQAPGKGLEWIGSIGGGGSAVYASWAKGRFTISKTSTTVDLR
ITSPTTEDTAMYFCGRGFYSIDLWGPGTLVTVSSGQPKAPSVFPLAPCCG
DTPSSTVTLGCLVKGYLPEPVTVTWNSGTLTNGVRTFPSVRQSSGLYSLS
SVVSVTSSSQPVTCNVAHPATNTKVDKTVAPSTCSKPTCPPPELLGGPSV
FIFPPKPKDTLMISRTPEVTCVVVDVSQDDPEVQFTWYINNEQVRTARPP
LREQQFNSTIRVVSTLPIAHQDWLRGKEFKCKVHNKALPAPIEKTISKAR
GQPLEPKVYTMGPPREELSSRSVSLTCMINGFYPSDISVEWEKNGKAEDN
YKTTPAVLDSDGSYFLYSKLSVPTSEWQRGDVFTCSVMHEALHNHYTQKS
ISRSPGK
1H17_LC
(SEQ ID NO: 559)
MDMRVPAQLLGLLLLWLRGARCAAVLTQTPSPVSAAVGGTVSASCQSSKS
VYNKNWLSWFQQKPGQPPKLLIYGASTLASGVPSRFKGSGSGTQFTLTIS
DVQCDDAATYYCAGGYSSSSDTFAFGGGTEVVVKGDPVAPTVLLFPPSSD
EVATGTVTIVCVANKYFPDVTVTWEVDGTTQTTGIENSKTPQNSADCTYN
LSSTLTLTSTQYNSHKEYTCKVTQGTTSVVQSFSRKNC
1H17_HC
(SEQ ID NO: 560)
MDMRVPAQLLGLLLLWLRGARCQEQLVESGGGLVKPGASLTLTCKASGFS
FSSGQLMCWVRQAPGKGLEWIACIGSGSNAISTFYASWAQGRFTISKSSS
TTVTLQLTSLTAADTATYFCARVGSDDYGDSDVFDPWGPGTLVTVSSGQP
KAPSVFPLAPCCGDTPSSTVTLGCLVKGYLPEPVTVTWNSGTLTNGVRTF
PSVRQSSGLYSLSSVVSVTSSSQPVTCNVAHPATNTKVDKTVAPSTCSKP
TCPPPELLGGPSVFIFPPKPKDTLMISRTPEVTCVVVDVSQDDPEVQFTW
YINNEQVRTARPPLREQQFNSTIRVVSTLPIAHQDWLRGKEFKCKVHNKA
LPAPIEKTISKARGQPLEPKVYTMGPPREELSSRSVSLTCMINGFYPSDI
SVEWEKNGKAEDNYKTTPAVLDSDGSYFLYSKLSVPTSEWQRGDVFTCSV
MHEALHNHYTQKSISRSPGK

### Competitive Binding

Whether or not an antibody or binding construct competes for binding to an antigen (such as MAGEB2) with another given antibody or binding construct can be measured in a competition assay such as a competitive ELISA. Avidin-coupled microparticles (beads) can also be used. Similar to an avidin-coated ELISA plate, when reacted with a biotinylated protein, each of these beads can be used as a substrate on which an assay can be performed. Antigen is coated onto a bead and then precoated with the first antibody. The second antibody is added, and any additional binding is determined. Read-out occurs via flow cytometry. Preferably a cell-based competition assay that allows binding to intracellularly expressed MAGEB2 is used, using either cells that naturally express MAGEB2, or cells that were stably or transiently transformed with MAGEB2. The term “competes for binding”, in the present context, means that competition occurs between the two tested antibodies of at least 20%, at least 30%, at least 40%, at least 50%, at least 60%, at least 70%, at least 80% or at least 90%, as determined by any one of the assays disclosed above, preferably the cell-based assay.

Competitive antibody binding assays include assays determining the competitive binding of two antibodies/binding constructs to a cell surface bound antigen. Common methods aim to detect binding of two antibodies/binding constructs, A and B, to the same antigen expressed by a cell may include steps of:
blocking of the antigen by pre-incubation of cells with antibody/binding construct A followed by a sub-maximal addition of labeled antibody/binding construct B and detecting the binding of B compared with binding in the absence of A;
titration (i.e. adding different amounts) of antibody/binding construct A in the presence of sub-maximal amounts of labeled antibody/binding construct B and detecting the effect on binding of B; or co-titration of A and B, wherein both antibodies/binding constructs are incubated together at maximal concentration and detecting whether the total binding equals or exceeds that of either A or B alone, i.e. a method which cannot be affected by the order of addition or relative amounts of the antibodies/binding constructs.

When two antibodies/binding constructs A and B compete for a antigen, the antibodies will very often compete with each other in blocking assays independently from the order of the addition of the antibodies. In other words, competition is detected if the assay is carried out in either direction. However, this is not always the case, and under certain circumstances the order of the addition of the antibodies or the direction of the assay may have an effect on the signal generated. This may be due to differences in affinities or avidities of the potentially competing antibodies/binding constructs. If the order of the addition has a significant effect on the signal generated, it is concluded that the two antibodies/binding constructs do compete if competition is detected in at least one order.

### Epitope Amino Acid Residues

Structural analysis of the interaction between the target polypeptide and binding domain of, for example, a construct contemplated herein can provide amino acid residues of the target epitope involved with the binding interaction. In certain embodiments, the crystal structure of the target-binder interaction can provide these amino acid residues. In other embodiments, various other analyses can be performed to ascertain amino acid residues involved in the binding interaction. For example, Xscan, alanine scanning, arginine scanning, and other techniques known to those skilled in the art. In the present invention, the binding constructs were generated using specific peptide immunogens, and must therefore bind to these specific peptide sequences.

In embodiments where a peptide is used as the immunogen, such as those provided herein in SEQ ID NOs 2, 3, 4, or 562, antibodies that are isolated will be expected to bind an epitope that comprises all of, or a fragment of, the peptide immunogen. This epitope can be a linear epitope with that comprises all of, or a fragment of, the peptide immunogen amino acid sequences. Alternatively, and more likely in the case of longer peptide immunogens, this epitope can be a conformational epitope that comprises discontinuous amino acid sequences from within the peptide immunogen. Accordingly, in one embodiment of the present invention, the epitope that the binding constructs (e.g., antibodies) bind to comprises the MAGEB2 peptide a.a.43-76: SSVSGGAASSSPAAGIPQEPQRAPTTAAAAAAGV (N-terminal region peptide) (SEQ ID NO: 2).

In another embodiment, the epitope that the binding constructs (e.g., antibodies) bind to comprises a fragment of the MAGEB2 peptide a.a.43-76: SSVSGGAASSSPAAGIPQEPQRAPTTAAAAAAGV (N-terminal region peptide) (SEQ ID NO: 2) that is 10, 11, 12, 13, 14, 15, 16, 17, 18, 19, 20, 21, 22, 23, 24, 25, 26, 27, 28, 29, 30, 31, 32, or 33 amino acids long.

In another embodiment, the epitope that the binding constructs (e.g., antibodies) bind to comprises at least two, at least three, at least four, or at least five discontinuous amino acid sequence fragments of the MAGEB2 peptide a.a.43-76: SSVSGGAASSSPAAGIPQEPQRAPTTAAAAAAGV (N-terminal region peptide) (SEQ ID NO: 2), wherein each discontinuous amino acid sequence fragment is 1, 2, 3, 4, 5, 6, 7, 8, 9, 10, 11, 12, 13, 14, 15, 16, 17, 18, 19, or 20 amino acids long

In another embodiment of the present invention, the epitope that the binding constructs of the invention bind to comprises the MAGEB2 peptide a.a.95-125: SSSQASTSTKSPSEDPLTRKSGSLVQFLLYK (MHD N-terminal helix peptide) (SEQ ID NO: 3).

In another embodiment, the epitope that the binding constructs (e.g., antibodies) bind to comprises a fragment of the MAGEB2 peptide a.a.95-125: SSSQASTSTKSPSEDPLTRKSGSLVQFLLYK (MHD N-terminal helix peptide) (SEQ ID NO: 3) that is 10, 11, 12, 13, 14, 15, 16, 17, 18, 19, 20, 21, 22, 23, 24, 25, 26, 27, 28, 29, or 30, amino acids long.

In another embodiment, the epitope that the binding constructs (e.g., antibodies) bind to comprises at least two, at least three, at least four, or at least five discontinuous amino acid sequence fragments of the MAGEB2 peptide a.a.95-125: SSSQASTSTKSPSEDPLTRKSGSLVQFLLYK (MHD N-terminal helix peptide) (SEQ ID NO: 3), wherein each discontinuous amino acid sequence fragment is 1, 2, 3, 4, 5, 6, 7, 8, 9, 10, 11, 12, 13, 14, 15, 16, 17, 18, 19, or 20 amino acids long.

In another embodiment of the present invention, the epitope that the binding constructs of the invention bind to comprises the MAGEB2 peptide a.a.185-200: DLTDEESLLSSWDFPR (MHD middle loop peptide) (SEQ ID NO: 562).

In another embodiment, the epitope that the binding constructs (e.g., antibodies) bind to comprises a fragment of the MAGEB2 peptide a.a.185-200: DLTDEESLLSSWDFPR (MHD middle loop peptide) (SEQ ID NO: 562) that is 10, 11, 12, 13, 14, or 15 amino acids long.

In another embodiment, the epitope that the binding constructs (e.g., antibodies) bind to comprises at least two, at least three, at least four, or at least five discontinuous amino acid sequence fragments of the MAGEB2 peptide a.a.185-200: DLTDEESLLSSWDFPR (MHD middle loop peptide) (SEQ ID NO: 562), wherein each discontinuous amino acid sequence fragment is 1, 2, 3, 4, 5, 6, 7, 8, 9, or 10 amino acids long.

In one embodiment, the invention provides a binding construct that binds to an epitope comprising the peptides of any of SEQ ID NOs: 2, 3, 4, 562, or 388-554.

The region of the binding domain that binds to the epitope is called a “paratope.” Specific binding is believed to be accomplished by specific motifs in the amino acid sequence of the binding domain and the antigen. Thus, binding is achieved as a result of their primary, secondary and/or tertiary structure as well as the result of potential secondary modifications of said structures.

The term “Fc portion” or “Fc monomer” means in connection with this invention a polypeptide comprising at least one domain having the function of a CH2 domain and at least one domain having the function of a CH3 domain of an immunoglobulin molecule. As apparent from the term “Fc monomer”, the polypeptide comprising those CH domains is a “polypeptide monomer”. An Fc monomer can be a polypeptide comprising at least a fragment of the constant region of an immunoglobulin excluding the first constant region immunoglobulin domain of the heavy chain (CH1), but maintaining at least a functional part of one CH2 domain and a functional part of one CH3 domain, wherein the CH2 domain is amino terminal to the CH3 domain.

In one embodiment of this definition, an Fc monomer can be a polypeptide constant region comprising a portion of the Ig-Fc hinge region, a CH2 region and a CH3 region, wherein the hinge region is amino terminal to the CH2 domain. It is envisaged that the hinge region of the present invention promotes dimerization. Such Fc polypeptide molecules can be obtained by papain digestion of an immunoglobulin region (of course resulting in a dimer of two Fc polypeptide), for example and not limitation. In another aspect of this definition, an Fc monomer can be a polypeptide region comprising a portion of a CH2 region and a CH3 region. Such Fc polypeptide molecules can be obtained by pepsin digestion of an immunoglobulin molecule, for example and not limitation.

In one embodiment, the polypeptide sequence of an Fc monomer is substantially similar to an Fc polypeptide sequence of: an IgG1 Fc region, an IgG2 Fc region, an IgG3 Fc region, an IgG4 Fc region, an IgM Fc region, an IgA Fc region, an IgD Fc region and an IgE Fc region. (See, e.g., Padlan, Molecular Immunology, 31(3), 169-217 (1993)). Because there is some variation between immunoglobulins, and solely for clarity, Fc monomer refers to the last two heavy chain constant region immunoglobulin domains of IgA, IgD, and IgG, and the last three heavy chain constant region immunoglobulin domains of IgE and IgM. As mentioned, the Fc monomer can also include the flexible hinge N-terminal to these domains. For IgA and IgM, the Fc monomer may include the J chain. For IgG, the Fc portion comprises immunoglobulin domains CH2 and CH3 and the hinge between the first two domains and CH2. Although the boundaries of the Fc portion may vary an example for a human IgG heavy chain Fc portion comprising a functional hinge, CH2 and CH3 domain can be defined e.g. to comprise residues D231 (of the hinge domain—corresponding to D234 in Table 4 below) to P476, respectively L476 (for IgG4) of the carboxyl-terminus of the CH3 domain, wherein the numbering is according to Kabat. The two Fc portion or Fc monomer, which are fused to each other via a peptide linker define the third domain of the binding construct of the invention, which may also be defined as scFc domain.

In one embodiment of the invention it is envisaged that a scFc domain as disclosed herein, respectively the Fc monomers fused to each other are comprised only in the third domain of the binding construct. In some embodiments an IgG hinge region can be identified by analogy using the Kabat numbering as set forth in Table 4. In line with the above, it is envisaged that for a hinge domain/region of the present invention the minimal requirement comprises the amino acid residues corresponding to the IgG1 sequence stretch of D231 D234 to P243 according to the Kabat numbering. It is likewise envisaged that a hinge domain/region of the present invention comprises or consists of the IgG1 hinge sequence DKTHTCPPCP (SEQ ID NO: 4) (corresponding to the stretch D234 to P243 as shown in Table 4 below—variations of said sequence are also envisaged provided that the hinge region still promotes dimerization). In a preferred embodiment of the invention the glycosylation site at Kabat position 314 of the CH2 domains in the third domain of the binding construct is removed by a N314X substitution, wherein X is any amino acid excluding Q. Said substitution is preferably a N314G substitution. In a more preferred embodiment, said CH2 domain additionally comprises the following substitutions (position according to Kabat) V321C and R309C (these substitutions introduce the intra domain cysteine disulfide bridge at Kabat positions 309 and 321).

TABLE 4
Kabat numbering of the amino acid residues of the hinge region

| IMGT numbering | IgG₁ amino acid | Kabat |
|---|---|---|
| for the hinge | translation | numbering |
| 1 | (E) | 226 |
| 2 | P | 227 |
| 3 | K | 228 |
| 4 | S | 232 |
| 5 | C | 233 |
| 6 | D | 234 |
| 7 | K | 235 |
| 8 | T | 236 |
| 9 | H | 237 |
| 10 | T | 238 |
| 11 | C | 239 |
| 12 | P | 240 |
| 13 | P | 241 |
| 14 | C | 242 |
| 15 | P | 243 |

In further embodiments of the present invention, the hinge domain/region comprises or consists of the IgG2 subtype hinge sequence ERKCCVECPPCP (SEQ ID NO: 563), the IgG3 subtype hinge sequence ELKTPLDTTHTCPRCP (SEQ ID NO: 564) or ELKTPLGDTTHTCPRCP (SEQ ID NO: 565), and/or the IgG4 subtype hinge sequence ESKYGPPCPSCP (SEQ ID NO: 567). The IgG1 subtype hinge sequence may be the following one EPKSCDKTHTCPPCP (SEQ ID NO: 568). These core hinge regions are thus also envisaged in the context of the present invention.

The location and sequence of the IgG CH2 and IgG CH3 domain can be identified by analogy using the Kabat numbering as set forth in Table 5:

TABLE 5
Kabat numbering of the amino acid residues
of the IgG CH2 and CH3 region

| IgG | CH2 aa | CH2 Kabat | CH3 aa | CH3 Kabat |
|---|---|---|---|---|
| subtype | translation | numbering | translation | numbering |
| IgG₁ | APE . . . KAK | 244 . . . 360 | GQP . . . PGK | 361 . . . 478 |
| IgG₂ | APP . . . KTK | 244 . . . 360 | GQP . . . PGK | 361 . . . 478 |
| IgG₃ | APE . . . KTK | 244 . . . 360 | GQP . . . PGK | 361 . . . 478 |
| IgG₄ | APE . . . KAK | 244 . . . 360 | GQP . . . LGK | 361 . . . 478 |

In one embodiment of the invention, the emphasized bold amino acid residues in the CH3 domain of the first or both Fc monomers are deleted.

In a classical full-length antibody or immunoglobulin, each light (L) chain is linked to a heavy (H) chain by one covalent disulfide bond, while the two H chains are linked to each other by one or more disulfide bonds depending on the H chain isotype. The heavy chain constant (CH) domain most proximal to VH is usually designated as CH1. The constant (“C”) domains are not directly involved in antigen binding, but exhibit various effector functions, such as antibody-dependent cell-mediated cytotoxicity (ADCC) and complement activation (complement dependent cytotoxicity, CDC). The Fc region of an antibody is the “tail” region of a classical antibody that interacts with cell surface receptors called Fc receptors and some proteins of the complement system. In IgG, IgA and IgD antibody isotypes, the Fc region is composed of two identical protein fragments, derived from the second and third constant domains (CH2 and CH3) of the antibody's two heavy chains. IgM and IgE Fc regions contain three heavy chain constant domains (CH2, CH3 and CH4) in each polypeptide chain. The Fc regions also contains part of the so-called “hinge” region held together by one or more disulfides and noncovalent interactions. The Fc region of a naturally occurring IgG bears a highly conserved N-glycosylation site. Glycosylation of the Fc fragment is essential for Fc receptor-mediated activity.

In some embodiments of the invention the CH2 domain of one or preferably each (both) polypeptide monomers of the third domain comprises an intra domain cysteine disulfide bridge. As known in the art the term “cysteine disulfide bridge” refers to a functional group with the general structure R—S—S—R. The linkage is also called an SS-bond or a disulfide bridge or a cysteine clamp and is derived by the coupling of two thiol groups of cysteine residues. In certain embodiments, the cysteines forming the cysteine disulfide bridge in the mature binding construct are introduced into the amino acid sequence of the CH2 domain corresponding to 309 and 321 (Kabat numbering). In other embodiments, the cysteine clamps are introduced in other domains of the binding constructs. See also, e.g. US 2016/0193295.

In one embodiment of the invention a glycosylation site in Kabat position 314 of the CH2 domain is removed. It is preferred that this removal of the glycosylation site is achieved by a N314X substitution, wherein X is any amino acid excluding Q. Said substitution is preferably a N314G. In a more preferred embodiment, said CH2 domain additionally comprises the following substitutions (position according to Kabat) V321C and R309C (these substitutions introduce the intra domain cysteine disulfide bridge at Kabat positions 309 and 321).

Covalent modifications of the binding constructs are also included within the scope of this invention, and are generally, but not always, done post-translationally. For example, several types of covalent modifications of the binding construct are introduced into the molecule by reacting specific amino acid residues of the binding construct with an organic derivatizing agent that is capable of reacting with selected side chains or the N- or C-terminal residues.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0. Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form 0-acetyl tyrosyl species and 3-nitro derivatives, respectively. Tyrosyl residues are iodinated using 125I or 131I to prepare labeled proteins for use in radioimmunoassay, the chloramine T method described above being suitable.

Carboxyl side groups (aspartyl or glutamyl) are selectively modified by reaction with carbodiimides (R′—N═C═N—R′), where R and R′ are optionally different alkyl groups, such as 1-cyclohexyl-3-(2-morpholinyl-4-ethyl) carbodiimide or 1-ethyl-3-(4-azonia-4,4-dimethylpentyl) carbodiimide.

Furthermore, aspartyl and glutamyl residues are converted to asparaginyl and glutaminyl residues by reaction with ammonium ions.

Derivatization with bifunctional agents is useful for crosslinking the binding constructs of the present invention to a water-insoluble support matrix or surface for use in a variety of methods. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates as described in U.S. Pat. Nos. 3,969,287; 3,691,016; 4,195,128; 4,247,642; 4,229,537; and 4,330,440 are employed for protein immobilization. Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, 1983, pp. 79-86), acetylation of the N-terminal amine, and amidation of any C-terminal carboxyl group.

Another type of covalent modification of the binding constructs included within the scope of this invention comprises altering the glycosylation pattern of the protein. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the binding construct is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the amino acid sequence of a binding construct is preferably altered through changes at the DNA level, particularly by mutating the DNA encoding the polypeptide at preselected bases such that codons are generated that will translate into the desired amino acids.

Another means of increasing the number of carbohydrate moieties on the binding construct is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330, and in Aplin and Wriston, 1981, CRC Crit. Rev. Biochem., pp. 259-306.

Removal of carbohydrate moieties present on the starting binding construct may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131. Enzymatic cleavage of carbohydrate moieties on polypeptides can be achieved by the use of a variety of endo- and exo-glycosidases as described by Thotakura et al., 1987, Meth. Enzymol. 138:350. Glycosylation at potential glycosylation sites may be prevented by the use of the compound tunicamycin as described by Duskin et al., 1982, J. Biol. Chem. 257:3105. Tunicamycin blocks the formation of protein-N-glycoside linkages.

Other modifications of the binding construct are also contemplated herein. For example, another type of covalent modification of the binding construct comprises linking the binding construct to various non-proteinaceous polymers, including, but not limited to, various polyols such as polyethylene glycol, polypropylene glycol, polyoxyalkylenes, or copolymers of polyethylene glycol and polypropylene glycol, in the manner set forth in U.S. Pat. Nos. 4,640,835; 4,496,689; 4,301,144; 4,670,417; 4,791,192 or 4,179,337. In addition, as is known in the art, amino acid substitutions may be made in various positions within the binding construct, e.g. in order to facilitate the addition of polymers such as PEG.

In some embodiments, the covalent modification of the binding constructs of the invention comprises the addition of one or more labels. The labelling group may be coupled to the binding construct via spacer arms of various lengths to reduce potential steric hindrance. Various methods for labelling proteins are known in the art and can be used in performing the present invention. The term “label” or “labelling group” refers to any detectable label. In general, labels fall into a variety of classes, depending on the assay in which they are to be detected—the following examples include, but are not limited to:
isotopic labels, which may be radioactive or heavy isotopes, such as radioisotopes or radionuclides (e.g., ³H, ¹⁴C, ¹⁵N, ³⁵S ⁸⁹Zr, ⁹⁰Y, ⁹⁹Tc, ¹¹¹In, ¹²⁵I, ¹³¹I)
magnetic labels (e.g., magnetic particles)
redox active moieties
optical dyes (including, but not limited to, chromophores, phosphors and fluorophores) such as fluorescent groups (e.g., FITC, rhodamine, lanthanide phosphors), chemiluminescent groups, and fluorophores which can be either “small molecule” fluors or proteinaceous fluors
enzymatic groups (e.g. horseradish peroxidase, β-galactosidase, luciferase, alkaline phosphatase)
biotinylated groups
predetermined polypeptide epitopes recognized by a secondary reporter (e.g., leucine zipper pair sequences, binding sides for secondary antibodies, metal binding domains, epitope tags, etc.)

By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, OR), FITC, Rhodamine, and Texas Red (Pierce, Rockford, IL), Cy5, Cy5.5, Cy7 (Amersham Life Science, Pittsburgh, PA). Suitable optical dyes, including fluorophores, are described in Molecular Probes Handbook by Richard P. Haugland.

Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank Accession Number U55762), blue fluorescent protein (BFP, Quantum Biotechnologies, Inc. 1801 de Maisonneuve Blvd. West, 8th Floor, Montreal, Quebec, Canada H3H 1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), β galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658; 5,418,155; 5,683,888; 5,741,668; 5,777,079; 5,804,387; 5,874,304; 5,876,995; 5,925,558).

Leucine zipper domains are peptides that promote oligomerization of the proteins in which they are found. Leucine zippers were originally identified in several DNA-binding proteins (Landschulz et al., 1988, Science 240:1759), and have since been found in a variety of different proteins. Among the known leucine zippers are naturally occurring peptides and derivatives thereof that dimerize or trimerize. Examples of leucine zipper domains suitable for producing soluble oligomeric proteins are described in PCT application WO 94/10308, and the leucine zipper derived from lung surfactant protein D (SPD) described in Hoppe et al., 1994, FEBS Letters 344:191. The use of a modified leucine zipper that allows for stable trimerization of a heterologous protein fused thereto is described in Fanslow et al., 1994, Semin. Immunol. 6:267-78.

The binding construct of the invention may also comprise additional domains, which are e.g. helpful in the isolation of the molecule or relate to an adapted pharmacokinetic profile of the molecule. Domains helpful for the isolation of a binding construct may be selected from peptide motives or secondarily introduced moieties, which can be captured in an isolation method, e.g. an isolation column. Non-limiting embodiments of such additional domains comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g. Strepli-tag) and His-tag. All herein disclosed binding constructs may comprise a His-tag domain, which is generally known as a repeat of consecutive His residues in the amino acid sequence of a molecule, preferably of five, and more preferably of six His residues (hexa-histidine) (SEQ ID NO: 569). The His-tag may be located e.g. at the N- or C-terminus of the binding construct, preferably it is located at the C-terminus. Most preferably, a hexa-histidine tag (HHHHHH) (SEQ ID NO: 569) is linked via peptide bond to the C-terminus of the binding construct according to the invention. Additionally, a conjugate system of PLGA-PEG-PLGA may be combined with a poly-histidine tag for sustained release application and improved pharmacokinetic profile.

### Diagnostic Methods

The binding constructs that are provided herein are useful for detecting MAGEB2 in biological samples and can be used for diagnostic purposes to detect, diagnose, or monitor diseases and/or conditions associated with MAGEB2.

The disclosed binding constructs provide a means for the detection of the presence of MAGEB2 in a sample using, for example, classical immunohistological methods known to those of skill in the art (e.g., Tijssen, 1993, Practice and Theory of Enzyme Immunoassays, Vol 15 (Eds R. H. Burdon and P. H. van Knippenberg, Elsevier, Amsterdam); Zola, 1987, Monoclonal Antibodies: A Manual of Techniques, pp. 147-158 (CRC Press, Inc.); Jalkanen et al., 1985, J. Cell. Biol. 101:976-985; Jalkanen et al., 1987, J. Cell Biol. 105:3087-3096). The detection of MAGEB2 can be performed in vivo or in vitro, but is preferably done in vitro on a sample that comprises cells obtained from a patient. The methods used to detect MAGEB2 expression in human tissue, e.g., tumor tissue, can be qualitative, semi-quantitative, or quantitative. For example, art-recognized antibody-based methods for detecting protein levels in biological samples include, but are not limited to, enzyme-linked immunosorbent assay (ELISA), radioimmunoassays, electrochemiluninescence (ECL) assays, surface plasmon resonance, western blot, immunoprecipitation, fluorescence-activated cell sorting (FACS), immunofluorescence, immunohistochemistry, and the like. Methods for detecting MAGEB2 expression in a biological sample may include control samples (negative and positive controls). For example, a negative control sample may be a sample containing no MAGEB2 protein, and a positive controls sample is a sample containing MAGEB2 protein. Comparing the results with the negative and positive controls can confirm the presence or absence of MAGEB2 in the biological sample.

In a specific embodiment, the binding construct can be used in an immunohistochemistry (IHC) assay. These IHC assays can be performed on modern, higher throughput platforms that in some instances are automated. For example, the Roche-Ventana BenchMark ULTRA system.

IHC is a commonly used assay that involves the use of binding constructs such as antibodies to specifically bind to a target antigen in a tissue sample. This allows the assay user to ascertain whether the target antigen is or is not present in the tissue sample.

Immunohistochemical methods are well-known in the art and are described in, e.g., Immunohistochemical Staining Methods 6th Edition 2013, Taylor C R, Rudbeck L, eds., Dako North America, available as an Education Guide at www.dako.com; “Immunohistochemical Staining Method guide”). Immunohistochemical staining may be conducted on fixed tissue sections or on frozen tissue sections. When using fixed tissue sections, e.g., formalin fixed paraffin embedded (FFPE) tissue sections, the procedure can generally use the following exemplary steps: obtaining a tumor tissue sample (e.g., by biopsy), fixation of the tumor sample; embedding (e.g., in paraffin); sectioning and mounting; antigen retrieval; incubation with a primary antibody (e.g., a MAGEB2 antibody described herein), detection (e.g., after amplification of the antigen/antibody complex signal), and interpretation by a skilled practitioner (e.g., using an art-recognized scoring system).

Suitable, non-limiting fixatives include, for example, paraformaldehyde, glutaraldehyde, formaldehyde, acetic acid, acetone, osmium tetroxide, chromic acid, mercuric chloride, picric acid, alcohols (e.g., methanol, ethanol), Gendre's fluid, Rossman's fluid, B5 fixative, Bouin's fluid, Carnoy's fixative, and methacarn. In one embodiment, the tumor sample is fixed in formaldehyde (e.g., 10% neutral buffered formalin, which corresponds to 4% formaldehyde in a buffered solution). Once fixed, the tumor sample can be serially dehydrated using alcohol or xylene, embedded in paraffin, and cut with a microtome to generate tumor tissue sections (e.g., with a thickness of about 4-5 μm), which can then be mounted onto microscope slides (e.g., adhesive-coated glass slides). Exemplary embedding materials include paraffin, celloidin, OCT™ compound, agar, plastics, or acrylics. Another exemplary material, and as described in the Examples herein, is Histogel containing agarose/glycerine (ThermoFisher Scientific).

Provided herein are methods for detecting MAGEB2 expression in a biological sample, e.g., a human tumor tissue sample, by contacting the sample with an anti-MAGEB2 antigen binding construct, such as an anti-MAGEB2 antibody comprising the heavy and light chain variable region CDR sequences set forth herein, or the heavy and light chain variable region sequences set forth herein, wherein the antigen binding construct specifically binds to human MAGEB2, and detecting the binding of the antibody to MAGEB2 in the sample, e.g., by immunohistochemistry.

Accordingly, the invention provides a method of diagnosing a subject with a tumor, comprising: a) determining the level of MAGEB2 in a sample obtained from the subject; and b) diagnosing the subject as having a MAGEB2 positive tumor when the level of MAGEB2 is increased relative to a control. Provided herein is a method of identifying a subject having a MAGEB2 positive tumor comprising: a) determining the level of MAGEB2 in a sample obtained from the subject; and b) identifying the subject as having a MAGEB2 positive tumor when the level of MAGEB2 is increased relative to a control. Provided herein is a method of identifying a subject as needing an anti-MAGEB2 therapeutic comprising: a) determining the level of MAGEB2 in a sample obtained from the subject; and b) identifying the subject as needing the anti-MAGEB2 therapeutic when the level of MAGEB2 is increased relative to a control.

Provided herein is a method of determining treatment for a subject with a MAGEB2 positive tumor comprising: determining the level of MAGEB2 in a sample obtained from the subject; and determining the treatment as comprising an anti-MAGEB2 therapeutic when the level of MAGEB2 is increased, relative to a control.

In certain embodiments, exemplary biological samples include blood samples, serum samples, cells, surgically-resected tissue, and biopsied tissue (e.g. cancer tissue) obtained from the cancer patient. Biological samples for use in the methods described herein can be fresh, frozen, or fixed. Biological samples, e.g., tumor samples, can be obtained from the patient using routine methods, such as, but not limited to, biopsy, surgical resection, or aspiration.

MAGEB2 protein expression in tissue, e.g., tumor, samples can be detected using direct or indirect methods. For example, in assays where the binding construct is an antibody, the primary antibody may comprise a detectable moiety, such as the enzyme horseradish peroxidase (HRP) or a fluorescent label (e.g., FITC, TRITC), as described herein. In certain embodiments, the primary antibody does not itself comprise a detectable moiety, but is instead detected by binding of a secondary antibody to it, for indirect immunohistochemistry. Accordingly, in certain embodiments, the secondary antibody comprises a detectable label, e.g., an enzymatic, chromogenic, or fluorescent label. In certain embodiments, the primary antibody is a chimeric antibody comprising human variable region sequences and a non-human Fc region. A secondary antibody can be used to recognize the non-human Fc region of the primary antibody in order to reduce background staining.

In other embodiments, MAGEB2 protein expression can be detected using the avidin-biotin complex method (ABC method). In these embodiments, for example when the binding construct is an antibody, the secondary antibody is biotinylated and can serve as a bridge between the primary antibody and biotin-avidin-peroxidase complex. Other suitable non-limiting methods for immunohistochemistry include those described in Chapter 6 of the Immunohistochemical Staining Method guide, e.g., methods described in Chilosi et al., Biotech Histochem 1994; 69:235; Sabattini et al., J Clin Pathol 1998; 51:506-11; and Gross et al., JBC 1959; 234:1622.

General methods for preparing and staining frozen tissue sections are well known in the art, and are described in, e.g., the Immunohistochemical Staining Method guide, Chapter 3 of Immunohistochemistry and Methods (Buchwalow and Bocker, Springer-Verlag Berlin Heidelberg 2010), and Chapter 21 of Theory and Practice of Histological Techniques (Bancroft and Gamble, 6.sup.th Edition, Elsevier Ltd., 2008). In some embodiments, slides stained with, e.g., the anti-MAGEB2 antibodies described herein can be further counterstained with, e.g., hematoxylin and/or eosin, using methods well known in the art.

MAGEB2 protein expression detected by immunohistochemistry can be digitally scanned and analyzed (e.g., as described herein in the Examples), or otherwise evaluated and scored using art-recognized scoring methods. Non-limiting examples of scoring methods are described below.

For example, in certain embodiments, MAGEB2 expression can be evaluated using the H-score (histochemical score) system, which is widely used in the art and is useful given its dynamic range and use of weighted percentiles. The H-score is a semiquantitative scoring system based on the formula: 3× percentage of strongly staining cells (3+ staining)+2× percentage of moderately staining (2+ staining) cells+1× percentage of weakly staining (1+ staining) cells+0× percentage of non-stained (0 staining) cells, giving a score range of 0 to 300. See, e.g., McCarty et al., Cancer Res 1986; 46:4244-8; Bosman et al., J Clin Pathol 1992; 45:120-4; Dieset et al., Analyt Quant Cytol Histol 1996; 18:351-4. Control tissue can include, e.g., matching non-tumor tissue from the same subject. Accordingly, in certain embodiments, the number of cells staining positive for MAGEB2 using the binding constructs herein (e.g., antibodies), and the intensity of staining, can be used to determine an H-score.

In other embodiments, MAGEB2 expression can be evaluated using the Allred scoring system (Allred et al., Mod Pathol 1998; 11:155-68; Harvey et al., J Clin Oncol 1999; 17:1474-91). This scoring system involves adding proportion and intensity scores to obtain a total score. Accordingly, in some embodiments, the proportion score is obtained based on the estimated proportion of tumor cells that are positive for MAGEB2 (0: none, 1: < 1/100, 2: 1/100 to 1/10, 3: 1/10 to ⅓; 4: ⅓ to ⅔; and 5: >⅔), and the intensity score is obtained based on the average intensity of MAGEB2 expression in positive tumor cells (0: none, 1: weak, 2: intermediate, 3: strong). Allred scores range from 0 to 8, with a score ranging from 3 to 8 considered positive (i.e., positive detection). Accordingly, in certain embodiments, tumors with an Allred score of 3 to 8, for example, an Allred score of 3, 4, 5, 6, 7, or 8, for MAGEB2 staining are considered MAGEB2-positive tumors.

In other embodiments, and as discussed herein above, the scoring system can be automated, e.g., computerized, and quantified by image analysis. Automated methods are well known in the art. For example, an average threshold measure (ATM) score, which obtains an average of 255 staining intensity levels, can be calculated as described in, e.g., Choudhury et al., J Histochem Cytochem 2010; 58:95-107, Rizzardi et al., Diagnostic Pathology 2012; 7:42-52. Another automated scoring system is AQUA® (automated quantitative analysis), which is performed using, e.g., tissue microarrays (TMAs), on a continuous scale. AQUA® is a hybrid of standard immunohistochemistry and flow cytometry that provides an objective numeric score ranging from 1-255, and involves antigen retrieval, use of primary and secondary antibodies, and multiplexed fluorescent detection. Optimal cutoff points can be determined as described in Camp et al. Clin Cancer Res 2004; 10:7252-9. The AQUA® scoring system is described in detail in Camp et al., Nat Med 2002; 8:1323-7; Camp et al., Cancer Res 2003; 63:1445-8; Ghosh et al., Hum Pathol 2008; 39:1835-43; Bose et al., BMC Cancer 2012; 12:332; Mascaux et al., Clin Cancer Res 2011; 17:7796-807. Other suitable automated immunohistochemistry platforms include commercially available platforms, such as the Leica BOND RX staining platform. The platform detects target protein expression in tumor tissue on the basis of staining intensity on the following scale: minimal, <1 cells per 20× objective field; mild, 1-10 cells per 20× objective field; moderate, 10-50 cells per 20× objective field, marked, 50-200 cells per 20× objective; and intense, >200 cells per 20× objective field.

In some embodiments, a tumor is considered MAGEB2-positive and likely to benefit from a therapeutic that targets MAGEB2 if a sample of the tumor has more MAGEB2-positive cells than a matching normal tissue from the same cancer patient.

In certain embodiments, the cancer patient is likely to or predicted to benefit from a therapeutic that targets MAGEB2 if the number of MAGEB2-positive cells in the tumor sample exceeds a threshold level. A threshold level may be the lowest level of MAGEB2 as determined with a given detection system, e.g., immunohistochemistry, as further described herein, such as in the Examples. In certain embodiments, the cancer patient is likely to or predicted to benefit from a therapeutic that targets MAGEB2 if the number of MAGEB2-positive cells in the tumor sample exceeds a threshold number or proportion using an art-recognized scoring system described supra. Accordingly, in some embodiments, if the number or proportion of MAGEB2-positive cells exceeds a certain threshold number or proportion using a scoring system described herein, then the sample can be defined as being “MAGEB2-positive.”

In certain embodiments, a tumor sample having at least 1%, for example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 90%, or at least 95% of cells in the tumor expressing MAGEB2 is designated as being “MAGEB2-positive” and indicates that the cancer patient is likely to or predicted to respond to a therapeutic that targets MAGEB2.

In some embodiments, an H score can be calculated for the tumor sample. Accordingly, in certain embodiments, a tumor sample with an H score of at least 5, such as at least 10, at least 15, at least 20, at least 25, at least 30, at least 40, at least 50, at least 60, at least 70, at least 80, at least 90, at least 100, at least 125, at least 150, at least 175, at least 200, at least 225, at least 250, at least 275, or at least 290 is designated as being “MAGEB2-positive” and indicates that the cancer patient is likely to or predicted to respond to a therapeutic that targets MAGEB2.

In some embodiments, an Allred score can be calculated for the tumor sample. Accordingly, in certain embodiments, a tumor sample with an Allred score of at least 3, such as at least 4, at least 5, at least 6, at least 7, or 8 is designated as being “MAGEB2-positive” and indicates that the cancer patient is likely to or predicted to respond to a therapeutic that targets MAGEB2.

In some embodiments, an AQUA score can be calculated. Accordingly, in certain embodiments, a tumor sample with an AQUA score of at least 5, such as at least 10, at least 15, at least 20, at least 25, at least 30, at least 40, at least 50, at least 60, at least 70, at least 80, at least 90, at least 100, at least 125, at least 150, at least 175, at least 200, at least 225, or at least 250 is designated as being “MAGEB2-positive” and indicates that the cancer patient is likely to or predicted to respond to a therapeutic that targets MAGEB2.

In certain embodiments, a combination of one of the scoring systems above with another detection modality, such as FISH, is used to increase the accuracy of determining whether a tumor is MAGEB2-positive or MAGEB2-negative.

In certain embodiments, the patient has a cancer selected from the group consisting of squamous cell carcinoma, small-cell lung cancer, non-small cell lung cancer, squamous non-small cell lung cancer (NSCLC), non squamous NSCLC, glioma, gastrointestinal cancer, renal cancer (e.g. clear cell carcinoma), ovarian cancer, liver cancer, colorectal cancer, endometrial cancer, kidney cancer (e.g., renal cell carcinoma (RCC)), prostate cancer (e.g. hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, T-cell lymphoma, environmentally-induced cancers including those induced by asbestos, virus-related cancers or cancers of viral origin (e.g., human papilloma virus (HPV-related or -originating tumors)), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (M0), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B cell hematologic malignancy, e.g., B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukaemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, as well as any combinations of said cancers. In certain embodiments, the cancer is a metastatic cancer, refractory cancer, or recurrent cancer.

### Treatment of Patients

Once a cancer patient has been identified as being likely to benefit from treatment with a therapeutic that targets MAGEB2 expressing tumors (i.e., has a MAGEB2-positive tumor), the patient can be treated with an anti-MAGEB2 therapeutic (e.g., an anti-MAGEB2 immunotherapeutic such as those found in U.S. Patent Application Nos. 63/027,148 filed on May 19, 2020 and entitled “MAGEB2 Binding Constructs” and 63/128,773 filed on Dec. 21, 2020 and entitled “MAGEB2 Binding Constructs.” Accordingly, provided herein is a method for treating a tumor in a subject, said method comprising: determining the subject as responsive to treatment with an anti-MAGEB2 therapeutic by obtaining a sample from the subject, wherein the sample comprises a cell from the tumor, measuring the level of MAGEB2 in the sample, and determining the subject as responsive to treatment with an anti-MAGEB2 therapeutic, and administering to the subject an effective amount of the anti-MAGEB2 therapeutic. Provided herein are methods of treating a cancer patient comprising: (a) determining whether a tumor of a cancer patient is MAGEB2-positive using the methods described herein, e.g., contacting a tumor sample from the patient with binding construct described herein, wherein the antibody or antigen-binding fragment thereof specifically binds to human MAGEB2, detecting the binding of the antibody to MAGEB2 in the sample, and determining the level of MAGEB2 protein expression in the sample, wherein a level of MAGEB2 protein above a threshold level in tumor cells indicates that the tumor is MAGEB2-positive, and (b) if the tumor is determined to be MAGEB2-positive, then administering a therapeutically effective amount of an agent that targets cells that express MAGEB2 and kills them. Provided herein is a method of treating a tumor in a subject, wherein the subject's tumor has been tested for MAGEB2 levels and the subject's tumor is MAGEB2 positive, comprising administering an anti-MAGEB2 therapeutic to the subject.

Provided herein is a method of determining efficacy of treatment with an anti-MAGEB2 therapeutic in a subject comprising: determining the level of MAGEB2 in a sample obtained from the subject before treatment with an anti-MAGEB2 therapeutic and after treatment with an anti-MAGEB2 therapeutic; and determining the treatment as effective when the level of MAGEB2 positive tumor cells is decreased after treatment with the anti-MAGEB2 therapeutic.

Provided herein is a method of monitoring treatment with an anti-MAGEB2 therapeutic in a subject comprising: determining the level of MAGEB2 in a sample obtained from the subject at a first timepoint; determining the level of MAGEB2 in a sample obtained from the subject at a second timepoint; and optionally continuing treatment with an anti-MAGEB2 therapeutic when the level of MAGEB2 is decreased at the second timepoint relative to the level at the first timepoint.

Provided herein is a method of identifying a subject as responsive to treatment with an anti-MAGEB2 therapeutic comprising: determining the level of MAGEB2 in a sample obtained from the subject; identifying the subject as responsive to treatment with an anti-MAGEB2 therapeutic when the level of MAGEB2 decreases upon treatment with an anti-MAGEB2 therapeutic.

In a specific embodiment of the above embodiments, the subject or patient is treated with a therapeutically-effective amount of any of the molecules disclosed in U.S. Patent Application Nos. 63/027,148 filed on May 19, 2020 and entitled “MAGEB2 Binding Constructs” and 63/128,773 filed on Dec. 21, 2020 and entitled “MAGEB2 Binding Constructs,” the contents of which are herein incorporated by reference.

In a further specific embodiment of the above embodiments, various reference levels of MAGEB2 in a patient sample can be compared to each other. For example, a first reference level taken at a first time point can be compared to a second level taken at a second time point. Further, e.g., MAGEB2 levels can be compared prior to drug treatment and after drug treatment. In this example, it can be envisaged that successful treatment with a drug will yield a lower measured MAGEB2 level after treatment as compared to the reference MAGEB2 level measured before treatment. The first and the second tissue sample can be obtained, e.g., within 3-7 days, 1 week to 3 weeks, or 1 month to 3 months from each other, or even a longer duration.

In certain embodiments, a method for monitoring a MAGEB2-expressing tumor in a cancer patient comprises: (a) detecting MAGEB2 protein expression in a tissue (e.g., tumor) sample at a first time point by using an anti-MAGEB2 antibody, or antigen-binding portion thereof, (b) determining the level of MAGEB2 protein expression at the first time point, (c) detecting MAGEB2 protein expression in a tissue (e.g., tumor, which may be the same tumor as the one that was biopsied at the first time point) sample at a second time point using the same antibody used in step (a), and (d) determining the level of MAGEB2 protein expression in the tumor from the second time point.

Provided herein are methods for monitoring the efficacy of anti-MAGEB2 immunotherapy (e.g., one of the molecules provided in U.S. Patent Application Nos. 63/027,148 filed on May 19, 2020 and entitled “MAGEB2 Binding Constructs” and 63/128,773 filed on Dec. 21, 2020 and entitled “MAGEB2 Binding Constructs”) in a patient having a MAGEB2-positive tumor comprising: (a) detecting MAGEB2 protein expression in the MAGEB2-positive tumor at a first time point before or after initiating anti-MAGEB2 immunotherapy by using an anti-MAGEB2 antibody, (b) determining the level of MAGEB2 protein expression in the tumor from the first time point, (c) detecting MAGEB2 protein expression in the MAGEB2-positive tumor at a second time point after initiating anti-MAGEB2 immunotherapy using the same antibody from step (a), (d) determining the level of MAGEB2 protein expression in the tumor from the second time point; (e) comparing the levels of MAGEB2 protein expression determined at the first and second time points, wherein a higher level at the first time point relative to the second time point may be indicative of effective anti-MAGEB2 immunotherapy, a lower score at the first time point relative to the second time point is indicative of ineffective anti-MAGEB2 immunotherapy, and an unchanged score at the first time point relative to the second time point may be indicative of anti-MAGEB2 immunotherapy being stabilizing. MAGEB2 expression may be determined in the tumor, tumor tissue, or tumor cells.

In certain embodiments, the status of a MAGEB2-expressing tumor can be monitored repeatedly after initial diagnosis (i.e., after making a determination that the tumor is MAGEB2-positive), such as one month after initial diagnosis, two months after initial diagnosis, three months after initial diagnosis, four months after initial diagnosis, five months after initial diagnosis, six months after initial diagnosis, one year after initial diagnosis, etc. In other embodiments, the efficacy of anti-MAGEB2 immunotherapy against a MAGEB2-expressing tumor can be monitored repeatedly after initiating anti-MAGEB2 immunotherapy, such as one month after initiation of therapy, two months after initiation of therapy, three months after initiation of therapy, four months after initiation of therapy, five months after initiation of therapy, six months after initiation of therapy, one year after initiation of therapy, etc.

### Sequence Modifications of MAGEB2 Binding Constructs

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gin or Q); glutamic acid (Glu or E); glycine (Gly or G); histidine (His or H); isoleucine (Ile or I): leucine (Leu or L); lysine (Lys or K); methionine (Met or M); phenylalanine (Phe or F); proline (Pro or P); serine (Ser or S); threonine (Thr or T); tryptophan (Trp or W); tyrosine (Tyr or Y); and valine (Val or V), although modified, synthetic, or rare amino acids may be used as desired. There are basically four different classes of amino acids determined by different side chains:
(1) non-polar and neutral (uncharged): Ala, Gly, Ile, Leu, Met, Phe, Pro, Val
(2) polar and neutral (uncharged): Asn, Cys (being only slightly polar), Gln, Ser, Thr, Trp (being only slightly polar), Tyr
(3) acidic and polar (negatively charged): Asp and Glu
(4) basic and polar (positively charged): Arg, His, Lys

Hydrophobic amino acids can be divided according to whether they have aliphatic or aromatic side chains. Phe and Trp (being very hydrophobic), Tyr and His (being less hydrophobic) are classified as aromatic amino acids. Strictly speaking, aliphatic means that the side chain contains only hydrogen and carbon atoms. By this strict definition, the amino acids with aliphatic side chains are alanine, isoleucine, leucine (also norleucine), proline and valine. Alanine's side chain, being very short, means that it is not particularly hydrophobic, and proline has an unusual geometry that gives it special roles in proteins. It is often convenient to consider methionine in the same category as isoleucine, leucine and valine, although it also contains a sulphur atom. The unifying theme is that these amino acids contain largely non-reactive and flexible side chains. The amino acids alanine, cysteine, glycine, proline, serine and threonine are often grouped together for the reason that they are all small in size. Gly and Pro may influence chain orientation.

Amino acid modifications include, for example, deletions of residues from, insertions of residues into, and/or substitutions of residues within the amino acid sequences of the binding constructs. Any combination of deletion, insertion, and/or substitution is made to arrive at a final binding construct, provided that the final construct possesses the desired characteristics, e.g. the biological activity of the unmodified parental molecule (such as binding to MAGEB2). The amino acid changes may also alter post-translational processes of the binding constructs, such as changing the number or position of glycosylation sites.

For example, 1, 2, 3, 4, 5, or 6 amino acids may be inserted, deleted and/or substituted in each of the CDRs (of course, dependent on their respective length), while 1, 2, 3, 4, 5, 6, 7, 8, 9, 10, 11, 12, 13, 14, 15, 16, 17, 18, 19, 20, or 25 amino acids may be inserted, deleted and/or substituted in each of the FRs. Amino acid sequence insertions also include N-terminal and/or C-terminal additions of amino acids ranging in length from e.g. 1, 2, 3, 4, 5, 6, 7, 8, 9 or 10 residues to polypeptides containing more than 10, e.g. one hundred or more residues, as well as intra-sequence insertions of single or multiple amino acid residues. An insertional variant of the binding construct of the invention includes the fusion of a polypeptide which increases or extends the serum half-life of the binding construct to the N-terminus or to the C-terminus of the binding construct. It is also conceivable that such insertion occurs within the binding construct, e.g. between the first and the second domain.

The sites of greatest interest for amino acid modifications, in particular for amino acid substitutions, include the the hypervariable regions, in particular the individual CDRs of the heavy and/or light chain, but FR alterations in the heavy and/or light chain are also contemplated. The substitutions can be conservative substitutions as described herein. Preferably, 1, 2, 3, 4, 5, 6, 7, 8, 9, or 10 amino acids may be substituted in a CDR, while 1, 2, 3, 4, 5, 6, 7, 8, 9, 10, 11, 12, 13, 14, 15, 16, 17, 18, 19, 20, or 25 amino acids may be substituted in the framework regions (FRs), depending on the length of the CDR or FR, respectively. For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

A useful method for the identification of certain residues or regions within the binding constructs that are preferred locations for mutagenesis is called “alanine scanning mutagenesis” and is described e.g. in Cunningham B. C. and Wells J. A. (Science. 1989 Jun. 2; 244(4908):1081-5). Here, a residue or group of residues within the binding construct is/are identified (e.g. charged residues such as Arg, His, Lys, Asp, and Glu) and replaced by a neutral or non-polar amino acid (most preferably alanine or polyalanine) to affect the interaction of the respective amino acid(s) with the epitope of the target protein. Alanine scanning is a technique used to determine the contribution of a specific residue to the stability or function of given protein. Alanine is used because of its non-bulky, chemically inert, methyl functional group that nevertheless mimics the secondary structure preferences that many of the other amino acids possess. Sometimes bulky amino acids such as valine or leucine can be used in cases where conservation of the size of mutated residues is needed. This technique can also be useful to determine whether the side chain of a specific residue plays a significant role in bioactivity. Alanine scanning is usually accomplished by site-directed mutagenesis or randomly by creating a PCR library. Furthermore, computational methods to estimate thermodynamic parameters based on a theoretical alanine substitutions have been developed. The data can be tested by IR, NMR Spectroscopy, mathematical methods, bioassays, etc.

Those amino acid locations demonstrating functional sensitivity to the substitutions (as determined e.g. by alanine scanning) can then be refined by introducing further or other variants at, or for, the sites of substitution. Thus, while the site or region for introducing an amino acid sequence variation is predetermined, the nature of the mutation per se needs not to be predetermined. For example, to analyze or optimize the performance of a mutation at a given site, alanine scanning or random mutagenesis may be conducted at a target codon or region, and the expressed binding construct variants are screened for the optimal combination of desired activity. Techniques for making substitution mutations at predetermined sites in the DNA having a known sequence are well known, for example, M13 primer mutagenesis and PCR mutagenesis. Screening of the mutants is done e.g. using assays of antigen (e.g. MAGEB2) binding activity and/or of cytotoxic activity.

Generally, if amino acids are substituted in one or more or all of the CDRs of the heavy and/or light chain, it is envisaged that the then-obtained “substituted” sequence is at least 60% or 65%, more preferably 70% or 75%, even more preferably 80% or 85%, and particularly preferably 90% or 95% identical/homologous to the “original” or “parental” CDR sequence. This means that the degree of identity/homology between the original and the substituted sequence depends on the length of the CDR. For example, a CDR having 5 amino acids in total and comprising one amino acid substitution is 80% identical to the “original” or “parental” CDR sequence, while a CDR having 10 amino acids in total and comprising one amino acid substitution is 90% identical to the “original” or “parental” CDR sequence. Accordingly, the substituted CDRs of the binding construct of the invention may have different degrees of identity to their original sequences, e.g., CDRL1 may have 80%, while CDRL3 may have 90% of homology. The same considerations apply to the framework regions and to the entire VH and VL regions.

A “variant CDR” is a CDR with a specific sequence homology, similarity, or identity to the parent CDR of the invention, and shares biological function with the parent CDR, including, but not limited to, at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR. Generally, the amino acid homology, similarity, or identity between individual variant CDRs is at least 60% to the parent sequences depicted herein, and more typically with increasing homologies, similarities or identities of at least 65% or 70%, preferably at least 75% or 80%, more preferably at least 85%, 90%, 91%, 92%, 93%, 94%, and most preferably 95%, 96%, 97%, 98%, 99%, and almost 100%. The same applies to “variant VH” and “variant VL”. According to one embodiment, the sequence variations within a “variant VH” and/or a “variant VL” do not extend to the CDRs. The present invention is hence directed to a binding construct as defined herein, comprising VH and VL sequences having a certain sequence homology (see above) to the specific sequences as defined herein (the “parental” VH and VL), wherein the CDR sequences are 100% identical to the specific CDR sequences as defined herein (the “parental” CDRs).

Preferred substitutions (or replacements) are conservative substitutions. However, any substitution (including non-conservative substitutions or one or more from the “exemplary substitutions” listed in Table 6, below) is envisaged, as long as the binding construct retains its capacity to bind to MAGEB2, and/or provided its CDRs, FRs, VH and/or VL sequences have a degree of identity to the original or parental sequence of at least 60% or 65%, more preferably at least 70% or 75%, even more preferably at least 80% or 85%, and particularly preferably at least 90% or 95%.

A conservative replacement (also called a conservative mutation or a conservative substitution) is an amino acid replacement that changes a given amino acid to a different amino acid with similar biochemical properties (e.g. charge, hydrophobicity, size). Conservative replacements in proteins often have a smaller effect on protein function than non-conservative replacements. Conservative substitutions are shown in Table 6. Exemplary conservative substitutions are shown as “exemplary substitutions”. If such substitutions result in a change in biological activity, then more substantial changes, as further described herein in reference to amino acid classes, may be introduced and the products screened for a desired characteristic.

TABLE 6
Amino acid substitutions (aa = amino acid)

| Original | | |
|---|---|---|
| aa | Conservative substitutions | Exemplary Substitutions |
| Ala (A) | Small aa | Gly, Ser, Thr |
| Arg (R) | Polar aa, in particular Lys | Lys, Gln, Asn |
| Asn (N) | Polar aa, in particular Asp | Asp, Gln, His, Lys, Arg |
| Asp (D) | Glu or other polar aa, | Glu, Asn |
| | in particular Asn | |
| Cys (C) | Small aa | Ser, Ala |
| Gln (Q) | Polar aa, in particular Glu | Glu, Asn |
| Glu (E) | Asp or other polar aa, | Asp, Gln |
| | in particular Gln | |
| Gly (G) | Small aa, such as Ala | Ala |
| His (H) | | Asn, Gln, Arg, Lys, Tyr |
| Ile (I) | Hydrophobic, in particular | Ala, Val, Met, Leu, Phe |
| | aliphatic aa | |
| Leu (L) | Hydrophobic, in particular | Norleucine, Ile, Ala, Val, Met |
| | aliphatic aa | |
| Lys (K) | Polar aa, in particular Arg | Arg, Gln, Asn |
| Met (M) | Hydrophobic, in particular | Leu, Ala, Ile, Val, Phe |
| | aliphatic aa | |
| Phe (F) | Aromatic or hydrophobic | Tyr, Trp, Leu, Val, Ile, Ala |
| | aa, in particular Tyr | |
| Pro (P) | Small aa | Ala |
| Ser (S) | Polar or small aa, in | Thr |
| | particular Thr | |
| Thr (T) | Polar aa, in particular Ser | Ser |
| Trp (W) | Aromatic aa | Tyr, Phe |
| Tyr (Y) | Aromatic aa, in particular Phe | Phe, Trp, Thr, Ser |
| Val (V) | Hydrophobic, in particular | Leu, Ile, Ala, Met, Phe |
| | aliphatic aa | |

Substantial modifications in the biological properties of the binding construct of the present invention are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Non-conservative substitutions will usually entail exchanging a member of one of the above defined amino acid classes (such as polar, neutral, acidic, basic, aliphatic, aromatic, small . . . ) for another class. Any cysteine residue not involved in maintaining the proper conformation of the binding construct may be substituted, generally with serine, to improve the oxidative stability of the binding construct.

In addition to the above described substitutions, other substitutions within the CDRs that contribute to binding can be made. For example, the consensus sequences set forth in Table 25 herein provide such substitutions. In certain embodiments, deletions as guided by the provided consensus sequences may also be made. With the guidance provided herein by the consensus sequences set forth in Table 25, various amino acid substitutions, and functional equivalents thereof, or deletions, can be readily made by one of ordinary skill in the art.

Sequence identity, homology and/or similarity of amino acid sequences is determined by using standard techniques known in the art, including, but not limited to, the local sequence identity algorithm of Smith and Waterman, 1981, Adv. Appl. Math. 2:482, the sequence identity alignment algorithm of Needleman and Wunsch (J Mol Biol. 1970 March; 48(3):443-53), the search for similarity method of Pearson and Lipman (Proc Natl Acad Sci USA. 1988 April; 85(8):2444-8), computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al. (Nucleic Acids Res. 1984 Jan. 11; 12(1 Pt 1):387-95), preferably using the default settings, or by inspection. It is envisaged that percent identity is calculated by FastDB based upon the following parameters: mismatch penalty of 1; gap penalty of 1; gap size penalty of 0.33; and joining penalty of 30. See also “Current Methods in Sequence Comparison and Analysis,” Macromolecule Sequencing and Synthesis, Selected Methods and Applications, pp 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng and Doolittle (J Mol Evol. 1987; 25(4):351-60); the method is similar to that described by Higgins and Sharp (Comput Appl Biosci. 1989 April; 5(2):151-3). Useful PILEUP parameters include a default gap weight of 3.00, a default gap length weight of 0.10, and weighted end gaps. Another example of a useful algorithm is the BLAST algorithm, described in: Altschul et al. (J Mol Biol. 1990 Oct. 5; 215(3):403-10.); Altschul et al., (Nucleic Acids Res. 1997 Sep. 1; 25(17):3389-402); and Karlin and Altschul (Proc Natl Acad Sci USA. 1993 Jun. 15; 90(12):5873-7). A particularly useful BLAST program is the WU-Blast-2 program which was obtained from Altschul et al., (Methods Enzymol. 1996; 266:460-80). WU-Blast-2 uses several search parameters, most of which are set to the default values. The adjustable parameters are set with the following values: overlap span=1, overlap fraction=0.125, word threshold (T)=II. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al. (Nucleic Acids Res. 1997 Sep. 1; 25(17):3389-402). Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to about 22 bits.

### Nucleotides Encoding the Binding Constructs

The invention provides a polynucleotide/nucleic acid molecule encoding a binding construct of the invention. Nucleic acid molecules are biopolymers composed of nucleotides. A polynucleotide is a biopolymer composed of 13 or more nucleotide monomers covalently bonded in a chain. DNA (such as cDNA) and RNA (such as mRNA) are examples of polynucleotides/nucleic acid molecules with distinct biological function. Nucleotides are organic molecules that serve as the monomers or subunits of nucleic acid molecules like DNA or RNA. The nucleic acid molecule or polynucleotide of the present invention can be double stranded or single stranded, linear or circular. It is envisaged that the nucleic acid molecule or polynucleotide is comprised in a vector. It is furthermore envisaged that such vector is comprised in a host cell. Said host cell is, e.g. after transformation or transfection with the vector or the polynucleotide/nucleic acid molecule of the invention, capable of expressing the binding construct. For this purpose, the polynucleotide or nucleic acid molecule is operatively linked with control sequences.

The genetic code is the set of rules by which information encoded within genetic material (nucleic acids) is translated into proteins. Biological decoding in living cells is accomplished by the ribosome which links amino acids in an order specified by mRNA, using tRNA molecules to carry amino acids and to read the mRNA three nucleotides at a time. The code defines how sequences of these nucleotide triplets, called codons, specify which amino acid will be added next during protein synthesis. With some exceptions, a three-nucleotide codon in a nucleic acid sequence specifies a single amino acid. Because the vast majority of genes are encoded with exactly the same code, this particular code is often referred to as the canonical or standard genetic code.

Degeneracy of codons is the redundancy of the genetic code, exhibited as the multiplicity of three-base pair codon combinations that specify an amino acid. Degeneracy results because there are more codons than encodable amino acids. The codons encoding one amino acid may differ in any of their three positions; however, more often than not, this difference is in the second or third position. For instance, codons GAA and GAG both specify glutamic acid and exhibit redundancy; but, neither specifies any other amino acid and thus demonstrate no ambiguity. The genetic codes of different organisms can be biased towards using one of the several codons that encode the same amino acid over the others—that is, a greater frequency of one will be found than expected by chance. For example, leucine is specified by six distinct codons, some of which are rarely used. Codon usage tables detailing genomic codon usage frequencies for most organisms are available. Recombinant gene technologies commonly take advantage of this effect by implementing a technique termed codon optimization, in which those codons are used to design a polynucleotide which are preferred by the respective host cell (such as a cell of human hamster origin, an Escherichia coli cell, or a Saccharomyces cerevisiae cell), e.g. in order to increase protein expression. It is hence envisaged that the polynucleotides/nucleic acid molecules of the present disclosure are codon optimized. Nevertheless, the polynucleotide/nucleic acid molecule encoding a binding construct of the invention may be designed using any codon that encodes the desired amino acid.

In line herewith, the term “percent (%) nucleic acid sequence identity/homology/similarity” with respect to the nucleic acid sequence encoding the binding constructs identified herein is defined as the percentage of nucleotide residues in a candidate sequence that are identical with the nucleotide residues in the coding sequence of the binding construct. One method to align two sequences and thereby determine their homology uses the BLASTN module of WU-Blast2 set to the default parameters, with overlap span and overlap fraction set to 1 and 0.125, respectively. Generally, the nucleic acid sequence homology, similarity, or identity between the nucleotide sequences encoding individual variant CDRs and the nucleotide sequences depicted herein are at least 60%, and more typically with increasing homologies, similarities or identities of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%, and almost 100%. Again, the same applies to nucleic acid sequence encoding the “variant VH” and/or “variant VL”.

In one embodiment, the percentage of identity to human germline of the binding constructs according to the invention, or of the first and second domain (binding domains) of these binding constructs, is 70% or 75%, more preferably 80% or 85%, even more preferably 90%, and most preferably 91%, 92%, 93%, 94%, 95% or even 96%. Identity to human antibody germline gene products is thought to be an important feature to reduce the risk of therapeutic proteins to elicit an immune response against the drug in the patient during treatment. Hwang W. Y. and Foote J. (Methods. 2005 May; 36(1):3-10) demonstrate that the reduction of non-human portions of drug binding constructs leads to a decrease of risk of inducing anti-drug antibodies in the patients during treatment. By comparing an exhaustive number of clinically evaluated antibody drugs and the respective immunogenicity data, the trend is shown that humanization of the variable regions of antibodies/binding constructs makes the protein less immunogenic (average 5.1% of patients) than antibodies/binding constructs carrying unaltered non-human variable regions (average 23.59% of patients). A higher degree of identity to human sequences is hence desirable for protein therapeutics based on variable regions and in the form of binding constructs. For the purpose of determining the germline identity, the V-regions of VL can be aligned with the amino acid sequences of human germline V segments and J segments (http://www2.mrc-imb.cam.ac.uk/vbase/) using Vector NTI software and the amino acid sequence calculated by dividing the identical amino acid residues by the total number of amino acid residues of the VL in percent. The same can be done for the VH segments (http://www2.mrc-imb.cam.ac.uk/vbase/) with the exception that the VH CDR3 may be excluded due to its high diversity and a lack of existing human germline VH CDR3 alignment partners. Recombinant techniques can then be used to increase sequence identity to human antibody germline genes. According to one embodiment, the polynucleotide/nucleic acid molecule of the present invention encoding the binding construct of the invention is in the form of one single molecule or in the form of two or more separate molecules. If the binding construct of the present invention is a single chain binding construct, the polynucleotide/nucleic acid molecule encoding such construct will most likely also be in the form of one single molecule.

The same applies for the vector comprising a polynucleotide/nucleic acid molecule of the present invention. If the binding construct of the present invention is a single chain binding construct, one vector may comprise the polynucleotide which encodes the binding construct in one single location (as one single open reading frame, ORF). One vector may also comprise two or more polynucleotides/nucleic acid molecules at separate locations (with individual ORFs), each one of them encoding a different component of the binding construct of the invention. It is envisaged that the vector comprising the polynucleotide/nucleic acid molecule of the present invention is in the form of one single vector or two or more separate vectors. In one embodiment, and for the purpose of expressing the binding construct in a host cell, the host cell of the invention should comprise the polynucleotide/nucleic acid molecule encoding the binding construct or the vector comprising such polynucleotide/nucleic acid molecule in their entirety, meaning that all components of the binding construct—whether encoded as one single molecule or in separate molecules/locations—will assemble after translation and form together the biologically active binding construct of the invention.

The invention also provides a vector comprising a polynucleotide/nucleic acid molecule of the invention. A vector is a nucleic acid molecule used as a vehicle to transfer (foreign) genetic material into a cell, usually for the purpose of replication and/or expression. The term “vector” encompasses—but is not restricted to—plasmids, viruses, cosmids, and artificial chromosomes. Some vectors are designed specifically for cloning (cloning vectors), others for protein expression (expression vectors). So-called transcription vectors are mainly used to amplify their insert. The manipulation of DNA is normally conducted on E. coli vectors, which contain elements necessary for their maintenance in E. coli. However, vectors may also have elements that allow them to be maintained in another organism such as yeast, plant or mammalian cells, and these vectors are called shuttle vectors. Insertion of a vector into the target or host cell is usually called transformation for bacterial cells and transfection for eukaryotic cells, while insertion of a viral vector is often called transduction.

In general, engineered vectors comprise an origin of replication, a multicloning site and a selectable marker. The vector itself is generally a nucleotide sequence, commonly a DNA sequence, that comprises an insert (transgene) and a larger sequence that serves as the “backbone” of the vector. While the genetic code determines the polypeptide sequence for a given coding region, other genomic regions can influence when and where these polypeptides are produced. Modern vectors may therefore encompass additional features besides the transgene insert and a backbone: promoter, genetic marker, antibiotic resistance, reporter gene, targeting sequence, protein purification tag. Vectors called expression vectors (expression constructs) specifically are for the expression of the transgene in the target cell, and generally have control sequences.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, a Kozak sequence and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the nucleotide sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Transfection” is the process of deliberately introducing nucleic acid molecules or polynucleotides (including vectors) into target cells. The term is mostly used for non-viral methods in eukaryotic cells. Transduction is often used to describe virus-mediated transfer of nucleic acid molecules or polynucleotides. Transfection of animal cells typically involves opening transient pores or “holes” in the cell membrane, to allow the uptake of material. Transfection can be carried out using biological particles (such as viral transfection, also called viral transduction), chemical-based methods (such as using calcium phosphate, lipofection, Fugene, cationic polymers, nanoparticles) or physical treatment (such as electroporation, microinjection, gene gun, cell squeezing, magnetofection, hydrostatic pressure, impalefection, sonication, optical transfection, heat shock).

The term “transformation” is used to describe non-viral transfer of nucleic acid molecules or polynucleotides (including vectors) into bacteria, and also into non-animal eukaryotic cells, including plant cells. Transformation is hence the genetic alteration of a bacterial or non-animal eukaryotic cell resulting from the direct uptake through the cell membrane(s) from its surroundings and subsequent incorporation of exogenous genetic material (nucleic acid molecules). Transformation can be effected by artificial means. For transformation to happen, cells or bacteria must be in a state of competence, which might occur as a time-limited response to environmental conditions such as starvation and cell density, and can also be artificially induced.

Moreover, the invention provides a host cell transformed or transfected with the polynucleotide/nucleic acid molecule of the invention or with the vector of the invention.

As used herein, the terms “host cell” or “recipient cell” are intended to include any individual cell or cell culture that can be or has been recipient of vectors, exogenous nucleic acid molecules and/or polynucleotides encoding the binding construct of the present invention; and/or recipients of the binding construct itself. The introduction of the respective material into the cell is carried out by way of transformation, transfection and the like (vide supra). The term “host cell” is also intended to include progeny or potential progeny of a single cell. Because certain modifications may occur in succeeding generations due to either natural, accidental, or deliberate mutation or due to environmental influences, such progeny may not, in fact, be completely identical (in morphology or in genomic or total DNA complement) to the parent cell, but is still included within the scope of the term as used herein. Suitable host cells include prokaryotic or eukaryotic cells, and also include—but are not limited to—bacteria (such as E. coli), yeast cells, fungi cells, plant cells, and animal cells such as insect cells and mammalian cells, e.g., hamster, murine, rat, macaque or human.

In addition to prokaryotes, eukaryotic microbes such as filamentous fungi or yeast are suitable cloning or expression hosts for the binding construct of the invention. Saccharomyces cerevisiae, or common baker's yeast, is the most commonly used among lower eukaryotic host microorganisms. However, a number of other genera, species, and strains are commonly available and useful herein, such as Schizosaccharomyces pombe, Kluyveromyces hosts such as K. lactis, K. fragilis (ATCC 12424), K. bulgaricus (ATCC 16045), K. wickeramii (ATCC 24178), K. waltii (ATCC 56500), K. drosophilarum (ATCC 36906), K. thermotolerans, and K. marxianus; Yarrowia (EP 402 226); Pichia pastoris (EP 183 070); Candida; Trichoderma reesia (EP 244 234); Neurospora crassa; Schwanniomyces such as Schwanniomyces occidentalis; and filamentous fungi such as Neurospora, Penicillium, Tolypocladium, and Aspergillus hosts such as A. nidulans and A. niger.

Suitable host cells for the expression of a glycosylated binding construct are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, Arabidopsis and tobacco can also be used as hosts. Cloning and expression vectors useful in the production of proteins in plant cell culture are known to those of skill in the art. See e.g. Hiatt et al., Nature (1989) 342: 76-78, Owen et al. (1992) Bio/Technology 10: 790-794, Artsaenko et al. (1995) The Plant J 8: 745-750, and Fecker et al. (1996) Plant Mol Biol 32: 979-986.

However, interest has been greatest in vertebrate cells, and propagation of vertebrate cells in culture (cell culture) has become a routine procedure. Examples of useful mammalian host cell lines are monkey kidney CV1 line transformed by SV40 (such as COS-7, ATCC CRL 1651); human embryonic kidney line (such as 293 or 293 cells subcloned for growth in suspension culture, Graham et al., J. Gen Virol. 36: 59 (1977)); baby hamster kidney cells (such as BHK, ATCC CCL 10); Chinese hamster ovary cells/-DHFR (such as CHO, Urlaub et al., Proc. Natl. Acad. Sci. USA 77: 4216 (1980)); mouse sertoli cells (such as TM4, Mather, Biol. Reprod. 23: 243-251 (1980)); monkey kidney cells (such as CVI ATCC CCL 70); African green monkey kidney cells (such as VERO-76, ATCC CRL1587); human cervical carcinoma cells (such as HELA, ATCC CCL 2); canine kidney cells (such as MDCK, ATCC CCL 34); buffalo rat liver cells (such as BRL 3A, ATCC CRL 1442); human lung cells (such as W138, ATCC CCL 75); human liver cells (such as Hep G2,1413 8065); mouse mammary tumor (such as MMT 060562, ATCC CCL-51); TRI cells (Mather et al., Annals N. Y Acad. Sci. (1982) 383: 44-68); MRC 5 cells; FS4 cells; and a human hepatoma line (such as Hep G2).

### Production of Binding Constructs

In a further embodiment, the invention provides a process for producing a binding construct of the invention, said process comprising culturing a host cell of the invention under conditions allowing the expression of the binding construct of the invention and recovering the produced binding construct from the culture.

As used herein, the term “culturing” refers to the in vitro maintenance, differentiation, growth, proliferation and/or propagation of cells under suitable conditions in a medium. Cells are grown and maintained in a cell growth medium at an appropriate temperature and gas mixture. Culture conditions vary widely for each cell type. Typical growth conditions are a temperature of about 37° C., a CO2 concentration of about 5% and a humidity of about 95%. Recipes for growth media can vary e.g. in pH, concentration of the carbon source (such as glucose), nature and concentration of growth factors, and the presence of other nutrients (such as amino acids or vitamins). The growth factors used to supplement media are often derived from the serum of animal blood, such as fetal bovine serum (FBS), bovine calf serum (FCS), equine serum, and porcine serum. Cells can be grown either in suspension or as adherent cultures. There are also cell lines that have been modified to be able to survive in suspension cultures so they can be grown to a higher density than adherent conditions would allow.

The term “expression” includes any step involved in the production of a binding construct of the invention including, but not limited to, transcription, post-transcriptional modification, translation, folding, post-translational modification, targeting to specific subcellular or extracellular locations, and secretion. The term “recovering” refers to a series of processes intended to isolate the binding construct from the cell culture. The “recovering” or “purification” process may separate the protein and non-protein parts of the cell culture, and finally separate the desired binding construct from all other polypeptides and proteins. Separation steps usually exploit differences in protein size, physico-chemical properties, binding affinity and biological activity. Preparative purifications aim to produce a relatively large quantity of purified proteins for subsequent use, while analytical purification produces a relatively small amount of a protein for a variety of research or analytical purposes.

When using recombinant techniques, the binding construct can be produced intracellularly, in the periplasmic space, or directly secreted into the medium. If the binding construct is produced intracellularly, as a first step, the particulate debris, either host cells or lysed fragments, are removed, for example, by centrifugation or ultrafiltration. The binding construct of the invention may e.g. be produced in bacteria such as E. coli. After expression, the construct is isolated from the bacterial cell paste in a soluble fraction and can be purified e.g. via affinity chromatography and/or size exclusion. Final purification can be carried out in a manner similar to the process for purifying a binding construct expressed in mammalian cells and secreted into the medium. Carter et al. (Biotechnology (NY) 1992 February; 10(2):163-7) describe a procedure for isolating antibodies which are secreted to the periplasmic space of E. coli.

Where the antibody is secreted into the medium, supernatants from such expression systems are generally first concentrated using a commercially available protein concentration filter, for example, an ultrafiltration unit.

The binding construct of the invention prepared from the host cells can be recovered or purified using, for example, hydroxylapatite chromatography, gel electrophoresis, dialysis, and affinity chromatography. Other techniques for protein purification such as fractionation on an ion-exchange column, mixed mode ion exchange, HIC, ethanol precipitation, size exclusion chromatography, reverse phase HPLC, chromatography on silica, chromatography on heparin sepharose, chromatography on an anion or cation exchange resin (such as a polyaspartic acid column), immunoaffinity (such as Protein A/G/L) chromatography, chromato-focusing, SDS-PAGE, ultracentrifugation, and ammonium sulfate precipitation are also available depending on the binding construct to be recovered.

A protease inhibitor may be included in any of the foregoing steps to inhibit proteolysis, and antibiotics may be included to prevent the growth of contaminants.

### Formulations

Moreover, the invention provides a composition or formulation comprising a binding construct of the invention or a binding construct produced according to the process of the invention.

Compositions of the invention include, but are not limited to, liquid, frozen, and lyophilized compositions.

The compositions may comprise a pharmaceutically acceptable carrier. In general, as used herein, “pharmaceutically acceptable carrier” means any and all aqueous and non-aqueous solutions, sterile solutions, solvents, buffers, e.g. phosphate buffered saline (PBS) solutions, water, suspensions, emulsions, such as oil/water emulsions, various types of wetting agents, liposomes, dispersion media and coatings, which are compatible with pharmaceutical administration, in particular with parenteral administration. The use of such media and agents in pharmaceutical compositions is well known in the art, and the compositions comprising such carriers can be formulated by well-known conventional methods.

Certain embodiments provide compositions comprising the binding construct of the invention and further one or more excipients such as those illustratively described in this section and elsewhere herein. Excipients can be used in the invention for a wide variety of purposes, such as adjusting physical, chemical, or biological properties of formulations, such as adjustment of viscosity, and or processes of the invention to improve effectiveness and/or to stabilize such formulations and processes against degradation and spoilage e.g. due to stresses that occur during manufacturing, shipping, storage, pre-use preparation, administration, and thereafter. Excipients should in general be used in their lowest effective concentrations.

In certain embodiments, the composition may contain formulation materials for the purpose of modifying, maintaining or preserving certain characteristics of the composition such as the pH, osmolarity, viscosity, clarity, color, isotonicity, odor, sterility, stability, rate of dissolution or release, adsorption or penetration (see, Remington's Pharmaceutical Sciences, 18″ Edition, 1990, Mack Publishing Company). In such embodiments, suitable formulation materials may include, but are not limited to, e.g., amino acids, antimicrobials such as antibacterial and antifungal agents, antioxidants, buffers, buffer systems and buffering agents which are used to maintain the composition at physiological pH or at a slightly lower pH, typically within a pH range of from about 5 to about 8 or 9, non-aqueous solvents, vegetable oils, and injectable organic esters, aqueous carriers including water, alcoholic/aqueous solutions, emulsions or suspensions, including saline and buffered media, biodegradable polymers such as polyesters, bulking agents, chelating agents, isotonic and absorption delaying agents, complexing agents, fillers, carbohydrates, (low molecular weight) proteins, polypeptides or proteinaceous carriers, preferably of human origin, coloring and flavouring agents, sulfur containing reducing agents, diluting agents, emulsifying agents, hydrophilic polymers, salt-forming counter-ions, preservatives, metal complexes, solvents and co-solvents, sugars and sugar alcohols, suspending agents, surfactants or wetting agents, stability enhancing agents, tonicity enhancing agents, parenteral delivery vehicles, or intravenous delivery vehicles.

Different constituents of the composition can have different effects, for example, and amino acid can act as a buffer, a stabilizer and/or an antioxidant; mannitol can act as a bulking agent and/or a tonicity enhancing agent; sodium chloride can act as delivery vehicle and/or tonicity enhancing agent; etc. Salts may be used in accordance with certain embodiments of the invention, e.g. in order to adjust the ionic strength and/or the isotonicity of a composition or formulation and/or to improve the solubility and/or physical stability of a binding construct or other ingredient of a composition in accordance with the invention. Ions can stabilize the native state of proteins by binding to charged residues on the protein's surface and by shielding charged and polar groups in the protein and reducing the strength of their electrostatic interactions, attractive, and repulsive interactions. Ions also can stabilize the denatured state of a protein by binding to, in particular, the denatured peptide linkages (—CONH) of the protein. Furthermore, ionic interaction with charged and polar groups in a protein also can reduce intermolecular electrostatic interactions and, thereby, prevent or reduce protein aggregation and insolubility.

Ionic species differ significantly in their effects on proteins. A number of categorical rankings of ions and their effects on proteins have been developed that can be used in formulating pharmaceutical compositions in accordance with the invention. One example is the Hofmeister series, which ranks ionic and polar non-ionic solutes by their effect on the conformational stability of proteins in solution. Stabilizing solutes are referred to as “kosmotropic”. Destabilizing solutes are referred to as “chaotropic”. Kosmotropes are commonly used at high concentrations to precipitate proteins from solution (“salting-out”). Chaotropes are commonly used to denature and/or to solubilize proteins (“salting-in”). The relative effectiveness of ions to “salt-in” and “salt-out” defines their position in the Hofmeister series.

Free amino acids can be used in formulations or compositions comprising the binding construct of the invention in accordance with various embodiments of the invention as bulking agents, stabilizers, and antioxidants, as well as for other standard uses. Certain amino acids can be used for stabilizing proteins in a formulation, others are useful during lyophilization to ensure correct cake structure and properties of the active ingredient. Some amino acids may be useful to inhibit protein aggregation in both liquid and lyophilized formulations, and others are useful as antioxidants.

Polyols are kosmotropic and are useful as stabilizing agents in both liquid and lyophilized formulations to protect proteins from physical and chemical degradation processes. Polyols are also useful for adjusting the tonicity of formulations and for protecting against freeze-thaw stresses during transport or the preparation of bulks during the manufacturing process. Polyols can also serve as cryoprotectants in the context of the present invention.

Certain embodiments of the formulation or composition comprising the binding construct of the invention can comprise surfactants. Proteins may be susceptible to adsorption on surfaces and to denaturation and resulting aggregation at air-liquid, solid-liquid, and liquid-liquid interfaces. These deleterious interactions generally scale inversely with protein concentration and are typically exacerbated by physical agitation, such as that generated during the shipping and handling of a product. Surfactants are routinely used to prevent, minimize, or reduce surface adsorption.

Surfactants also are commonly used to control protein conformational stability. The use of surfactants in this regard is protein specific, since one specific surfactant will typically stabilize some proteins and destabilize others.

Certain embodiments of the formulation or composition comprising the binding construct of the invention can comprise one or more antioxidants. To some extent deleterious oxidation of proteins can be prevented in pharmaceutical formulations by maintaining proper levels of ambient oxygen and temperature and by avoiding exposure to light. Antioxidant excipients can also be used to prevent oxidative degradation of proteins. It is envisaged that antioxidants for use in therapeutic protein formulations in accordance with the present invention can be water-soluble and maintain their activity throughout the shelf life of the product (the composition comprising the binding construct). Antioxidants can also damage proteins and should hence—among other things—be selected in a way to eliminate or sufficiently reduce the possibility of antioxidants damaging the binding construct or other proteins in the formulation.

Formulations in accordance with the invention may include metal ions that are protein co-factors and that are necessary to form protein coordination complexes, such as zinc necessary to form certain insulin suspensions. Metal ions also can inhibit some processes that degrade proteins. However, metal ions also catalyze physical and chemical processes that degrade proteins. Magnesium ions (10-120 mM) can be used to inhibit isomerization of aspartic acid to isoaspartic acid. Ca+2 ions (up to 100 mM) can increase the stability of human deoxyribonuclease. Mg+2, Mn+2, and Zn+2, however, can destabilize rhDNase. Similarly, Ca+2 and Sr+2 can stabilize Factor VIII, it can be destabilized by Mg+2, Mn+2 and Zn+2, Cu+2 and Fe+2, and its aggregation can be increased by Al+3 ions.

Certain embodiments of the formulation or composition comprising the binding construct of the invention can comprise one or more preservatives. Preservatives are necessary for example when developing multi-dose parenteral formulations that involve more than one extraction from the same container. Their primary function is to inhibit microbial growth and ensure product sterility throughout the shelf-life or term of use of the drug product. Although preservatives have a long history of use with small-molecule parenterals, the development of protein formulations that include preservatives can be challenging. Preservatives very often have a destabilizing effect (aggregation) on proteins, and this has become a major factor in limiting their use in multi-dose protein formulations. Several aspects need to be considered during the formulation and development of preserved dosage forms. The effective preservative concentration in the product must be optimized. This requires testing a given preservative in the dosage form with concentration ranges that confer anti-microbial effectiveness without compromising protein stability.

Development of liquid formulations containing preservatives are more challenging than lyophilized formulations. Freeze-dried products can be lyophilized without the preservative and reconstituted with a preservative containing diluent at the time of use. This shortens the time during which a preservative is in contact with the binding construct, significantly minimizing the associated stability risks. With liquid formulations, preservative effectiveness and stability should be maintained over the entire product shelf-life. An important point to note is that preservative effectiveness should be demonstrated in the final formulation containing the active drug and all excipient components. Once the pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration.

### Kits

In further embodiments, the invention provides a kit comprising a binding construct that binds to MAGEB2 and is useful for detection of MAGEB2 in a tumor sample, wherein the binding construct comprises any of the binding constructs disclosed herein. In one embodiment, the binding construct that is useful for detection of MAGEB2 is a monoclonal antibody that binds to MAGEB2. In further embodiments, the kit further comprises a therapeutic that is effective for treating cancer that expresses MAGEB2. In certain embodiments, the kit can optionally provide a package insert comprising instructions for detecting MAGEB2 and administration of a cancer therapeutic.

In further embodiments, the kit further comprises binding constructs (e.g., antibodies) that are useful to detect different biomarkers other than MAGEB2, e.g., further comprising a MAGEA4 or a MAGEA8 binding construct.

As used herein, the singular forms “a”, “an”, and “the” include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within ±20%, preferably within ±15%, more preferably within ±10%, and most preferably within ±5% of a given value or range. It also includes the concrete value, e.g., “about 50” includes the value “50”.

Throughout this specification and the claims, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein, any of the terms “comprising”, “consisting essentially of” and “consisting of” may be replaced with either of the other two terms.

It should be understood that the above description and the below examples provide exemplary arrangements, but the present invention is not limited to the particular methodologies, techniques, protocols, material, reagents, substances, etc., described herein and as such can vary. The terminology used herein is for the purpose of describing particular embodiments only, and is not intended to limit the scope of the present invention, which is defined solely by the claims. Aspects of the invention are provided in the independent claims. Some optional features of the invention are provided in the dependent claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

A better understanding of the present invention and of its advantages will be obtained from the following examples, offered for illustrative purposes only. The examples are not intended and should not be construed as to limit the scope of the present invention in any way.

### EXAMPLES

### Example 1: MAGEB2 Immunogen Design

Protein sequences of 35 H. sapiens MAGE family members were downloaded from UniProtKB (www.uniprot.org): MAGEA1 (P43355), MAGEA2 (P43356), MAGEA3 (P43357), MAGEA4 (P43358), MAGEA5 (P43359), MAGEA6 (P43360), MAGEA8 (P43361), MAGEA9 (P43362), MAGEA10 (P43363), MAGEA11 (P43364), MAGEA12 (P43365), MAGEB1 (P43366), MAGEB2 (015479), MAGEB3 (015479), MAGEB4 (015481), MAGEB5 (Q9BZ81), MAGEB6 (Q8N7X4), MAGEB10 (Q96LZ2), MAGEB16 (A2A368), MAGEB17 (A8MXT2), MAGEB18 (Q96M61), MAGEC1 (060732), MAGEC2 (Q9UBF10), MAGEC3 (Q8TD91), MAGED1 (Q9Y5V3), MAGED2 (Q9UNF1), MAGED4 (Q96JG8), MAGEE1 (Q9HC15), MAGEE2 (Q8TD90), MAGEH1 (Q9H213), MAGEF1 (Q9HAY2), MAGEL2 (Q9UJ55), NSE3 (Q96MG7), TROP (Q12816), and NECD (Q99608). Clades were determined using the Neighbor-Joining Tree Builder algorithm in Geneious v10.2.

Shannon entropy, a quantitative metric describing the diversity of amino acids observed at a given position in an alignment, was used to select MAGEB2 peptide immunogens having low amino acid conservation across the entire MAGE family and MAGE-B clade (B1, B2, B3, B4, B5, B17). MAGE family or MAGE-B clade members were aligned using MUSCLE (R. C. Edgar. Nucleic Acids Res. 2004; 32(5): 1792-1797). The Shannon entropy was calculated at each position in the alignment using H_{S}(j)=−Σᵢfᵢⱼ log₂ fᵢⱼ, where HS(j) is the Shannon entropy at position j and fij is the frequency of AA i at position j. Shannon entropy was mapped onto the structure of the MAGE homology domain (MHD) of MAGEA4 (PDB ID 2wa0) or a Rosetta homology model of the MHD of MAGEB2 using a custom script that replaces the PDB b-factor of each atom with the normalized Shannon entropy value for its corresponding residue and then visualizing in PyMOL v1.8 (The PyMOL Molecular Graphics System, Version 1.8 Schrödinger, LLC) using color by b-factor.

A custom script was used to deterministically rank regions of length n (n=5-40 amino acids) in the alignment from highest to lowest average Shannon entropy (lowest to highest conservation) using a sliding window approach. For the entire MAGE family, regions of length 10-30 with lowest conservation across the family cluster to three distinct areas: two areas N-terminal to the MHD and one area within the helical N-terminus of the MHD. Analysis of the MAGE-B clade revealed a clade-specific area of low conservation located on a loop at the interface of two globular lobes of the MHD. MAGEB2 peptide immunogens were selected from the following three identified regions of low conservation: the region N-terminal to the MHD, the helical N-terminus of the MHD, and the MDH interface loop region. The peptide ranges within these regions were refined based on average Shannon entropy score, visual inspection of sequence alignments, consideration of structural features such as solvent accessibility and conservation of secondary structure as an isolated peptide as predicted by PSIPRED (Jones D T. (1999) Protein secondary structure prediction based on position-specific scoring matrices. J. Mol. Biol. 292: 195-202), and whether a sequence blast against the non-reductant H. sapiens proteome revealed hits with high homology in non-MAGE proteins. Regions of MAGEB2 selected as peptides immunogens were MAGEB2 a.a.43-76: SSVSGGAASSSPAAGIPQEPQRAPTTAAAAAAGV (N-terminal region peptide) (SEQ ID NO: 2); MAGEB2 a.a.95-125: SSSQASTSTKSPSEDPLTRKSGSLVQFLLYK (MHD N-terminal helix peptide) (SEQ ID NO: 3); and MAGEB2 a.a.185-200: DLTDEESLLSSWDFPR (MHD middle loop peptide) (SEQ ID NO: 562).

Peptides from MAGE homologs with >50% pairwise identity to the MAGEB2 immunogens were selected for experimental counter-screening. Additional MAGEB2 homolog peptides were selected for counter-screening. For MAGEB2 a.a.43-76 (N-terminal region peptide), selected counter screening peptides were derived from MAGE-B1, B4, B17, A4, A1, A5, A12, A6, A3, A2, All, and A8. For MAGE B2 a.a.95-125 (MHD N-terminal helix peptide), selected counter screening peptides were derived from MAGE-B4, B5, B17, C3, All, A8, A9, A3, A10, F1, A4, A5, A12, C2, A2, E1, and C1. For MAGEB2 a.a.185-200 (MHD middle loop peptide), a single counter screening peptide derived from MAGEB1 was selected.

### Example 2: Generation and Selection of Antibodies

### Immunizations

Rabbits were immunized according to standard protocols, and splenocytes were isolated and frozen from immune animals.

### Preparation of Peptides for Antigen-Specific Sorting

27 biotin-labelled MAGE counter-screening peptides (Table 7) were pooled together in equal molar concentration to create a working cocktail. The cocktail was complexed to neutravidin (Fisher: PI31000) at a molar ratio of 4:1 (neutravidin:peptide) in 2% FBS FACS buffer (calcium and magnesium free PBS with 2% v/v FBS). The mixture was left to incubate at 4° C. for 15 minutes. Each of the 3 biotin-labelled MAGEB2 target peptides (N-Terminal Region Peptide, Middle Loop Region Peptide, N-Terminal Helix Peptide, see Table 8) was complexed to a different fluorescently-tagged streptavidin using the same 4:1 (streptavidin:peptide) molar ratio in 2% FBS FACS buffer and then left to incubate at 4° C. for 15 minutes. MAGEB2 N-Terminal Peptide was complexed to BV421 streptavidin (BD: 563259), MAGEB2 Middle Loop Region Peptide was complexed to BV605 streptavidin (BD: 563855), and MAGEB2 N-Terminal Helix Peptide was complexed to Alexa Fluor 647 streptavidin (Jackson: 016-600-084).

Unbound streptavidin or neutravidin was quenched by adding excess amount of D-biotin (Fisher: BP232-1) at molar ratio of 600:1 (D-biotin:streptavidin or neutravidin), the peptides were then left to incubate for another 15 minutes at 4′C. The 3 MAGEB2 target peptides were pooled together just before use.

TABLE 7
27 MAGE counter-screening peptides.
MAGE Counter-Screening Peptides

| | | | SEQ ID |
|---|---|---|---|
| Item | Peptide Name | Peptide Sequence | NO: |
| 1 | A1_CTermHelix | {Biotin-NH} SYVKVLEYVIKVSARVRFFFPSLREAA | 570 |
| | | {COOH} | |
| 2 | A1_NTerminalPeptide | {Biotin-NH} | 571 |
| | | SSPLVLGTLEEVPTAGSTDPPQSPQGASAFPTTINFT | |
| | | {COOH} | |
| 3 | A2_NTermHelix | {Biotin-NH} PDLESEFQAAISRKMVELVHFLLLK | 572 |
| | | {COOH} | |
| 4 | A2_NTerminalPeptide | {Biotin-NH} | 573 |
| | | SSTLVEVTLGEVPAADSPSPPHSPQGASSFSTTINYT | |
| | | {COOH} | |
| 5 | A3_NTermHelix | {Biotin-NH} | 574 |
| | | STFPDLESEFQAALSRKVAELVHFLLLK{COOH} | |
| 6 | A3_NTerminalPeptide | {Biotin-NH} | 575 |
| | | SSTLVEVTLGEVPAAESPDPPQSPQGASSLPTTMNY | |
| | | {COOH} | |
| 7 | A5_NTermHelix | {Biotin-NH} STSPDPESVFRAALSKKVADLIHFLLLK | 576 |
| | | {COOH} | |
| 8 | A5_NTerminalPeptide | {Biotin-NH} | 577 |
| | | SSPLVPGTLGEVPAAGSPGPLKSPQGASAIPTAIDFT | |
| | | {COOH} | |
| 9 | A9_CTermHelix (C19S) | {Biotin-NH} SYEKVINYLVMLNAREPISYPSLYEEV | 578 |
| | | {COOH} | |
| 10 | A9_NTermHelix | {Biotin-NH} | 579 |
| | | SSSVDPAQLEFMFQEALKLKVAELVHFLLHK | |
| | | {COOH} | |
| 11 | A10_NTermHelix | {Biotin-NH} | 580 |
| | | STLQVLPDSESLPRSEIDEKVTDLVQFLLFK | |
| | | {COOH} | |
| 12 | A11_NTermHelix | {Biotin-NH} | 581 |
| | | STSPDLIDPESFSQDILHDKIIDLVHLLLRK {COOH} | |
| 13 | A11_NTerminalPeptide | {Biotin-NH} | 582 |
| | | SSTLNVGTLEELPAAESPSPPQSPQEESFSPTAMDAI | |
| | | {COOH} | |
| 14 | A12_NTermHelix | (Biotin-NH} | 583 |
| | | STFPDLETSFQVALSRKMAELVHFLLLK {COOH} | |
| 15 | A12_NTerminalPeptide | {Biotin-NH} | 584 |
| | | SSTLVEVTLREVPAAESPSPPHSPQGASTLPTTINYT | |
| | | {COOH} | |
| 16 | B1_MiddleLoopRegion | {Biotin-NH} NLTNDGNLSNDWDFPR {COOH} | 585 |
| 17 | B1_NTerminalPeptide | {Biotin-NH} | 586 |
| | | SSPVLGDTPTSSPAAGIPQKPQGAPPTTTAAAAV | |
| | | {COOH} | |
| 18 | B4_NTermHelix | {Biotin-NH} | 587 |
| | | SSSQASTSTERSLKDSLTRKTKMLVQFLLYK | |
| | | {COOH} | |
| 19 | B4_NTerminalPeptide | {Biotin-NH} | 588 |
| | | SSVLRDTASSSLAFGIPQEPQREPPTTSAAAA | |
| | | {COOH} | |
| 20 | B5_NtermHelix (C12S) | (Biotin-NH} | 589 |
| | | SSEVSPSTESSSSNFINIKVGLLEQFLLYK {COOH} | |
| 21 | B17_NTermHelix | {Biotin-NH} | 590 |
| | | NSFHGPSSSESTGRDLLNTKTGELVQFLLNK | |
| | | {COOH} | |
| 22 | B17_NTerminalPeptide | {Biotin-NH} | 591 |
| | (C5S) | SSPASQSPPQSFPNAGIPQESQRASYPSSPASAV | |
| | | {COOH} | |
| 23 | C1_NTermHelix | {Biotin-NH} | 592 |
| | | TDSESLIESEPLFTYTLDEKVDELARFLLLK | |
| | | {COOH} | |
| 24 | C2_NTermHelix (C3S) | {Biotin-NH} | 593 |
| | | GTSQGLPDSESSFTYTLDEKVAELVEFLLLK | |
| | | {COOH} | |
| 25 | C3_NTermHelix | {Biotin-NH} | 594 |
| | | HALPESESLPRYALDEKVAELVQFLLLK {COOH} | |
| 26 | E1_NTermHelix | {Biotin-NH} | 595 |
| | | NTSRVAITLKPQDPMEQNVAELLQFLLVK {COOH} | |
| 27 | F1_NTermHelix | (Biotin-NH} | 596 |
| | | ALAAKALARRRAYRRLNRTVAELVQFLLVK | |
| | | {COOH} | |

TABLE 8
3 MAGEB2 Target Peptides
MAGEB2 Target Peptides

| | | | SEQ ID |
|---|---|---|---|
| Item | Peptide Name | Peptide Sequence | NO |
| 1 | B2_MiddleLoopRegion | {Biotin-NH} DLTDEESLLSSWDFPR {COOH} | 597 |
| 2 | B2_NTermHelix | {Biotin-NH} | 598 |
| | | SSSQASTSTKSPSEDPLTRKSGSLVQFLLYK | |
| | | {COOH} | |
| 3 | B2_NTerminalPeptide | {Biotin-NH} | 599 |
| | | SSVSGGAASSSPAAGIPQEPQRAPTTAAAAAAGV | |
| | | {COOH} | |

### Preparation of Rabbit Splenocytes for Antigen-Specific Sorting

Rabbit splenocytes were thawed into 10% ICM (Immune Cell Culture Medium) and dead cells were removed using the Miltenyi Biotec MACS Dead Cell Removal Kit (Miltenyi: 130-090-101). The cells were then washed in 10 mL of cold 2% FBS FACS buffer. After a 4-minute centrifugation at 400G, the supernatant was removed using a serological pipette and the cocktail of neutravidin-complexed MAGE counter-screening peptides was added. The cells were left to incubate at 4° C. for 30 minutes, at which point the 3 streptavidin-complexed MAGEB2 target peptides along with 5 μg of FITC-conjugated goat anti-rabbit IgG Fc antibody (Serotec: STAR121F) were added to stain IgG-expressing, antigen-specific cells. The final concentrations of the peptides in the 1 mL reaction volume were: 120 nM for the cocktail of counter-screening peptides, and 16.5 nM for each target peptide. The cells were incubated for another 30 minutes at 4° C., then washed in 10 mL of cold 2% FBS FACS buffer and centrifuged again. After removing the supernatant, the cells were resuspended in 2 mL of 10% ICM and then put through a 40 μm cell strainer to remove any clumps. 10 μL of 7-AAD viability staining solution (Fisher: 00-6993-50) was added to the cells 10 minutes prior to sorting.

### Sorting and Culturing of Antigen-Specific Cells

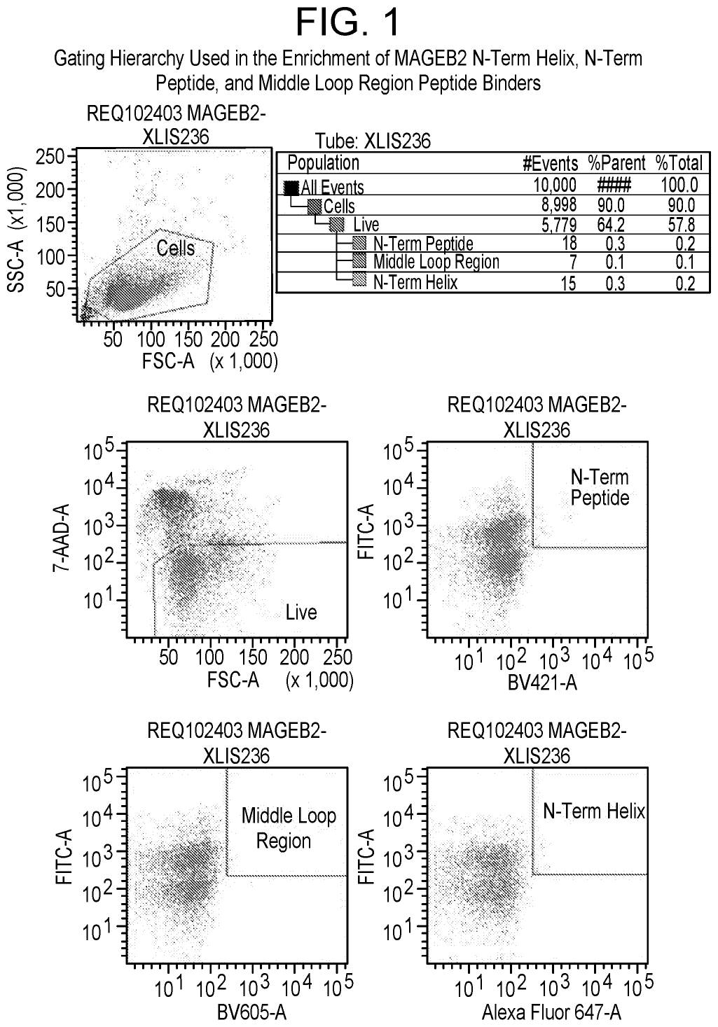

Cells were sorted on a BD FACSAria III equipped with 405 nm laser (detection: 450/50 nm, 610/20 nm), 488 nm laser (detection: 530/30 nm, 695/40 nm), and 640 nm laser (detection: 730/45 nm) using a 100 micron nozzle at 20 Psi sheath pressure. Dead cells were excluded by gating on population not fluorescent in 7-AAD, and MAGEB2 target peptide binders were sorted by drawing 3 gates using the following selection criteria: FITC+/BV421+, FITC+/BV605+, FITC+/Alexa Fluor 647+(see FIG. 1). An initial sort was performed using Yield precision mode to enrich target populations from ˜0.1% to ˜50%, this was followed by a second round of sorting using Purity precision mode to single cell sort MAGEB2 target peptide binders into 384-well tissue culture plates pre-filled with 10% ICM supplemented with 4% rabbit T-cell supernatant, 2 μL/mL of anti-rabbit IgG microbeads (Miltenyi: 130-048-602), and gamma-ray-irradiated EL4 cells at a density of 437,500 cells/mL. The plates were stored in tissue culture incubator (37° C., 5% CO2), and outgrowth was assessed 7 days later by visual scoring using an inverted microscope. After 8 days, supernatants were collected from the tissue culture plates and the cells were lysed in Buffer RLT (Qiagen: 79216) and frozen at −80° C.

### Initial Detection of MAGEB2 Antibodies

B-cell culture supernatants were screened for the presence of positive secreting antibodies using a rabbit IgG capture ELISA. Goat anti rabbit IgG (Fc) (Jackson, Cat. No. 111-005-046) was coated in 1× phosphate buffered saline (PBS) overnight at 4° C. onto Corning 3702 384 well polystyrene plates. Plates were washed 4 cycles using a Titertek plate washer and blocked with 1×PBS/1% milk. Then the plates were aspirated and B-cell culture supernatants were diluted at 1:5 final in 1×PBS/1% milk and incubated for 1 hour at room temperature. Plates were washed 4 cycles and then horseradish peroxidase (HRP) conjugated goat anti human IgG (Fc) (Jackson, Cat. No. 111-035-046) made up in 1×PBS/1% milk was added and incubated for 1 hour at room temperature. Plates were washed 4 cycles, then 1 step TMB (Neogen cat. No. 319177) was added for 30 minutes at room temperature and subsequently quenched with 1N hydrochloric acid. Plates were read on the Multiscan Ascent plate reader at OD 450 nm and a total of 129 rabbit antibodies were positive with the breakdown of hits per region represented in Table 9 below.

TABLE 9

| Binding region peptide | Total # Positive |
|---|---|
| MAGEB2 middle loop region peptide | 50 |
| MAGEB2 N-term helix peptide | 35 |
| MAGEB2 N-term peptide | 45 |

### Identification of MAGEB2 Specific Antibodies

MAGEB2 antibodies were tested for binding using biotinylated MAGEB2 peptides coated on xMAP LumAvidin Microspheres or Luminex beads (Luminex Cat No. L100-LXXX-01). These avidin coupled microparticles are color coded into different spectrally distinct regions, with unique fluorescent dye combinations allowing for non-covalent binding of biotinylated proteins. To validate the rabbit IgG capture ELISA results in the first bead-based FACS assay the panel of 129 rabbit antibodies was tested against the same 3 peptides that the rabbits were immunized with: MAGEB2 middle loop region peptide, MAGEB2 N-terminal helix peptide and MAGEB2 N-terminal region peptide. The three biotinylated peptides were coated using three different LumAvidin beads in FACS buffer [1×PBS+2% fetal bovine serum (Hyclone Cat No. SH30396.03)] for 30 minutes at room temperature in the dark. The beads were then washed twice in FACS buffer by centrifugation for 2 min at 3500 RPM. The coated beads were resuspended and pooled together (3-plex) and added to the B-cell culture supernatants at a 1:2 final dilution onto Corning 3897 96 well V-bottom polystyrene plates and incubated for 1 hour at room temperature in the dark. Another 2 washes in FACS buffer were done before the addition of Alexa 488 conjugated goat anti rabbit IgG (Fc) (Jackson, Cat No. 111-545-046) made up in FACS buffer was added and incubated for 15 minutes at room temperature in the dark. A final wash was done before plates were read on the iQue FACS machine with Intellicyte autosampler to obtain flow cytometry measurements according to the manufacturer's recommendations; data analysis was done on Intellicyt ForeCyt® Enterprise Client Edition 6.2 (R3). A total of 82 wells were identified as MAGEB2 positive with the breakdown of hits per region as presented in Table 10 below.

TABLE 10

| Binding region | Total # Positive |
|---|---|
| MAGEB2 middle loop region peptide | 21 |
| MAGEB2 N-term helix peptide | 24 |
| MAGEB2 N-term region peptide | 37 |

In the second bead-based FACS assay the panel of 129 rabbit antibodies was tested against 9 other peptides of similar sequence to determine MAGEB2 specificity: MAGEB1 middle loop region peptide, MAGEB4 N-term helix peptide, MAGEB5 N-term helix peptide, MAGE17 N-term helix peptide, MAGEF1 N-term helix peptide, MAGEE1 N-term helix peptide; MAGEB1 N-term peptide peptide, MAGEB4 N-term region peptide and MAGEB17 N-term region peptide. The nine biotinylated peptides were coated using nine different LumAvidin beads in FACS buffer [1×PBS+2% fetal bovine serum (FBS)] for 30 minutes at room temperature in the dark. The beads were then washed twice in FACS buffer by centrifugation for 2 min at 3500 RPM. The coated beads were resuspended and pooled together (9-plex) and added to the rabbit B-cell supernatants at a 1:2 final dilution onto Corning 3897 96 well V-bottom polystyrene plates and incubated for 1 hour at room temperature in the dark. Another 2 washes in FACS buffer were done before the addition of Alexa 488 conjugated goat anti rabbit IgG (Fc) (Jackson, Cat No. 111-545-046) made up in FACS buffer was added and incubated for 15 minutes at room temperature in the dark. A final wash was done before plates were read on the iQue FACS machine with Intellicyte autosampler to obtain flow cytometry measurements according to the manufacturer's recommendations; data analysis was done on Intellicyt ForeCyt® Enterprise Client Edition 6.2 (R3). A total of 43 wells were identified as MAGEB2 specific with the breakdown of hits per region as represented in Table 11 below. All 43 wells underwent heavy chain and light chain sequencing.

TABLE 11

| Binding region | Total # Positive |
|---|---|
| MAGEB2 middle loop region peptide | 12 |
| MAGEB2 N-term helix peptide | 19 |
| MAGEB2 N-term region peptide | 12 |

### Identification of MAGEB2 Highest Affinity Antibodies

To determine which of the 43 MAGEB2 specific antibodies to take forward to cloning, a limited antigen (LA) kinetics FACS assay done on LumAvidin beads using full length MAGEB2 protein, to rank the panel and select the highest affinity antibodies.

huMAGEB2(1-319) in pET23 was transformed into BL21(DE)-star pLysS strain (Invitrogen). Single colonies were used to inoculate 2 mL Luria Broth supplemented with carbenicillin (50 μg/mL) and chloramphenicol (34 μg/mL) and grown shaking at 37° C. for several hours until turbid. Two hundred microliters of culture were used to inoculate 400 mL of Luria Broth (Teknova) supplemented with selection antibiotics and antifoam 204 (Sigma). These cultures were grown shaking at 24° C. overnight. Turbid cultures were used to seed 1-liter Terrific Broth (Teknova) supplemented with 1% (w/v) glucose, 2 mM MgSO₄, 17 μg/L carbenicillin, 12 μg/L chloramphenicol and antifoam 204 (in 4 L baffled shake flasks) to an OD₆₀₀=0.2. These cultures were grown shaking at 37° C. until an OD₆₀₀=1.0, then were shifted to 16° C. and continued to grow to OD₆₀₀=2.0. At this point, protein expression was induced, with 100 μM IPTG, for 16 hrs. The cells were pelleted by centrifugation and stored at −80° C. until processing.

The MAGEB2 pellets were resuspended in Lysis Buffer (100 mM TRIS pH 8.0, 500 mM NaCl, 15 mM bME, 5 mM Benzamidine, 0.01 mg/mL lysozyme, 0.05 mg/mL Dnase1, 5% (v/v) glycerol, and Halt Protease Inhibitor Tablets (Pierce)) using a Polytron PT2100 homogenizer. The homogenized cells were lysed using Microfluidizer M-110Y (Mircofluidics) in three passes. The lysate was clarified by centrifugation at 13,000 rpm in a Beckman Centrifuge in a JLA-16.250 rotor. The clarified lysate was filtered through 0.22 μm filter before being applied to HisTrap Excel column (Cytiva). The resin was washed with 25 mM TRIS pH 8.5, 0.5M NaCl, 10 mM BME, 5% glycerol, 10 mM imidazole and MAGEB2 was eluted with 25 mM TRIS pH 8.5, 0.5M NaCl, 10 mM BME, 5% glycerol, 500 mM imidazole.

The affinity captured MAGEB2 was dialyzed against 20 mM TRIS pH 8.5, 0.5M NaCl, 5% (v/v) glycerol, 1 mM TCEP and diluted 25-fold into 20 mM TRIS pH 8.5, 5% (v/v) glycerol, 1 mM TCEP before ion-exchange chromatography using Q-sepharose HP (Cytiva). Bound MAGEB2 was eluted of the column using a 20 mM to 500 mM NaCl gradient over 20 CV. Peak fractions were analyzed by SDS-PAGE and pooled based on the absence of low-molecular weight bands. The n-terminal 6×His tag SEQ ID NO: 569) was removed from MAGEB2 using caspase 3 protease at a 1:100 (w/w) ratio, after the addition of 1 mM EDTA and 0.1% CHAPS. The digest proceeded overnight at 4° C. The digest was dialyzed against 20 mM TRIS pH 8.5, 0.25M NaCl, 1 mM TCEP to remove CHAPS and EDTA before ion-exchange chromatography.

Dialyzed MAGEB2 was diluted 10-fold into 20 mM TRIS pH 8.5, 1 mM TCEP and applied to Q-sepharose HP resin (Cytiva). Bound MAGEB2 was eluted of the column using a 20 mM to 500 mM NaCl gradient over 20 CV. Peak fractions were analyzed by SDS-PAGE and pooled based on the absence of high-molecular weight bands. This pool was biotinylated using recombinant BirA with 50 mM Bicine pH 8.3, 10 mM Mg-acetate, 10 mM ATP and 50 μM d-biotin. The reaction proceeded overnight at 4° C. and 5 μg of protein was analyzed by Mass spec (ask Dylan for the about instrument and methods) to monitor biotin addition to MAGEB2.

The biotinylation reaction was dialyzed against 20 mM TRIS pH 8.5, 150 mM NaCl, 1 mM TCEP to remove excess ATP and biotin. After dialysis the sample was diluted 10-fold, in 20 mm TRIS pH 8.5, 1 mM TCEP, and applied to Q-sepharose HP (Cytiva). Bound MAGEB2 was eluted off the column using a 20 mM to 500 mM NaCl gradient over 20 CV. Peak fractions were analyzed by SDS-PAGE and pooled based on the presence of a 37 Kd band. The pooled fractions were concentrated using 10 Kd MWCO spin filters (Sartorius). The concentrated MAGEB2 was dialyzed against 30 mM HEPES pH 8.2, 0.15M NaCl, 1 mM TCEP, filtered through 0.22 μm Posidyne syringe filter (Pall) and stored at −80° C.

During purification, mass spec analysis was used to confirm the identity of intact MAGEB2, caspase-3 cleaved MAGEB2, and biotinylated MAGEB2. The homogeneity of purification intermediates and final protein lot was measured by sizing HPLC analysis on Agilent 1200 LC system using a YARA 3 μm SEC 2000 column (Phenomenex). Protein concentration was determined by A280 absorbance using Spectramax spectrophotometer (Molecular Devices).

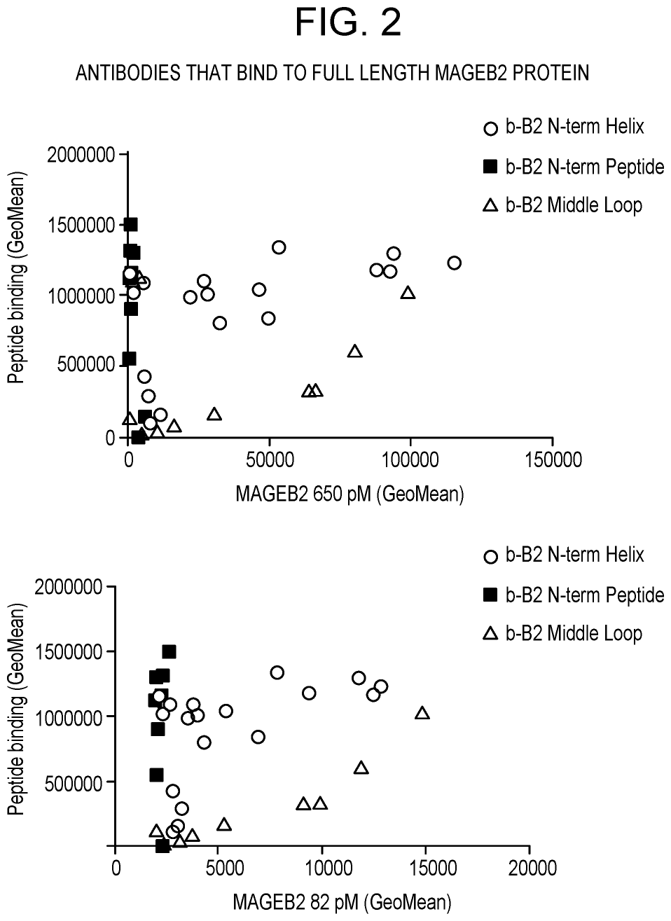

Biotinylated MAGEB2 full length protein was titrated 1:2 across 6 different limited antigen concentrations and then coated onto six different LumAvidin beads in FACS buffer [1×PBS+2% fetal bovine serum (Hyclone Cat No. SH30396.03)] for 30 minutes at room temperature in the dark. The beads were then washed twice in FACS buffer by centrifugation for 2 min at 3500 RPM. The coated beads were resuspended and pooled together (6-plex) and added to the rabbit B-cell supernatants at a 1:2 final dilution onto Corning 3897 96 well V-bottom polystyrene plates and incubated overnight at room temperature in the dark. The next day 2 washes were done in FACS buffer before the addition of Alexa 488 conjugated goat anti rabbit IgG (Fc) (Jackson, Cat No. 111-545-046) made up in FACS buffer was added and incubated for 15 minutes at room temperature in the dark. A final wash was done before plates were read on the iQue FACS machine with Intellicyte autosampler to obtain flow cytometry measurements according to the manufacturer's recommendations; data analysis was done on Intellicyt ForeCyt® Enterprise Client Edition 6.2 (R3). The LA data was aligned with the sequence data and a total of 24 highest affinity, sequence diverse antibodies were identified, with the breakdown of hits per region as shown in Table 12 below and in FIG. 2 herein.

TABLE 12

| Binding region | Total # Positive |
|---|---|
| MAGEB2 middle loop region peptide | 6 |
| MAGEB2 N-term helix peptide | 16 |
| MAGEB2 N-term region peptide | 2 |

### Heavy and Light Chain Sequencing of Antibodies

Messenger RNA (mRNA) was purified from wells containing the antibody-producing cells using the mRNA catcher plus kit (Invitrogen). Purified RNA was used to amplify the antibody heavy and light chain variable region (V) genes using cDNA synthesis via reverse transcription, followed by a polymerase chain reaction (RT-PCR). The rabbit antibody gamma heavy chain was obtained using the Qiagen One Step Reverse Transcriptase PCR kit (Qiagen). This method was used to generate the first strand cDNA from the RNA template and then to amplify the variable region of the gamma heavy chain using multiplex PCR. The 5′ gamma chain-specific primer annealed to the signal sequence of the antibody heavy chain, while the 3′ primer annealed to a region of the gamma constant domain. The rabbit kappa light chain was obtained using the One Step Reverse Transcriptase PCR kit (Qiagen). This method was used to generate the first strand cDNA from the RNA template and then to amplify the variable region of the kappa light chain using multiplex PCR. The 5′ kappa light chain-specific primer annealed to the signal sequence of the antibody light chain while the 3′ primer annealed to a region of the kappa constant domain.

The PCR product was sequenced and amino acid sequences were deduced from the corresponding nucleic acid sequences bioinformatically. Two additional, independent RT-PCR amplification and sequencing cycles were completed for each sample in order to confirm that any mutations observed were not a consequence of the PCR. The derived amino acid sequences were then analyzed to determine the germline sequence origin of the antibodies and to identify deviations from the germline sequence. A comparison of each of the heavy and light chain sequences to their original germline sequences are indicated in sequence Table 4 herein. The amino acid sequences were used to group the clones by similarity.

The variable region kappa light chain DNA sequences for the selected antibodies were made as a synthetic genes. These synthetic genes were cloned with the rabbit kappa constant region in to a pTTS based expression vector. The variable region heavy chain DNA sequences for the selected antibodies were made as synthetic genes. These synthetic genes were cloned with a rabbit IgG constant region in to a pTTS based expression vector. After cloning, these antibodies were expressed in HEK2936E cells transfected with PEI.

### Example 3: Immunohistochemical Assays

### Objectives

To assess the specificity of twenty-four rabbit anti-human MAGEB2 antibodies to human MAGEB2, and potential cross reactivity for other MAGE-B and MAGE-A family members by immunohistochemical (IHC)staining.

### Overview Summary

Twenty-four rabbit-anti human MAGEB2 supernatants were assessed for binding to human MAGEB2 on transfected human MAGEB2+ control cells by IHC. After initial screening assays, small batch purified antibody aliquots from four MAGEB2 clones (4G17, 1J15, 1C3, 1I14) with desired attributes were assessed on transfected human MAGE-A+ and MAGE-B+ family member control cells, normal human testes tissues, normal human tissue TMAs, and thirteen human cancer cell lines with known endogenous MAGEB2 RNA expression level by IHC. MAGEB2 antibody clones 1I14 and 1C3 displayed properties that were desired for IHC.

### Materials and Methods

### Control Cells and Tissues

Negative control cells included parental Chinese hamster ovary (CHO) cells, CHO cells stably transfected with mCherry Vector Control (minus Myc/DDK tags), CHO cells stably transfected with mCherry-human DCAF4L2 (plus Myc/DDK tags). The cross-reactivity control cells included CHO cells transfected with mCherry Vector Control (plus Myc/DDK tags) with one of the human MAGE-B or MAGE-A family members (MAGEA1, MAGEA2, MAGEA3, MAGEA4, MAGEA5, MAGEA6, MAGEA8, MAGEA9, MAGEA11, MAGEA12, MAGEB1, MAGEB3, MAGEB4, MAGEB5, MAGEB6, MAGEB10, MAGEB16, MAGEB17, MAGEB18). Positive control cell lines included CHO cells transfected with mCherry Vector (plus Myc/DDK tags) with human MAGE-B2 or CHO cells transfected with mCherry Vector (open reading frame (ORF); minus Myc/DDK tags) with human MAGEB2, and thirteen human cancer cell lines with known endogenous RNA expression level of MAGEB2 (U266B1, T98G, KMM-1, U20S, CFPAC-1, SCaBER, UACC-257, CFPAC-1, VMRC-LCD, NCI-H1703, UM-UC-3, NCI-H1395, NCIH82).

Cell pellets were fixed in 10% neutral buffered formalin, suspended in Histogel™ containing agarose/glycerine (cat #HG-4000-12, Thermo Fisher Scientific, Waltham, MA), and processed to paraffin blocks. Two FFPE normal human testes tissues, and one FFPE normal human liver tissue were used as positive and negative controls, respectively, obtained from Human Tissue Science Center (HTSC).

TABLE 13
Cells and Tissues

| | HTSC | |
|---|---|---|
| | Inventory Code | |
| Species | (Block ID) | Descriptions |
| Hamster | 694001 | Amgen in house TMA contain CHO, CHO− mCherry, |
| | | CHO− mCherry− human DCAF4L2+, CHO− MAGE-A1+, |
| | | MAGE-A2+, MAGE-A3+, MAGE-A4+ (variant 2), |
| | | MAGE-A5+, MAGE-A6+, and MAGE-A8+ cell line |
| Hamster | 697782 | Amgen in house TMA contain CHO, CHO− mCherry, |
| | | CHO− mCherry− human DCAF4L2+, CHO− MAGE-A4+ |
| | | (variant 4), MAGE-A9+, MAGE-A10+, MAGE-A11+ |
| | | (variant 1), MAGE-A11+ (variant 2), MAGE-A12+ |
| | | cell lines |
| Hamster | 697548 | Amgen in house TMA contain CHO, CHO− mCherry, |
| | | CHO− mCherry− human DCAF4L2+, CHO− MAGE-B1+ |
| | | (variant 1), MAGE-B1+ (variant 3), MAGE-B2+ |
| | | (ORF = minus Myc/DDK tags), MAGE-B2 MAGE-B3+, |
| | | MAGE-B5+ (variant 1) cell lines |
| Hamster | 697639 | Amgen in house TMA contain CHO, CHO− mCherry, |
| | | CHO− mCherry− human DCAF4L2+, CHO− MAGE-B5+ |
| | | (variant 2), MAGE-B6+, MAGE-B10+, MAGE-B16+, |
| | | MAGE-17+, MAGE-18+ cell lines |
| Hamster | 694686 | Amgen in house TMA contain CHO, CHO− mCherry, |
| | | CHO− mCherry− human DCAF4L2+, CHO− MAGE-B5+ |
| | | (variant 2), MAGE-B6+, MAGE-B10+, MAGE-B16+, |
| | | MAGE-17+, MAGE-18+ cell lines |
| Human | 627446 | Amgen in house TMA contain U266B1, KMM-1, |
| | | NCI-H1703, SCaBER, T98G, UACC-257 and |
| | | CFPAC-1 cell |
| Human | 685358 | Amgen in house TMA contain NCI-H1703, |
| | | NCI-H1395, VMRC-LCD, SCaBER cell |
| Human | 685359 | Amgen in house TMA contain U2OS, UM-UC-3, |
| | | NCI-H82, CFPAC-1 cell |
| Human | 14749 | Normal Testis |
| Human | 8497 | Normal Testis |
| Human | 356137 | Normal Liver |

### Tissues Microarray

FFPE normal human TMA slides (normal human multiple organ tissue microarray) were obtained from US BioMax (FDA999x, Crabbs Branch Way, Derwood, MD; https://www.biomax.us/FDA999x).

### Antibodies

Twenty-four human MAGEB2 clone supernatants (monoclonal rabbit IgG1 antibody) were assessed for binding to human MAGEB2 and potential cross reactivity for other MAGE-A and MAGE-B family members on control cells by IHC staining. Four of the twenty-four human MAGEB2 clones (4G17, 1J15, 1C3, 1I14) were the most promising candidates for further assessment of MAGEB2 specificity and sensitivity by IHC staining. Small batch purified antibody aliquots from the four MAGEB2 clones (4G17, 1J15, 1C3, 1I14) were assessed on MAGE-A and MAGE-B family cross-reactivity control cells, control testes tissues, and normal human tissue TMAs by IHC. Rabbit Monoclonal IgG1 isotype control (EPR25A) was purchased from Abcam (cat #ab172730, Lot #GR3235749-11, Cambridge, United Kingdom; https://www.abcam.com/rabbit-igg-monoclonal-epr25a-isotype-control-ab172730.html).

### Immunohistochemistry Assay

FFPE samples were sectioned at 4 μm and mounted on positive charged glass slides. After mounting, paraffin sections were air-dried at room temperature. FFPE TMA samples were received as freshly sectioned and mounted on glass slides. The fully automated IHC assay was performed on a Discovery Ultra Stainer (Ventana Medical Systems, Tucson, AZ) using Ventana Anti-Rabbit HQ (cat #760-4815), Ventana Anti-HQ HRP (cat #760-4820), and Ventana Antibody Diluent with Casein (cat #760-219) (Ventana Medical Systems, Tucson, AZ). The slides were baked on the Discovery Ultra Stainer at 60° C. for 8 minutes, de-paraffinized with Ventana Discovery wash (cat #950-510) for 3 cycles (8 minutes each) at 69° C., and underwent cell conditioning (target retrieval) at 95° C. for 16 minutes (for cell arrays) or 32 minutes (tissues or TMAs) in CC1 buffer (cat #950-500). Following this, slides were incubated with Background Sniper (cat #BS966M, Biocare Medical, Pacheco, CA) in a Ventana Option Prep kit (cat #771-751) for 28 minutes, incubated with CM inhibitor (a part of the Ventana DAB kit cat #760-159) for 12 minutes, and incubated for 1 h at RT with MAGE-B2 antibody diluted with Ventana diluent with Casein in a Ventana Prep kit (cat #770-001) (working concentration at 0.1 μg/ml for cell pellet, or 1-5 ug/ml for tissues) or with a matched concentration of isotype control Rabbit IgG1 antibody. Following this, slides were incubated with the Ventana Anti-Rabbit HQ (cat #760-4815) for 28 minutes at RT, Ventana Anti-HQ HRP (cat #760-4820) for 28 minutes at RT, and signals detected with Ventana DAB chromogen kit (cat #760-159) for 5 minutes at RT. Slides were counterstained with Ventana Hematoxylin II (cat #5266726001) for 8 minutes and Ventana Bluing reagent (cat #526676900) for 4 minutes. Slides were coverslipped and digitized using an Aperio AT2 scanner (Leica Biosystems Inc, Buffalo Grove, IL).

TABLE 14
Assay steps.

| Steps on Ventana Ultra | Vendor/Catalog | Time |
|---|---|---|
| Baking | Ventana/NA | 60° C., 8 minutes |
| Deparaffinization | Ventana/950-510 | 69° C., 3 cycles |
| | | (8 minutes each) |
| Antigen Retrieval | Ventana/950-500 | CC1, 95° C., 16 |
| | | minutes (for FFPE cell |
| | | pellets) or 32 minutes |
| | | (for FFPE tissues) |
| Background Sniper | Biocare | RT, 28 minutes |
| | Medical/BS966M | |
| Peroxidase | Ventana/760-159 | RT, 12 minutes |
| inhibitor (CM) | | |
| In house MAGEB2 | Ventana/760-219 | RT, 60 minutes |
| antibody at 0.1 | | |
| (cells) or 1-5 ug/ml | | |
| (tissues) in Ventana | | |
| diluent with Casein | | |
| Anti-Rabbit HQ | Ventana/760-4815 | RT, 28 minutes |
| Anti-HQ HRP | Ventana/760-4820 | RT, 28 minutes |
| DAB+ Chromogen | Ventana/760-159 | RT, 5 minutes |
| Hematoxylin II | Ventana/5266726001 | RT, 8 minutes |
| Bluing reagent | Ventana/526676900 | RT, 4 minutes |

### Results

Twenty-four rabbit-anti human MAGEB2 antibody supernatants were assessed for binding to MAGEB2 and potential cross reactivity for other MAGE-A and MAGE-B family members by IHC. Five of twenty-four human MAGEB2 antibody supernatants (5H20, 1J13, 1J14, 714, 7J6) did not detect MAGEB2 by IHC on human MAGEB2+ transfected cells. Nineteen of the twenty-four antibody supernatants detected MAGEB2 by IHC on human MAGEB2+ transfected cells and were assessed on other human MAGE-A+ and MAGE-B+ family member cross-reactivity control cells. Four of nineteen supernatants (4G17, 1J15, 1C3, 1I14) showed the strongest IHC signal on human MAGEB2+ transfected control cells, and the lowest/or no cross reactivity on other MAGE-A and MAGE-B family members and selected as candidates for further assessment.

Note that the twenty-four MAGEB2 antibody supernatants were not purified antibodies. A false positive cross reactivity result with one of the discarded clones may be due to non-specific staining by the supernatants. Small batch purified antibody aliquots from four MAGEB2 clones (4G17, 1J15, 1C3, 1I14) were re-tested on transfected human MAGEA+ and MAGE-B+ family member control cells, and tested on control testes tissues, normal tissue TMAs, and thirteen human cancer cell lines with known endogenous RNA expression level of MAGEB2 by IHC.

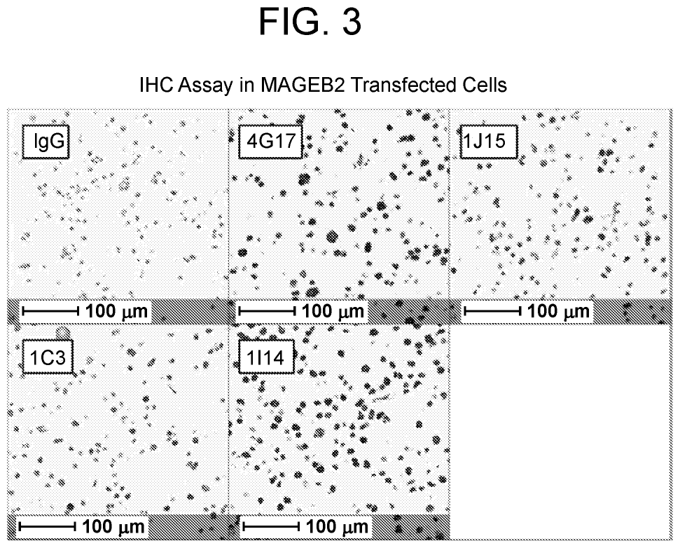

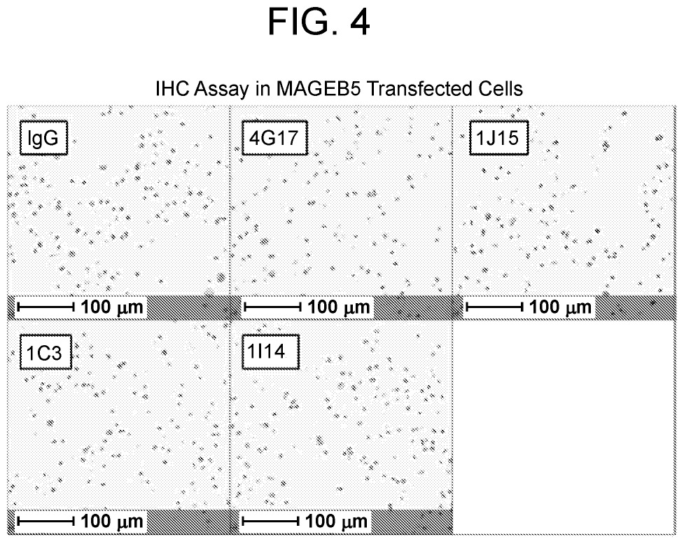

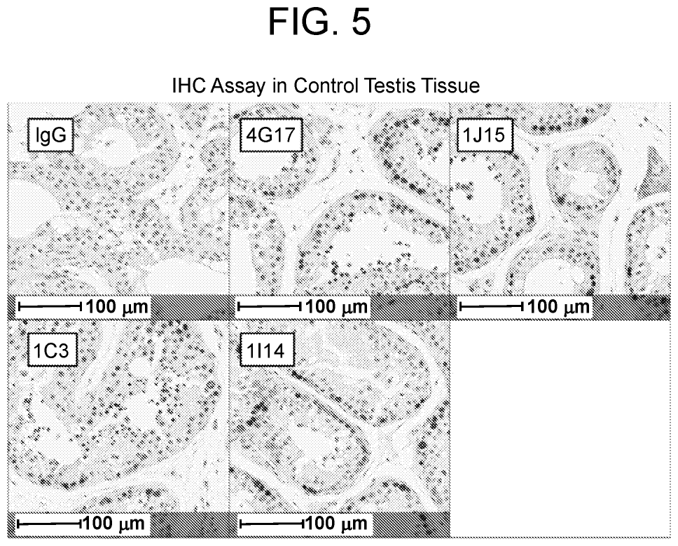

There was intense membranous/cytoplasmic staining in the transfected human MAGEB2+ cells with the four anti-MAGEB2 antibodies (4G17, 1J15, 1C3, 1I14) (FIG. 3), no cross reactivity on other human MAGE-A+ and MAGE-B+ family member cross reactivity control cells (FIG. 4), (Table 15). There was intense MAGEB2 IHC staining in testis, characterized by cytoplasmic to membrane staining of spermatogonia cells. Nuclear staining in a subset of testis germ cells was observed with 4G17, 1J15, and 1C3 clones, and immunostaining in Leydig cells was observed with 1J15, and 4G17 clones. Leydig staining is likely non-specific (FIG. 5). Nuclear staining was considered likely specific because it was observed only in testis tissues, and nuclear localization is thought to play a role in enhancement of E2F transcriptional activity, cell proliferation, and resistance to ribotoxin stress (Peche et al 2015).

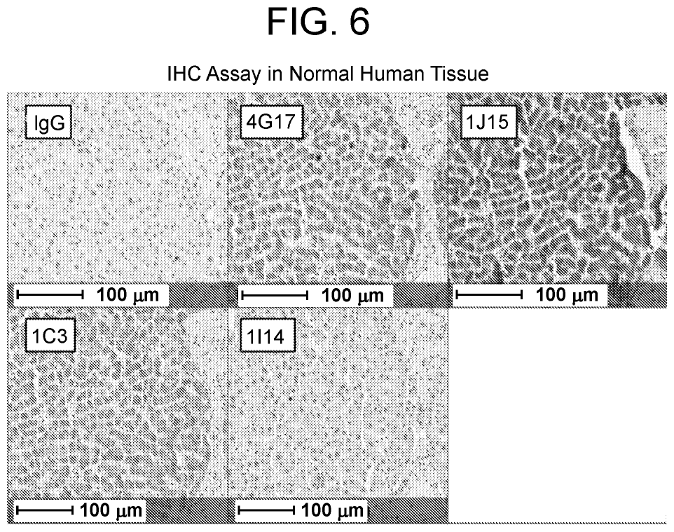

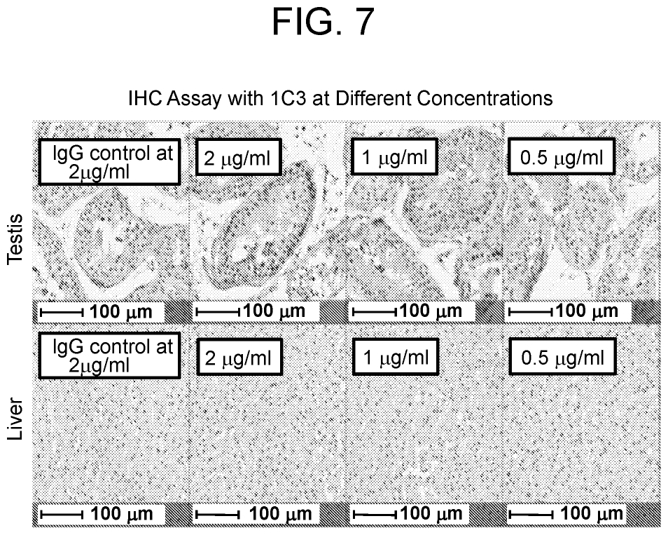

The four anti-MAGEB2 antibodies (4G17, 1J15, 1C3, 1I14) were assessed on normal tissue TMA. MAGEB2 specific immunostaining was observed only in testis cores. Immunostaining observed with 4G17, 1J15, and 1C3 clones in other tissues such as liver, adrenal gland, and intestines were presumed nonspecific. The MAGEB2 antibody (1I14) showed the lowest non-specific/background immunostaining when compared to Immunoglobulin G (IgG) isotype control (Tables 16, 17, 18, 19). Antibody clone 1I14 displays properties that indicate it will be an excellent IHC reagent for detecting MAGEB2 (FIG. 6). Furthermore, down titration of MAGEB2 (1C3) antibody completely mitigated the nonspecific background immunostaining that was observed in liver tissues, while still retaining MAGEB2 specific immunostaining in testis (FIG. 7).

Anti-MAGEB2 antibodies 4G17, 1J15, 1C3, 1I14 detected varying levels of MAGEB2 expression in the thirteen human cancer cell lines. MAGEB2 IHC expression frequency was high (˜90%) in U266B1 cells, moderate (˜70%) in T98G, KMM-1, U20S cells, and undetectable in CFPAC-1, UACC-257, CFPAC-1, VMRC-LCD, NCI-H1703, UM-UC-3, NCI-H1395, NCI-H82 cell lines. MAGEB2 IHC expression frequency in SCaBER cells were inconsistent with the four MAGEB2 clones (4G17, 1J15, 1C3, 1i14) and maybe due to different binding intensity of each antibody or unoptimized IHC assay conditions (Table 20).

### CONCLUSIONS

Twenty-four rabbit anti-human MAGEB2 antibodies were assessed for specificity to MAGEB2, and potential cross reactivity for other MAGE family members by IHC staining on FFPE tissues. Nineteen of the twenty-four detected MAGEB2 by IHC on control MAGEB2+ cells. Four of the nineteen antibodies (4G17, 1J15, 1C3, 1I14) were tested thoroughly and showed no cross reactivity to other MAGE-A or MAGE-B family members, strong IHC signal on control testis tissues, and detected the MAGEB2 IHC signal in thirteen human cancer cell lines with known endogenous RNA expression level of MAGEB2. Three of the four MAGEB2 antibodies (4G17, 1J15, 1C3) showed some non-specific background IHC staining on liver and adrenal gland in normal human tissue TMA. Data is summarized in Tables 15-20 herein.

SEQUENCES

Human MAGEB2 Amino Acid Sequence (SEQ ID NO: 1):
>sp|O15479|MAGB2_HUMAN Melanoma-associated antigen B2 OS = Homo sapiens OX = 9606
GN = MAGEB2 PE = 1 SV = 3
MPRGQKSKLRAREKRRKARDETRGLNVPQVTEAEEEEAPCCSSSVSGGAASSSPAAGIPQ
EPQRAPTTAAAAAAGVSSTKSKKGAKSHQGEKNASSSQASTSTKSPSEDPLTRKSGSLVQ
FLLYKYKIKKSVTKGEMLKIVGKRFREHFPEILKKASEGLSVVFGLELNKVNPNGHTYTF
IDKVDLTDEESLLSSWDFPRRKLLMPLLGVIFLNGNSATEEEIWEFLNMLGVYDGEEHSV
FGEPWKLITKDLVQEKYLEYKQVPSSDPPRFQFLWGPRAYAETSKMKVLEFLAKVNGTTP
CAFPTHYEEALKDEEKAGV
Full amino acid sequences for three exemplary monoclonal antibodies light chains and heavy chains
follow:
>1C3_LC (SEQ ID NO: 555)
MDMRVPAQLLGLLLLWLRGARCDIVMTQTPSSVEAAVGGTVTIKCQASQNISSYLAWYQQKPGQPPKLLIYRASTLA
SGVPSRFKGSGSGTQFTLTISDLECADAATYYCQSYDDSRSSNFFYAFGGGTEVVVKGDPVAPTVLLFPPSSDEVATGT
VTIVCVANKYFPDVTVTWEVDGTTQTTGIENSKTPQNSADCTYNLSSTLTLTSTQYNSHKEYTCKVTQGTTSVVQSFSR
KNC
>1C3_HC (SEQ ID NO: 556)
MDMRVPAQLLGLLLLWLRGARCQSLEESGGGLVQPEGSLTLTCTAFGVTLTNYYICWVRQAPGKGLEWVGCIDNAN
GRTYYASWAKGRFTISKTSSTTGTLQMTSLTAADTATYFCARSLATPLWGPGTLVTVSSGQPKAPSVFPLAPCCGDTP
SSTVTLGCLVKGYLPEPVTVTWNSGTLTNGVRTFPSVRQSSGLYSLSSVVSVTSSSQPVTCNVAHPATNTKVDKTVAPS
TCSKPTCPPPELLGGPSVFIFPPKPKDTLMISRTPEVTCVVVDVSQDDPEVQFTWYINNEQVRTARPPLREQQFNSTIR
VVSTLPIAHQDWLRGKEFKCKVHNKALPAPIEKTISKARGQPLEPKVYTMGPPREELSSRSVSLTCMINGFYPSDISVE
WEKNGKAEDNYKTTPAVLDSDGSYFLYSKLSVPTSEWQRGDVFTCSVMHEALHNHYTQKSISRSPGK
>1I14_LC (SEQ ID NO: 557)
MDMRVPAQLLGLLLLWLRGARCDVVMTQTPASVEATVGGTVTIKCQSSQSVYDNNALAWYQQNAGQRPRLLIYG
ASTLASGVPSRFSASGSGTEFTLTISDLECADAATYYCQCTYYVSSYQNDFGGGTEVVVKGDPVAPTVLLFPPSSDEVA
TGTVTIVCVANKYFPDVTVTWEVDGTTQTTGIENSKTPQNSADCTYNLSSTLTLTSTQYNSHKEYTCKVTQGTTSVVQ
SFSRKNC
>1I14_HC (SEQ ID NO: 558)
MDMRVPAQLLGLLLLWLRGARCQSVEESGGRLVTPGTPLTLTCTISGFSLSSYAMSWVRQAPGKGLEWIGSIGGGGS
AVYASWAKGRFTISKTSTTVDLRITSPTTEDTAMYFCGRGFYSIDLWGPGTLVTVSSGQPKAPSVFPLAPCCGDTPSST
VTLGCLVKGYLPEPVTVTWNSGTLTNGVRTFPSVRQSSGLYSLSSVVSVTSSSQPVTCNVAHPATNTKVDKTVAPSTC
SKPTCPPPELLGGPSVFIFPPKPKDTLMISRTPEVTCVVVDVSQDDPEVQFTWYINNEQVRTARPPLREQQFNSTIRVV
STLPIAHQDWLRGKEFKCKVHNKALPAPIEKTISKARGQPLEPKVYTMGPPREELSSRSVSLTCMINGFYPSDISVEWE
KNGKAEDNYKTTPAVLDSDGSYFLYSKLSVPTSEWQRGDVFTCSVMHEALHNHYTQKSISRSPGK
>1H17_LC (SEQ ID NO: 559)
MDMRVPAQLLGLLLLWLRGARCAAVLTQTPSPVSAAVGGTVSASCQSSKSVYNKNWLSWFQQKPGQPPKLLIYGA
STLASGVPSRFKGSGSGTQFTLTISDVQCDDAATYYCAGGYSSSSDTFAFGGGTEVVVKGDPVAPTVLLFPPSSDEVAT
GTVTIVCVANKYFPDVTVTWEVDGTTQTTGIENSKTPQNSADCTYNLSSTLTLTSTQYNSHKEYTCKVTQGTTSVVQS
FSRKNC
>1H17_HC (SEQ ID NO: 560)
MDMRVPAQLLGLLLLWLRGARCQEQLVESGGGLVKPGASLTLTCKASGFSFSSGQLMCWVRQAPGKGLEWIACIGS
GSNAISTFYASWAQGRFTISKSSSTTVTLQLTSLTAADTATYFCARVGSDDYGDSDVFDPWGPGTLVTVSSGQPKAPS
VFPLAPCCGDTPSSTVTLGCLVKGYLPEPVTVTWNSGTLTNGVRTFPSVRQSSGLYSLSSVVSVTSSSQPVTCNVAHPA
TNTKVDKTVAPSTCSKPTCPPPELLGGPSVFIFPPKPKDTLMISRTPEVTCVVVDVSQDDPEVQFTWYINNEQVRTAR
PPLREQQFNSTIRVVSTLPIAHQDWLRGKEFKCKVHNKALPAPIEKTISKARGQPLEPKVYTMGPPREELSSRSVSLTC
MINGFYPSDISVEWEKNGKAEDNYKTTPAVLDSDGSYFLYSKLSVPTSEWQRGDVFTCSVMHEALHNHYTQKSISRS
PGK

Additional exemplary sequences of the present invention are provided in Tables 21-25 herein.

TABLE 15
MAGEB2 Immunohistochemistry Staining
in MAGE-A/B control cells

| | | | | Other | Other |
|---|---|---|---|---|---|
| | HTSC | | MAGE- | MAGE-B | MAGE-A |
| Antibody | Inventory Code | CHO | B2 | family | family |
| ID | (Block ID) | cells | cells | members | members |
| 4G17 | 694001, 697782, | negative | positive | negative | negative |
| | 697548, 697639, | | | | |
| | 694686 | | | | |
| 1J15 | 694001, 697782, | negative | positive | negative | negative |
| | 697548, 697639, | | | | |
| | 694686 | | | | |
| 1C3 | 694001, 697782, | negative | positive | negative | negative |
| | 697548, 697639, | | | | |
| | 694686 | | | | |
| 1i14 | 694001, 697782, | negative | positive | negative | negative |
| | 697548, 697639, | | | | |
| | 694686 | | | | |
| IgG | 694001, 697782, | negative | negative | negative | negative |
| | 697548, 697639, | | | | |
| | 694686 | | | | |

TABLE 16
MAGE-B2 (1i14) Immunohistochemistry Staining
in Normal Human Tissue TMA (FDA999x)

| Tissue identification | | | | | | |
|---|---|---|---|---|---|---|
| | | | Tissue | IHC Isotype Control | | |
| Position | No. | Organ | ID | Negative | Positive | Comment |
| A1 | 1 | Cerebrum | Nct05N012 | X | | |
| A2 | 2 | Cerebrum | Nct05N016 | X | | |
| A3 | 3 | Cerebrum | Nct08N046 | X | | |
| A4 | 4 | Cerebellum | Ncb05N008 | X | | |
| A5 | 5 | Cerebellum | Ncb15N146 | X | | |
| A6 | 6 | Cerebellum | Ncb05N011 | X | | |
| A7 | 7 | Adrenal | Eag06N019 | X | | Non-specific |
| | | gland | | | | particulate |
| | | | | | | staining in |
| | | | | | | cortex (Zona |
| | | | | | | fasiculata |
| | | | | | | and zona |
| | | | | | | reticularis) |
| A8 | 8 | Adrenal | Eag06N010 | X | | Non-specific |
| | | gland | | | | particulate |
| | | | | | | staining in |
| | | | | | | cortex (Zona |
| | | | | | | fasiculata |
| | | | | | | and zona |
| | | | | | | reticularis) |
| A9 | 9 | Adrenal | Eag15N145 | X | | Non-specific |
| | | gland | | | | particulate |
| | | | | | | staining in |
| | | | | | | cortex (Zona |
| | | | | | | fasiculata |
| | | | | | | and zona |
| | | | | | | reticularis) |
| A10 | 10 | Ovary | Fov07N034 | X | | |
| A11 | 11 | Ovary | Fov13N015 | X | | |
| A12 | 12 | Ovary | Fov13N023 | X | | |
| B1 | 13 | Pancreas | Dpa15N145 | TI | | Tissue |
| | | | | | | autolyzed |
| B2 | 14 | Pancreas | Dpa06N023 | X | | |
| B3 | 15 | Pancreas | Dpa11N012 | X | | |
| B4 | 16 | Lymph | Ily06N015 | X | | |
| | | node | | | | |
| B5 | 17 | Lymph | Ily08N047 | X | | Non-specific |
| | | node | | | | staining in |
| | | | | | | exfoliated |
| | | | | | | cells |
| B6 | 18 | Lymph | Ily08N014 | X | | |
| | | node | | | | |
| B7 | 19 | Hypophysis | Ept14N001 | X | | |
| B8 | 20 | Hypophysis | Ept13N019 | X | | |
| B9 | 21 | Hypophysis | Ept13N020 | X | | |
| B10 | 22 | Testis | Mtt09N048 | X | | |
| B11 | 23 | Testis | Mtt08N001 | X | | |
| B12 | 24 | Testis | Mtt08N009 | X | | |
| C1 | 25 | Thyroid | Etg07N002 | X | | Non-specific |
| | | gland | | | | staining in |
| | | | | | | colloid |
| C2 | 26 | Thyroid | Etg08N047 | X | | Non-specific |
| | | gland | | | | staining in |
| | | | | | | colloid |
| C3 | 27 | Thyroid | Etg06N003 | X | | Non-specific |
| | | gland | | | | staining in |
| | | | | | | colloid |
| C4 | 28 | Breast | Fmg08N034 | X | | |
| C5 | 29 | Breast | Fmg50N034 | X | | |
| C6 | 30 | Breast | Fmg07N040 | X | | |
| C7 | 31 | Spleen | Isp15N145 | X | | |
| C8 | 32 | Spleen | Isp15N146 | X | | |
| C9 | 33 | Spleen | Isp11N001 | X | | |
| C10 | 34 | Tonsil | Rts13N011 | TI | | Only adipose |
| | | | | | | tissue present |
| C11 | 35 | Tonsil | Rts50N001 | X | | |
| C12 | 36 | Tonsil | Rts08N042 | X | | Minimal |
| | | | | | | lymphoid |
| | | | | | | tissue present |
| D1 | 37 | Thymus | Ith06N024 | X | | |
| | | gland | | | | |
| D2 | 38 | Thymus | Ith06N002 | X | | |
| | | gland | | | | |
| D3 | 39 | Thymus | Ith15N145 | X | | |
| | | gland | | | | |
| D4 | 40 | Bone | Ibm06N024 | X | | Non-specific |
| | | marrow | | | | staining in |
| | | | | | | exfoliated |
| | | | | | | cells |
| D5 | 41 | Bone | Ibm11N015 | X | | Non-specific |
| | | marrow | | | | staining in |
| | | | | | | exfoliated |
| | | | | | | cells |
| D6 | 42 | Bone | Ibm12N001 | X | | Non-specific |
| | | marrow | | | | staining in |
| | | | | | | exfoliated |
| | | | | | | cells |
| D7 | 43 | Lung | Rln06N004 | X | | |
| D8 | 44 | Lung | Rln06N027 | X | | |
| D9 | 45 | Lung | Rln06N023 | X | | |
| D10 | 46 | Heart | Cht11N009 | X | | |
| D11 | 47 | Heart | Cht05N005 | X | | |
| D12 | 48 | Heart | Cht11N001 | X | | |
| E1 | 49 | Esophagus | Des06N027 | X | | |
| E2 | 50 | Esophagus | Des17N004 | X | | Minimal |
| | | | | | | epithelial |
| | | | | | | tissue |
| E3 | 51 | Esophagus | Des15N117 | X | | |
| E4 | 52 | Stomach | Dst07N016 | X | | Hazy non- |
| | | | | | | specific |
| | | | | | | staining in |
| | | | | | | gastric gland |
| | | | | | | cells |
| E5 | 53 | Stomach | Dst15N003 | X | | Hazy non- |
| | | | | | | specific |
| | | | | | | staining in |
| | | | | | | gastric gland |
| | | | | | | cells |
| E6 | 54 | Stomach | Dst06N017 | X | | Hazy non- |
| | | | | | | specific |
| | | | | | | staining in |
| | | | | | | gastric gland |
| | | | | | | cells |
| E7 | 55 | Small | Din07N016 | X | | Weak non- |
| | | intestine | | | | specific |
| | | | | | | mucosal |
| | | | | | | staining |
| E8 | 56 | Small | Din08N014 | X | | |
| | | intestine | | | | |
| E9 | 57 | Small | Din08N007 | X | | |
| | | intestine | | | | |
| E10 | 58 | Colon | Dco15N083 | X | | |
| E11 | 59 | Colon | Dco06N022 | X | | |
| E12 | 60 | Colon | Dco15N082 | TI | | No mucosal |
| | | | | | | tissue |
| F1 | 61 | Liver | Dlv05N015 | | X | |
| F2 | 62 | Liver | Dlv15N146 | X | | Non-specific |
| | | | | | | particulate |
| | | | | | | staining in |
| | | | | | | hepatocytes |
| F3 | 63 | Liver | Dlv15N145 | X | | |
| F4 | 64 | Salivary | Doc11N007 | X | | |
| | | gland | | | | |
| F5 | 65 | Salivary | Doc06N023 | X | | |
| | | gland | | | | |
| F6 | 66 | Salivary | Doc06N022 | X | | |
| | | gland | | | | |
| F7 | 67 | Kidney | Ukn08N047 | X | | |
| F8 | 68 | Kidney | Ukn08N035 | X | | |
| F9 | 69 | Kidney | Ukn06N004 | X | | |
| F10 | 70 | Prostate | Mpr08N004 | X | | |
| F11 | 71 | Prostate | Mpr08N005 | X | | |
| F12 | 72 | Prostate | Mpr11N013 | X | | |
| G1 | 73 | Uterus | Fur17N003 | X | | |
| G2 | 74 | Uterus | Fur50N029 | X | | |
| G3 | 75 | Uterus | Fur07N040 | X | | |
| G4 | 76 | Cervix | Fdu50N020 | X | | |
| G5 | 77 | Cervix | Fdu17N003 | X | | |
| G6 | 78 | Cervix | Fdu50N019 | X | | |
| G7 | 79 | Bladder | Ubd08N007 | X | | |
| G8 | 80 | Bladder | Ubd08N001 | X | | |
| G9 | 81 | Bladder | Ubd08N009 | X | | |
| G10 | 82 | Skeletal | Srm15N008 | X | | |
| | | muscle | | | | |
| G11 | 83 | Skeletal | Srm15N009 | X | | |
| | | muscle | | | | |
| G12 | 84 | Skeletal | Srm05N014 | X | | |
| | | muscle | | | | |
| H1 | 85 | Skin | Kin15N007 | X | | |
| H2 | 86 | Skin | Kin07N025 | X | | |
| H3 | 87 | Skin | Kin15N009 | X | | |
| H4 | 88 | Nerve | Scf10N003 | X | | |
| H5 | 89 | Nerve | Scf08N010 | X | | |
| H6 | 90 | Nerve | Scf11N005 | X | | |
| H7 | 91 | Artery | Sbv07N029 | X | | |
| H8 | 92 | Pericardium | Apr15N145 | X | | |
| H9 | 93 | Pericardium | Apr15N146 | X | | |
| H10 | 94 | Eye | Vey130002 | TI | | Only pigmented |
| | | | | | | retina present |
| H11 | 95 | Eye | Vey140001 | TI | | Only pigmented |
| | | | | | | retina present |
| H12 | 96 | Eye | Vey070007 | X | | Intense |
| | | | | | | pigmentation |
| 11 | 97 | Larynx | Rla13N011 | X | | |
| 12 | 98 | Larynx | Rla13N020 | X | | |
| 13 | 99 | Larynx | Rla07N010 | X | | |

Target IHC

| | | Cell | Intensity | | |
|---|---|---|---|---|---|
| Negative | Positive | type | (0-3+) | Localization | Comment |
| X | | | | | |
| X | | | | | |
| X | | | | | Very weak |
| | | | | | non-specific |
| | | | | | staining in |
| | | | | | the vessels |
| X | | | | | |
| X | | | | | Very weak |
| | | | | | non-specific |
| | | | | | staining in |
| | | | | | the vessels |
| X | | | | | Very weak |
| | | | | | non-specific |
| | | | | | staining in |
| | | | | | the vessels |
| X | | | | | |
| X | | | | | Non-specific |
| | | | | | particulate |
| | | | | | staining in |
| | | | | | cortex (Zona |
| | | | | | fasiculata |
| | | | | | and zona |
| | | | | | reticularis) |
| | | | | | as observed |
| | | | | | in isotype |
| X | | | | | |
| X | | | | | |
| X | | | | | |
| X | | | | | |
| TI | | | | | Tissue |
| | | | | | autolyzed |
| X | | | | | |
| X | | | | | |
| X | | | | | |
| X | | | | | Non-specific |
| | | | | | staining in |
| | | | | | exfoliated |
| | | | | | cells as |
| | | | | | observed in |
| | | | | | isotype |
| X | | | | | |
| X | | | | | |
| X | | | | | |
| X | | | | | |
| X | X | Spermatogonia | 3 | Cytoplasmic/ | |
| | | cells | | membrane | |
| X | X | Spermatogonia | 3 | Cytoplasmic/ | |
| | | cells | | membrane | |
| X | X | Spermatogonia | 3 | Cytoplasmic/ | |
| | | cells | | membrane | |
| | | | | | Non-specific |
| | | | | | staining as |
| | | | | | observed in |
| | | | | | isotype |
| | | | | | Non-specific |
| | | | | | staining as |
| | | | | | observed in |
| | | | | | sotype |
| | | | | | Non-specific |
| | | | | | staining as |
| | | | | | observed in |
| | | | | | isotype |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | Non-specific |
| | | | | | staining as |
| | | | | | observed in |
| | | | | | isotype |
| X | | | | | Non-specific |
| | | | | | staining as |
| | | | | | observed in |
| | | | | | isotype |
| X | | | | | Non-specific |
| | | | | | staining as |
| | | | | | observed in |
| | | | | | isotype |
| X | | | | | |
| X | | | | | |
| X | | | | | |
| X | | | | | |
| X | | | | | |
| X | | | | | |
| X | | | | | Intense |
| | | | | | non-specific |
| | | | | | staining in |
| | | | | | superficial |
| | | | | | epithelium |
| X | | | | | |
| X | | | | | |
| X | | | | | Non-specific |
| | | | | | staining as |
| | | | | | observed in |
| | | | | | isotype |
| X | | | | | Non-specific |
| | | | | | staining as |
| | | | | | observed in |
| | | | | | isotype |
| X | | | | | |
| X | | | | | Non-specific |
| | | | | | cytoplasmic |
| | | | | | staining in |
| | | | | | epithelial |
| | | | | | cells as |
| | | | | | observed in |
| | | | | | isotype |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| TI | | | | | Only pigmented |
| | | | | | retina present |
| TI | | | | | Only pigmented |
| | | | | | retina present |
| X | | | | | Intense |
| | | | | | pigmentation |
| X | | | | | |
| X | | | | | |
| X | | | | | |

MAGE-B2 specific IHC staining with MAGE-B2 (1114) was observed only in testis tissues characterized by cytoplasmic to membrane staining of spermatogonia cells. Staining in other tissues were either observed in isotype or deemed non-specific.

TABLE 17
MAGE-B2 (IJ15) Immunohistochemistry Staining in Normal Human Tissue TMA (FDA999x)

| Tissue identification | | | | Target IHC | | | | | |
|---|---|---|---|---|---|---|---|---|---|
| | | | Tissue | | | Cell | Intensity | | |
| Position | No. | Organ | ID | Negative | Positive | type | (0-3+) | Localization | Comment |
| A1 | 1 | Cerebrum | Nct03N002 | X | | | | | |
| A2 | 2 | Cerebrum | Nct03N005 | X | | | | | Non-specific staining in |
| | | | | | | | | | blood vessels |
| A3 | 3 | Cerebrum | Nct04N001 | X | | | | | Non-specific staining in |
| | | | | | | | | | blood vessels |
| A4 | 4 | Cerebrum | Nct11N001 | X | | | | | |
| A5 | 5 | Cerebrum | Nct07N015 | X | | | | | Non-specific staining in |
| | | | | | | | | | blood vessels |
| A6 | 6 | Cerebrum | Nct05N006 | X | | | | | Non-specific staining in |
| | | | | | | | | | blood vessels |
| A7 | 7 | Cerebellum | Ncb04N001 | X | | | | | |
| A8 | 8 | Cerebellum | Ncb05N006 | X | | | | | Non-specific staining in |
| | | | | | | | | | blood vessels |
| A9 | 9 | Cerebellum | Ncb05N011 | X | | | | | |
| B1 | 10 | Adrenal | Eag07N001 | | X | Epithelial | 3+ | Cytoplasmic/ | Intense staining in |
| | | gland | | | | cells | | membrane | cortex and likely non- |
| | | | | | | | | | specific |
| B2 | 11 | Adrenal | Eag06N010 | | X | Epithelial | 3+ | Cytoplasmic/ | Intense staining in |
| | | gland | | | | cells | | membrane | cortex and likely non- |
| | | | | | | | | | specific |
| B3 | 12 | Adrenal | Eag06N009 | | X | Epithelial | 3+ | Cytoplasmic/ | Intense staining in |
| | | gland | | | | cells | | membrane | cortex and likely non- |
| | | | | | | | | | specific |
| B4 | 13 | Ovary | Fov110136 | X | | | | | |
| B5 | 14 | Ovary | Fov160119 | X | | | | | |
| B6 | 15 | Ovary | Fdu100022 | X | | | | | |
| B7 | 16 | Pancreas | Dpa05N010 | | X | Epithelial | 2+ | Cytoplasmic/ | Weak to mild staining in |
| | | | | | | cells | | membrane | acinar epithelium and |
| | | | | | | | | | likely non-specific |
| B8 | 17 | Pancreas | Dpa03N009 | X | | | | | Non-specific staining in |
| | | | | | | | | | blood vessels |
| B9 | 18 | Pancreas | Dpa08N040 | | X | Epithelial | 1+ | Cytoplasmic/ | Very weak staining in |
| | | | | | | cells | | membrane | acinar epithelium and |
| | | | | | | | | | likely non-specific |
| C1 | 19 | Lymph | Ily08N047 | X | | | | | Non-specific staining in |
| | | node | | | | | | | exfoliated cells and |
| | | | | | | | | | some blood vessels |
| C2 | 20 | Lymph | Ily11N004 | X | | | | | Non-specific staining in |
| | | node | | | | | | | exfoliated cells and |
| | | | | | | | | | some blood vessels |
| C3 | 21 | Lymph | Ily07N015 | X | | | | | |
| | | node | | | | | | | |
| C4 | 22 | Hypophysis | Ept17N003 | X | | | | | Non-specific staining in |
| | | | | | | | | | blood vessels |
| C5 | 23 | Hypophysis | Ept17N003 | X | | | | | Non-specific staining in |
| | | | | | | | | | blood vessels |
| C6 | 24 | Hypophysis | Ept17N003 | X | | | | | Non-specific staining in |
| | | | | | | | | | blood vessels |
| C7 | 25 | Testis | Mtt07N016 | | X | Spermatogonia | 3+ | Cytoplasmic/ | Leydig cells staining is |
| | | | | | | cells; | | membrane; | likely non-specific |
| | | | | | | Leydig cells | | nuclear | |
| | | | | | | | | staining in | |
| | | | | | | | | some | |
| C8 | 26 | Testis | Mtt11N004 | | X | Spermatogonia | 2+ | Cytoplasmic/ | Leydig cells staining is |
| | | | | | | cells; | | membrane; | likely non-specific |
| | | | | | | Leydig cells | | nuclear | |
| | | | | | | | | staining in | |
| | | | | | | | | some | |
| C9 | 27 | Testis | Mtt07N026 | | X | Spermatogonia | 3+ | Cytoplasmic/ | Leydig cells staining is |
| | | | | | | cells; | | membrane; | likely non-specific |
| | | | | | | Leydig cells | | nuclear | |
| | | | | | | | | staining in | |
| | | | | | | | | some | |
| D1 | 28 | Thyroid | Etg06N003 | X | | | | | Non-specific staining in |
| | | gland | | | | | | | colloid and serum |
| D2 | 29 | Thyroid | Etg06N010 | X | | | | | Non-specific staining in |
| | | gland | | | | | | | colloid and serum |
| D3 | 30 | Thyroid | Etg05N007 | X | | | | | Non-specific staining in |
| | | gland | | | | | | | colloid and serum |
| D4 | 31 | Breast | Fmg140091 | X | | | | | |
| D5 | 32 | Breast | Fmg07N034 | X | | | | | |
| D6 | 33 | Breast | Fmg12N003 | X | | | | | |
| D7 | 34 | Spleen | Isp06N023 | X | | | | | Non-specific staining in |
| | | | | | | | | | exfoliated cells and |
| | | | | | | | | | some blood vessels |
| D8 | 35 | Spleen | Isp05N009 | X | | | | | Non-specific staining in |
| | | | | | | | | | exfoliated cells and |
| | | | | | | | | | some blood vessels |
| D9 | 36 | Spleen | Isp07N028 | X | | | | | Non-specific staining in |
| | | | | | | | | | exfoliated cells and |
| | | | | | | | | | some blood vessels |
| E1 | 37 | Tonsil | Doc041188 | X | | | | | |
| E2 | 38 | Tonsil | Doc041188 | X | | | | | |
| E3 | 39 | Tonsil | Doc041188 | X | | | | | |
| E4 | 40 | Thymus | Ith06N002 | X | | | | | Non-specific staining in |
| | | gland | | | | | | | endothelial cells |
| E5 | 41 | Thymus | Ith06N024 | X | | | | | Non-specific staining in |
| | | gland | | | | | | | endothelial cells |
| E6 | 42 | Thymus | Ith06N025 | X | | | | | Non-specific staining in |
| | | gland | | | | | | | endothelial cells |
| E7 | 43 | Bone | Ibm07N029 | X | | | | | Non-specific staining in |
| | | marrow | | | | | | | exfoliated cells |
| E8 | 44 | Bone | Ibm07N023 | X | | | | | Non-specific staining in |
| | | marrow | | | | | | | exfoliated cells |
| E9 | 45 | Bone | Ibm07N026 | X | | | | | Non-specific staining in |
| | | marrow | | | | | | | exfoliated cells |
| F1 | 46 | Lung | Rln05N023 | X | | | | | Non-specific staining in |
| | | | | | | | | | alveolar septum, |
| | | | | | | | | | exfoliated cells |
| F2 | 47 | Lung | Rln05N010 | X | | | | | Non-specific staining in |
| | | | | | | | | | alveolar septum, |
| | | | | | | | | | exfoliated cells |
| F3 | 48 | Lung | Rln08N014 | X | | | | | Non-specific staining in |
| | | | | | | | | | alveolar septum, |
| | | | | | | | | | exfoliated cells |
| F4 | 49 | Heart | Cht05N005 | X | | | | | Non-specific staining in |
| | | | | | | | | | serum and blood |
| | | | | | | | | | vessels |
| F5 | 50 | Heart | Cht03N009 | X | | | | | Non-specific staining in |
| | | | | | | | | | serum and blood |
| | | | | | | | | | vessels |
| F6 | 51 | Heart | Cht11N005 | X | | | | | Non-specific staining in |
| | | | | | | | | | serum and blood |
| | | | | | | | | | vessels |
| F7 | 52 | Esophagus | Des06N003 | | X | | 3+ | Cytoplasmic/ | Staining in superficial |
| | | | | | | | | membrane | epithelium, blood |
| | | | | | | | | | vessels and connective |
| | | | | | | | | | tissues are likely non- |
| | | | | | | | | | specific |
| F8 | 53 | Esophagus | Des15N145 | | X | | 2+ | Cytoplasmic/ | Staining in superficial |
| | | | | | | | | membrane | epithelium, blood |
| | | | | | | | | | vessels and connective |
| | | | | | | | | | tissues are likely non- |
| | | | | | | | | | specific |
| F9 | 54 | Esophagus | Des06N009 | X | | | | | Non-specific staining n |
| | | | | | | | | | blood vessels and |
| | | | | | | | | | connective tissue |
| G1 | 55 | Stomach | Dst08N033 | | X | | 2+ | Cytoplasmic/ | Non-specific staining in |
| | | | | | | | | membrane | glandular epithelial cells |
| G2 | 56 | Stomach | Dst08N011 | | X | Epithelium | 3+ | Cytoplasmic/ | Non-specific staining in |
| | | | | | | | | membrane | glandular epithelial cells |
| G3 | 57 | Stomach | Dst17N005 | X | | | | | |
| G4 | 58 | Small | Din07N016 | X | | | | | Non-specific staining in |
| | | intestine | | | | | | | blood vessels; Weak |
| | | | | | | | | | hazy cytoplasmic |
| | | | | | | | | | staining in mucosal |
| | | | | | | | | | epithelium |
| G5 | 59 | Small | Din09N051 | X | | | | | Non-specific staining in |
| | | intestine | | | | | | | blood vessels; Weak |
| | | | | | | | | | hazy cytoplasmic |
| | | | | | | | | | staining in mucosal |
| | | | | | | | | | epithelium |
| G6 | 60 | Small | Din06N022 | X | | | | | Non-specific staining in |
| | | intestine | | | | | | | blood vessels; Weak |
| | | | | | | | | | hazy cytoplasmic |
| | | | | | | | | | staining in mucosal |
| | | | | | | | | | epithelium |
| G7 | 61 | Colon | Dco15N142 | X | | | | | |
| G8 | 62 | Colon | Dco15N103 | X | | | | | |
| G9 | 63 | Colon | Dco15N115 | X | | | | | |
| H1 | 64 | Liver | Dlv05N015 | X | X | Hepatocytes | 2+ | Cytoplasmic/ | Mild to intense staining |
| | | | | | | | | membrane | and likely non-specific |
| H2 | 65 | Liver | Dlv15N145 | X | X | Hepatocytes | 2+ | Cytoplasmic/ | Mild staining and likely |
| | | | | | | | | membrane | non-specific |
| H3 | 66 | Liver | Dlv03N005 | X | X | Hepatocytes | 1+ | Cytoplasmic/ | Very weak staining and |
| | | | | | | | | membrane | likely non-specific |
| H4 | 67 | Salivary | Doc120045 | X | | | | | Non-specific staining in |
| | | gland | | | | | | | blood vessels and |
| | | | | | | | | | serum |
| H5 | 68 | Salivary | Doc05N009 | X | | | | | Non-specific staining in |
| | | gland | | | | | | | blood vessels and |
| | | | | | | | | | serum |
| H6 | 69 | Salivary | Doc06N019 | X | | | | | Non-specific staining in |
| | | gland | | | | | | | blood vessels and |
| | | | | | | | | | serum |
| H7 | 70 | Kidney | Ukn06N006 | X | | | | | Non-specific staining in |
| | | | | | | | | | vasculature serum |
| | | | | | | | | | proteins |
| H8 | 71 | Kidney | Ukn05N003 | X | | | | | Non-specific staining in |
| | | | | | | | | | vasculature serum |
| | | | | | | | | | proteins |
| H9 | 72 | Kidney | Ukn06N023 | | X | Tubular | 2+ | C/M | Staining in vasculature |
| | | | | | | epithelium | | | serum proteins and |
| | | | | | | | | | tubular epithelium likely |
| | | | | | | | | | non-specific |
| I1 | 73 | Prostate | Mpr07N029 | TI | | | | | Tissue inadequate |
| I2 | 74 | Prostate | Mpr11N008 | X | | | | | Weak hazy non-specific |
| | | | | | | | | | staining in stroma and |
| | | | | | | | | | glandular epithelium |
| I3 | 75 | Prostate | Mpr09N052 | X | | | | | Weak hazy non-specific |
| | | | | | | | | | staining in stroma and |
| | | | | | | | | | glandular epithelium |
| I4 | 76 | Uterus | Fur07N010 | X | | | | | |
| I5 | 77 | Uterus | Fur07N001 | X | | | | | Non-specific staining in |
| | | | | | | | | | blood vessels |
| I6 | 78 | Uterus | Fur150196 | X | | | | | Non-specific nuclear |
| | | | | | | | | | staining in some stromal |
| | | | | | | | | | cells; hazy cytoplasmic |
| | | | | | | | | | staining in glandular |
| | | | | | | | | | epithelium that is likely |
| | | | | | | | | | non-specific |
| I7 | 79 | Cervix | Fur170174 | X | | | | | |
| I8 | 80 | Cervix | Fdu060889 | X | | | | | |
| I9 | 81 | Cervix | Fdu170148 | X | | | | | |
| J1 | 82 | Skeletal | Srm06N015 | X | | | | | Very weak non-specific |
| | | muscle | | | | | | | hazy staining |
| J2 | 83 | Skeletal | Srm05N005 | X | | | | | Very weak non-specific |
| | | muscle | | | | | | | hazy staining |
| J3 | 84 | Skeletal | Srm05N007 | X | | | | | Very weak non-specific |
| | | muscle | | | | | | | hazy staining |
| J4 | 85 | Skin | Kin07N017 | X | | | | | |
| J5 | 86 | Skin | Kin06N025 | X | | | | | |
| J6 | 87 | Skin | Kin08N034 | X | | | | | |
| J7 | 88 | Nerve | Scf15N098 | X | | | | | |
| J8 | 89 | Nerve | Scf15N009 | X | | | | | |
| J9 | 90 | Nerve | Scf07N017 | X | | | | | |
| K1 | 91 | Pericardium | Apr07N027 | TI | | | | | Tissue inadequate |
| K2 | 92 | Diaphragm | Srm17N006 | X | | | | | |
| K3 | 93 | Pericardium | Apr07N026 | X | | | | | |
| K4 | 94 | Eye | Vey090007 | X | | | | | Intense pigmentation |
| K5 | 95 | Eye | Vey140007 | TI | | | | | Tissue inadequate |
| K6 | 96 | Eye | Vey090002 | X | | | | | |
| K7 | 97 | Larynx | Rla17N006 | X | | | | | Non-specific staining in |
| | | | | | | | | | superficial epithelium |
| K8 | 98 | Larynx | Rla17N005 | X | | | | | |
| K9 | 99 | Larynx | Rla06N023 | TI | | | | | Tissue inadequate |

MAGE-B2 IHC staining with MAGE-B2 (IJ15) is observed in testis tissues characterized by cytoplasmic to membrane staining of spermatogonia cells, nuclear staining in some and staining in Leydig cells. Leydig staining is likely non-specific. Staining in other tissues were either observed in isotype or deemed non-specific.

TABLE 18
MAGE-B2 (IC3) Immunohistochemistry Staining in Normal Human Tissue TMA (FDA999x)

| | | Tissue | IHC Isotype Control | | | Target IHC | |
|---|---|---|---|---|---|---|---|
| No. | Organ | ID | Negative | Positive | Comment | Negative | Positive |
| 1 | Cerebrum | Nct05N012 | X | | | X | |
| 2 | Cerebrum | Nct05N016 | X | | | X | |
| 3 | Cerebrum | Nct08N046 | X | | | X | |
| 4 | Cerebellum | Ncb05N008 | X | | | X | |
| 5 | Cerebellum | Ncb15N146 | X | | | X | |
| 6 | Cerebellum | Ncb05N011 | X | | | | |
| 7 | Adrenal | Eag06N019 | X | | Non-specific | | X |
| | gland | | | | particulate staining | | |
| | | | | | in cortex (Zona | | |
| | | | | | fasiculata and zona | | |
| | | | | | reticularis) | | |
| 8 | Adrenal | Eag06N010 | X | | Non-specific | | X |
| | gland | | | | particulate staining | | |
| | | | | | in cortex (Zona | | |
| | | | | | fasiculata and zona | | |
| | | | | | reticularis) | | |
| 9 | Adrenal | Eag15N145 | X | | Non-specific | | X |
| | gland | | | | particulate staining | | |
| | | | | | in cortex (Zona | | |
| | | | | | fasiculata and zona | | |
| | | | | | reticularis) | | |
| 10 | Ovary | Fov07N034 | X | | | X | |
| 11 | Ovary | Fov13N015 | X | | | X | |
| 12 | Ovary | Fov13N023 | X | | | X | |
| 13 | Pancreas | Dpa15N145 | TI | | Tissue autolyzed | TI | |
| 14 | Pancreas | Dpa06N023 | X | | | | X |
| 15 | Pancreas | Dpa11N012 | X | | | | X |
| 16 | Lymph node | Ily06N015 | X | | | | |
| 17 | Lymph node | Ily08N047 | X | | Non-specific | X | |
| | | | | | staining in | | |
| | | | | | exfoliated cells | | |
| 18 | Lymph node | Ily08N014 | X | | | X | |
| 19 | Hypophysis | Ept14N001 | X | | | X | |
| 20 | Hypophysis | Ept13N019 | X | | | X | |
| 21 | Hypophysis | Ept13N020 | X | | | X | |
| 22 | Testis | Mtt09N048 | X | | | | X |
| 23 | Testis | Mtt08N001 | X | | | | X |
| 24 | Testis | Mtt08N009 | X | | | | X |
| 25 | Thyroid | Etg07N002 | X | | Hazy non-specific | | |
| | gland | | | | staining in colloid | | |
| 26 | Thyroid | Etg08N047 | X | | Hazy non-specific | | |
| | land | | | | staining in colloid | | |
| 27 | Thyroid | Etg06N003 | X | | Hazy non-specific | | |
| | gland | | | | staining in colloid | | |
| 28 | Breast | Fmg08N034 | X | | | X | |
| 29 | Breast | Fmg50N034 | X | | | X | |
| 30 | Breast | Fmg07N040 | X | | | X | |
| 31 | Spleen | Isp15N145 | X | | | X | |
| 32 | Spleen | Isp15N146 | X | | | X | |
| 33 | Spleen | Isp11N001 | X | | | X | |
| 34 | Tonsil | Rts13N011 | TI | | Only adipose tissue | TI | |
| | | | | | present | | |
| 35 | Tonsil | Rts50N001 | X | | | X | |
| 36 | Tonsil | Rts08N042 | X | | Minimal lymphoid | X | |
| | | | | | tissue present | | |
| 37 | Thymus | Ith06N024 | X | | | X | |
| | gland | | | | | | |
| 38 | Thymus | Ith06N002 | X | | | X | |
| | gland | | | | | | |
| 39 | Thymus | Ith15N145 | X | | | X | |
| | gland | | | | | | |
| 40 | Bone | Ibm06N024 | X | | Non-specific | X | |
| | marrow | | | | staining in | | |
| | | | | | exfoliated cells | | |
| 41 | Bone | Ibm11N015 | X | | Non-specific | X | |
| | marrow | | | | staining in | | |
| | | | | | exfoliated cells | | |
| 42 | Bone | Ibm12N001 | X | | Non-specific | X | |
| | marrow | | | | staining in | | |
| | | | | | exfoliated cells | | |
| 43 | Lung | Rln06N004 | X | | | X | |
| 44 | Lung | Rln06N027 | X | | | X | |
| 45 | Lung | Rln06N023 | X | | | X | |
| 46 | Heart | Cht11N009 | X | | | X | |
| 47 | Heart | Cht05N005 | X | | | X | |
| 48 | Heart | Cht11N001 | X | | | X | |
| 49 | Esophagus | Des06N027 | X | | | X | |
| 50 | Esophagus | Des17N004 | X | | Minimal epithelial | X | |
| | | | | | tissue | | |
| 51 | Esophagus | Des15N117 | X | | | X | |
| 52 | Stomach | Dst07N016 | X | | Hazy non-specific | X | |
| | | | | | staining in gastric | | |
| | | | | | gland cells | | |
| 53 | Stomach | Dst15N003 | X | | Hazy non-specific | X | |
| | | | | | staining in gastric | | |
| | | | | | gland cells | | |
| 54 | Stomach | Dst06N017 | X | | Hazy non-specific | X | |
| | | | | | staining in gastric | | |
| | | | | | gland cells | | |
| 55 | Small | Din07N016 | X | | Weak non-specific | X | |
| | intestine | | | | mucosal staining | | |
| 56 | Small | Din08N014 | X | | | X | |
| | intestine | | | | | | |
| 57 | Small | Din08N007 | X | | | X | |
| | intestine | | | | | | |
| 58 | Colon | Dco15N083 | X | | | X | |
| 59 | Colon | Dco06N022 | X | | | X | |
| 60 | Colon | Dco15N082 | TI | | No mucosal tissue | TI | |
| 61 | Liver | Dlv05N015 | X | | | | X |
| 62 | Liver | Dlv15N146 | X | | Non-specific | | X |
| | | | | | particulate staining | | |
| | | | | | in hepatocytes | | |
| 63 | Liver | Dlv15N145 | X | | | | X |
| 64 | Salivary | Doc11N007 | X | | | | |
| | gland | | | | | | |
| 65 | Salivary | Doc06N023 | X | | | X | |
| | gland | | | | | | |
| 66 | Salivary | Doc06N022 | X | | | X | |
| | gland | | | | | | |
| 67 | Kidney | Ukn08N047 | X | | | X | |
| 68 | Kidney | Ukn08N035 | X | | | X | |
| 69 | Kidney | Ukn06N004 | X | | | X | |
| 70 | Prostate | Mpr08N004 | X | | | X | |
| 71 | Prostate | Mpr08N005 | X | | | X | |
| 72 | Prostate | Mpr11N013 | X | | | X | |
| 73 | Uterus | Fur17N003 | X | | | X | |
| 74 | Uterus | Fur50N029 | X | | | X | |
| 75 | Uterus | Fur07N040 | X | | | X | |
| 76 | Cervix | Fdu50N020 | X | | | X | |
| 77 | Cervix | Fdu17N003 | X | | | X | |
| 78 | Cervix | Fdu50N019 | X | | | X | |
| 79 | Bladder | Ubd08N007 | X | | | X | |
| 80 | Bladder | Ubd08N001 | X | | | X | |
| 81 | Bladder | Ubd08N009 | X | | | X | |
| 82 | Skeletal | Srm15N008 | X | | | X | |
| | muscle | | | | | | |
| 83 | Skeletal | Srm15N009 | X | | | X | |
| | muscle | | | | | | |
| 84 | Skeletal | Srm05N014 | X | | | X | |
| | muscle | | | | | | |
| 85 | Skin | Kin15N007 | X | | | X | |
| 86 | Skin | Kin07N025 | X | | | X | |
| 87 | Skin | Kin15N009 | X | | | X | |
| 88 | Nerve | Scf10N003 | X | | | X | |
| 89 | Nerve | Scf08N010 | X | | | X | |
| 90 | Nerve | Scf11N005 | X | | | X | |
| 91 | Artery | Sbv07N029 | X | | | X | |
| 92 | Pericardium | Apr15N145 | X | | | X | |
| 93 | Pericardium | Apr15N146 | X | | | X | |
| 94 | Eye | Vey130002 | TI | | Only pigmented | TI | |
| | | | | | retina present | | |
| 95 | Eye | Vey140001 | TI | | Only pigmented | TI | |
| | | | | | retina present | | |
| 96 | Eye | Vey070007 | X | | Intense | X | |
| | | | | | pigmentation | | |
| 97 | Larynx | Rla13N011 | X | | | X | |
| 98 | Larynx | Rla13N020 | X | | | X | |
| 99 | Larynx | Rla07N010 | X | | | X | |

| | Target IHC | | | |
|---|---|---|---|---|
| | Cell | Intensity | | |
| No. | type | (0-3+) | Localization | Comment |
| 1 | | | | |
| 2 | | | | Scattered glial cell staining that is likely |
| | | | | non- specific due to high antibody |
| | | | | concentration |
| 3 | | | | Scattered glial cell staining that is likely |
| | | | | non- specific due to high antibody |
| | | | | concentration |
| 4 | | | | |
| 5 | | | | |
| 6 | | | | |
| 7 | Epithelial cells | 3+ | Cytoplasmic/ | Intense staining in cortex and likely |
| | | | membrane | non- specific |
| 8 | Epithelial cells | 3+ | Cytoplasmic/ | Intense staining in cortex and likely |
| | | | membrane | non- specific |
| 9 | Epithelial cells | 3+ | Cytoplasmic/ | Intense staining in cortex and likely |
| | | | membrane | non- specific |
| 10 | | | | |
| 11 | | | | |
| 12 | | | | |
| 13 | | | | Severe autolysis and non-specific |
| | | | | staining |
| 14 | Epithelial cells | 3+ | C/M | Intense staining and likely non-specific |
| 15 | Epithelial cells | 1+ | C/M | Very weak staining and likely non- |
| | | | | specific |
| 16 | | | | |
| 17 | | | | Non-specific staining in exfoliated cells |
| 18 | | | | |
| 19 | | | | |
| 20 | | | | Non-specific nuclear staining in |
| | | | | glandular cells |
| 21 | | | | Non-specific nuclear staining in |
| | | | | glandular cells |
| 22 | Spermatogonia | 3+ | C/M in Leydig; | Intense nuclear staining suggests non- |
| | cells; | | N in others | specific staining with this antibody clone |
| | spermatids; | | | at this high concentration |
| | Leydig cells | | | |
| 23 | Spermatogonia | 3+ | C/M in Leydig; | Intense nuclear staining suggests non- |
| | cells; | | N in others | specific staining with this antibody clone |
| | spermatids; | | | at this high concentration |
| | Leydig cells | | | |
| 24 | Spermatogonia | 3+ | C/M in Leydig; | ntense nuclear staining suggests non- |
| | cells; | | N in others | specific staining with this antibody clone |
| | spermatids; | | | at this high concentration |
| | Leydig cells | | | |
| 25 | | | | Non-specific staining in colloid and |
| | | | | serum |
| 26 | | | | Non-specific staining in colloid and |
| | | | | serum |
| 27 | | | | Non-specific staining in colloid and |
| | | | | serum |
| 28 | | | | |
| 29 | | | | |
| 30 | | | | |
| 31 | | | | Hazy non-specific staining in red pulp |
| 32 | | | | Hazy non-specific staining in red pulp |
| 33 | | | | |
| 34 | | | | Only adipose tissue present |
| 35 | | | | Few nuclear staining in superficial |
| | | | | epithelium |
| 36 | | | | Minimal lymphoid tissue present |
| 37 | | | | |
| 38 | | | | |
| 39 | | | | |
| 40 | | | | Non-specific staining in exfoliated cells |
| 41 | | | | Non-specific staining in exfoliated cells |
| 42 | | | | Non-specific staining in exfoliated cells |
| 43 | | | | Non-specific staining in exfoliated cells |
| | | | | and alveolar macrophages |
| 44 | | | | |
| 45 | | | | Non-specific staining in exfoliated cells |
| | | | | and alveolar macrophages |
| 46 | | | | Non-specific hazy staining in |
| | | | | cardiomyocytes |
| 47 | | | | Non-specific hazy staining in |
| | | | | cardiomyocytes |
| 48 | | | | |
| 49 | | | | Non-specific cytoplasmic staining in |
| | | | | squamous epithelial |
| 50 | | | | Minimal epithelial tissue |
| 51 | | | | |
| 52 | | | | Intense non-specific staining in gastric |
| | | | | gland cells |
| 53 | | | | Weak non-specific staining in gastric |
| | | | | gland cells |
| 54 | | | | Intense non-specific staining in gastric |
| | | | | gland cells |
| 55 | | | | Non-specific cytoplasmic staining in |
| | | | | epithelial cells as observed in isotype |
| 56 | | | | |
| 57 | | | | |
| 58 | | | | |
| 59 | | | | |
| 60 | | | | No mucosal tissue |
| 61 | Hepatocytes | 3+ | C/M | Very intense staining and likely non- |
| | | | | specific |
| 62 | Hepatocytes | 3+ | C/M | Very intense staining and likely non- |
| | | | | specific |
| 63 | Hepatocytes | 3+ | C/M | Very intense staining and likely non- |
| | | | | specific |
| 64 | | | | |
| 65 | | | | |
| 66 | | | | |
| 67 | | | | |
| 68 | | | | |
| 69 | | | | |
| 70 | | | | |
| 71 | | | | |
| 72 | | | | |
| 73 | | | | |
| 74 | | | | on-specific nuclear staining in glandular |
| | | | | epithelial cells and stromal cells |
| 75 | | | | |
| 76 | | | | Non-specific nuclear staining in |
| | | | | epithelial and stromal cells |
| 77 | | | | |
| 78 | | | | |
| 79 | | | | Non-specific nuclear staining in |
| | | | | epithelial and stromal cells |
| 80 | | | | Non-specific nuclear staining in |
| | | | | epithelial and stromal cells |
| 81 | | | | |
| 82 | | | | |
| 83 | | | | |
| 84 | | | | Hazy non-specific staining in cytoplasm |
| 85 | | | | |
| 86 | | | | |
| 87 | | | | |
| 88 | | | | |
| 89 | | | | |
| 90 | | | | |
| 91 | | | | |
| 92 | | | | |
| 93 | | | | |
| 94 | | | | Only pigmented retina present |
| 95 | | | | Only pigmented retina present |
| 96 | | | | Intense pigmentation |
| 97 | | | | |
| 98 | | | | Non-specific nuclear staining in |
| | | | | epithelial and stromal cells |
| 99 | | | | Non-specific nuclear staining in |
| | | | | epithelial and stromal cells |

MAGE-B2 IHC staining with MAGE-B2 (IC3) is observed in testes tissues characterized by cytoplasmic to membrane staining of spermatogonia cells, and nuclear staining in some cells. There are MAGE-B2 IHC staining in other tissues such as liver, adrenal gland, intestines that are most likely non-specific.

TABLE 19
MAGE-B2 (4G17) Immunohistochemistry Staining
in Normal Human Tissue TMA (FDA999x)

| Tissue identification | | | | IHC Isotype Control | | |
|---|---|---|---|---|---|---|
| Position | No. | Organ | Tissue ID | Negative | Positive | Comment |
| A1 | 1 | Cerebrum | Nct05N012 | X | | |
| A2 | 2 | Cerebrum | Nct05N016 | X | | |
| A3 | 3 | Cerebrum | Nct08N046 | X | | |
| A4 | 4 | Cerebellum | Ncb05N008 | X | | |
| A5 | 5 | Cerebellum | Ncb15N146 | X | | |
| A6 | 6 | Cerebellum | Ncb05N011 | X | | |
| A7 | 7 | Adrenal gland | Eag06N019 | X | | Non-specific |
| | | | | | | particulate staining |
| | | | | | | in cortex (Zona |
| | | | | | | fasiculata and |
| | | | | | | zona reticularis) |
| A8 | 8 | Adrenal gland | Eag06N010 | X | | Non-specific |
| | | | | | | particulate staining |
| | | | | | | in cortex (Zona |
| | | | | | | fasiculata and |
| | | | | | | zona reticularis) |
| A9 | 9 | Adrenal gland | Eag15N145 | X | | Non-specific |
| | | | | | | particulate staining |
| | | | | | | in cortex (Zona |
| | | | | | | fasiculata and |
| | | | | | | zona reticularis) |
| A10 | 10 | Ovary | Fov07N034 | X | | |
| A11 | 11 | Ovary | Fov13N015 | X | | |
| A12 | 12 | Ovary | Fov13N023 | X | | |
| B1 | 13 | Pancreas | Dpa15N145 | TI | | Tissue autolyzed |
| B2 | 14 | Pancreas | Dpa06N023 | X | | |
| B3 | 15 | Pancreas | Dpa11N012 | X | | |
| B4 | 16 | Lymph node | Ily06N015 | X | | |
| B5 | 17 | Lymph node | Ily08N047 | X | | Non-specific |
| | | | | | | staining in |
| | | | | | | exfoliated cells |
| B6 | 18 | Lymph node | Ily08N014 | X | | |
| B7 | 19 | Hypophysis | Ept14N001 | X | | |
| B8 | 20 | Hypophysis | Ept13N019 | X | | |
| B9 | 21 | Hypophysis | Ept13N020 | X | | |
| B10 | 22 | Testis | Mtt09N048 | X | | |
| B11 | 23 | Testis | Mtt08N001 | X | | |
| B12 | 24 | Testis | Mtt08N009 | X | | |
| C1 | 25 | Thyroid gland | Etg07N002 | X | | Hazy non-specific |
| | | | | | | staining in colloid |
| C2 | 26 | Thyroid gland | Etg08N047 | X | | Hazy non-specific |
| | | | | | | staining in colloid |
| C3 | 27 | Thyroid gland | Etg06N003 | X | | Hazy non-specific |
| | | | | | | staining in colloid |
| C4 | 28 | Breast | Fmg08N034 | X | | |
| C5 | 29 | Breast | Fmg50N034 | X | | |
| C6 | 30 | Breast | Fmg07N040 | X | | |
| C7 | 31 | Spleen | Isp15N145 | X | | |
| C8 | 32 | Spleen | Isp15N146 | X | | |
| C9 | 33 | Spleen | Isp11N001 | X | | |
| C10 | 34 | Tonsil | Rts13N011 | TI | | Only adipose |
| | | | | | | tissue present |
| C11 | 35 | Tonsil | Rts50N001 | X | | |
| C12 | 36 | Tonsil | Rts08N042 | X | | Minimal lymphoid |
| | | | | | | tissue present |
| D1 | 37 | Thymus gland | Ith06N024 | X | | |
| D2 | 38 | Thymus gland | Ith06N002 | X | | |
| D3 | 39 | Thymus gland | Ith15N145 | X | | |
| D4 | 40 | Bone marrow | Ibm06N024 | X | | Non-specific |
| | | | | | | staining in |
| | | | | | | exfoliated cells |
| D5 | 41 | Bone marrow | Ibm11N015 | X | | Non-specific |
| | | | | | | staining in |
| | | | | | | exfoliated cells |
| D6 | 42 | Bone marrow | Ibm12N001 | X | | Non-specific |
| | | | | | | staining in |
| | | | | | | exfoliated cells |
| D7 | 43 | Lung | Rln06N004 | X | | |
| D8 | 44 | Lung | Rln06N027 | X | | |
| D9 | 45 | Lung | Rln06N023 | X | | |
| D10 | 46 | Heart | Cht11N009 | X | | |
| D11 | 47 | Heart | Cht05N005 | X | | |
| D12 | 48 | Heart | Cht11N001 | X | | |
| E1 | 49 | Esophagus | Des06N027 | X | | |
| E2 | 50 | Esophagus | Des17N004 | X | | Minimal epithelial |
| | | | | | | tissue |
| E3 | 51 | Esophagus | Des15N117 | X | | |
| E4 | 52 | Stomach | Dst07N016 | X | | Hazy non-specific |
| | | | | | | staining in gastric |
| | | | | | | gland cells |
| E5 | 53 | Stomach | Dst15N003 | X | | Hazy non-specific |
| | | | | | | staining in gastric |
| | | | | | | gland cells |
| E6 | 54 | Stomach | Dst06N017 | X | | Hazy non-specific |
| | | | | | | staining in gastric |
| | | | | | | gland cells |
| E7 | 55 | Small intestine | Din07N016 | X | | Weak non-specific |
| | | | | | | mucosal staining |
| E8 | 56 | Small intestine | Din08N014 | X | | |
| E9 | 57 | Small intestine | Din08N007 | X | | |
| E10 | 58 | Colon | Dco15N083 | X | | |
| E11 | 59 | Colon | Dco06N022 | X | | |
| E12 | 60 | Colon | Dco15N082 | TI | | No mucosal tissue |
| F1 | 61 | Liver | Dlv05N015 | X | | |
| F2 | 62 | Liver | Dlv15N146 | X | | Non-specific |
| | | | | | | particulate staining |
| | | | | | | in hepatocytes |
| F3 | 63 | Liver | Dlv15N145 | X | | |
| F4 | 64 | Salivary gland | Doc11N007 | X | | |
| F5 | 65 | Salivary gland | Doc06N023 | X | | |
| F6 | 66 | Salivary gland | Doc06N022 | X | | |
| F7 | 67 | Kidney | Ukn08N047 | X | | |
| F8 | 68 | Kidney | Ukn08N035 | | | |
| F9 | 69 | Kidney | Ukn06N004 | | | |
| F10 | 70 | Prostate | Mpr08N004 | | | |
| F11 | 71 | Prostate | Mpr08N005 | X | | |
| F12 | 72 | Prostate | Mpr11N013 | X | | |
| G1 | 73 | Uterus | Fur17N003 | X | | |
| G2 | 74 | Uterus | Fur50N029 | X | | |
| G3 | 75 | Uterus | Fur07N040 | X | | |
| G4 | 76 | Cervix | Fdu50N020 | X | | |
| G5 | 77 | Cervix | Fdu17N003 | X | | |
| G6 | 78 | Cervix | Fdu50N019 | X | | |
| G7 | 79 | Bladder | Ubd08N007 | X | | |
| G8 | 180 | Bladder | Ubd08N001 | X | | |
| G9 | 81 | Bladder | Ubd08N009 | X | | |
| G10 | 82 | Skeletal muscle | Srm15N008 | X | | |
| G11 | 83 | Skeletal muscle | Srm15N009 | X | | |
| G12 | 84 | Skeletal muscle | Srm05N014 | X | | |
| H1 | 85 | Skin | Kin15N007 | X | | |
| H2 | 86 | Skin | Kin07N025 | X | | |
| H3 | 87 | Skin | Kin15N009 | X | | |
| H4 | 188 | Nerve | Scf10N003 | X | | |
| H5 | 89 | Nerve | Scf08N010 | X | | |
| H6 | 90 | Nerve | Scf11N005 | X | | |
| H7 | 91 | Artery | Sbv07N029 | X | | |
| H8 | 92 | Pericardium | Apr15N145 | X | | |
| H9 | 93 | Pericardium | Apr15N146 | X | | |
| H10 | 94 | Eye | Vey130002 | TI | | Only pigmented retina present |
| H11 | 95 | Eye | Vey140001 | TI | | Only pigmented retina present |
| H12 | 196 | Eye | Vey070007 | X | | Intense pigmentation |
| I1 | 197 | Larynx | Rla13N011 | X | | |
| I2 | 98 | Larynx | Rla13N020 | X | | |
| I3 | 199 | Larynx | Rla07N010 | X | | |

Target IHC

| | | Cell | Intensity | Local- | |
|---|---|---|---|---|---|
| Negative | Positive | type | (0-3+) | ization | Comment |
| X | | | | | |
| X | | | | | Non-specific staining in |
| | | | | | few glial cells |
| X | | | | | Non-specific staining in |
| | | | | | few capillaries |
| X | | | | | |
| X | | | | | Non-specific staining in |
| | | | | | few capillaries |
| X | | | | | Non-specific staining in |
| | | | | | few capillaries |
| X | | | | | Non-specific staining as |
| | | | | | observed in isotype but |
| | | | | | with increased intensity |
| X | | | | | Non-specific staining as |
| | | | | | observed in isotype but |
| | | | | | with increased intensity |
| X | | | | | Non-specific staining as |
| | | | | | observed in isotype but |
| | | | | | with increased intensity |
| X | | | | | Non-specific particulate |
| | | | | | staining in cortex (Zona |
| | | | | | fasiculata and zona |
| | | | | | reticularis) as observed |
| | | | | | in isotype |
| X | | | | | |
| X | | | | | |
| TI | | | | | Tissue autolyzed |
| | X | Epithelial | 1+ | Cytoplasmic/ | Weak to mild staining and |
| | | cells | | membrane | likely non-specific |
| X | | | | | |
| X | | | | | |
| X | | | | | Non-specific staining as |
| | | | | | in isotype, but with |
| | | | | | increased intensity |
| X | | | | | |
| X | | | | | |
| | X | Epithelial | 2+ | Cytoplasmic/ | Predominantly in loosely |
| | | cells | | membrane | adhered cells and likely |
| | | | | | non-specific |
| | X | Epithelial | 2+ | Cytoplasmic/ | Predominantly in loosely |
| | | cells | | membrane | adhered cells and likely |
| | | | | | non-specific |
| | X | Spermatogonia | 3 | Cytoplasmic/ | There is nuclear staining |
| | | cells | | membrane, | in some spermatids and |
| | | | | Nuclear | hazy cytoplasmic staining |
| | | | | | in some Leydig cells. Both |
| | | | | | are likely non-specific |
| | X | Spermatogonia | 3 | Cytoplasmic/ | There is nuclear staining |
| | | cells | | membrane, | in some spermatids and |
| | | | | Nuclear | hazy cytoplasmic staining |
| | | | | | in some Leydig cells. Both |
| | | | | | are likely non-specific |
| | X | Spermatogonia | 3 | C/M; N | There is nuclear staining |
| | | cells | | | in some spermatids and |
| | | | | | hazy cytoplasmic staining |
| | | | | | in some Leydig cells. Both |
| | | | | | are likely non-specific |
| | | | | | Non-specific staining as |
| | | | | | observed in isotype but |
| | | | | | with increased intensity |
| | | | | | Non-specific staining as |
| | | | | | observed in isotype but |
| | | | | | with increased intensity |
| | | | | | Non-specific staining as |
| | | | | | observed in isotype but |
| | | | | | with increased intensity |
| X | | | | | |
| X | | | | | |
| X | | | | | |
| X | | | | | Weak non-specific staining in |
| | | | | | exfoliated cells in red pulp |
| | | | | | Weak non-specific staining in |
| | | | | | exfoliated cells in red pulp |
| X | | | | | Weak non-specific staining in |
| | | | | | exfoliated cells in red pulp |
| TI | | | | | Only adipose tissue present |
| X | | | | | Non-specific nuclear staining |
| | | | | | in some cells |
| X | | | | | |
| X | | | | | |
| X | | | | | |
| X | | | | | |
| X | | | | | Non-specific staining as |
| | | | | | observed in isotype but |
| | | | | | with increased intensity |
| X | | | | | Non-specific staining as |
| | | | | | observed in isotype but |
| | | | | | with increased intensity |
| X | | | | | Non-specific staining as |
| | | | | | observed in isotype but |
| | | | | | with increased intensity |
| X | | | | | Non-specific staining in |
| | | | | | some degenerating alveolar |
| | | | | | macrophages |
| X | | | | | |
| X | | | | | |
| X | | | | | |
| X | | | | | |
| X | | | | | |
| X | | | | | |
| X | | | | | |
| X | | | | | |
| X | | | | | Non-specific staining as |
| | | | | | observed in isotype but |
| | | | | | with increased intensity |
| X | | | | | Non-specific staining as |
| | | | | | observed in isotype but |
| | | | | | with increased intensity |
| X | | | | | Non-specific staining as |
| | | | | | observed in isotype but |
| | | | | | with increased intensity |
| X | | | | | Non-specific cytoplasmic |
| | | | | | staining in epithelial |
| | | | | | cells as observed in |
| | | | | | isotype |
| X | | | | | |
| X | | | | | |
| X | | | | | |
| | X | Hepatocytes | 2+ | Cytoplasmic/ | Weak to mild staining in |
| | | | | membrane | hepatocytes, likely non- |
| | | | | | specific |
| | X | Hepatocytes | 2+ | Cytoplasmic/ | Weak to mild staining in |
| | | | | membrane | hepatocytes, likely non- |
| | | | | | specific |
| | X | Hepatocytes | 2+ | Cytoplasmic/ | Weak to mild staining in |
| | | | | membrane | hepatocytes, likely non- |
| | | | | | specific |
| X | | | | | |
| X | | | | | |
| X | | | | | |
| X | | | | | |
| X | | | | | |
| X | | | | | |
| X | | | | | |
| X | X | Epithelial | 1+ | Cytoplasmic/ | Granular cytoplasmic |
| | | | | membrane | staining in few prostate |
| | | | | | gland epithelial cells, |
| | | | | | likely non-specific |
| X | | | | | Non-specific nuclear |
| | | | | | staining in some stromal |
| | | | | | cells and epithelial cells |
| X | | | | | |
| X | | | | | |
| X | | | | | |
| X | | | | | Non-specific nuclear |
| | | | | | staining in some stromal |
| | | | | | cells and epithelial cells |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| X | | | | | |
| TI | | | | | Only pigmented retina present |
| TI | | | | | Only pigmented retina present |
| X | | | | | Intense pigmentation |
| X | | | | | |
| X | | | | | |
| X | | | | | |

MAGE-B2 IHC staining with MAGE-B2 (4G17) was observed in testes, characterized by cytoplasmic to membrane staining of spermatogonia cells, nuclear staining in some and staining in Leydig cells. Leydig staining is likely non-specific. There was MAGE-B2 IHC staining in other tissues such as liver, adrenal gland, intestines that was most likely non-specific.

TABLE 20
Testing the 4 MAGE-B2 antibodies (4G17, 1J15, 1C3, 114) on thirteen human
cancer cell lines with knownendogenous RNA expression level by IHC.

| | | RNASeq | | | | | | | | | |
|---|---|---|---|---|---|---|---|---|---|---|---|
| | Tron | (exon | | | | | | | | | |
| | Database | specific | CCLE | | | | | | | | |
| | [RPKM] | FPKM) | (FPKM) | IHC (1J15) | | IHC (1i14) | | IHC(1C3) | | IHC (4G17) | |
| | MAGEB2 | MAGEB2 | MAGEB2 | | % | | % | | % | | % |
| Cell line | expression | expression | expression | Intensity | Frequency | Intensity | Frequency | Intensity | Frequency | Intensity | Frequency |
| T98G | 0.05 | 0 | 0.01 | 1 | 70 | 2 | 70 | 2 | 70 | 2 | 70 |
| UACC-257 | 0.04 | 0.01 | 0.01 | 0 | 0 | 0/1 | 0 | 0/1 | 0 | 0 | 0 |
| KMM-1 | 56.91 | 31.58 | 42.69 | 0/1 | 70 | 1 | 70 | 1 | 70 | 2 | 70 |
| U266B1 | 102.32 | 103.73 | 89.35 | 1 | 90 | 3 | 90 | 2 | 90 | 2 | 80 |
| CFPAC-1 | 0.45 | 0.18 | 0.40 | 0/1 | 0 | 0/1 | 0 | 0/1 | 0 | 0/1 | 0 |
| SCaBER | 0.01 | 0.01 | 0.02 | 2 | 50 | 0 | 0 | 2 | 70 | 2 | 70 |
| VMRC-LCD | 0 | | | 0 | 0 | 0 | 0 | 0 | 0 | 0 | 0 |
| NCI-H1703 | 0 | 0 | 0.00 | 0/1 | 0 | 0/1 | 0 | 0/1 | 0 | 0 | 0 |
| UM-UC-3 | | | 0.02 | 0 | 0 | 0 | | 0/1 | | 0 | 0 |
| NCI-H1395 | | | 3.52 | 0 | 0 | 0 | 0 | 0 | 0 | 0 | 0 |
| U20S | | | 45.42 | 1 | 50 | 2 | 70 | 2 | 60 | 2 | 60 |
| NCI-H82 | | 6.33 | 0.06 | 0 | 0 | 0 | 0 | 1 | 0 | 0 | 0 |

MAGEB2 IHC expression frequency with the four MAGE-B2 (4G17, 1J15, 1C3, 1i14) was high (~90%) in U266B1 cells, and moderate (~70%) in T98G, KMM-1, U2OS cells; and undetectable in CFPAC-1, UACC-257, CFPAC-1, VMRC-LCD, NCI-H1703, UM-UC-3, NCI-H1395, NCI-H82 cell lines with the 4 MAGE-B2 clones (4G17, 1J15, 1C3, 1i14). MAGEB2 IHC expression frequencies in SCaBER cells were inconsistent with the four MAGE-B2 clones (4G17, 1J15, 1C3, 1i14).

TABLE 21
Standard IgG Antibody VL CDRs

| Ab | Type | CDR1 | CDR2 | CDR3 |
|---|---|---|---|---|
| 7M5 | NA | CAGGCCAGTCAGAGCATTAGGAAT | GCTGCATCCAAACTGGCCTCT | CAATGCAGTTATGTTAGTAGTAGTGGTACT |
| | | GAATTATTT | (SEQ ID NO: 6) | TATGGAAATGTT |
| | | (SEQ ID NO: 5) | | (SEQ ID NO: 7) |
| | AA | QASQSIRNELF | AASKLAS | QCSYVSSSGTYGNV |
| | | (SEQ ID NO: 7) | (SEQ ID NO: 8) | (SEQ ID NO: 9) |
| 1J14 | NA | CAGGCCAGTGAAAGCATTAGCAAC | TGGGCATCCACTCTGGCATCT | CAACAGGGTTATAGTAGTAGTAATGTTGAT |
| | | TACTTATCC | (SEQ ID NO: 11) | AATCTT |
| | | (SEQ ID NO: 10) | | (SEQ ID NO: 12) |
| | AA | QASESISNYLS | WASTLAS | QQGYSSSNVDNL |
| | | (SEQ ID NO: 13) | (SEQ ID NO: 14) | (SEQ ID NO: 15) |
| 7F15 | NA | CAGTCCAGTCAGAGTGTTTGGCATA | GGTGCATCCACTTTGGCATCT | GCAGGCGGTTATGGACGTAGTAGTGAAAA |
| | | ACGACTACTTATCC | (SEQ ID NO: 17) | TGGT |
| | | (SEQ ID NO: 16) | | (SEQ ID NO: 18) |
| | AA | QSSQSVWHNDYLS | GASTLAS | AGGYGRSSENG |
| | | (SEQ ID NO: 19) | (SEQ ID NO: 20) | (SEQ ID NO: 21) |
| 5I9 | NA | CAGTCCAGTAAGAGTGTTTATAATA | GATGCATCGACTCTAGATTCT | GTAGGCGGTTATAGTAGTCGTAGTGATAAT |
| | | ACAACTGGTTAGCC | (SEQ ID NO: 23) | GGT |
| | | (SEQ ID NO: 22) | | (SEQ ID NO: 24) |
| | AA | QSSKSVYNNNWLA | DASTLDS | VGGYSSRSDNG |
| | | (SEQ ID NO: 25) | (SEQ ID NO: 26) | (SEQ ID NO: 27) |
| 1J15 | NA | CAGGCCAGTCAGAGTATTAGTAGTT | AGGGCATCCACTCTGGCATCT | CAAAGCTATGATGATAGTAGTGATAATAAT |
| | | ACTTATCC | (SEQ ID NO: 29) | TTTTTTTATGGT |
| | | (SEQ ID NO: 28) | | (SEQ ID NO: 30) |
| | AA | QASQSISSYLS | RASTLAS | QSYDDSSDNNFFYG |
| | | (SEQ ID NO: 31) | (SEQ ID NO: 32) | (SEQ ID NO: 33) |
| 4G17 | NA | CAGGCCAGTCAGAACATTGATAGT | AGGGCATCCACTCTGGCATCT | CAAAGCTATGATGATAGTAGGAGTAGTAG |
| | | TACTTAGCC | (SEQ ID NO: 35) | TTTTTTTTATGGT |
| | | (SEQ ID NO: 34) | | (SEQ ID NO: 36) |
| | AA | QASQNIDSYLA | RASTLAS | QSYDDSRSSSFFYG |
| | | (SEQ ID NO: 37) | (SEQ ID NO: 38) | (SEQ ID NO: 39) |
| 4A15 | NA | CAGGCCAGTCAGAACATTAATAGT | AGGGCATCCACTCTGGCATCT | CAAAGCTATGATGATAGTAGGAGTATTAGT |
| | | TACTTAGCC | (SEQ ID NO: 41) | TTTTTTTATGCT |
| | | (SEQ ID NO: 40) | | (SEQ ID NO: 42) |
| | AA | QASQNINSYLA | RASTLAS | QSYDDSRSISFFYA |
| | | SEQ ID NO: 43) | (SEQ ID NO: 44) | (SEQ ID NO: 45) |
| 1J16 | NA | CAGGCCAGTCAGAGCATTAGTAGC | GCTGCATCCACTCTGGCATCT | CAAAGCTATGATGATAGTAGGAGTAGTAG |
| | | TACTTAGCC | (SEQ ID NO: 47) | TTTTTTTTATGCT |
| | | (SEQ ID NO: 46) | | (SEQ ID NO: 48) |
| | AA | QASQSISSYLA | AASTLAS | QSYDDSRSSSFFYA |
| | | (SEQ ID NO: 49) | (SEQ ID NO: 50) | (SEQ ID NO: 51) |
| 1C18 | NA | CAGGCCAGTCAGAGCATTAGCAGT | AGGGCAACCACTCTGGCATCT | CAAAGTTATGATGATAGTAGTAGTAGTAAT |
| | | TGGTTATCC | (SEQ ID NO: 53) | TTTTTTTATGCT |
| | | (SEQ ID NO: 52) | | (SEQ ID NO: 54) |
| | AA | QASQSISSWLS | RATTLAS | QSYDDSSSSNFFYA |
| | | (SEQ ID NO: 55) | (SEQ ID NO: 56) | SEQ ID NO: 57) |
| 7H4 | NA | CAGTCCAGTCAGAGTGTTTATAGTA | AAGGCATCCACTCTGGCATCT | CAAGGCTATTATAGTGGTGTTATTTATATG |
| | | ACAACCTCTTATCT | (SEQ ID NO: 59) | (SEQ ID NO: 60) |
| | | (SEQ ID NO: 58) | | |
| | AA | QSSQSVYSNNLLS | KASTLAS | QGYYSGVIYM |
| | | (SEQ ID NO: 61) | (SEQ ID NO: 62) | (SEQ ID NO: 63) |
| 1H24 | NA | CAGGCCAGTCAGAGCATTAGTAGT | AGGGCATCCACTCTGGCATCT | CAAAGCTATGATGATAGTAGTAGTAATAAT |
| | | TACTTATCC | (SEQ ID NO: 65) | TTTTTTTATGGT |
| | | (SEQ ID NO: 64) | | (SEQ ID NO: 66) |
| | AA | QASQSISSYLS | RASTLAS | QSYDDSSSNNFFYG |
| | | (SEQ ID NO: 67) | (SEQ ID NO: 68) | (SEQ ID NO: 69) |
| 1C3 | NA | CAGGCCAGTCAGAACATTAGTAGC | AGGGCATCCACTCTGGCATCT | CAAAGCTATGATGATAGTAGGAGTAGTAA |
| | | TACTTAGCC | (SEQ ID NO: 71) | TTTTTTTTATGCT |
| | | (SEQ ID NO: 70) | | (SEQ ID NO: 72) |
| | AA | QASQNISSYLA | RASTLAS | QSYDDSRSSNFFYA |
| | | (SEQ ID NO: 73) | (SEQ ID NO: 74) | (SEQ ID NO: 75) |
| 5H20 | NA | CAGTCCAGTGAGAGCGTTTATAATC | GATGCATCCACTCTGGCATCT | CAAGGCTATTATCAAACTAGTGTTTGGGCT |
| | | ACAACTGGTTAGGC | (SEQ ID NO: 77) | (SEQ ID NO: 78) |
| | | (SEQ ID NO: 76) | | |
| | AA | QSSESVYNHNWLG | DASTLAS | QGYYQTSVWA |
| | | (SEQ ID NO: 79) | (SEQ ID NO: 80) | (SEQ ID NO: 81) |
| 1I14 | NA | CAGTCCAGTCAGAGTGTTTATGATA | GGTGCATCCACTCTGGCATCT | CAATGTACTTATTATGTTAGTAGTTATCAA |
| | | ACAATGCTTTAGCC | (SEQ ID NO: 83) | AATGAT |
| | | (SEQ ID NO: 82) | | (SEQ ID NO: 84) |
| | AA | QSSQSVYDNNALA | GASTLAS | QCTYYVSSYQND |
| | | (SEQ ID NO: 85) | (SEQ ID NO: 86) | (SEQ ID NO: 87) |
| 1H17 | NA | CAGTCCAGTAAGAGTGTTTATAATA | GGTGCATCCACTCTGGCATCT | GCAGGCGGTTATAGTAGTAGTAGTGATACA |
| | | AGAACTGGTTATCC | (SEQ ID NO: 89) | TTTGCT |
| | | (SEQ ID NO: 88) | | (SEQ ID NO: 90) |
| | AA | QSSKSVYNKNWLS | GASTLAS | AGGYSSSSDTFA |
| | | (SEQ ID NO: 91) | SEQ ID NO: 92) | (SEQ ID NO: 93) |
| 7B20 | NA | GCGACCTCTTATCC | AAGGCATCCACTCTGGCATCT | CAAGGCTACTATAGTGGTGTGGTTTATATT |
| | | CAGTCCAGTCAGAGCGTTTATAGTA | (SEQ ID NO: 95) | (SEQ ID NO: 96) |
| | | (SEQ ID NO: 94) | | |
| | AA | QSSQSVYSSDLLS | KASTLAS | QGYYSGVVYI |
| | | (SEQ ID NO: 97) | SEQ ID NO: 98) | (SEQ ID NO: 99) |
| 7J6 | NA | CAGTCCAGTCAGAGCGTTTATAGTA | AAGGCATCCACTCTGGCATCT | CAAGGCTACTATAGTGGTGTGGTTTATATT |
| | | GCGACCTCTTATCC | (SEQ ID NO: 101) | |
| | | (SEQ ID NO: 100) | | (SEQ ID NO: 102) |
| | AA | QSSQSVYSSDLLS | KASTLAS | QGYYSGVVYI |
| | | (SEQ ID NO: 103) | (SEQ ID NO: 104) | (SEQ ID NO: 105) |
| 1D2 | NA | CAGGCCAGTCAGAGCATTAGTAGT | AGGGCATCCCCTCTGGCATCT | CAAAGCTACGATGATAGTAGTAGTAATAAT |
| | | TACTTATCT | (SEQ ID NO: 107) | TTTTTTTATGGT |
| | | (SEQ ID NO: 106) | | (SEQ ID NO: 108) |
| | AA | QASQSISSYLS | RASPLAS | QSYDDSSSNNFFYG |
| | | (SEQ ID NO: 109) | SEQ ID NO: 110) | (SEQ ID NO: 111) |
| 4G8 | NA | AGAACTGGTTATCC | GGTGCATCCACTCTGGCATCT | TTTGCT |
| | | CAGTCCAGTAAGAGTGTTTATAATA | (SEQ ID NO: 113) | GCAGGCGGTTATAGTAGTAGTAGTGATACG |
| | | (SEQ ID NO: 112) | | (SEQ ID NO: 114) |
| | AA | QSSKSVYNKNWLS | GASTLAS | AGGYSSSSDTFA |
| | | (SEQ ID NO: 115) | SEQ ID NO: 116) | (SEQ ID NO: 117) |
| 7I4 | NA | CAGTCCAGTCAGAGCCTTTATAATA | AAGGCATCCACTCTGGCATCT | CAAGGCTACTATAGTGGTGTGGTTTATATT |
| | | GCGACCTCTTATCC | (SEQ ID NO: 119) | (SEQ ID NO: 120) |
| | | (SEQ ID NO: 118) | | |
| | AA | QSSQSLYNSDLLS | KASTLAS | QGYYSGVVYI |
| | | (SEQ ID NO: 121) | (SEQ ID NO: 122) | (SEQ ID NO: 123) |
| 1I23 | NA | CAGTCCAGTAAGAGTGTTTATAATA | GGTGCATCCACTCTGGCATCT | GCAGGCGGTTATAGTAGTAGTAGTGATACA |
| | | AGAACTGGTTATCC | (SEQ ID NO: 125) | TTTGCT |
| | | (SEQ ID NO: 124) | | (SEQ ID NO: 126) |
| | AA | QSSKSVYNKNWLS | GASTLAS | AGGYSSSSDTFA |
| | | (SEQ ID NO: 127) | (SEQ ID NO: 128) | (SEQ ID NO: 129) |
| 1J13 | NA | CAGGCCCTTCAGAGTGTTTATGATA | GGTGCATCCACTCTGGCATCT | CAATGTACTTATTATGTTAGTAGTTATCAA |
| | | ACAATGCTTTATCC | (SEQ ID NO: 131) | AATGAT |
| | | (SEQ ID NO: 130) | | (SEQ ID NO: 132) |
| | AA | QALQSVYDNNALS | GASTLAS | QCTYYVSSYQND |
| | | (SEQ ID NO: 133) | (SEQ ID NO: 134) | (SEQ ID NO: 135) |
| 1G24 | NA | CAGGCCAGTGAGAGCATTGGCAAT | AGGGCATCCACTCTGGCATCT | CAAAGCTATGATGATAGTAGTAGTAGTAGT |
| | | GCATTAGCC | (SEQ ID NO: 137) | TTTTTTTATGCT |
| | | (SEQ ID NO: 136) | | (SEQ ID NO: 138) |
| | AA | QASESIGNALA | RASTLAS | QSYDDSSSSSFFYA |
| | | (SEQ ID NO: 139) | (SEQ ID NO: 140) | (SEQ ID NO: 141) |
| 4A7 | NA | CAGTCCAGTAAGAGTGTTTATAATA | GGTGCATCCACTCTGGCATCT | GCAGGCGGTTATAGTAGTAGTAGTGATACG |
| | | AGAACTGGTTATCC | (SEQ ID NO: 143) | TTTGCT |
| | | (SEQ ID NO: 142) | | (SEQ ID NO: 144) |
| | AA | QSSKSVYNKNWLS | GASTLAS | AGGYSSSSDTFA |
| | | (SEQ ID NO: 145) | (SEQ ID NO: 146) | (SEQ ID NO: 147) |

TABLE 22
Standard IgG Antibody VH CDRs

| Ab | Type | CDR1 | CDR2 | CDR3 |
|---|---|---|---|---|
| 7M5 | NA | AGCCATGCAATGATC | ACCATTGGGAGTCGTGATACTATATATTAT | AACGCCTTG |
| | | (SEQ ID NO: 148) | GCGAGCTGGGCGAAAGGC | (SEQ ID NO: 150) |
| | | | (SEQ ID NO: 149) | |
| | | SHAMI | TIGSRDTIYYASWAKG | NAL |
| | AA | (SEQ ID NO: 151) | (SEQ ID NO: 152) | (SEQ ID NO: 153) |
| 1J14 | NA | AGCTACGACATGAGC | ATTATTTATGCTAGTGGTAGCACATACTAC | GACCCTGCTGGTTATAGCATTAGCTTTGGC |
| | | (SEQ ID NO: 154) | GCGAGCTGGGCGAAAGGC | TTG |
| | | | (SEQ ID NO: 155) | (SEQ ID NO: 156) |
| | AA | SYDMS | IIYASGSTYYASWAKG | DPAGYSISFGL |
| | | (SEQ ID NO: 157) | (SEQ ID NO: 158) | (SEQ ID NO: 159) |
| 7F15 | NA | AGCTACAACATGGGC | ATCATTGGTGCTAGTGATAGCGCATTGTAC | GGTGGTCTTGGTTTGAGTACTGGTTTTGCGT |
| | | (SEQ ID NO: 160) | GCGAGCTGGGCAAAAGGC | TG |
| | | | (SEQ ID NO: 161) | (SEQ ID NO: 162) |
| | AA | SYNMG | IIGASDSALYASWAKG | GGLGLSTGFAL |
| | | (SEQ ID NO: 163) | (SEQ ID NO: 164) | (SEQ ID NO: 165) |
| 5I9 | NA | AGCTACGACATGAGC | TATATTGCTACTGATGGTAGGCCATATTAC | GGGGGGTATGCTGGTGGCTTG |
| | | (SEQ ID NO: 166) | GCGAGCTGGGCGAAAGGC | (SEQ ID NO: 168) |
| | | | (SEQ ID NO: 167) | |
| | AA | SYDMS | YIATDGRPYYASWAKG | GGYAGGL |
| | | (SEQ ID NO: 169) | (SEQ ID NO: 170) | (SEQ ID NO: 171) |
| 1J15 | NA | AATTATTATATTTGC | TGCATTGACAATGCTAATGGTAGGACTTAC | TCATTGTCTACTCCCTTG |
| | | (SEQ ID NO: 172) | TACGCGAGCTGGGCGAAAGGC | (SEQ ID NO: 174) |
| | | | (SEQ ID NO: 173) | |
| | AA | NYYIC | CIDNANGRTYYASWAKG | SLSTPL |
| | | (SEQ ID NO: 175) | (SEQ ID NO: 176) | (SEQ ID NO: 177) |
| 4G17 | NA | AACTATTATATTTGT | TGCATTGACAATGTTAATGGTAGGACCTAC | TCCTTGGCTACTCCCTTG |
| | | (SEQ ID NO: 178) | TACGCGAGCTGGGCGAAAGGC | (SEQ ID NO: 180) |
| | | | (SEQ ID NO: 179) | |
| | AA | NYYIC | CIDNVNGRTYYASWAKG | SLATPL |
| | | (SEQ ID NO: 181) | (SEQ ID NO: 182) | (SEQ ID NO: 183) |
| 4A15 | NA | AACTACTACATCTGC | TGCATTGACAATGTTAATGGTAGGACTTAC | TCCTTGGCTACTCCCTTG |
| | | (SEQ ID NO: 184) | TACGCGAGCTGGGCGAAAGGC | (SEQ ID NO: 186) |
| | | | (SEQ ID NO: 185) | |
| | AA | NYYIC | CIDNVNGRTYYASWAKG | SLATPL |
| | | (SEQ ID NO: 187) | (SEQ ID NO: 188) | (SEQ ID NO: 189) |
| 1J16 | NA | AACTACTACATCTGC | TGCATTGACAATATTAATGGTAGGACTTAC | TCCTTGGCTACTCCCTTG |
| | | (SEQ ID NO: 190) | TACGCGAGCTGGGCGAAAGGC | (SEQ ID NO: 192) |
| | | | (SEQ ID NO: 191) | |
| | AA | NYYIC | CIDNINGRTYYASWAKG | SLATPL |
| | | (SEQ ID NO: 193) | (SEQ ID NO: 194) | (SEQ ID NO: 195) |
| 1C18 | NA | AATTATTATATATGT | TGTATTGATAACGCTAATGGTAGGACTTAC | TCATTGTCTACTAACTTG |
| | | (SEQ ID NO: 196) | TACGCGACCTGGGCGAAAGGC | (SEQ ID NO: 198) |
| | | | (SEQ ID NO: 197) | |
| | AA | NYYIC | CIDNANGRTYYATWAKG | SLSTNL |
| | | (SEQ ID NO: 199) | (SEQ ID NO: 200) | (SEQ ID NO: 201) |
| 7H4 | NA | AGCTACTACTACATATGC | TGTATTGGTGGTGGTAATACCGATGCCACT | GGCGGTCCTGATAATAATGTCCAATTTAAC |
| | | (SEQ ID NO: 202) | GCCTACGCGAGGTGGGCGAAAGGC | TTG |
| | | | (SEQ ID NO: 203) | (SEQ ID NO: 204) |
| | AA | SYYYIC | CIGGGNTDATAYARWAKG | GGPDNNVQFNL |
| | | (SEQ ID NO: 205) | (SEQ ID NO: 206) | (SEQ ID NO: 207) |
| 1H24 | NA | AATTATTATATTTGC | TGCATTGACAATAGTAATGGTAGGACTTAC | TCATTGTCTACTCCCTTG |
| | | (SEQ ID NO: 208) | TACGCGAGCTGGGCGAAAGGC | (SEQ ID NO: 210) |
| | | | (SEQ ID NO: 209) | |
| | AA | NYYIC | CIDNSNGRTYYASWAKG | SLSTPL |
| | | (SEQ ID NO: 211) | (SEQ ID NO: 212) | (SEQ ID NO: 213) |
| 1C3 | NA | AACTACTACATCTGC | TGTATTGACAATGCTAATGGTAGGACTTAC | TCCTTGGCTACTCCCTTG |
| | | (SEQ ID NO: 214) | TACGCGAGCTGGGCGAAAGGC | (SEQ ID NO: 216) |
| | | | (SEQ ID NO: 215) | |
| | AA | NYYIC | CIDNANGRTYYASWAKG | SLATPL |
| | | (SEQ ID NO: 217) | (SEQ ID NO: 218) | (SEQ ID NO: 219) |
| 5H20 | NA | AGCAGTGCAGTGACC | TTCCTCCAAGCTGGGGATGGTAGCGCATAC | CATAAGGGTAATAGTTACGTGCCTAACTTG |
| | | (SEQ ID NO: 220) | TACGCGAGCTGGGCGAAAGGC | (SEQ ID NO: 222) |
| | | | (SEQ ID NO: 221) | |
| | AA | SSAVT | FLQAGDGSAYYASWAKG | HKGNSYVPNL |
| | | (SEQ ID NO: 223) | (SEQ ID NO: 224) | (SEQ ID NO: 225) |
| 1I14 | NA | AGCTATGCAATGAGC | AGCATTGGTGGTGGTGGTAGCGCAGTCTAC | GGATTTTATAGTATAGACTTG |
| | | (SEQ ID NO: 226) | GCGAGCTGGGCGAAAGGC | (SEQ ID NO: 228) |
| | | | (SEQ ID NO: 227) | |
| | AA | SYAMS | SIGGGGSAVYASWAKG | GFYSIDL |
| | | (SEQ ID NO: 229) | (SEQ ID NO: 230) | (SEQ ID NO: 231) |
| 1H17 | NA | AGCGGCCAACTCATGTGC | TGCATTGGTTCTGGTAGTAATGCTATTAGC | GTGGGCTCCGATGACTATGGTGACTCTGAT |
| | | (SEQ ID NO: 232) | ACTTTCTACGCGAGCTGGGCGCAAGGC | GTTTTTGATCCC |
| | | | (SEQ ID NO: 233) | (SEQ ID NO: 234) |
| | AA | SGQLMC | CIGSGSNAISTFYASWAQG | VGSDDYGDSDVFDP |
| | | (SEQ ID NO: 235) | (SEQ ID NO: 236) | (SEQ ID NO: 237) |
| 7B20 | NA | AGCGCCTACTACATATGC | TGTATTGGTGGTGTTAATCGCGTTGCCACT | GGCGGTCCTGATAATAATGTCCAATTTAAC |
| | | (SEQ ID NO: 238) | GCCTACGCGACCTGGGCGAAAGGC | TTG |
| | | | (SEQ ID NO: 239) | (SEQ ID NO: 240) |
| | AA | SAYYIC | CIGGVNRVATAYATWAKG | GGPDNNVQFNL |
| | | (SEQ ID NO: 241) | (SEQ ID NO: 242) | (SEQ ID NO: 243) |
| 7J6 | NA | AGGTACTACTACAGTTGC | TGTGTTGGTGGTGTTAATCGCGATGCCACT | GGCGGTCCTGATAATAATGTCCAATTTAAC |
| | | (SEQ ID NO: 244) | GCCTACGCGACCTGGGCGAAAGGC | TTG |
| | | | (SEQ ID NO: 245) | (SEQ ID NO: 246) |
| | AA | RYYYSC | CVGGVNRDATAYATWAKG | GGPDNNVQFNL |
| | | (SEQ ID NO: 247) | (SEQ ID NO: 248) | (SEQ ID NO: 249) |
| 1D2 | NA | AATTATTATATTTGT | TGTATTGACAATGTTAATGGTAGGACTTAC | TCATTGTCTACTCCCTTG |
| | | (SEQ ID NO: 250) | TACGCGAGCTGGGCGAAAGGC | (SEQ ID NO: 252) |
| | | | (SEQ ID NO: 251) | |
| | AA | NYYIC | CIDNVNGRTYYASWAKG | SLSTPL |
| | | (SEQ ID NO: 253) | (SEQ ID NO: 254) | (SEQ ID NO: 255) |
| 4G8 | NA | AGCAGCTACTTCATGTGC | TGCATTGCTGTTGGTAGTAGTGGTAGCACT | GTGGGCTACGATGACTATGGTGACTCTGAT |
| | | (SEQ ID NO: 256) | TACTACGCGAGCTGGGCGAAAGGC | GCTTTTGATCCC |
| | | | (SEQ ID NO: 257) | (SEQ ID NO: 258) |
| | AA | SSYFMC | CIAVGSSGSTYYASWAKG | VGYDDYGDSDAFDP |
| | | (SEQ ID NO: 259) | (SEQ ID NO: 260) | (SEQ ID NO: 261) |
| 7I4 | NA | AGGTACTACTACAGTTGC | TGTGTTGGTGGTGTTAATCGCGATGCCACT | GGCGGTCCTGATAATAATGTCCAATTTAAC |
| | | (SEQ ID NO: 262) | GCCTACGCGACCTGGGCGAAAGGC | TTG |
| | | | (SEQ ID NO: 263) | (SEQ ID NO: 264) |
| | AA | RYYYSC | CVGGVNRDATAYATWAKG | GGPDNNVQFNL |
| | | (SEQ ID NO: 265) | (SEQ ID NO: 266) | (SEQ ID NO: 267) |
| 1I23 | NA | AGCAGCTACTTCATGTGC | TGCATTGGTTCTGGTAGTAGTGCTATTAGC | GTGGGCTACGATGACTATGGTGACTCTGAT |
| | | (SEQ ID NO: 268) | ACTTTCTACGCGAGCTGGGCGCAAGGC | GCTTTTGATCCC |
| | | | (SEQ ID NO: 269) | (SEQ ID NO: 270) |
| | | SSYFMC | CIGSGSSAISTFYASWAQG | VGYDDYGDSDAFDP |
| | AA | (SEQ ID NO: 271) | (SEQ ID NO: 272) | (SEQ ID NO: 273) |
| 1J13 | NA | AGCTATGGAATGACT | TACATTTGGACTGATGGGAGGACATACTAC | CCCTTTGATGGTAATTATAGGGACATC |
| | | (SEQ ID NO: 274) | GCAAACTGGGCGAAAGGC | (SEQ ID NO: 276) |
| | | | (SEQ ID NO: 275) | |
| | AA | SYGMT | YIWTDGRTYYANWAKG | PFDGNYRDI |
| | | (SEQ ID NO: 277) | (SEQ ID NO: 278) | (SEQ ID NO: 279) |
| 1G24 | NA | AACTACTACATCTGC | TGTATTGATAATGCCAATGGTCGGACTTAC | TCCTTGGCTACTCCCTTG |
| | | (SEQ ID NO: 280) | TACGCGAACTGGGCGAAAGGC | (SEQ ID NO: 282) |
| | | | (SEQ ID NO: 281) | |
| | AA | NYYIC | CIDNANGRTYYANWAKG | SLATPL |
| | | (SEQ ID NO: 283) | (SEQ ID NO: 284) | (SEQ ID NO: 285) |
| 4A7 | NA | AGCAGCTACTTCATGTGC | TGCATTGGTGTTGGTGGTAGTGGTAGCACT | GTGGCCTACGATGACGATGGTGACTCTGAT |
| | | (SEQ ID NO: 286) | TACTACGCGAACTGGGCGAAAGGC | GCTTTTGATCCC |
| | | | (SEQ ID NO: 287) | (SEQ ID NO: 288) |
| | AA | SSYFMC | CIGVGGSGSTYYANWAKG | VAYDDDGDSDAFDP |
| | | (SEQ ID NO: 289) | (SEQ ID NO: 290) | (SEQ ID NO: 291) |

TABLE 23
Standard IgG Antibody Variable Region Sequences

| Ab | Type | LC V-region | HC V-region |
|---|---|---|---|
| 7M5 | NA | GATGTTGTGATGACCCAGACTCCAGCCTCCGTGTCTGAACCTG | CAGTCGCTGGAGGAGTCCGGGGGTCGCCTGGTAACGCCTGGAGGATC |
| | | TGGGAGGCACAGTCACCATCAAGTGCCAGGCCAGTCAGAGCA | CCTGACACTCACCTGCACAGTCTCTGGAATCGACCTCAGTAGCCATG |
| | | TTAGGAATGAATTATTTTGGTGGCAGCAGAAACCAGGGCAGCC | CAATGATCTGGGTCCGCCAGGCTCCAGGGGAGGGGCTGGAATGGAT |
| | | TCCCAAGCTCCTGATCTATGCTGCATCCAAACTGGCCTCTGGGG | CGGAACCATTGGGAGTCGTGATACTATATATTATGCGAGCTGGGCG |
| | | TCCCATCGCGGTTCAGCGGCAGTGGATCTGGGACAGAGTTCAC | AAAGGCCGATTCACCATCTCCAAAACCTCGTCGACCACAATGGATC |
| | | TCTCACCATCAGCGACCTGGAGTGTGCCGATGCTGCCACTTACT | TGAAAATGACCAGTCTGACAATCGAGGACACGGCCACCTATTTCTG |
| | | ACTGTCAATGCAGTTATGTTAGTAGTAGTGGTACTTATGGAAA | TGTCAGAAACGCCTTGTGGGGCCCAGGCACCCTGGTCACC |
| | | TGTTTTCGGCGGAGGGACCGAGGTGGTGGTCAAA | GTCTCCTCA |
| | | (SEQ ID NO: 292) | (SEQ ID NO: 293) |
| | AA | DVVMTQTPASVSEPVGGTVTIKCQASQSIRNELFWWQQKPGQPPKLL | QSLEESGGRLVTPGGSLTLTCTVSGIDLSSHAMIWVRQAPGEGLEWIGTIGSR |
| | | IYAASKLASGVPSRFSGSGSGTEFTLTISDLECADAATYYCQCSYVSSS | DTIYYASWAKGRFTISKTSSTTMDLKMTSLTIEDTATYFCVRNALWGPGTL |
| | | GTYGNVFGGGTEVVVK | VTVSS |
| | | (SEQ ID NO: 294) | (SEQ ID NO: 295) |
| 1J14 | NA | GCCTATGATATGACCCAGACTCCAGCCTCCGTGGAGGCAGCTGTG | CAGTCGCTGGAGGAGTCCGGGGGTCGCCTGGTCACGCCTGGGACACCCC |
| | | GGAGGCACAGTCACCATCAATTGCCAGGCCAGTGAAAGCATTAGCA | TGACACTCACCTGCAAAGTCTCTGGATTCTCCCTCAGCAGCTACGACATG |
| | | ACTACTTATCCTGGTATCAGCAGAAACCAGGGCAGCCTCCCAAGC | AGCTGGGTCCGCCAGGCTCCAGGGAAGGGGCTGGAATGGATCGGAATTA |
| | | TCCTGATCTACTGGGCATCCACTCTGGCATCTGGGGTCTCATCGCG | TTTATGCTAGTGGTAGCACATACTACGCGAGCTGGGCGAAAGGCCGATT |
| | | GTTCAAAGGCAGTGGATCTGGGACACAGTTCACTCTCACCATCAGC | CACCATCTCCAAAACCTCGACCACGGTGGATCTGAAAATCGCCAGTCCG |
| | | GGCGTGGAGTGTGCCGATGCTGCCACTTACTACTGTCAACAGGGTT | ACAACCGAGGACACGGCCACCTATTTCTGTGCCAGAGACCCTGCTGGTT |
| | | ATAGTAGTAGTAATGTTGATAATCTTTTCGGCGGAGGGACCG | ATAGCATTAGCTTTGGCTTGTGGGGCCCAGGCACCCTGGTCACCGTCTC |
| | | AGGTGGTGGTCAAA | CTCA |
| | | (SEQ ID NO: 296) | (SEQ ID NO: 297) |
| | AA | AYDMTQTPASVEAAVGGTVTINCQASESISNYLSWYQQKPGQPP | QSLEESGGRLVTPGTPLTLTCKVSGFSLSSYDMSWVRQAPGKGLEWIGII |
| | | KLLIYWASTLASGVSSRFKGSGSGTQFTLTISGVECADAATYYCQQ | YASGSTYYASWAKGRFTISKTSTTVDLKIASPTTEDTATYFCARDPAGYS |
| | | GYSSSNVDNLFGGGTEVVVK | ISFGLWGPGTLVTVSS |
| | | (SEQ ID NO: 298) | (SEQ ID NO: 299) |
| 7F15 | NA | GCCGCCGTGCTGACCCAGACTCCATCTCCCGTGTCTGCAG | CAGGAGCAGCTGAAGGAGTCCGGAGGAGGCCTGGTTGCGCCTGGAG |
| | | CTGTGGGAGGCACAGTCAGCATCAGTTGCCAGTCCAGTC | GAACCCTGACACTCACCTGCGCAGTCTCTGGATTCTCCCTCAGTAGCTA |
| | | AGAGTGTTTGGCATAACGACTACTTATCCTGGTATCAAC | CAACATGGGCTGGGTCCGCCAGGCTCCAGGGGAGGGGCTGGAATAC |
| | | AGAAACCAGGGCAGCCTCCCAAGCTCCTGATCTATGGT | ATCGGAATCATTGGTGCTAGTGATAGCGCATTGTACGCGAGCTGGGC |
| | | GCATCCACTTTGGCATCTGGGGTCCCATCGCGGTTCAAA | AAAAGGCCGATTCACCATCTCCAAAACCTCGACCACGGTGGATCTG |
| | | GGCAGTGGATCTGGGACACAGTTCAGTCTCACCATCAGC | AAAATCACCAGTCCGACAACCGAGGACACGGCCACCTATTTCTGTGC |
| | | GGCGTGCAGTGTGACGATGCTGCCACTTACTACTGTGCA | CAGAGGTGGTCTTGGTTTGAGTACTGGTTTTGCGTTGTGGGGCCCAG |
| | | GGCGGTTATGGACGTAGTAGTGAAAATGGTTTCGGCGG | GCACCCTGGTCACCGTCTCCTCA |
| | | AGGGACCGAGGTGGTGGTCAAA | (SEQ ID NO: 301) |
| | | (SEQ ID NO: 300) | |
| | AA | AAVLTQTPSPVSAAVGGTVSISCQSSQSVWHNDYLSWYQQ | QEQLKESGGGLVAPGGTLTLTCAVSGFSLSSYNMGWVRQAPGEGLEYI |
| | | KPGQPPKLLIYGASTLASGVPSRFKGSGSGTQFSLTISGVQCD | GIIGASDSALYASWAKGRFTISKTSTTVDLKITSPTTEDTATYFCARGGL |
| | | DAATYYCAGGYGRSSENGFGGGTEVVVK | GLSTGFALWGPGTLVTVSS |
| | | (SEQ ID NO: 302) | (SEQ ID NO: 303) |
| 5I9 | NA | GCCGCCGTGCTGACCCAGACTCCATCTCCCGTGTCTGCAG | CAGTCGCTGGAGGAGTCCGGGGGTCGCCTGGTAACGCCTGGAGGAC |
| | | CTGTGGGAGGCACAGTCAGCATCAGTTGCCAGTCCAGTA | CCCTGACACTCACCTGCACAGTCTCTGGATTCTCCCTCAGCAGCTACGA |
| | | AGAGTGTTTATAATAACAACTGGTTAGCCTGGTATCAGC | CATGAGCTGGGTCCGCCAGGCTCCAGGGAAGGGGCTGGAGTGGATC |
| | | AGAAACCAGGGCAGCGTCCCAAGCTCCTGATCTACGAT | GGATATATTGCTACTGATGGTAGGCCATATTACGCGAGCTGGGCGAA |
| | | GCATCGACTCTAGATTCTGGGGTCTCATCGCGGTTCAAA | AGGCCGATTCACCATCTCCAAACCCTCGTCGACCACGGTGGATCTGAA |
| | | GGCAGTGGATCTGGGACACAGTTCACTCTCACCATCAG | AATCACCAGTCCGACAACCGAGGACACGGCCACCTATTTCTGTGTCAG |
| | | CGACGTGCAGTGTGACGATGGTGCCACTTACTACTGTGT | AGGGGGGTATGCTGGTGGCTTGTGGGGCCCAGGCACCCTGGTCACC |
| | | AGGCGGTTATAGTAGTCGTAGTGATAATGGTTTCGGCG | GTTTCCTCA |
| | | GAGGGACCGAGGTGGTGGTCAAA | (SEQ ID NO: 305) |
| | | (SEQ ID NO: 304) | |
| | AA | AAVLTQTPSPVSAAVGGTVSISCQSSKSVYNNNWLAWYQ | QSLEESGGRLVTPGGPLTLTCTVSGFSLSSYDMSWVRQAPGKGLEWIGY |
| | | QKPGQRPKLLIYDASTLDSGVSSRFKGSGSGTQFTLTISDVQ | IATDGRPYYASWAKGRFTISKPSSTTVDLKITSPTTEDTATYFCVRGGYA |
| | | CDDGATYYCVGGYSSRSDNGFGGGTEVVVK | GGLWGPGTLVTVSS |
| | | (SEQ ID NO: 306) | (SEQ ID NO: 307) |
| 1J15 | NA | GACATTGTGATGACCCAGACTCCAGCCTCCGTGGAGGC | CAGTCGTTGGAGGAGTCCGGGGGAGGCCTGGTCCAGCCTGAGGGAT |
| | | AGCTGTGGGAGGCACAGTCACCATCAAGTGCCAGGCCA | CCCTGACACTCACCTGCACAGCTTTTGGATTCACCCTCAATAATTATT |
| | | GTCAGAGTATTAGTAGTTACTTATCCTGGTATCAGCAGA | ATATTTGCTGGGTCCGCCAGGCTCCAGGGAAGGGACTGGAGTGGAT |
| | | AACCAGGGCAGCCTCCCAAGCTCCTGATCTACAGGGCA | CGCATGCATTGACAATGCTAATGGTAGGACTTACTACGCGAGCTGGG |
| | | TCCACTCTGGCATCTGGGGTCCCATCGCGGTTCAGAGGC | CGAAAGGCCGATTCACCATCTCCAAAACCTCGTCGACCACGGTGACTC |
| | | AGTGGATCTGGGACACAGTTCACTCTCACCATCAGCGA | TACAAATGACCAGTCTGACAGCCGCGGACACGGCCACCTATTTCTGTG |
| | | CCTGGAGTGTGCCGATGCTGCCACTTATTACTGTCAAAG | CGAGGTCATTGTCTACTCCCTTGTGGGGCCCAGGCACCCTGGTCACCG |
| | | CTATGATGATAGTAGTGATAATAATTTTTTTTATGGTTTCG | TCTCCTCA |
| | | TCTCCTCAGCGGAGGGACCGAGGTGGTGGTCAGA | (SEQ ID NO: 309) |
| | | (SEQ ID NO: 308) | |
| | AA | DIVMTQTPASVEAAVGGTVTIKCQASQSISSYLSWYQQKP | QSLEESGGGLVQPEGSLTLTCTAFGFTLNNYYICWVRQAPGKGLEWIAC |
| | | GQPPKLLIYRASTLASGVPSRFRGSGSGTQFTLTISDLECAD | IDNANGRTYYASWAKGRFTISKTSSTTVTLQMTSLTAADTATYFCARSL |
| | | AATYYCQSYDDSSDNNFFYGFGGGTEVVVR | STPLWGPGTLVTVSS |
| | | (SEQ ID NO: 310) | (SEQ ID NO: 311) |
| 4G17 | NA | GACATTGTGATGACCCAGACTCCAGCCTCCGTGGAGGC | CAGTCGTTGGAGGAGTCCGGGGGAGGCCTGGTCCAGCCTGAGGGAT |
| | | AGCTGTGGGAGGCACAGTCACCATCAAGTGCCAGGCCA | CCCTGACACTCACCTGCACAGCTTCTGGAGTCACCCTCAGTAACTAT |
| | | GTCAGAACATTGATAGTTACTTAGCCTGGTATCAGCAG | TATATTTGTTGGGTCCGCCAGGCTCCAGGGAAGGGGCTGGAGTGGAT |
| | | AAACCAGGGCAGCCTCCCAAGCTCCTGATCTACAGGGC | CGGGTGCATTGACAATGTTAATGGTAGGACCTACTACGCGAGCTGGG |
| | | AAGCTATGATGATAGTAGGAGTAGTAGTTTTTTTTATGG | CGAAAGGCCGATTCACCATCTCCAAGACCTCGTCGACCACAGGGACTC |
| | | ATCCACTCTGGCATCTGGGGTCCCATCGCGGTTCAAAG | TACAAATGACCAGTCTGACAGCCGCGGACACGGCCACCTATTTCTGTG |
| | | GCAGTGGATCTGGGACAGAGTTCACTCTCACCATCAGC | CGAGGTCCTTGGCTACTCCCTTGTGGGGCCCAGGCACCCTAGTCACC |
| | | GACCTGGAGTGTGCCGATGCTGCCACTTACTACTGTCA | GTCTCCTCA |
| | | TTTCGGCGGAGGGACCGAGGTGGTGGTCAAA | (SEQ ID NO: 313) |
| | | (SEQ ID NO: 312) | |
| | AA | DIVMTQTPASVEAAVGGTVTIKCQASQNIDSYLAWYQQKP | QSLEESGGGLVQPEGSLTLTCTASGVTLSNYYICWVRQAPGKGLEWIG |
| | | GQPPKLLIYRASTLASGVPSRFKGSGSGTEFTLTISDLECADA | CIDNVNGRTYYASWAKGRFTISKTSSTTGTLQMTSLTAADTATYFCAR |
| | | ATYYCQSYDDSRSSSFFYGFGGGTEVVVK | SLATPLWGPGTLVTVSS |
| | | (SEQ ID NO: 314) | (SEQ ID NO: 315) |
| 4A15 | NA | GACATTGTGATGACCCAGACTCCAGCCTCCGTGGAGGC | CAGTCGTTGGAGGAGTCCGGGGGAGGCCTGGTCCAGCCTGAGGGAT |
| | | AGCTGTGGGAGGCACAGTCACCATCAAGTGCCAGGCCA | CCCTGACACTCACCTGCACAGCTTCTGGAGTCACCCTCACTAACTACT |
| | | GTCAGAACATTAATAGTTACTTAGCCTGGTATCAGCAGA | ACATCTGCTGGGTCCGCCAGGCTCCAGGGAAGGGGCTGGAGTGGATC |
| | | AACCAGGGCAGCCTCCCAAGCTCCTGATCTACAGGGCA | GGGTGCATTGACAATGTTAATGGTAGGACTTACTACGCGAGCTGGGC |
| | | TCCACTCTGGCATCTGGGGTCCCATCGCGGTTCAAAGGC | GAAAGGCCGATTCACCATCTCCAAGGCCTCGTCGACCACAGGGACTC |
| | | AGTGGATCTGGGACAGAGTTCACTCTCACCATCAGCGA | TACAAATGACCAGTCTGACAGCCGCGGACACGGCCACCTATTTCTGTG |
| | | CCTGGAGTGTGCCGATGCTGCCACTTACTACTGTCAAAG | CGAGGTCCTTGGCTACTCCCTTGTGGGGCCCAGGCACCCTGGTCACC |
| | | CTATGATGATAGTAGGAGTATTAGTTTTTTTTATGCTTTCG | GTCTCCTCA |
| | | GCGGAGGGACCGAGGTGGTGGTCAAA | (SEQ ID NO: 317) |
| | | (SEQ ID NO: 316) | |
| | AA | DIVMTQTPASVEAAVGGTVTIKCQASQNINSYLAWYQQK | QSLEESGGGLVQPEGSLTLTCTASGVTLTNYYICWVRQAPGKGLEWIG |
| | | PGQPPKLLIYRASTLASGVPSRFKGSGSGTEFTLTISDLECAD | CIDNVNGRTYYASWAKGRFTISKASSTTGTLQMTSLTAADTATYFCAR |
| | | AATYYCQSYDDSRSISFFYAFGGGTEVVVK | SLATPLWGPGTLVTVSS |
| | | (SEQ ID NO: 318) | (SEQ ID NO: 319) |
| 1J16 | NA | GACATTGTGATGACCCAGACTCCAGCCTCCGTGGAGGC | CAGTCGTTGGAGGAGTCCGGGGGAGGCCTGGTCCAGCCTGAGGGAT |
| | | AGCTGTGGGAGGCACAGTCACCATCAAGTGCCAGGCCA | CCCTGACAGTCACCTGCACAGCTTTTGGAGTCACCCTCACTAACTACT |
| | | GTCAGAGCATTAGTAGCTACTTAGCCTGGTATCACCAGA | ACATCTGCTGGGTCCGCCAGGCTCCAGGGAAGGGGCTGGAGTGGATC |
| | | AACCAGGGCAGCCTCCCAAGCTCCTGATCTATGCTGCATC | GGGTGCATTGACAATATTAATGGTAGGACTTACTACGCGAGCTGGGC |
| | | CACTCTGGCATCTGGGGTCCCATCGCGGTTCGAAGGCA | GAAAGGCCGATTCACCATCTCCAAGACCTCGTCGACCACAGGGACTC |
| | | CTGGAGTGTGCCGATGCTGCCACTTACTACTGTCAAAGC | TGCAAATGACCAGTCTGACAGCCGCGGACACGGCCACCTATTTCTGTG |
| | | GTGGATCTGGGACACAGTTCACTCTCACCATCAGCGAC | CGAGGTCCTTGGCTACTCCCTTGTGGGGCCCAGGCACCCTGGTCACC |
| | | TATGATGATAGTAGGAGTAGTAGTTTTTTTTATGCTTTC | GTCTCCTCA |
| | | GGCGGAGGGACCGAGGTGGTGGTCAAA | (SEQ ID NO: 321) |
| | | (SEQ ID NO: 320) | |
| | AA | DIVMTQTPASVEAAVGGTVTIKCQASQSISSYLAWYHQKP | QSLEESGGGLVQPEGSLTVTCTAFGVTLTNYYICWVRQAPGKGLEWIGC |
| | | GQPPKLLIYAASTLAS | IDNINGR |
| | | GVPSRFEGSGSGTQFTLTISDLECADAATYYCQSYDDSRSS | TYYASWAKGRFTISKTSSTTGTLQMTSLTAADTATYFCARSLATPLWGP |
| | | SFFYAFGGGTEVVVK | GTLVTVSS |
| | | (SEQ ID NO: 322) | (SEQ ID NO: 323) |
| 1C18 | NA | GACATTGTGATGACCCAGACTCCAGCCTCCGTGGAGGC | CAGTCGTTGGAGGAGTCCGGGGGAGGCCTGGTCCAGCCTGAGGGAT |
| | | AGCTGTGGGAGGCA | CCCTGAC |
| | | CAGTCACCATCAAGTGCCAGGCCAGTCAGAGCATTAGC | ACTCACCTGCACAGCTTCTGGATTCACCCTCACTAATTATTATATATG |
| | | AGTTGGTTATCCTGG | TTGGGT |
| | | TATCAGCAGAAACCAGGGCAGCCTCCCAAGCTCCTGCTCT | CCGCCAGGCTCCAGGGAAGGGACTGGAGTGGATCGCATGTATTGAT |
| | | ACAGGGCAACCA | AACGCTA |
| | | CTCTGGCATCTGGGGTCCCATCGCGGTTCAAAGGCAGT | ATGGTAGGACTTACTACGCGACCTGGGCGAAAGGCCGATTCACCATC |
| | | GGATCTGGGACACA | TCCAAA |
| | | GTTCACTCTCACCATCAGCGACCTGGAGTGTGCCGATG | ACCTCGTCGACCACGGTGACTCTGCAAATGCCCAGTCTGACAGCCGCG |
| | | CTGCCACTTATTACT | GACAC |
| | | GTCAAAGTTATGATGATAGTAGTAGTAGTAATTTTTTTTA | GGCCACCTATTTCTGTGCGAGGTCATTGTCTACTAACTTGTGGGGCCCA |
| | | TGCTTTCGGCGGAG GGACCGAGGTGGTGGTCAGA | GGCAC CCTGGTCACCGTCTCCTCA |
| | | (SEQ ID NO: 324) | (SEQ ID NO: 325) |
| | AA | DIVMTQTPASVEAAVGGTVTIKCQASQSISSWLSWYQQKP | QSLEESGGGLVQPEGSLTLTCTASGFTLTNYYICWVRQAPGKGLEWIAC |
| | | GQPPKLLLYRATTLA | IDNANGR |
| | | SGVPSRFKGSGSGTQFTLTISDLECADAATYYCQSYDDSSS | TYYATWAKGRFTISKTSSTTVTLQMPSLTAADTATYFCARSLSTNLWGP |
| | | SNFFYAFGGGTEVVVR | GTLVTVSS |
| | | (SEQ ID NO: 326) | (SEQ ID NO: 327) |
| 7H4 | NA | CAAGCCGTGGTGACCCAGACTCCATCGTCCGTGTCTGC | CAGTCGTTGGAGGAGTCCGGGGGAGGCCTGGTCAAGCCTGGGGCAT |
| | | AGCTGTGGGAGGCACAGTCACCATCAGTTGCCAGTCCA | CCCTGACACTCACCTGCAAAGCCTCTGGGTCAGACTTCAGAAGCTAC |
| | | GTCAGAGTGTTTATAGTAACAACCTCTTATCTTGGTATC | TACTACATATGCTGGGTCCGCCAGGCTCCAGGGAAGGGGCTGGAGT |
| | | AGCAGAAACCAGGGCAGCCTCCCAAGCTCCTGATCTAC | GGGTCGCATGTATTGGTGGTGGTAATACCGATGCCACTGCCTACGCGA |
| | | AAGGCATCCACTCTGGCATCTGGGGTCCCATCGCGGTTG | GGTGGGCGAAAGGCCGATTCACCATCTCCAAAACCTCGGCGACCACGG |
| | | AAAGGCAGTGGATCTGGGACACAGTTCACTCTCACAAT | TGGCTCTCCAAATGACCAGTCTGACAGCCGCGGACACGGCCACCTATT |
| | | CAGCGAAGTACAGTGTGACGATGCTGCCACTTATTACTG | TCTGTGTGAGAGGCGGTCCTGATAATAATGTCCAATTTAACTTGTGGG |
| | | TCAAGGCTATTATAGTGGTGTTATTTATATGTTCGGCGG | GCCCAGGCACCCTGGTCACCGTCTCCTCA |
| | | AGGGACCGAGGTGGTGGTCAAA | (SEQ ID NO: 329) |
| | | (SEQ ID NO: 328) | |
| | AA | QAVVTQTPSSVSAAVGGTVTISCQSSQSVYSNNLLSWYQQK | QSLEESGGGLVKPGASLTLTCKASGSDFRSYYYICWVRQAPGKGLEWV |
| | | PGQPPKLLIYKASTLASGVPSRLKGSGSGTQFTLTISEVQCD | ACIGGGNTDATAYARWAKGRFTISKTSATTVALQMTSLTAADTATYFC |
| | | DAATYYCQGYYSGVIYMFGGGTEVVVK | VRGGPDNNVQFNLWGPGTLVTVSS |
| | | (SEQ ID NO: 330) | (SEQ ID NO: 331) |
| 1H24 | NA | GACATTGTGATGACCCAGACTCCATTCTCCGTGGAGGC | CAGTCGTTGGAGGAGTCCGGGGGAGGCCTGGTCCAGCCTGAGGGAT |
| | | AGCTGTGGGAGGCACAGTCACCATCAAGTGCCAGGCCA | CCCTGACACTCACCTGCACAGCTTCTGGATTCACCCTCAATAATTATTA |
| | | GTCAGAGCATTAGTAGTTACTTATCCTGGTATCGGCAGA | TATTTGCTGGGTCCGCCAGGCTCCAGGGAAGGGACTGGAGTGGATCG |
| | | AACCAGGGCAGCCTCCCAAGCTCCTGATCTACAGGGCA | CATGCATTGACAATAGTAATGGTAGGACTTACTACGCGAGCTGGGCG |
| | | TCCACTCTGGCATCTGGGGTCCCATCGCGGTTCAAAGGC | AAAGGCCGTTTCACCATCTCCAAAACCTCGTCGACCACGGTGACTCTG |
| | | AGTGGATCTGGGACACAGTTCACTCTCACCATCAGCGA | CAAATGACCAGTCTGACAGCCGCGGACACGGCCACCTATTTCTGTGCG |
| | | CCTGGAGTGTGCCGATGCTGCCACTTACTACTGTCAAAG | AGGTCATTGTCTACTCCCTTGTGGGGCCCAGGCACCCTGGTCACCGTC |
| | | CTATGATGATAGTAGTAGTAATAATTTTTTTTATGGTTTCG | TCCTCA |
| | | GCGGAG GGACCGAGGTGGTGGTCAGA | (SEQ ID NO: 333) |
| | | (SEQ ID NO: 332) | |
| | AA | DIVMTQTPFSVEAAVGGTVTIKCQASQSISSYLSWYRQKP | QSLEESGGGLVQPEGSLTLTCTASGFTLNNYYICWVRQAPGKGLEWIAC |
| | | GQPPKLLIYRASTLASGVPSRFKGSGSGTQFTLTISDLECAD | IDNSNGRTYYASWAKGRFTISKTSSTTVTLQMTSLTAADTATYFCARSL |
| | | AATYYCQSYDDSSSNNFFYGFGGGTEVVVR | STPLWGPGTLVTVSS |
| | | (SEQ ID NO: 334) | (SEQ ID NO: 335) |
| 1C3 | NA | GACATTGTGATGACCCAGACTCCATCCTCCGTGGAGGC | CAGTCGTTGGAGGAGTCCGGGGGAGGCCTGGTCCAGCCTGAGGGAT |
| | | AGCTGTGGGAGGCACAGTCACCATCAAGTGCCAGGCCA | CCCTGACACTCACCTGCACAGCTTTTGGAGTCACCCTCACTAACTACTA |
| | | GTCAGAACATTAGTAGCTACTTAGCCTGGTATCAGCAG | CATCTGCTGGGTCCGCCAGGCTCCAGGAAAGGGGCTGGAGTGGGTCG |
| | | AAACCAGGGCAGCCTCCCAAGCTCCTGATCTACAGGGC | GGTGTATTGACAATGCTAATGGTAGGACTTACTACGCGAGCTGGGCG |
| | | ATCCACTCTGGCATCTGGGGTCCCATCGCGGTTCAAAG | AAAGGCCGATTCACCATCTCCAAGACCTCGTCGACCACAGGGACTCT |
| | | GCAGTGGATCTGGGACACAGTTCACTCTCACCATCAGCG | GAGGTCCTTGGCTACTCCCTTGTGGGGCCCAGGCACCCTGGTCACCG |
| | | ACCTGGAGTGTGCCGATGCTGCCACTTACTACTGTCAAAG | GCAAATGACCAGTCTGACAGCCGCGGACACGGCCACCTATTTCTGTGC |
| | | CTATGATGATAGTAGGAGTAGTAATTTTTTTTATGCTTTCG | TCTCCTCA |
| | | GCGGAGGGACCGAGGTGGTGGTCAAA | |
| | | (SEQ ID NO: 336) | (SEQ ID NO: 337) |
| | AA | DIVMTQTPSSVEAAVGGTVTIKCQASQNISSYLAWYQQKP | QSLEESGGGLVQPEGSLTLTCTAFGVTLTNYYICWVRQAPGKGLEWVG |
| | | GQPPKLLIYRASTLASGVPSRFKGSGSGTQFTLTISDLECAD | CIDNANGRTYYASWAKGRFTISKTSSTTGTLQMTSLTAADTATYFCARS |
| | | AATYYCQSYDDSRSSNFFYAFGGGTEVVVK | LATPLWGPGTLVTVSS |
| | | (SEQ ID NO: 338) | (SEQ ID NO: 339) |
| 5H20 | NA | GCGCAAGCGCTGACCCAGACTCCATCCCCTGTGTCTGC | CAGTCGGTGGAGGAGTCCGGGGGTCGCCTGGTCACGCCTGGGACACCC |
| | | AGCTGTGGGAGGCACAGTCACCATCAGTTGCCAGTCCA | CTGACACTCACCTGCACAGTTTCTGGAGTCGACCTCAGTAGCAGTGCA |
| | | GTGAGAGCGTTTATAATCACAACTGGTTAGGCTGGTATC | GTGACCTGGGTCCGCCAGGCTCCAGGGATGGGACTGGAATACATCGGA |
| | | AGCAGAAACCAGGGCAGCCTCCCAAACTCCTGATCTAT | TTCCTCCAAGCTGGGGATGGTAGCGCATACTACGCGAGCTGGGCGAAA |
| | | GATGCATCCACTCTGGCATCTGGGGTCCCATCGCGGTTCA | GGCCGATTCACCATCTCCAAAACCTCGTCGACCACAGTGGATCTGAAA |
| | | AAGGCAGTGGATCTGGGACACAGTTCACTCTCACAATCA | ATGACCAGTCTCACAACCGAGGACACGGCCACCTATTTCTGTGCCAGA |
| | | GCGAAAGTCAGTGTGACGATGCTGCCATTTACTACTGTCA | CATAAGGGTAATAGTTACGTGCCTAACTTGTGGGGCCCAGGCACCCT |
| | | AGGCTATTATCAAACTAGTGTTTGGGCTTTCGGCGGAGG | GGTCACCGTCTCCTCA |
| | | GACCG AGGTGGTGGTCAAA | (SEQ ID NO: 341) |
| | | (SEQ ID NO: 340) | |
| | AA | AQALTQTPSPVSAAVGGTVTISCQSSESVYNHNWLGWYQQ | QSVEESGGRLVTPGTPLTLTCTVSGVDLSSSAVTWVRQAPGMGLEYIGFL |
| | | KPGQPPKLLIYDASTLASGVPSRFKGSGSGTQFTLTISESQCD | QAGDGSAYYASWAKGRFTISKTSSTTVDLKMTSLTTEDTATYFCARHKG |
| | | DAAIYYCQGYYQTSVWAFGGGTEVVVK | NSYVPNLWGPGTLVTVSS |
| | | (SEQ ID NO: 342) | (SEQ ID NO: 343) |
| 1I14 | NA | GATGTTGTGATGACCCAGACTCCAGCCTCCGTGGAGGC | CAGTCGGTGGAGGAGTCCGGGGGTCGCCTGGTCACGCCTGGGACACCC |
| | | AACTGTGGGAGGCACAGTCACCATCAAGTGCCAGTCCA | CTGACACTCACCTGCACAATCTCTGGATTCTCCCTCAGTAGCTATGCAA |
| | | GTCAGAGTGTTTATGATAACAATGCTTTAGCCTGGTATC | TGAGCTGGGTCCGCCAGGCTCCAGGGAAGGGGCTGGAATGGATCGGA |
| | | AGCAGAATGCAGGACAGCGTCCCAGACTCCTGATCTAT | AGCATTGGTGGTGGTGGTAGCGCAGTCTACGCGAGCTGGGCGAAAGGC |
| | | GGTGCATCCACTCTGGCATCTGGGGTCCCATCGCGGTTCA | CGATTCACCATCTCCAAAACCTCGACCACGGTGGATCTGAGAATCACC |
| | | GTGCCAGTGGATCTGGGACAGAGTTCACTCTCACCATCAG | AGTCCGACAACCGAGGACACGGCCATGTATTTCTGTGGCAGGGGATT |
| | | CGACCTGGAGTGTGCCGATGCAGCCACTTACTACTGTCA | TTATAGTATAGACTTGTGGGGCCCAGGCACCCTGGTCACCGTCTCCT |
| | | ATGTACTTATTATGTTAGTAGTTATCAAAATGATTTCGG | CA |
| | | CGGAG GGACCGAGGTGGTGGTCAAA | (SEQ ID NO: 345) |
| | | (SEQ ID NO: 344) | |
| | AA | DVVMTQTPASVEATVGGTVTIKCQSSQSVYDNNALAWYQ | GGGGSAVYASWAKGRFTISKTSTTVDLRITSPTTEDTAMYFCGRGFYSI |
| | | QNAGORPRLLIYGASTLASGVPSRFSASGSGTEFTLTISDLEC | QSVEESGGRLVTPGTPLTLTCTISGFSLSSYAMSWVRQAPGKGLEWIGSI |
| | | ADAATYYCQCTYYVSSYQNDFGGGTEVVVK | DLWGPGTLVTVSS |
| | | (SEQ ID NO: 346) | (SEQ ID NO: 347) |
| 1H17 | NA | GCCGCCGTGCTGACCCAGACTCCATCTCCCGTGTCTGCAG | CAGGAGCAGCTGGTGGAGTCCGGGGGAGGCCTGGTCAAGCCTGGGG |
| | | CTGTGGGAGGCACAGTCAGCGCCAGTTGCCAGTCCAGTA | CATCCCTGACACTCACCTGCAAAGCCTCTGGATTCTCCTTCAGTAGCGG |
| | | AGAGTGTTTATAATAAGAACTGGTTATCCTGGTTTCAGC | CCAACTCATGTGCTGGGTCCGCCAGGCTCCAGGGAAGGGGCTGGAGT |
| | | AGAAACCAGGGCAGCCTCCCAAGCTCCTGATCTATGGTG | GGATCGCATGCATTGGTTCTGGTAGTAATGCTATTAGCACTTTCTAC |
| | | CATCCACTCTGGCATCTGGGGTCCCATCGCGGTTCAAAG | GCGAGCTGGGCGCAAGGCCGATTCACCATCTCCAAATCCTCGTCGA |
| | | GCAGTGGATCTGGGACACAGTTCACTCTCACCATCAGCG | CCACGGTGACTCTGCAATTGACCAGTCTGACAGCCGCGGACACGGCC |
| | | ACGTGCAGTGTGACGATGCTGCCACTTACTACTGTGCAG | ACCTATTTCTGTGCGAGAGTGGGCTCCGATGACTATGGTGACTCTGAT |
| | | GCGGTTATAGTAGTAGTAGTGATACATTTGCTTTCGGCG | GTTTTTGATCCCTGGGGCCCAGGCACCCTGGTCACCGTCTCCTCA |
| | | GAGGG ACCGAGGTGGTGGTCAAG | (SEQ ID NO: 349) |
| | | (SEQ ID NO: 348) | |
| | AA | AAVLTQTPSPVSAAVGGTVSASCQSSKSVYNKNWLSWFQ | QEQLVESGGGLVKPGASLTLTCKASGFSFSSGQLMCWVRQAPGKGLE |
| | | QKPGQPPKLLIYGASTLASGVPSRFKGSGSGTQFTLTISDV | WIACIGSGSNAISTFYASWAQGRFTISKSSSTTVTLQLTSLTAADTATYF |
| | | QCDDAATYYCAGGYSSSSDTFAFGGGTEVVVK | CARVGSDDYGDSDVFDPWGPGTLVTVSS |
| | | (SEQ ID NO: 350) | (SEQ ID NO: 351) |
| 7B20 | NA | GCCCAAGTGCTGACCCAGACTCCAGCCTCCGTGTCTGC | CTGACACTCACCTGCAAAGCCTCTGGGTCAGACCTCAGTAGCGCCTA |
| | | AGCTGTGGGAGGCACAGTCACCATCAGTTGCCAGTCCAG | CTACATATGCTGGATCCGCCAGGCTCCAGGGAAGGGGCTGGAGTGG |
| | | TCAGAGCGTTTATAGTAGCGACCTCTTATCCTGGTATCAG | GTCGCATGTATTGGTGGTGTTAATCGCGTTGCCACTGCCTACGCGAC |
| | | CAGAAACCAGGGCAGCCTCCCAAGCTCCTGATCTACAA | CTGGGCGAAAGGCCGATTCACCATCTCCAAAACCTCGTCGACCACGG |
| | | GGCATCCACTCTGGCATCTGGGGTCCCATCGCGGTTCAAA | CAGTCGTTGGAGGAGTCCGGGGGAGGCCTGGTCAAGCCTGGGGCATCC |
| | | GGCAGTGGATCTGGGACACAGTTCACTCTCACAATCAGC | TGACTCTGCAAATGACCAGTCTGACAGCCGCGGACACGGCCACTTATT |
| | | GAACTACAGTGTGACGATGCTGCCACTTATTACTGTCAA | TCTGTGTGAGAGGCGGTCCTGATAATAATGTCCAATTTAACTTGTGG |
| | | GGCTACTATAGTGGTGTGGTTTATATTTTCGGCGGAGGG | GGCCCAGGCACCCTGGTCACCGTCTCCTCA |
| | | ACCGAGGTGGTGGTCAAG | (SEQ ID NO: 353) |
| | | (SEQ ID NO: 352) | |
| | AA | AQVLTQTPASVSAAVGGTVTISCQSSQSVYSSDLLSWYQQ | QSLEESGGGLVKPGASLTLTCKASGSDLSSAYYICWIRQAPGKGLEWVA |
| | | KPGQPPKLLIYKASTLASGVPSRFKGSGSGTQFTLTISELQC | CIGGVNRVATAYATWAKGRFTISKTSSTTVTLQMTSLTAADTATYFCVR |
| | | DDAATYYCQGYYSGVVYIFGGGTEVVVK | GGPDNNVQFNLWGPGTLVTVSS |
| | | (SEQ ID NO: 354) | (SEQ ID NO: 355) |
| 7J6 | NA | GCCCAAGTGCTGACCCAGACTCCAGCCTCCGTGTCTGC | CAGTCGTTGGAGGAGTCCGGGGGAGGCCTGGTCAAGCCTGGGGCATCC |
| | | AGCTGTGGGAGGCACAGTCACCATCAGTTGCCAGTCCAG | CTGACACTCACCTGCAAAGCCTCTGGGTCAGACCTCAGTAGGTACTA |
| | | TCAGAGCGTTTATAGTAGCGACCTCTTATCCTGGTATCAG | CTACAGTTGCTGGATCCGCCAGGCTCCAGGGAAGGGGCTGGAGTGG |
| | | CAAAAACCAGGGCAGCCTCCCAAGCTCCTGATCTACAA | GTCGCATGTGTTGGTGGTGTTAATCGCGATGCCACTGCCTACGCGACC |
| | | GGCATCCACTCTGGCATCTGGGGTCCCATCGCGGTTCAAA | TGGGCGAAAGGCCGATTCACCATCTCCAAAACCTCGTCGACCACGGTG |
| | | GGCAGTGGATCTGGGACACAGTTCACTCTCACAATCAGC | ACTCTGCAAATGACCAGTCTGACAGCCGCGGACACGGCCACTTATTTC |
| | | GAAGTACAGTGTGACGATGCTGCCACTTATTACTGTCAA | TGTGTGAGAGGCGGTCCTGATAATAATGTCCAATTTAACTTGTGGGG |
| | | GGCTACTATAGTGGTGTGGTTTATATTTTCGGCGGAGGG | CCCAGGCACCCTGGTCACCGTCTCCTCA |
| | | ACCGAGGTGGTGGTCAAG | (SEQ ID NO: 357) |
| | | (SEQ ID NO: 356) | |
| | AA | AQVLTQTPASVSAAVGGTVTISCQSSQSVYSSDLLSWYQQ | QSLEESGGGLVKPGASLTLTCKASGSDLSRYYYSCWIRQAPGKGLEWV |
| | | KPGQPPKLLIYKASTLASGVPSRFKGSGSGTQFTLTISEVQC | ACVGGVNRDATAYATWAKGRFTISKTSSTTVTLQMTSLTAADTATYFC |
| | | DDAATYYCQGYYSGVVYIFGGGTEVVVK | VRGGPDNNVQFNLWGPGTLVTVSS |
| | | (SEQ ID NO: 358) | (SEQ ID NO: 359) |
| 1D2 | NA | GACATTGTGATGACCCAGACTCCATTCTCCGTGGAGGC | CAGTCGTTGGAGGAGTCCGGGGGAGGCCTGGTCCAGCCTGAGGGAT |
| | | AGCTGTGGGAGGCACAGTCACCATCAAGTGCCAGGCCA | CCCTGACACTCACCTGCACAGCTTCTGGATTCACCCTCAATAATTATT |
| | | GTCAGAGCATTAGTAGTTACTTATCTTGGTATCAGCAGA | ATATTTGTTGGGTCCGCCAGGCTCCAGGGAAGGGACTGGAGTGGATC |
| | | AACCAGGGCAGCCTCCCAAGCTCCTGATCTACAGGGCAT | GCATGTATTGACAATGTTAATGGTAGGACTTACTACGCGAGCTGGGC |
| | | CCCCTCTGGCATCTGGGGTCCCATCGCGGTTCAAAGGCAG | GAAAGGCCGATTCACCATCTCCAGAACCTCGTCGACCACGGTGACTCT |
| | | TGGATCTGGGACACAGTTCACTCTCACCATCAGCGACCTG | GCAAATGACCAGTCTGACAGCCGCGGACACGGCCACCTATTTCTGTGC |
| | | GAGTGTGCCGATGCTGCCACTTACTACTGTCAAAGCTAC | GAGGTCATTGTCTACTCCCTTGTGGGGCCCAGGCACCCTGGTCACCGT |
| | | GATGATAGTAGTAGTAATAATTTTTTTTATGGTTTCGGC | CTCCTCG |
| | | GGAGGGACCGAGGTGGTGGTCAGA | (SEQ ID NO: 361) |
| | | (SEQ ID NO: 360) | |
| | AA | DIVMTQTPFSVEAAVGGTVTIKCQASQSISSYLSWYQQKP | QSLEESGGGLVQPEGSLTLTCTASGFTLNNYYICWVRQAPGKGLEWIAC |
| | | GQPPKLLIYRASPLASGVPSRFKGSGSGTQFTLTISDLECAD | IDNVNGRTYYASWAKGRFTISRTSSTTVTLQMTSLTAADTATYFCARSL |
| | | AATYYCQSYDDSSSNNFFYGFGGGTEVVVR | STPLWGPGTLVTVSS |
| | | (SEQ ID NO: 362) | (SEQ ID NO: 363) |
| 4G8 | NA | GCCGCCGTGCTGACCCAGACTCCATCTCCCGTGTCTGCAG | CAGGAGCAGCTGGTGGAGTCCGGGGGAGGCCTGGTCCAGCCTGAGG |
| | | CTGTGGGAGGCACAGTCACCATCAATTGCCAGTCCAGTA | GATCCCTGACACTCACCTGCGCAGCTTCTGGATTCTCCTTCAGTAGCA |
| | | AGAGTGTTTATAATAAGAACTGGTTATCCTGGTTTCAGC | GCTACTTCATGTGCTGGGTCCGCCAGGCTCCAGGGAAGGGGCTGGAG |
| | | AGAAACCAGGGCAGCCTCCCAAGCTCCTGATCTATGGTG | TGGATCGCATGCATTGCTGTTGGTAGTAGTGGTAGCACTTACTACGC |
| | | CATCCACTCTGGCATCTGGGGTCCCATCGCGGTTCAAAG | GAGCTGGGCGAAAGGCCGATTCACCATCTCCAAAACCTCGTCGACCA |
| | | GCAGTGGATCTGGGACACAGTTCACTCTCACCATCAGCG | CGGTGACTCTGCAAATGACCAGTCTGACAGCCGCGGACACGGCCACC |
| | | ACGTGCAGTGTGACGATGTTGCCACTTACTACTGTGCAGG | TATTTCTGTGCGAGAGTGGGCTACGATGACTATGGTGACTCTGATGC |
| | | CGGTTATAGTAGTAGTAGTGATACGTTTGCTTTCGGCGG | TTTTGATCCCTGGGGCCCAGGCACCCTGGTCACCGTCTCCTCA |
| | | AGGGACCGAGGTGGTGGTCAAA | (SEQ ID NO: 365) |
| | | (SEQ ID NO: 364) | |
| | AA | AAVLTQTPSPVSAAVGGTVTINCQSSKSVYNKNWLSWFQ | QEQLVESGGGLVQPEGSLTLTCAASGFSFSSSYFMCWVRQAPGKGLEWI |
| | | QKPGQPPKLLIYGASTLASGVPSRFKGSGSGTQFTLTISDV | ACIAVGSSGSTYYASWAKGRFTISKTSSTTVTLQMTSLTAADTATYFCA |
| | | QCDDVATYYCAGGYSSSSDTFAFGGGTEVVVK | RVGYDDYGDSDAFDPWGPGTLVTVSS |
| | | (SEQ ID NO: 366) | (SEQ ID NO: 367) |
| 7I4 | NA | GCCCAAGTGCTGACCCAGACTCCAGCCTCCGTGTCTGC | CAGTCGTTGGAGGAGTCCGGGGGAGGCCTGGTCAAGCCTGGGGCATCC |
| | | AGCTGTGGGAGGCACAGTCACCATCAGTTGCCAGTCCAG | CTGACACTCACCTGCAAAGCCTCTGGGTCAGACCTCAGTAGGTACTA |
| | | TCAGAGCCTTTATAATAGCGACCTCTTATCCTGGTATCAG | CTACAGTTGCTGGATCCGCCAGGCTCCAGGGAAGGGGCTGGAGTGG |
| | | CAAAAACCAGGGCAGCCTCCCAAGCTCCTGATCTACAA | GTCGCATGTGTTGGTGGTGTTAATCGCGATGCCACTGCCTACGCGACC |
| | | GGCATCCACTCTGGCATCTGGGGTCCCATCGCGGTTCAAA | TGGGCGAAAGGCCGATTCACCATCTCCAAAACCTCGTCGACCACGGTG |
| | | GGCAGTGGATCTGGGACACAGTTCACTCTCACAATCAGC | ACTCTGCAAATGACCAGTCTGACAGCCGCGGACACGGCCACTTATTTC |
| | | GAAGTACAGTGTGACGATGCTGCCACTTATTACTGTCAA | TGTGTGAGAGGCGGTCCTGATAATAATGTCCAATTTAACTTGTGGGG |
| | | GGCTACTATAGTGGTGTGGTTTATATTTTCGGCGGAGGG | CCCAGGCACCCTGGTCACCGTCTCCTCA |
| | | ACCG AGGTGGTGGTCAAG | (SEQ ID NO: 369) |
| | | (SEQ ID NO: 368) | |
| | AA | AQVLTQTPASVSAAVG | QSLEESGGGLVKPGASLTLTCKASGSDLSRYYYSCWIRQAPGKGLEWV |
| | | GTVTISCQSSQSLYNSDLLSWYQQKPGQPPKLLIYKASTLA | ACVGGVNRDATAYATWAKGRFTISKTSSTTVTLQMTSLTAADTATYFC |
| | | SGVPSRFKGSGSGTQFTLTISEVQCDDAATYYCQGYYSGVV | VRGGPDNNVQFNLWGPGTLVTVSS |
| | | YIFGGGTEVVVK | (SEQ ID NO: 371) |
| | | (SEQ ID NO: 370) | |
| 1I23 | NA | GCCGCCGTGCTGACCCAGACTCCATCTCCCGTGTCTGCAG | CAGGAGCAGCTGGTGGAGTCCGGGGGAGGCCTGGTCCAGCCTAAGG |
| | | CTGTGGGAGGCACAGTCAGCATCAGTTGCCAGTCCAGTA | GATCCCTGACACTCACCTGCGCAGCTTCTGGATTCTCCTTCAGTAGCA |
| | | AGAGTGTTTATAATAAGAACTGGTTATCCTGGTTTCAGC | GCTACTTCATGTGCTGGGTCCGCCAGGCTCCAGGGAAGGGGCTGGAG |
| | | AGAAACCAGGGCAGCCTCCCAAGCTCCTGATCTATGGTG | TGGATCGCATGCATTGGTTCTGGTAGTAGTGCTATTAGCACTTTCTAC |
| | | CATCCACTCTGGCATCTGGGGTCCCATCGCGGTTCAAAG | GCGAGCTGGGCGCAAGGCCGATTCACCATCTCCAAAACCTCGTCGA |
| | | GCAGTGGATCTGGGACACAGTTCACTCTCACCATCAGCG | CCACGGTGACTCTGCAAATGACCAGTCTGACAGCCGCGGACACGGC |
| | | ACGTGCAGTGTGACGATGCTGCCACTTACTACTGTGCAG | CACCTATTTCTGTGCGAGAGTGGGCTACGATGACTATGGTGACTCTG |
| | | GCGGTTATAGTAGTAGTAGTGATACATTTGCTTTCGGCG | ATGCTTTTGATCCCTGGGGCCCAGGCACCCTGGTCACCGTCTCCTCA |
| | | GAGGG ACCGAGGTGGTGGTCAAA | (SEQ ID NO: 373) |
| | | (SEQ ID NO: 372) | |
| | AA | AAVLTQTPSPVSAAVGGTVSISCQSSKSVYNKNWLSWFQ | QEQLVESGGGLVQPKGSLTLTCAASGFSFSSSYFMCWVRQAPGKGLEW |
| | | QKPGQPPKLLIYGASTLASGVPSRFKGSGSGTQFTLTISDVQ | IACIGSGSSAISTFYASWAQGRFTISKTSSTTVTLQMTSLTAADTATYFCA |
| | | CDDAATYYCAGGYSSSSDTFAFGGGTEVVVK | RVGYDDYGDSDAFDPWGPGTLVTVSS |
| | | (SEQ ID NO: 374) | (SEQ ID NO: 375) |
| 1J13 | NA | GATGTTGTGATGACCCACACTCCAGCCTCCCTCTTTGAAA | CAGTCGCTGGAGGAGTCCGGGGGTCGCCTGGTAACGCCTGGGACACCC |
| | | CTGTGGGAGGCACAGTCACCATCAAGTGCCAGGCCCTTC | CTGACACTCACCTGCACAGTCTCTGGAATCGACCTCAGTAGCTATGGA |
| | | AGAGTGTTTATGATAACAATGCTTTATCCTGGTATCAACA | ATGACTTGGGTCCGCCAGGCTCCAGGGAAGGGGCTGGAGTGGATCGG |
| | | AAATGCAGGACAGCGTCCCATACTCCTGATCTATGGTGCA | ATACATTTGGACTGATGGGAGGACATACTACGCAAACTGGGCGAAAG |
| | | TCCACTCTGGCATCTGGGGCCCCATCGCGGTTCAGTGCC | GCCGATTCACCATCTCCAAAACCTCGACCACAGTGGATCTCAAGATCA |
| | | AGTGGATCTGGGACAGATTTCACTCTCACCATCATCGAC | CCAGTCCGACAGCCGAGGACACGGCCACCTATTTCTGTGCCAGACCCT |
| | | CTGGAGTGTGCCGATGCTTCCACTTACTACTGTCAATGT | TTGATGGTAATTATAGGGACATCTGGGGCCCAGGCACCCTGGTCACG |
| | | ACTTATTATGTTAGTAGTTATCAAAATGATTTCGGCGGA | GTCTCCTTA |
| | | GGG ACCGAGGTGGTGGTCAAA | (SEQ ID NO: 377) |
| | | (SEQ ID NO: 376) | |
| | AA | DVVMTHTPASLFETVGGTVTIKCQALQSVYDNNALSWYQ | QSLEESGGRLVTPGTPLTLTCTVSGIDLSSYGMTWVRQAPGKGLEWIGY |
| | | QNAGORPILLIYGASTLASGAPSRFSASGSGTDFTLTIIDLEC | IWTDGRTYYANWAKGRFTISKTSTTVDLKITSPTAEDTATYFCARPFDG |
| | | ADASTYYCQCTYYVSSYQNDFGGGTEVVVK | NYRDIWGPGTLVTVSL |
| | | (SEQ ID NO: 378) | (SEQ ID NO: 379) |
| 1G24 | NA | GACATTGTGATGACCCAGACTCCAGCCTCCGTGGAGGC | CAGTCGTTGGAGGAGTCCGGGGGAGGCCTGGTCCAGCCTGAGGGAT |
| | | AGCTGTGGGAGGCACAGTCACCATCAAGTGCCAGGCCA | CCCTGACACTCACCTGCACAGCTTCTGGATTCACCCTCAATAACTACT |
| | | GTGAGAGCATTGGCAATGCATTAGCCTGGTATCAGCAGA | ACATCTGCTGGGTCCGCCAGGCTCCAGGGAAGGGGCTGGAGTGGATC |
| | | AACCAGGGCAGCCTCCCAGTCTCCTGATCTACAGGGCA | GCATGTATTGATAATGCCAATGGTCGGACTTACTACGCGAACTGGGC |
| | | TCCACTCTGGCATCTGGAATCCCATCGCGGGTCAAAGG | GAAAGGCCGATTCACCATCTCCAAGACCTCGTCGACCACGGTGACTC |
| | | CAGTGGATCTGGGACACAGTTCACTCTCACCATCAGCGA | TGCAAATGACCAGTCTGACAGCCGCGGACACGGCCACCTATTTCTGTG |
| | | CCTGGAGTGTGCCGATGCTGCCACTTACTATTGTCAAAGC | CGAGGTCCTTGGCTACTCCCTTGTGGGGCCCAGGCACCCTGGTCACC |
| | | TATGATGATAGTAGTAGTAGTAGTTTTTTTTATGCTTTCG | GTCTCCTCA |
| | | GCGGA GGGACCGAGGTGGTGGTCAAA | (SEQ ID NO: 381) |
| | | (SEQ ID NO: 380) | |
| | AA | DIVMTQTPASVEAAVGGTVTIKCQASESIGNALAWYQQKP | QSLEESGGGLVQPEGSLTLTCTASGFTLNNYYICWVRQAPGKGLEWIAC |
| | | GQPPSLLIYRASTLASGIPSRVKGSGSGTQFTLTISDLECADA | IDNANGRTYYANWAKGRFTISKTSSTTVTLQMTSLTAADTATYFCARSL |
| | | ATYYCQSYDDSSSSSFFYAFGGGTEVVVK | ATPLWGPGTLVTVSS |
| | | (SEQ ID NO: 382) | (SEQ ID NO: 383) |
| 4A7 | NA | GCCGCCGTGCTGACCCAGACTCCATCTCCCGTGTCTGCAG | CAGGAGCAGCTGGTGGAGTCCGGGGGAGGCCTGGTCCAGCCTGAGG |
| | | CTGTGGGAGGCACAGTCAGCATCAGTTGCCAGTCCAGTA | GATCCCTGACACTCACCTGCGCAGCTTCTGGATTCTCCTTCAGTAGCA |
| | | AGAGTGTTTATAATAAGAACTGGTTATCCTGGTTTCAGC | GCTACTTCATGTGCTGGGTCCGCCAGGCTCCAGGGAAGGGGCTGGAG |
| | | AGAAACCAGGGCAGCCTCCCAAGCTCCTGATCTATGGTG | TGGATCGCATGCATTGGTGTTGGTGGTAGTGGTAGCACTTACTACGC |
| | | CATCCACTCTGGCATCTGGGGTCCCATCGCGGTTCAAAG | GAACTGGGCGAAAGGCCGATTCACCATCTCCAAAACCTCGTCGACCA |
| | | GCAGTGGATCTGGGACACAGTTCACTCTCACCATCAGCG | CGGTGACTCTGCAAATGACCAGTCTGACAGCCGCGGACACGGCCACC |
| | | GCGTGCAGTGTGACGATGCTGCCACTTACTACTGTGCAG | TATTTCTGTGCGAGAGTGGCCTACGATGACGATGGTGACTCTGATGC |
| | | GCGGTTATAGTAGTAGTAGTGATACGTTTGCTTTCGGCG | TTTTGATCCCTGGGGCCCAGGCACCCTGGTCACCGTCTCCTCA |
| | | GAGGGACCGAGGTGGTGGTCAAA | (SEQ ID NO: 385) |
| | | (SEQ ID NO: 384) | |
| | AA | AAVLTQTPSPVSAAVGGTVSISCQSSKSVYNKNWLSWFQ | QEQLVESGGGLVQPEGSLTLTCAASGFSFSSSYFMCWVRQAPGKGLEWI |
| | | QKPGQPPKLLIYGASTLASGVPSRFKGSGSGTQFTLTISGV | ACIGVGGSGSTYYANWAKGRFTISKTSSTTVTLQMTSLTAADTATYFC |
| | | QCDDAATYYCAGGYSSSSDTFAFGGGTEVVVK | ARVAYDDDGDSDAFDPWGPGTLVTVSS |
| | | (SEQ ID NO: 386) | (SEQ ID NO: 387) |

TABLE 24
Standard IgG Antibody Variable Region Protein Alignment
CDRs defined by Kabat
Alignment numbering defined by [symbol]
KAPPA_VARIABLE

| | | Germline | K_FR1 | K_CDR1 |
|---|---|---|---|---|
| | [symbol] | | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| | | Germline | K_FR1 | K_CDR1 |
| | [symbol] | | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| | | Germline | K_FR1 | K_CDR1 |
| | [symbol] | | | |
| [symbol] | [symbol] | [symbol] | [symbol] | [symbol] |
| K_FR2 | K_CDR2 | K_FR3 | K_CDR3 | K_FR4 |
| [symbol] | | | | |
| [symbol] | | | | |
| [symbol] | | | | |
| [symbol] | | | | |
| K_FR2 | K_CDR2 | K_FR3 | K_CDR3 | K_FR4 |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| K_FR2 | K_CDR2 | K_FR3 | K_CDR3 | K_FR4 |
| [symbol] | [symbol] | [symbol] | [symbol] | [symbol] |
| [symbol] | [symbol] | [symbol] | [symbol] | [symbol] |
| | | Germline | K_FR1 | K_CDR1 |
| | [symbol] | | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| | | Germline | K_FR1 | K_CDR1 |
| | [symbol] | | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| | | Germline | K_FR1 | K_CDR1 |
| | [symbol] | | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |

HEAVY VARIABLE

| | | Germline | H_FR1 | H_CDR1 |
|---|---|---|---|---|
| | [symbol] | | [symbol] | [symbol] |
| [symbol] | [symbol] | [symbol] | [symbol] | [symbol] |
| | | Germline | H_FR1 | H_CDR1 |
| | [symbol] | | [symbol] | [symbol] |
| [symbol] | [symbol] | [symbol] | [symbol] | [symbol] |
| | | Germline | H_FR1 | H_CDR1 |
| K_FR2 | K_CDR2 | K_FR3 | K_CDR3 | K_FR4 |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| K_FR2 | K_CDR2 | K_FR3 | K_CDR3 | K_FR4 |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| K_FR2 | K_CDR2 | K_FR3 | K_CDR3 | K_FR4 |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| H_FR2 | H_CDR2 | H_FR3 | H_CDR3 | H_FR4 |
| [symbol] | [symbol] | [symbol] | [symbol] | [symbol] |
| [symbol] | [symbol] | [symbol] | [symbol] | [symbol] |
| H_FR2 | H_CDR2 | H_FR3 | H_CDR3 | H_FR4 |
| [symbol] | [symbol] | [symbol] | [symbol] | [symbol] |
| [symbol] | [symbol] | [symbol] | [symbol] | [symbol] |
| H_FR2 | H_CDR2 | H_FR3 | H_CDR3 | H_FR4 |
| | [symbol] | | [symbol] | [symbol] |
| [symbol] | [symbol] | [symbol] | [symbol] | [symbol] |
| | | Germline | H_FR1 | H_CDR1 |
| | [symbol] | | [symbol] | [symbol] |
| [symbol] | [symbol] | [symbol] | [symbol] | [symbol] |
| | | Germline | H_FR1 | H_CDR1 |
| | [symbol] | | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| | | Germline | H_FR1 | H_CDR1 |
| | [symbol] | | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| | | Germline | H_FR1 | H_CDR1 |
| | [symbol] | | [symbol] | [symbol] |
| [symbol] | [symbol] | [symbol] | [symbol] | [symbol] |
| | | Germline | H_FR1 | H_CDR1 |
| | [symbol] | | [symbol] | [symbol] |
| [symbol] | [symbol] | [symbol] | [symbol] | [symbol] |
| | | Germline | H_FR1 | H_CDR1 |
| | [symbol] | | [symbol] | [symbol] |
| [symbol] | [symbol] | [symbol] | [symbol] | [symbol] |
| | | Germline | H_FR1 | H_CDR1 |
| | [symbol] | | [symbol] | [symbol] |
| [symbol] | [symbol] | [symbol] | [symbol] | [symbol] |
| [symbol] | [symbol] | [symbol] | [symbol] | [symbol] |
| | | Germline | H_FR1 | H_CDR1 |
| | [symbol] | | [symbol] | [symbol] |
| [symbol] | [symbol] | [symbol] | [symbol] | [symbol] |
| | | Germline | H_FR1 | H_CDR1 |
| | [symbol] | | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| H_FR2 | H_CDR2 | H_FR2 | H_CDR3 | H_FR4 |
| [symbol] | [symbol] | [symbol] | [symbol] | [symbol] |
| [symbol] | [symbol] | [symbol] | [symbol] | [symbol] |
| H_FR2 | H_CDR2 | H_FR2 | H_CDR3 | H_FR4 |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| H_FR2 | H_CDR2 | H_FR2 | H_CDR3 | H_FR4 |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| H_FR2 | H_CDR2 | H_FR2 | H_CDR3 | H_FR4 |
| [symbol] | [symbol] | [symbol] | [symbol] | [symbol] |
| [symbol] | [symbol] | [symbol] | [symbol] | [symbol] |
| H_FR2 | H_CDR2 | H_FR2 | H_CDR3 | H_FR4 |
| [symbol] | [symbol] | [symbol] | [symbol] | [symbol] |
| H_FR2 | H_CDR2 | H_FR2 | H_CDR3 | H_FR4 |
| [symbol] | [symbol] | [symbol] | [symbol] | [symbol] |
| [symbol] | [symbol] | [symbol] | [symbol] | [symbol] |
| H_FR2 | H_CDR2 | H_FR2 | H_CDR3 | H_FR4 |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| H_FR2 | H_CDR2 | H_FR2 | H_CDR3 | H_FR4 |
| [symbol] | [symbol] | [symbol] | [symbol] | [symbol] |
| [symbol] | [symbol] | [symbol] | [symbol] | [symbol] |
| H_FR2 | H_CDR2 | H_FR2 | H_CDR3 | H_FR4 |
| [symbol] | [symbol] | [symbol] | [symbol] | [symbol] |
| [symbol] | [symbol] | [symbol] | [symbol] | [symbol] |

[symbol]

indicates data missing or illegible when filed

TABLE 25
below discloses SEQ ID NOS 600-644, 645, 644 and 646-664, respectively, in order
of appearance.
Standard IgG Antibody Variable Region Consensus Protein Alignment
CDRs defined by Kabat
Alignment numbering defined by [symbol]
KAPPA VARIABLE

| | | Germline | K_FR1 | K_CDR1 |
|---|---|---|---|---|
| | [symbol] | | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| | | Germline | K_FR1 | K_CDR1 |
| | [symbol] | | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| | | Germline | K_FR1 | K_CDR1 |
| | [symbol] | | [symbol] | [symbol] |
| [symbol] | [symbol] | [symbol] | [symbol] | [symbol] |
| | | Germline | K_FR1 | K_CDR1 |
| | [symbol] | | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| | | Germline | K_FR1 | K_CDR1 |
| | [symbol] | | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| K_FR2 | K_CDR2 | K_FR3 | K_CDR3 | K_FR4 |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| K_FR2 | K_CDR2 | K_FR3 | K_CDR3 | K_FR4 |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| K_FR2 | K_CDR2 | K_FR3 | K_CDR3 | K_FR4 |
| [symbol] | [symbol] | [symbol] | [symbol] | [symbol] |
| [symbol] | [symbol] | [symbol] | [symbol] | [symbol] |
| K_FR2 | K_CDR2 | K_FR3 | K_CDR3 | K_FR4 |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| K_FR2 | K_CDR2 | K_FR3 | K_CDR3 | K_FR4 |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| | | Germline | K_FR1 | K_CDR1 |
| | [symbol] | | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |

HEAVY_VARIABLE

| | | Germline | H_FR1 | H_CDR1 |
|---|---|---|---|---|
| | [symbol] | | [symbol] | [symbol] |
| [symbol] | [symbol] | [symbol] | [symbol] | [symbol] |
| | | Germline | H_FR1 | H_CDR1 |
| | [symbol] | | [symbol] | [symbol] |
| [symbol] | [symbol] | [symbol] | [symbol] | [symbol] |
| | | Germline | H_FR1 | H_CDR1 |
| | [symbol] | | [symbol] | [symbol] |
| [symbol] | [symbol] | [symbol] | [symbol] | [symbol] |
| | | Germline | H_FR1 | H_CDR1 |
| | [symbol] | | [symbol] | [symbol] |
| [symbol] | [symbol] | [symbol] | [symbol] | [symbol] |
| | | Germline | H_FR1 | H_CDR1 |
| | [symbol] | | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| K_FR2 | K_CDRR2 | K_FR3 | K_CDR3 | K_FR4 |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| H_FR2 | H_CDRR2 | H_FR3 | H_CDR3 | H_FR4 |
| [symbol] | [symbol] | [symbol] | [symbol] | [symbol] |
| [symbol] | [symbol] | [symbol] | [symbol] | [symbol] |
| H_FR2 | H_CDRR2 | H_FR3 | H_CDR3 | H_FR4 |
| [symbol] | [symbol] | [symbol] | [symbol] | [symbol] |
| [symbol] | [symbol] | [symbol] | [symbol] | [symbol] |
| H_FR2 | H_CDRR2 | H_FR3 | H_CDR3 | H_FR4 |
| [symbol] | [symbol] | [symbol] | [symbol] | [symbol] |
| [symbol] | [symbol] | [symbol] | [symbol] | [symbol] |
| H_FR2 | H_CDRR2 | H_FR3 | H_CDR3 | H_FR4 |
| [symbol] | [symbol] | [symbol] | [symbol] | [symbol] |
| [symbol] | [symbol] | [symbol] | [symbol] | [symbol] |
| H_FR2 | H_CDRR2 | H_FR3 | H_CDR3 | H_FR4 |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| | | Germline | H_FR1 | H_CDR1 |
| | [symbol] | | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| | | Germline | H_FR1 | H_CDR1 |
| | [symbol] | | [symbol] | [symbol] |
| [symbol] | [symbol] | [symbol] | [symbol] | [symbol] |
| | | Germline | H_FR1 | H_CDR1 |
| | [symbol] | | [symbol] | [symbol] |
| [symbol] | [symbol] | [symbol] | [symbol] | [symbol] |
| | | Germline | H_FR1 | H_CDR1 |
| | [symbol] | | [symbol] | [symbol] |
| [symbol] | [symbol] | [symbol] | [symbol] | [symbol] |
| | | Germline | H_FR1 | H_CDR1 |
| | [symbol] | | [symbol] | [symbol] |
| [symbol] | [symbol] | [symbol] | [symbol] | [symbol] |
| [symbol] | [symbol] | [symbol] | [symbol] | [symbol] |
| | | Germline | H_FR1 | H_CDR1 |
| | [symbol] | | [symbol] | [symbol] |
| [symbol] | [symbol] | [symbol] | [symbol] | [symbol] |
| | | Germline | H_FR1 | H_CDR1 |
| | [symbol] | | [symbol] | [symbol] |
| [symbol] | [symbol] | [symbol] | [symbol] | [symbol] |
| [symbol] | [symbol] | [symbol] | [symbol] | [symbol] |
| H_FR2 | H_CDR2 | H_FR3 | H_CDR3 | H_FR4 |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| H_FR2 | H_CDR2 | H_FR3 | H_CDR3 | H_FR4 |
| [symbol] | [symbol] | [symbol] | [symbol] | [symbol] |
| [symbol] | [symbol] | [symbol] | [symbol] | [symbol] |
| H_FR2 | H_CDR2 | H_FR3 | H_CDR3 | H_FR4 |
| [symbol] | [symbol] | [symbol] | [symbol] | [symbol] |
| [symbol] | [symbol] | [symbol] | [symbol] | [symbol] |
| H_FR2 | H_CDR2 | H_FR3 | H_CDR3 | H_FR4 |
| [symbol] | [symbol] | [symbol] | [symbol] | [symbol] |
| [symbol] | [symbol] | [symbol] | [symbol] | [symbol] |
| H_FR2 | H_CDR2 | H_FR3 | H_CDR3 | H_FR4 |
| [symbol] | [symbol] | [symbol] | [symbol] | [symbol] |
| [symbol] | [symbol] | [symbol] | [symbol] | [symbol] |
| [symbol] | [symbol] | [symbol] | [symbol] | [symbol] |
| H_FR2 | H_CDR2 | H_FR3 | H_CDR3 | H_FR4 |
| [symbol] | [symbol] | [symbol] | [symbol] | [symbol] |
| [symbol] | [symbol] | [symbol] | [symbol] | [symbol] |
| H_FR2 | H_CDR2 | H_FR3 | H_CDR3 | H_FR4 |
| [symbol] | [symbol] | [symbol] | [symbol] | [symbol] |
| [symbol] | [symbol] | [symbol] | [symbol] | [symbol] |

[symbol]

indicates data missing or illegible when filed

What is claimed is:

1. An isolated antigen binding construct that binds MAGEB2, wherein the binding construct binds to an epitope comprising a sequence selected from SEQ ID NOs: 2, 3, 4, 562, or 388-554.

2. The isolated antigen binding construct of claim 1, wherein:
a) the antigen binding construct comprises a CDRL1, a CDRL2, a CDRL3, a CDRH1, a CDRH2, and a CDRH3, wherein the CDRL1 comprises a sequence set forth in SEQ ID NO: 85; the CDRL2 comprises a sequence set forth in SEQ ID NO: 86; the CDRL3 comprises a sequence set forth in SEQ ID NO: 87; the CDRH1 comprises a sequence set forth in SEQ ID NO: 229; the CDRH2 comprises a sequence set forth in SEQ ID NO: 230; and the CDRH3 comprises a sequence set forth in SEQ ID NO: 231; or
b) the antigen binding construct comprises a CDRL1, a CDRL2, a CDRL3, a CDRH1, a CDRH2, and a CDRH3, wherein the CDRL1 comprises a sequence set forth in SEQ ID NO: 73; the CDRL2 comprises a sequence set forth in SEQ ID NO: 74; the CDRL3 comprises a sequence set forth in SEQ ID NO: 75; the CDRH1 comprises a sequence set forth in SEQ ID NO: 217; the CDRH2 comprises a sequence set forth in SEQ ID NO: 218; and the CDRH3 comprises a sequence set forth in SEQ ID NO: 219; or
c) the antigen binding construct comprises a CDRL1, a CDRL2, a CDRL3, a CDRH1, a CDRH2, and a CDRH3, wherein the CDRL1 comprises a sequence set forth in SEQ ID NO: 91; the CDRL2 comprises a sequence set forth in SEQ ID NO: 92; the CDRL3 comprises a sequence set forth in SEQ ID NO: 93; the CDRH1 comprises a sequence set forth in SEQ ID NO: 235; the CDRH2 comprises a sequence set forth in SEQ ID NO: 236; and the CDRH3 comprises a sequence set forth in SEQ ID NO: 237.

3. (canceled)

4. (canceled)

5. The isolated antigen binding construct of claim 1, wherein:
a) the antigen binding construct comprises a light chain variable region comprising a sequence set forth in SEQ ID NO: 346 and a heavy chain variable region comprising a sequence set forth in SEQ ID NO: 347; or
b) the antigen binding construct comprises a light chain variable region comprising a sequence set forth in SEQ ID NO: 338 and a heavy chain variable region comprising a sequence set forth in SEQ ID NO: 339; or
c) the antigen binding construct comprises a light chain variable region comprising a sequence set forth in SEQ ID NO: 350 and a heavy chain variable region comprising a sequence set forth in SEQ ID NO: 351.

6. (canceled)

7. (canceled)

8. The isolated antigen binding construct of claim 1, wherein the antigen binding construct is a monoclonal antibody or an antibody fragment thereof.

9. The isolated antibody of claim 8, wherein the antibody is of the IgG1 or the IgG2 isotype.

10. The isolated antigen binding construct of claim 1, wherein the antigen binding construct is coupled to a labeling group.

11. A nucleic acid molecule encoding the antigen binding construct, or fragment thereof, according to claim 1.

12. A vector comprising a nucleic acid molecule according to claim 11.

13. A host cell comprising the nucleic acid molecule according to claim 12.

14. An antigen binding construct, or fragment thereof, produced by the host cell of claim 13.

15. A method of making the antigen binding construct, or fragment thereof, according to claim 1, comprising the step of preparing the antibody or fragment thereof from a host cell that secretes the antibody.

16. An isolated antibody or fragment thereof that competes for binding to MAGEB2 with an antibody or fragment thereof of claim 1.

17. A method of making an antibody that binds to MAGEB2 comprising immunizing an animal with a peptide comprising a sequence selected from SEQ ID NO: 2, 3, 4, or 562, and isolating from said animal antibodies that bind to MAGEB2.

18. An antibody made by the method of claim 17.

19. A method for treating a tumor in a subject, said method comprising: determining the subject as responsive to treatment with an anti-MAGEB2 therapeutic by obtaining a sample from the subject, wherein the sample comprises a cell from the tumor, measuring the level of MAGEB2 in the sample using an antigen binding construct of claim 1, and determining the subject as responsive to treatment with an anti-MAGEB2 therapeutic, and administering to the subject an effective amount of the anti-MAGEB2 therapeutic.

20. A method of identifying a subject as needing an anti-MAGEB2 therapeutic comprising: a) determining the level of MAGEB2 in a sample obtained from the subject using an antigen binding construct of claim 1; and b) identifying the subject as needing the anti-MAGEB2 therapeutic when the level of MAGEB2 is increased relative to a control.

21. A method of determining treatment for a subject with a MAGEB2 positive tumor comprising: determining the level of MAGEB2 in a sample obtained from the subject using an antigen binding construct of claim 1; and determining the treatment as comprising an anti-MAGEB2 therapeutic when the level of MAGEB2 is increased, relative to a control.

22. A method of determining efficacy of treatment with an anti-MAGEB2 therapeutic in a subject comprising: determining the level of MAGEB2 in a sample obtained from the subject using an antigen binding construct of claim 1 before treatment with an anti-MAGEB2 therapeutic and after treatment with an anti-MAGEB2 therapeutic; and determining the treatment as effective when the level of MAGEB2 positive tumor cells is decreased after treatment with the anti-MAGEB2 therapeutic.

23. A method of diagnosing a subject with a tumor, comprising: a) determining the level of MAGEB2 in a sample obtained from the subject using an antigen binding construct of claim 1; and b) diagnosing the subject as having a MAGEB2 positive tumor when the level of MAGEB2 is increased relative to a control.

24. A method of identifying a subject having a MAGEB2 positive tumor comprising: a) determining the level of MAGEB2 in a sample obtained from the subject using an antigen binding construct of claim 1; and b) identifying the subject as having a MAGEB2 positive tumor when the level of MAGEB2 is increased relative to a control.

25. A method of identifying a subject as needing an anti-MAGEB2 therapeutic comprising: a) determining the level of MAGEB2 in a sample obtained from the subject using an antigen binding construct of claim 1; and b) identifying the subject as needing the anti-MAGEB2 therapeutic when the level of MAGEB2 is increased relative to a control.

26. A method of determining treatment for a subject with a MAGEB2 positive tumor comprising: determining the level of MAGEB2 in a sample obtained from the subject using an antigen binding construct of claim 1; and determining the treatment as comprising an anti-MAGEB2 therapeutic when the level of MAGEB2 is increased, relative to a control.

27. The method of claim 19, wherein MAGEB2 is detected using an IHC assay.


SEQUENCE LISTING (664 sequences)

SEQ ID NO: 1 - protein, 319 aa, Homo sapiens
MPRGQKSKLRAREKRRKARDETRGLNVPQVTEAEEEEAPCCSSSVSGGAASSSPAAGIPQEPQRAPTTAAAAAAGVSSTKSKKGAKSHQGEKNASSSQASTSTKSPSEDPLTRKSGSLVQFLLYKYKIKKSVTKGEMLKIVGKRFREHFPEILKKASEGLSVVFGLELNKVNPNGHTYTFIDKVDLTDEESLLSSWDFPRRKLLMPLLGVIFLNGNSATEEEIWEFLNMLGVYDGEEHSVFGEPWKLITKDLVQEKYLEYKQVPSSDPPRFQFLWGPRAYAETSKMKVLEFLAKVNGTTPCAFPTHYEEALKDEEKAGV

SEQ ID NO: 2 - protein, 34 aa, Homo sapiens
SSVSGGAASSSPAAGIPQEPQRAPTTAAAAAAGV

SEQ ID NO: 3 - protein, 31 aa, Homo sapiens
SSSQASTSTKSPSEDPLTRKSGSLVQFLLYK

SEQ ID NO: 4 - protein, 10 aa, Artificial Sequence
  Description of Artificial Sequence Synthetic peptide
DKTHTCPPCP

SEQ ID NO: 5 - DNA, 33 bp, Artificial Sequence
  Description of Artificial Sequence Synthetic oligonucleotide
caggccagtcagagcattaggaatgaattattt

SEQ ID NO: 6 - DNA, 21 bp, Artificial Sequence
  Description of Artificial Sequence Synthetic oligonucleotide
gctgcatccaaactggcctct

SEQ ID NO: 7 - DNA, 42 bp, Artificial Sequence
  Description of Artificial Sequence Synthetic oligonucleotide
caatgcagttatgttagtagtagtggtacttatggaaatgtt

SEQ ID NO: 8 - protein, 7 aa, Artificial Sequence
  Description of Artificial Sequence Synthetic peptide
AASKLAS

SEQ ID NO: 9 - protein, 14 aa, Artificial Sequence
  Description of Artificial Sequence Synthetic peptide
QCSYVSSSGTYGNV

SEQ ID NO: 10 - DNA, 33 bp, Artificial Sequence
  Description of Artificial Sequence Synthetic oligonucleotide
caggccagtgaaagcattagcaactacttatcc

SEQ ID NO: 11 - DNA, 21 bp, Artificial Sequence
  Description of Artificial Sequence Synthetic oligonucleotide
tgggcatccactctggcatct

SEQ ID NO: 12 - DNA, 36 bp, Artificial Sequence
  Description of Artificial Sequence Synthetic oligonucleotide
caacagggttatagtagtagtaatgttgataatctt

SEQ ID NO: 13 - protein, 11 aa, Artificial Sequence
  Description of Artificial Sequence Synthetic peptide
QASESISNYLS

SEQ ID NO: 14 - protein, 7 aa, Artificial Sequence
  Description of Artificial Sequence Synthetic peptide
WASTLAS

SEQ ID NO: 15 - protein, 12 aa, Artificial Sequence
  Description of Artificial Sequence Synthetic peptide
QQGYSSSNVDNL

SEQ ID NO: 16 - DNA, 39 bp, Artificial Sequence
  Description of Artificial Sequence Synthetic oligonucleotide
cagtccagtcagagtgtttggcataacgactacttatcc

SEQ ID NO: 17 - DNA, 21 bp, Artificial Sequence
  Description of Artificial Sequence Synthetic oligonucleotide
ggtgcatccactttggcatct

SEQ ID NO: 18 - DNA, 33 bp, Artificial Sequence
  Description of Artificial Sequence Synthetic oligonucleotide
gcaggcggttatggacgtagtagtgaaaatggt

SEQ ID NO: 19 - protein, 13 aa, Artificial Sequence
  Description of Artificial Sequence Synthetic peptide
QSSQSVWHNDYLS

SEQ ID NO: 20 - protein, 7 aa, Artificial Sequence
  Description of Artificial Sequence Synthetic peptide
GASTLAS

SEQ ID NO: 21 - protein, 11 aa, Artificial Sequence
  Description of Artificial Sequence Synthetic peptide
AGGYGRSSENG

SEQ ID NO: 22 - DNA, 39 bp, Artificial Sequence
  Description of Artificial Sequence Synthetic oligonucleotide
cagtccagtaagagtgtttataataacaactggttagcc

SEQ ID NO: 23 - DNA, 21 bp, Artificial Sequence
  Description of Artificial Sequence Synthetic oligonucleotide
gatgcatcgactctagattct

SEQ ID NO: 24 - DNA, 33 bp, Artificial Sequence
  Description of Artificial Sequence Synthetic oligonucleotide
gtaggcggttatagtagtcgtagtgataatggt

SEQ ID NO: 25 - protein, 13 aa, Artificial Sequence
  Description of Artificial Sequence Synthetic peptide
QSSKSVYNNNWLA

SEQ ID NO: 26 - protein, 7 aa, Artificial Sequence
  Description of Artificial Sequence Synthetic peptide
DASTLDS

SEQ ID NO: 27 - protein, 11 aa, Artificial Sequence
  Description of Artificial Sequence Synthetic peptide
VGGYSSRSDNG

SEQ ID NO: 28 - DNA, 33 bp, Artificial Sequence
  Description of Artificial Sequence Synthetic oligonucleotide
caggccagtcagagtattagtagttacttatcc

SEQ ID NO: 29 - DNA, 21 bp, Artificial Sequence
  Description of Artificial Sequence Synthetic oligonucleotide
agggcatccactctggcatct

SEQ ID NO: 30 - DNA, 42 bp, Artificial Sequence
  Description of Artificial Sequence Synthetic oligonucleotide
caaagctatgatgatagtagtgataataattttttttatggt

SEQ ID NO: 31 - protein, 11 aa, Artificial Sequence
  Description of Artificial Sequence Synthetic peptide
QASQSISSYLS

SEQ ID NO: 32 - protein, 7 aa, Artificial Sequence
  Description of Artificial Sequence Synthetic peptide
RASTLAS

SEQ ID NO: 33 - protein, 14 aa, Artificial Sequence
  Description of Artificial Sequence Synthetic peptide
QSYDDSSDNNFFYG

SEQ ID NO: 34 - DNA, 33 bp, Artificial Sequence
  Description of Artificial Sequence Synthetic oligonucleotide
caggccagtcagaacattgatagttacttagcc

SEQ ID NO: 35 - DNA, 21 bp, Artificial Sequence
  Description of Artificial Sequence Synthetic oligonucleotide
agggcatccactctggcatct

SEQ ID NO: 36 - DNA, 42 bp, Artificial Sequence
  Description of Artificial Sequence Synthetic oligonucleotide
caaagctatgatgatagtaggagtagtagttttttttatggt

SEQ ID NO: 37 - protein, 11 aa, Artificial Sequence
  Description of Artificial Sequence Synthetic peptide
QASQNIDSYLA

SEQ ID NO: 38 - protein, 7 aa, Artificial Sequence
  Description of Artificial Sequence Synthetic peptide
RASTLAS

SEQ ID NO: 39 - protein, 14 aa, Artificial Sequence
  Description of Artificial Sequence Synthetic peptide
QSYDDSRSSSFFYG

SEQ ID NO: 40 - DNA, 33 bp, Artificial Sequence
  Description of Artificial Sequence Synthetic oligonucleotide
caggccagtcagaacattaatagttacttagcc

SEQ ID NO: 41 - DNA, 21 bp, Artificial Sequence
  Description of Artificial Sequence Synthetic oligonucleotide
agggcatccactctggcatct

SEQ ID NO: 42 - DNA, 42 bp, Artificial Sequence
  Description of Artificial Sequence Synthetic oligonucleotide
caaagctatgatgatagtaggagtattagttttttttatgct

SEQ ID NO: 43 - protein, 11 aa, Artificial Sequence
  Description of Artificial Sequence Synthetic peptide
QASQNINSYLA

SEQ ID NO: 44 - protein, 7 aa, Artificial Sequence
  Description of Artificial Sequence Synthetic peptide
RASTLAS

SEQ ID NO: 45 - protein, 14 aa, Artificial Sequence
  Description of Artificial Sequence Synthetic peptide
QSYDDSRSISFFYA

SEQ ID NO: 46 - DNA, 33 bp, Artificial Sequence
  Description of Artificial Sequence Synthetic oligonucleotide
caggccagtcagagcattagtagctacttagcc

SEQ ID NO: 47 - DNA, 21 bp, Artificial Sequence
  Description of Artificial Sequence Synthetic oligonucleotide
gctgcatccactctggcatct

SEQ ID NO: 48 - DNA, 42 bp, Artificial Sequence
  Description of Artificial Sequence Synthetic oligonucleotide
caaagctatgatgatagtaggagtagtagttttttttatgct

SEQ ID NO: 49 - protein, 11 aa, Artificial Sequence
  Description of Artificial Sequence Synthetic peptide
QASQSISSYLA

SEQ ID NO: 50 - protein, 7 aa, Artificial Sequence
  Description of Artificial Sequence Synthetic peptide
AASTLAS

SEQ ID NO: 51 - protein, 14 aa, Artificial Sequence
  Description of Artificial Sequence Synthetic peptide
QSYDDSRSSSFFYA

SEQ ID NO: 52 - DNA, 33 bp, Artificial Sequence
  Description of Artificial Sequence Synthetic oligonucleotide
caggccagtcagagcattagcagttggttatcc

SEQ ID NO: 53 - DNA, 21 bp, Artificial Sequence
  Description of Artificial Sequence Synthetic oligonucleotide
agggcaaccactctggcatct

SEQ ID NO: 54 - DNA, 42 bp, Artificial Sequence
  Description of Artificial Sequence Synthetic oligonucleotide
caaagttatgatgatagtagtagtagtaattttttttatgct

SEQ ID NO: 55 - protein, 11 aa, Artificial Sequence
  Description of Artificial Sequence Synthetic peptide
QASQSISSWLS

SEQ ID NO: 56 - protein, 7 aa, Artificial Sequence
  Description of Artificial Sequence Synthetic peptide
RATTLAS

SEQ ID NO: 57 - protein, 14 aa, Artificial Sequence
  Description of Artificial Sequence Synthetic peptide
QSYDDSSSSNFFYA

SEQ ID NO: 58 - DNA, 39 bp, Artificial Sequence
  Description of Artificial Sequence Synthetic oligonucleotide
cagtccagtcagagtgtttatagtaacaacctcttatct

SEQ ID NO: 59 - DNA, 21 bp, Artificial Sequence
  Description of Artificial Sequence Synthetic oligonucleotide
aaggcatccactctggcatct

SEQ ID NO: 60 - DNA, 30 bp, Artificial Sequence
  Description of Artificial Sequence Synthetic oligonucleotide
caaggctattatagtggtgttatttatatg

SEQ ID NO: 61 - protein, 13 aa, Artificial Sequence
  Description of Artificial Sequence Synthetic peptide
QSSQSVYSNNLLS

SEQ ID NO: 62 - protein, 7 aa, Artificial Sequence
  Description of Artificial Sequence Synthetic peptide
KASTLAS

SEQ ID NO: 63 - protein, 10 aa, Artificial Sequence
  Description of Artificial Sequence Synthetic peptide
QGYYSGVIYM

SEQ ID NO: 64 - DNA, 33 bp, Artificial Sequence
  Description of Artificial Sequence Synthetic oligonucleotide
caggccagtcagagcattagtagttacttatcc

SEQ ID NO: 65 - DNA, 21 bp, Artificial Sequence
  Description of Artificial Sequence Synthetic oligonucleotide
agggcatccactctggcatct

SEQ ID NO: 66 - DNA, 42 bp, Artificial Sequence
  Description of Artificial Sequence Synthetic oligonucleotide
caaagctatgatgatagtagtagtaataattttttttatggt

SEQ ID NO: 67 - protein, 11 aa, Artificial Sequence
  Description of Artificial Sequence Synthetic peptide
QASQSISSYLS

SEQ ID NO: 68 - protein, 7 aa, Artificial Sequence
  Description of Artificial Sequence Synthetic peptide
RASTLAS

SEQ ID NO: 69 - protein, 14 aa, Artificial Sequence
  Description of Artificial Sequence Synthetic peptide
QSYDDSSSNNFFYG

SEQ ID NO: 70 - DNA, 33 bp, Artificial Sequence
  Description of Artificial Sequence Synthetic oligonucleotide
caggccagtcagaacattagtagctacttagcc

SEQ ID NO: 71 - DNA, 21 bp, Artificial Sequence
  Description of Artificial Sequence Synthetic oligonucleotide
agggcatccactctggcatct

SEQ ID NO: 72 - DNA, 42 bp, Artificial Sequence
  Description of Artificial Sequence Synthetic oligonucleotide
caaagctatgatgatagtaggagtagtaattttttttatgct

SEQ ID NO: 73 - protein, 11 aa, Artificial Sequence
  Description of Artificial Sequence Synthetic peptide
QASQNISSYLA

SEQ ID NO: 74 - protein, 7 aa, Artificial Sequence
  Description of Artificial Sequence Synthetic peptide
RASTLAS

SEQ ID NO: 75 - protein, 14 aa, Artificial Sequence
  Description of Artificial Sequence Synthetic peptide
QSYDDSRSSNFFYA

SEQ ID NO: 76 - DNA, 39 bp, Artificial Sequence
  Description of Artificial Sequence Synthetic oligonucleotide
cagtccagtgagagcgtttataatcacaactggttaggc

SEQ ID NO: 77 - DNA, 21 bp, Artificial Sequence
  Description of Artificial Sequence Synthetic oligonucleotide
gatgcatccactctggcatct

SEQ ID NO: 78 - DNA, 30 bp, Artificial Sequence
  Description of Artificial Sequence Synthetic oligonucleotide
caaggctattatcaaactagtgtttgggct

SEQ ID NO: 79 - protein, 13 aa, Artificial Sequence
  Description of Artificial Sequence Synthetic peptide
QSSESVYNHNWLG

SEQ ID NO: 80 - protein, 7 aa, Artificial Sequence
  Description of Artificial Sequence Synthetic peptide
DASTLAS

SEQ ID NO: 81 - protein, 10 aa, Artificial Sequence
  Description of Artificial Sequence Synthetic peptide
QGYYQTSVWA

SEQ ID NO: 82 - DNA, 39 bp, Artificial Sequence
  Description of Artificial Sequence Synthetic oligonucleotide
cagtccagtcagagtgtttatgataacaatgctttagcc

SEQ ID NO: 83 - DNA, 21 bp, Artificial Sequence
  Description of Artificial Sequence Synthetic oligonucleotide
ggtgcatccactctggcatct

SEQ ID NO: 84 - DNA, 36 bp, Artificial Sequence
  Description of Artificial Sequence Synthetic oligonucleotide
caatgtacttattatgttagtagttatcaaaatgat

SEQ ID NO: 85 - protein, 13 aa, Artificial Sequence
  Description of Artificial Sequence Synthetic peptide
QSSQSVYDNNALA

SEQ ID NO: 86 - protein, 7 aa, Artificial Sequence
  Description of Artificial Sequence Synthetic peptide
GASTLAS

SEQ ID NO: 87 - protein, 12 aa, Artificial Sequence
  Description of Artificial Sequence Synthetic peptide
QCTYYVSSYQND

SEQ ID NO: 88 - DNA, 39 bp, Artificial Sequence
  Description of Artificial Sequence Synthetic oligonucleotide
cagtccagtaagagtgtttataataagaactggttatcc

SEQ ID NO: 89 - DNA, 21 bp, Artificial Sequence
  Description of Artificial Sequence Synthetic oligonucleotide
ggtgcatccactctggcatct

SEQ ID NO: 90 - DNA, 36 bp, Artificial Sequence
  Description of Artificial Sequence Synthetic oligonucleotide
gcaggcggttatagtagtagtagtgatacatttgct

SEQ ID NO: 91 - protein, 13 aa, Artificial Sequence
  Description of Artificial Sequence Synthetic peptide
QSSKSVYNKNWLS

SEQ ID NO: 92 - protein, 7 aa, Artificial Sequence
  Description of Artificial Sequence Synthetic peptide
GASTLAS

SEQ ID NO: 93 - protein, 12 aa, Artificial Sequence
  Description of Artificial Sequence Synthetic peptide
AGGYSSSSDTFA

SEQ ID NO: 94 - DNA, 39 bp, Artificial Sequence
  Description of Artificial Sequence Synthetic oligonucleotide
cagtccagtcagagcgtttatagtagcgacctcttatcc

SEQ ID NO: 95 - DNA, 21 bp, Artificial Sequence
  Description of Artificial Sequence Synthetic oligonucleotide
aaggcatccactctggcatct

SEQ ID NO: 96 - DNA, 30 bp, Artificial Sequence
  Description of Artificial Sequence Synthetic oligonucleotide
caaggctactatagtggtgtggtttatatt

SEQ ID NO: 97 - protein, 13 aa, Artificial Sequence
  Description of Artificial Sequence Synthetic peptide
QSSQSVYSSDLLS

SEQ ID NO: 98 - protein, 7 aa, Artificial Sequence
  Description of Artificial Sequence Synthetic peptide
KASTLAS

SEQ ID NO: 99 - protein, 10 aa, Artificial Sequence
  Description of Artificial Sequence Synthetic peptide
QGYYSGVVYI

SEQ ID NO: 100 - DNA, 39 bp, Artificial Sequence; identical to SEQ ID NO: 94
  Description of Artificial Sequence Synthetic oligonucleotide

SEQ ID NO: 101 - DNA, 21 bp, Artificial Sequence
  Description of Artificial Sequence Synthetic oligonucleotide
aaggcatccactctggcatct

SEQ ID NO: 102 - DNA, 30 bp, Artificial Sequence; identical to SEQ ID NO: 96
  Description of Artificial Sequence Synthetic oligonucleotide

SEQ ID NO: 103 - protein, 13 aa, Artificial Sequence
  Description of Artificial Sequence Synthetic peptide
QSSQSVYSSDLLS

SEQ ID NO: 104 - protein, 7 aa, Artificial Sequence
  Description of Artificial Sequence Synthetic peptide
KASTLAS

SEQ ID NO: 105 - protein, 10 aa, Artificial Sequence
  Description of Artificial Sequence Synthetic peptide
QGYYSGVVYI

SEQ ID NO: 106 - DNA, 33 bp, Artificial Sequence
  Description of Artificial Sequence Synthetic oligonucleotide
caggccagtcagagcattagtagttacttatct

SEQ ID NO: 107 - DNA, 21 bp, Artificial Sequence
  Description of Artificial Sequence Synthetic oligonucleotide
agggcatcccctctggcatct

SEQ ID NO: 108 - DNA, 42 bp, Artificial Sequence
  Description of Artificial Sequence Synthetic oligonucleotide
caaagctacgatgatagtagtagtaataattttttttatggt

SEQ ID NO: 109 - protein, 11 aa, Artificial Sequence
  Description of Artificial Sequence Synthetic peptide
QASQSISSYLS

SEQ ID NO: 110 - protein, 7 aa, Artificial Sequence
  Description of Artificial Sequence Synthetic peptide
RASPLAS

SEQ ID NO: 111 - protein, 14 aa, Artificial Sequence
  Description of Artificial Sequence Synthetic peptide
QSYDDSSSNNFFYG

SEQ ID NO: 112 - DNA, 39 bp, Artificial Sequence; identical to SEQ ID NO: 88
  Description of Artificial Sequence Synthetic oligonucleotide

SEQ ID NO: 113 - DNA, 21 bp, Artificial Sequence
  Description of Artificial Sequence Synthetic oligonucleotide
ggtgcatccactctggcatct

SEQ ID NO: 114 - DNA, 36 bp, Artificial Sequence
  Description of Artificial Sequence Synthetic oligonucleotide
gcaggcggttatagtagtagtagtgatacgtttgct

SEQ ID NO: 115 - protein, 13 aa, Artificial Sequence
  Description of Artificial Sequence Synthetic peptide
QSSKSVYNKNWLS

SEQ ID NO: 116 - protein, 7 aa, Artificial Sequence
  Description of Artificial Sequence Synthetic peptide
GASTLAS

SEQ ID NO: 117 - protein, 12 aa, Artificial Sequence
  Description of Artificial Sequence Synthetic peptide
AGGYSSSSDTFA

SEQ ID NO: 118 - DNA, 39 bp, Artificial Sequence
  Description of Artificial Sequence Synthetic oligonucleotide
cagtccagtcagagcctttataatagcgacctcttatcc

SEQ ID NO: 119 - DNA, 21 bp, Artificial Sequence
  Description of Artificial Sequence Synthetic oligonucleotide
aaggcatccactctggcatct

SEQ ID NO: 120 - DNA, 30 bp, Artificial Sequence; identical to SEQ ID NO: 96
  Description of Artificial Sequence Synthetic oligonucleotide

SEQ ID NO: 121 - protein, 13 aa, Artificial Sequence
  Description of Artificial Sequence Synthetic peptide
QSSQSLYNSDLLS

SEQ ID NO: 122 - protein, 7 aa, Artificial Sequence
  Description of Artificial Sequence Synthetic peptide
KASTLAS

SEQ ID NO: 123 - protein, 10 aa, Artificial Sequence
  Description of Artificial Sequence Synthetic peptide
QGYYSGVVYI

SEQ ID NO: 124 - DNA, 39 bp, Artificial Sequence; identical to SEQ ID NO: 88
  Description of Artificial Sequence Synthetic oligonucleotide

SEQ ID NO: 125 - DNA, 21 bp, Artificial Sequence
  Description of Artificial Sequence Synthetic oligonucleotide
ggtgcatccactctggcatct

SEQ ID NO: 126 - DNA, 36 bp, Artificial Sequence; identical to SEQ ID NO: 90
  Description of Artificial Sequence Synthetic oligonucleotide

SEQ ID NO: 127 - protein, 13 aa, Artificial Sequence
  Description of Artificial Sequence Synthetic peptide
QSSKSVYNKNWLS

SEQ ID NO: 128 - protein, 7 aa, Artificial Sequence
  Description of Artificial Sequence Synthetic peptide
GASTLAS

SEQ ID NO: 129 - protein, 12 aa, Artificial Sequence
  Description of Artificial Sequence Synthetic peptide
AGGYSSSSDTFA

SEQ ID NO: 130 - DNA, 39 bp, Artificial Sequence
  Description of Artificial Sequence Synthetic oligonucleotide
caggcccttcagagtgtttatgataacaatgctttatcc

SEQ ID NO: 131 - DNA, 21 bp, Artificial Sequence
  Description of Artificial Sequence Synthetic oligonucleotide
ggtgcatccactctggcatct

SEQ ID NO: 132 - DNA, 36 bp, Artificial Sequence; identical to SEQ ID NO: 84
  Description of Artificial Sequence Synthetic oligonucleotide

SEQ ID NO: 133 - protein, 13 aa, Artificial Sequence
  Description of Artificial Sequence Synthetic peptide
QALQSVYDNNALS

SEQ ID NO: 134 - protein, 7 aa, Artificial Sequence
  Description of Artificial Sequence Synthetic peptide
GASTLAS

SEQ ID NO: 135 - protein, 12 aa, Artificial Sequence
  Description of Artificial Sequence Synthetic peptide
QCTYYVSSYQND

SEQ ID NO: 136 - DNA, 33 bp, Artificial Sequence
  Description of Artificial Sequence Synthetic oligonucleotide
caggccagtgagagcattggcaatgcattagcc

SEQ ID NO: 137 - DNA, 21 bp, Artificial Sequence
  Description of Artificial Sequence Synthetic oligonucleotide
agggcatccactctggcatct

SEQ ID NO: 138 - DNA, 42 bp, Artificial Sequence
  Description of Artificial Sequence Synthetic oligonucleotide
caaagctatgatgatagtagtagtagtagttttttttatgct

SEQ ID NO: 139 - protein, 11 aa, Artificial Sequence
  Description of Artificial Sequence Synthetic peptide
QASESIGNALA

SEQ ID NO: 140 - protein, 7 aa, Artificial Sequence
  Description of Artificial Sequence Synthetic peptide
RASTLAS

SEQ ID NO: 141 - protein, 14 aa, Artificial Sequence
  Description of Artificial Sequence Synthetic peptide
QSYDDSSSSSFFYA

SEQ ID NO: 142 - DNA, 39 bp, Artificial Sequence; identical to SEQ ID NO: 88
  Description of Artificial Sequence Synthetic oligonucleotide

SEQ ID NO: 143 - DNA, 21 bp, Artificial Sequence
  Description of Artificial Sequence Synthetic oligonucleotide
ggtgcatccactctggcatct

SEQ ID NO: 144 - DNA, 36 bp, Artificial Sequence; identical to SEQ ID NO: 114
  Description of Artificial Sequence Synthetic oligonucleotide

SEQ ID NO: 145 - protein, 13 aa, Artificial Sequence
  Description of Artificial Sequence Synthetic peptide
QSSKSVYNKNWLS

SEQ ID NO: 146 - protein, 7 aa, Artificial Sequence
  Description of Artificial Sequence Synthetic peptide
GASTLAS

SEQ ID NO: 147 - protein, 12 aa, Artificial Sequence
  Description of Artificial Sequence Synthetic peptide
AGGYSSSSDTFA

SEQ ID NO: 148 - DNA, 15 bp, Artificial Sequence
  Description of Artificial Sequence Synthetic oligonucleotide
agccatgcaatgatc

SEQ ID NO: 149 - DNA, 48 bp, Artificial Sequence
  Description of Artificial Sequence Synthetic oligonucleotide
accattgggagtcgtgatactatatattatgcgagctgggcgaaaggc

SEQ ID NO: 150 - DNA, 9 bp, Artificial Sequence
  Description of Artificial Sequence Synthetic oligonucleotide
aacgccttg

SEQ ID NO: 151 - protein, 5 aa, Artificial Sequence
  Description of Artificial Sequence Synthetic peptide
SHAMI

SEQ ID NO: 152 - protein, 16 aa, Artificial Sequence
  Description of Artificial Sequence Synthetic peptide
TIGSRDTIYYASWAKG

SEQ ID NO: 153 - protein, 3 aa, Artificial Sequence
  Description of Artificial Sequence Synthetic peptide
NAL

SEQ ID NO: 154 - DNA, 15 bp, Artificial Sequence
  Description of Artificial Sequence Synthetic oligonucleotide
agctacgacatgagc

SEQ ID NO: 155 - DNA, 48 bp, Artificial Sequence
  Description of Artificial Sequence Synthetic oligonucleotide
attatttatgctagtggtagcacatactacgcgagctgggcgaaaggc

SEQ ID NO: 156 - DNA, 33 bp, Artificial Sequence
  Description of Artificial Sequence Synthetic oligonucleotide
gaccctgctggttatagcattagctttggcttg

SEQ ID NO: 157 - protein, 5 aa, Artificial Sequence
  Description of Artificial Sequence Synthetic peptide
SYDMS

SEQ ID NO: 158 - protein, 16 aa, Artificial Sequence
  Description of Artificial Sequence Synthetic peptide
IIYASGSTYYASWAKG

SEQ ID NO: 159 - protein, 11 aa, Artificial Sequence
  Description of Artificial Sequence Synthetic peptide
DPAGYSISFGL

SEQ ID NO: 160 - DNA, 15 bp, Artificial Sequence
  Description of Artificial Sequence Synthetic oligonucleotide
agctacaacatgggc

SEQ ID NO: 161 - DNA, 48 bp, Artificial Sequence
  Description of Artificial Sequence Synthetic oligonucleotide
atcattggtgctagtgatagcgcattgtacgcgagctgggcaaaaggc

SEQ ID NO: 162 - DNA, 33 bp, Artificial Sequence
  Description of Artificial Sequence Synthetic oligonucleotide
ggtggtcttggtttgagtactggttttgcgttg

SEQ ID NO: 163 - protein, 5 aa, Artificial Sequence
  Description of Artificial Sequence Synthetic peptide
SYNMG

SEQ ID NO: 164 - protein, 16 aa, Artificial Sequence
  Description of Artificial Sequence Synthetic peptide
IIGASDSALYASWAKG

SEQ ID NO: 165 - protein, 11 aa, Artificial Sequence
  Description of Artificial Sequence Synthetic peptide
GGLGLSTGFAL

SEQ ID NO: 166 - DNA, 15 bp, Artificial Sequence
  Description of Artificial Sequence Synthetic oligonucleotide
agctacgacatgagc

SEQ ID NO: 167 - DNA, 48 bp, Artificial Sequence
  Description of Artificial Sequence Synthetic oligonucleotide
tatattgctactgatggtaggccatattacgcgagctgggcgaaaggc

SEQ ID NO: 168 - DNA, 21 bp, Artificial Sequence
  Description of Artificial Sequence Synthetic oligonucleotide
ggggggtatgctggtggcttg

SEQ ID NO: 169 - protein, 5 aa, Artificial Sequence
  Description of Artificial Sequence Synthetic peptide
SYDMS

SEQ ID NO: 170 - protein, 16 aa, Artificial Sequence
  Description of Artificial Sequence Synthetic peptide
YIATDGRPYYASWAKG

SEQ ID NO: 171 - protein, 7 aa, Artificial Sequence
  Description of Artificial Sequence Synthetic peptide
GGYAGGL

SEQ ID NO: 172 - DNA, 15 bp, Artificial Sequence
  Description of Artificial Sequence Synthetic oligonucleotide
aattattatatttgc

SEQ ID NO: 173 - DNA, 51 bp, Artificial Sequence
  Description of Artificial Sequence Synthetic oligonucleotide
tgcattgacaatgctaatggtaggacttactacgcgagctgggcgaaaggc

SEQ ID NO: 174 - DNA, 18 bp, Artificial Sequence
  Description of Artificial Sequence Synthetic oligonucleotide
tcattgtctactcccttg

SEQ ID NO: 175 - protein, 5 aa, Artificial Sequence
  Description of Artificial Sequence Synthetic peptide
NYYIC

SEQ ID NO: 176 - protein, 17 aa, Artificial Sequence
  Description of Artificial Sequence Synthetic peptide
CIDNANGRTYYASWAKG

SEQ ID NO: 177 - protein, 6 aa, Artificial Sequence
  Description of Artificial Sequence Synthetic peptide
SLSTPL

SEQ ID NO: 178 - DNA, 15 bp, Artificial Sequence
  Description of Artificial Sequence Synthetic oligonucleotide
aactattatatttgt

SEQ ID NO: 179 - DNA, 51 bp, Artificial Sequence
  Description of Artificial Sequence Synthetic oligonucleotide
tgcattgacaatgttaatggtaggacctactacgcgagctgggcgaaaggc

SEQ ID NO: 180 - DNA, 18 bp, Artificial Sequence
  Description of Artificial Sequence Synthetic oligonucleotide
tccttggctactcccttg

SEQ ID NO: 181 - protein, 5 aa, Artificial Sequence
  Description of Artificial Sequence Synthetic peptide
NYYIC

SEQ ID NO: 182 - protein, 17 aa, Artificial Sequence
  Description of Artificial Sequence Synthetic peptide
CIDNVNGRTYYASWAKG

SEQ ID NO: 183 - protein, 6 aa, Artificial Sequence
  Description of Artificial Sequence Synthetic peptide
SLATPL

SEQ ID NO: 184 - DNA, 15 bp, Artificial Sequence
  Description of Artificial Sequence Synthetic oligonucleotide
aactactacatctgc

SEQ ID NO: 185 - DNA, 51 bp, Artificial Sequence
  Description of Artificial Sequence Synthetic oligonucleotide
tgcattgacaatgttaatggtaggacttactacgcgagctgggcgaaaggc

SEQ ID NO: 186 - DNA, 18 bp, Artificial Sequence
  Description of Artificial Sequence Synthetic oligonucleotide
tccttggctactcccttg

SEQ ID NO: 187 - protein, 5 aa, Artificial Sequence
  Description of Artificial Sequence Synthetic peptide
NYYIC

SEQ ID NO: 188 - protein, 17 aa, Artificial Sequence
  Description of Artificial Sequence Synthetic peptide
CIDNVNGRTYYASWAKG

SEQ ID NO: 189 - protein, 6 aa, Artificial Sequence
  Description of Artificial Sequence Synthetic peptide
SLATPL

SEQ ID NO: 190 - DNA, 15 bp, Artificial Sequence
  Description of Artificial Sequence Synthetic oligonucleotide
aactactacatctgc

SEQ ID NO: 191 - DNA, 51 bp, Artificial Sequence
  Description of Artificial Sequence Synthetic oligonucleotide
tgcattgacaatattaatggtaggacttactacgcgagctgggcgaaaggc

SEQ ID NO: 192 - DNA, 18 bp, Artificial Sequence
  Description of Artificial Sequence Synthetic oligonucleotide
tccttggctactcccttg

SEQ ID NO: 193 - protein, 5 aa, Artificial Sequence
  Description of Artificial Sequence Synthetic peptide
NYYIC

SEQ ID NO: 194 - protein, 17 aa, Artificial Sequence
  Description of Artificial Sequence Synthetic peptide
CIDNINGRTYYASWAKG

SEQ ID NO: 195 - protein, 6 aa, Artificial Sequence
  Description of Artificial Sequence Synthetic peptide
SLATPL

SEQ ID NO: 196 - DNA, 15 bp, Artificial Sequence
  Description of Artificial Sequence Synthetic oligonucleotide
aattattatatatgt

SEQ ID NO: 197 - DNA, 51 bp, Artificial Sequence
  Description of Artificial Sequence Synthetic oligonucleotide
tgtattgataacgctaatggtaggacttactacgcgacctgggcgaaaggc

SEQ ID NO: 198 - DNA, 18 bp, Artificial Sequence
  Description of Artificial Sequence Synthetic oligonucleotide
tcattgtctactaacttg

SEQ ID NO: 199 - protein, 5 aa, Artificial Sequence
  Description of Artificial Sequence Synthetic peptide
NYYIC

SEQ ID NO: 200 - protein, 17 aa, Artificial Sequence
  Description of Artificial Sequence Synthetic peptide
CIDNANGRTYYATWAKG

SEQ ID NO: 201 - protein, 6 aa, Artificial Sequence
  Description of Artificial Sequence Synthetic peptide
SLSTNL

SEQ ID NO: 202 - DNA, 18 bp, Artificial Sequence
  Description of Artificial Sequence Synthetic oligonucleotide
agctactactacatatgc

SEQ ID NO: 203 - DNA, 54 bp, Artificial Sequence
  Description of Artificial Sequence Synthetic oligonucleotide
tgtattggtggtggtaataccgatgccactgcctacgcgaggtgggcgaaaggc

SEQ ID NO: 204 - DNA, 33 bp, Artificial Sequence
  Description of Artificial Sequence Synthetic oligonucleotide
ggcggtcctgataataatgtccaatttaacttg

SEQ ID NO: 205 - protein, 6 aa, Artificial Sequence
  Description of Artificial Sequence Synthetic peptide
SYYYIC

SEQ ID NO: 206 - protein, 18 aa, Artificial Sequence
  Description of Artificial Sequence Synthetic peptide
CIGGGNTDATAYARWAKG

SEQ ID NO: 207 - protein, 11 aa, Artificial Sequence
  Description of Artificial Sequence Synthetic peptide
GGPDNNVQFNL

SEQ ID NO: 208 - DNA, 15 bp, Artificial Sequence
  Description of Artificial Sequence Synthetic oligonucleotide
aattattatatttgc

SEQ ID NO: 209 - DNA, 51 bp, Artificial Sequence
  Description of Artificial Sequence Synthetic oligonucleotide
tgcattgacaatagtaatggtaggacttactacgcgagctgggcgaaaggc

SEQ ID NO: 210 - DNA, 18 bp, Artificial Sequence
  Description of Artificial Sequence Synthetic oligonucleotide
tcattgtctactcccttg

SEQ ID NO: 211 - protein, 5 aa, Artificial Sequence
  Description of Artificial Sequence Synthetic peptide
NYYIC

SEQ ID NO: 212 - protein, 17 aa, Artificial Sequence
  Description of Artificial Sequence Synthetic peptide
CIDNSNGRTYYASWAKG

SEQ ID NO: 213 - protein, 6 aa, Artificial Sequence
  Description of Artificial Sequence Synthetic peptide
SLSTPL

SEQ ID NO: 214 - DNA, 15 bp, Artificial Sequence
  Description of Artificial Sequence Synthetic oligonucleotide
aactactacatctgc

SEQ ID NO: 215 - DNA, 51 bp, Artificial Sequence
  Description of Artificial Sequence Synthetic oligonucleotide
tgtattgacaatgctaatggtaggacttactacgcgagctgggcgaaaggc

SEQ ID NO: 216 - DNA, 18 bp, Artificial Sequence
  Description of Artificial Sequence Synthetic oligonucleotide
tccttggctactcccttg

SEQ ID NO: 217 - protein, 5 aa, Artificial Sequence
  Description of Artificial Sequence Synthetic peptide
NYYIC

SEQ ID NO: 218 - protein, 17 aa, Artificial Sequence
  Description of Artificial Sequence Synthetic peptide
CIDNANGRTYYASWAKG

SEQ ID NO: 219 - protein, 6 aa, Artificial Sequence
  Description of Artificial Sequence Synthetic peptide
SLATPL

SEQ ID NO: 220 - DNA, 15 bp, Artificial Sequence
  Description of Artificial Sequence Synthetic oligonucleotide
agcagtgcagtgacc

SEQ ID NO: 221 - DNA, 51 bp, Artificial Sequence
  Description of Artificial Sequence Synthetic oligonucleotide
ttcctccaagctggggatggtagcgcatactacgcgagctgggcgaaaggc

SEQ ID NO: 222 - DNA, 30 bp, Artificial Sequence
  Description of Artificial Sequence Synthetic oligonucleotide
cataagggtaatagttacgtgcctaacttg

SEQ ID NO: 223 - protein, 5 aa, Artificial Sequence
  Description of Artificial Sequence Synthetic peptide
SSAVT

SEQ ID NO: 224 - protein, 17 aa, Artificial Sequence
  Description of Artificial Sequence Synthetic peptide
FLQAGDGSAYYASWAKG

SEQ ID NO: 225 - protein, 10 aa, Artificial Sequence
  Description of Artificial Sequence Synthetic peptide
HKGNSYVPNL

SEQ ID NO: 226 - DNA, 15 bp, Artificial Sequence
  Description of Artificial Sequence Synthetic oligonucleotide
agctatgcaatgagc

SEQ ID NO: 227 - DNA, 48 bp, Artificial Sequence
  Description of Artificial Sequence Synthetic oligonucleotide
agcattggtggtggtggtagcgcagtctacgcgagctgggcgaaaggc

SEQ ID NO: 228 - DNA, 21 bp, Artificial Sequence
  Description of Artificial Sequence Synthetic oligonucleotide
ggattttatagtatagacttg

SEQ ID NO: 229 - protein, 5 aa, Artificial Sequence
  Description of Artificial Sequence Synthetic peptide
SYAMS

SEQ ID NO: 230 - protein, 16 aa, Artificial Sequence
  Description of Artificial Sequence Synthetic peptide
SIGGGGSAVYASWAKG

SEQ ID NO: 231 - protein, 7 aa, Artificial Sequence
  Description of Artificial Sequence Synthetic peptide
GFYSIDL

SEQ ID NO: 232 - DNA, 18 bp, Artificial Sequence
  Description of Artificial Sequence Synthetic oligonucleotide
agcggccaactcatgtgc

SEQ ID NO: 233 - DNA, 57 bp, Artificial Sequence
  Description of Artificial Sequence Synthetic oligonucleotide
tgcattggttctggtagtaatgctattagcactttctacgcgagctgggcgcaaggc

SEQ ID NO: 234 - DNA, 42 bp, Artificial Sequence
  Description of Artificial Sequence Synthetic oligonucleotide
gtgggctccgatgactatggtgactctgatgtttttgatccc

SEQ ID NO: 235 - protein, 6 aa, Artificial Sequence
  Description of Artificial Sequence Synthetic peptide
SGQLMC

SEQ ID NO: 236 - protein, 19 aa, Artificial Sequence
  Description of Artificial Sequence Synthetic peptide
CIGSGSNAISTFYASWAQG

SEQ ID NO: 237 - protein, 14 aa, Artificial Sequence
  Description of Artificial Sequence Synthetic peptide
VGSDDYGDSDVFDP

SEQ ID NO: 238 - DNA, 18 bp, Artificial Sequence
  Description of Artificial Sequence Synthetic oligonucleotide
agcgcctactacatatgc

SEQ ID NO: 239 - DNA, 54 bp, Artificial Sequence
  Description of Artificial Sequence Synthetic oligonucleotide
tgtattggtggtgttaatcgcgttgccactgcctacgcgacctgggcgaaaggc

SEQ ID NO: 240 - DNA, 33 bp, Artificial Sequence; identical to SEQ ID NO: 204
  Description of Artificial Sequence Synthetic oligonucleotide

SEQ ID NO: 241 - protein, 6 aa, Artificial Sequence
  Description of Artificial Sequence Synthetic peptide
SAYYIC

SEQ ID NO: 242 - protein, 18 aa, Artificial Sequence
  Description of Artificial Sequence Synthetic peptide
CIGGVNRVATAYATWAKG

SEQ ID NO: 243 - protein, 11 aa, Artificial Sequence
  Description of Artificial Sequence Synthetic peptide
GGPDNNVQFNL

SEQ ID NO: 244 - DNA, 18 bp, Artificial Sequence
  Description of Artificial Sequence Synthetic oligonucleotide
aggtactactacagttgc

SEQ ID NO: 245 - DNA, 54 bp, Artificial Sequence
  Description of Artificial Sequence Synthetic oligonucleotide
tgtgttggtggtgttaatcgcgatgccactgcctacgcgacctgggcgaaaggc

SEQ ID NO: 246 - DNA, 33 bp, Artificial Sequence; identical to SEQ ID NO: 204
  Description of Artificial Sequence Synthetic oligonucleotide

SEQ ID NO: 247 - protein, 6 aa, Artificial Sequence
  Description of Artificial Sequence Synthetic peptide
RYYYSC

SEQ ID NO: 248 - protein, 18 aa, Artificial Sequence
  Description of Artificial Sequence Synthetic peptide
CVGGVNRDATAYATWAKG

SEQ ID NO: 249 - protein, 11 aa, Artificial Sequence
  Description of Artificial Sequence Synthetic peptide
GGPDNNVQFNL

SEQ ID NO: 250 - DNA, 15 bp, Artificial Sequence
  Description of Artificial Sequence Synthetic oligonucleotide
aattattatatttgt

SEQ ID NO: 251 - DNA, 51 bp, Artificial Sequence
  Description of Artificial Sequence Synthetic oligonucleotide
tgtattgacaatgttaatggtaggacttactacgcgagctgggcgaaaggc

SEQ ID NO: 252 - DNA, 18 bp, Artificial Sequence
  Description of Artificial Sequence Synthetic oligonucleotide
tcattgtctactcccttg

SEQ ID NO: 253 - protein, 5 aa, Artificial Sequence
  Description of Artificial Sequence Synthetic peptide
NYYIC

SEQ ID NO: 254 - protein, 17 aa, Artificial Sequence
  Description of Artificial Sequence Synthetic peptide
CIDNVNGRTYYASWAKG

SEQ ID NO: 255 - protein, 6 aa, Artificial Sequence
  Description of Artificial Sequence Synthetic peptide
SLSTPL

SEQ ID NO: 256 - DNA, 18 bp, Artificial Sequence
  Description of Artificial Sequence Synthetic oligonucleotide
agcagctacttcatgtgc

SEQ ID NO: 257 - DNA, 54 bp, Artificial Sequence
  Description of Artificial Sequence Synthetic oligonucleotide
tgcattgctgttggtagtagtggtagcacttactacgcgagctgggcgaaaggc

SEQ ID NO: 258 - DNA, 42 bp, Artificial Sequence
  Description of Artificial Sequence Synthetic oligonucleotide
gtgggctacgatgactatggtgactctgatgcttttgatccc

SEQ ID NO: 259 - protein, 6 aa, Artificial Sequence
  Description of Artificial Sequence Synthetic peptide
SSYFMC

SEQ ID NO: 260 - protein, 18 aa, Artificial Sequence
  Description of Artificial Sequence Synthetic peptide
CIAVGSSGSTYYASWAKG

SEQ ID NO: 261 - protein, 14 aa, Artificial Sequence
  Description of Artificial Sequence Synthetic peptide
VGYDDYGDSDAFDP

SEQ ID NO: 262 - DNA, 18 bp, Artificial Sequence
  Description of Artificial Sequence Synthetic oligonucleotide
aggtactactacagttgc

SEQ ID NO: 263 - DNA, 54 bp, Artificial Sequence; identical to SEQ ID NO: 245
  Description of Artificial Sequence Synthetic oligonucleotide

SEQ ID NO: 264 - DNA, 33 bp, Artificial Sequence; identical to SEQ ID NO: 204
  Description of Artificial Sequence Synthetic oligonucleotide

SEQ ID NO: 265 - protein, 6 aa, Artificial Sequence
  Description of Artificial Sequence Synthetic peptide
RYYYSC

SEQ ID NO: 266 - protein, 18 aa, Artificial Sequence
  Description of Artificial Sequence Synthetic peptide
CVGGVNRDATAYATWAKG

SEQ ID NO: 267 - protein, 11 aa, Artificial Sequence
  Description of Artificial Sequence Synthetic peptide
GGPDNNVQFNL

SEQ ID NO: 268 - DNA, 18 bp, Artificial Sequence
  Description of Artificial Sequence Synthetic oligonucleotide
agcagctacttcatgtgc

SEQ ID NO: 269 - DNA, 57 bp, Artificial Sequence
  Description of Artificial Sequence Synthetic oligonucleotide
tgcattggttctggtagtagtgctattagcactttctacgcgagctgggcgcaaggc

SEQ ID NO: 270 - DNA, 42 bp, Artificial Sequence; identical to SEQ ID NO: 258
  Description of Artificial Sequence Synthetic oligonucleotide

SEQ ID NO: 271 - protein, 6 aa, Artificial Sequence
  Description of Artificial Sequence Synthetic peptide
SSYFMC

SEQ ID NO: 272 - protein, 19 aa, Artificial Sequence
  Description of Artificial Sequence Synthetic peptide
CIGSGSSAISTFYASWAQG

SEQ ID NO: 273 - protein, 14 aa, Artificial Sequence
  Description of Artificial Sequence Synthetic peptide
VGYDDYGDSDAFDP

SEQ ID NO: 274 - DNA, 15 bp, Artificial Sequence
  Description of Artificial Sequence Synthetic oligonucleotide
agctatggaatgact

SEQ ID NO: 275 - DNA, 48 bp, Artificial Sequence
  Description of Artificial Sequence Synthetic oligonucleotide
tacatttggactgatgggaggacatactacgcaaactgggcgaaaggc

SEQ ID NO: 276 - DNA, 27 bp, Artificial Sequence
  Description of Artificial Sequence Synthetic oligonucleotide
ccctttgatggtaattatagggacatc

SEQ ID NO: 277 - protein, 5 aa, Artificial Sequence
  Description of Artificial Sequence Synthetic peptide
SYGMT

SEQ ID NO: 278 - protein, 16 aa, Artificial Sequence
  Description of Artificial Sequence Synthetic peptide
YIWTDGRTYYANWAKG

SEQ ID NO: 279 - protein, 9 aa, Artificial Sequence
  Description of Artificial Sequence Synthetic peptide
PFDGNYRDI

SEQ ID NO: 280 - DNA, 15 bp, Artificial Sequence
  Description of Artificial Sequence Synthetic oligonucleotide
aactactacatctgc

SEQ ID NO: 281 - DNA, 51 bp, Artificial Sequence
  Description of Artificial Sequence Synthetic oligonucleotide
tgtattgataatgccaatggtcggacttactacgcgaactgggcgaaaggc

SEQ ID NO: 282 - DNA, 18 bp, Artificial Sequence
  Description of Artificial Sequence Synthetic oligonucleotide
tccttggctactcccttg

SEQ ID NO: 283 - protein, 5 aa, Artificial Sequence
  Description of Artificial Sequence Synthetic peptide
NYYIC

SEQ ID NO: 284 - protein, 17 aa, Artificial Sequence
  Description of Artificial Sequence Synthetic peptide
CIDNANGRTYYANWAKG

SEQ ID NO: 285 - protein, 6 aa, Artificial Sequence
  Description of Artificial Sequence Synthetic peptide
SLATPL

SEQ ID NO: 286 - DNA, 18 bp, Artificial Sequence
  Description of Artificial Sequence Synthetic oligonucleotide
agcagctacttcatgtgc

SEQ ID NO: 287 - DNA, 54 bp, Artificial Sequence
  Description of Artificial Sequence Synthetic oligonucleotide
tgcattggtgttggtggtagtggtagcacttactacgcgaactgggcgaaaggc

SEQ ID NO: 288 - DNA, 42 bp, Artificial Sequence
  Description of Artificial Sequence Synthetic oligonucleotide
gtggcctacgatgacgatggtgactctgatgcttttgatccc

SEQ ID NO: 289 - protein, 6 aa, Artificial Sequence
  Description of Artificial Sequence Synthetic peptide
SSYFMC

SEQ ID NO: 290 - protein, 18 aa, Artificial Sequence
  Description of Artificial Sequence Synthetic peptide
CIGVGGSGSTYYANWAKG

SEQ ID NO: 291 - protein, 14 aa, Artificial Sequence
  Description of Artificial Sequence Synthetic peptide
VAYDDDGDSDAFDP

SEQ ID NO: 292 - DNA, 336 bp, Artificial Sequence
  Description of Artificial Sequence Synthetic polynucleotide
gatgttgtgatgacccagactccagcctccgtgtctgaacctgtgggaggcacagtcaccatcaagtgccaggccagtcagagcattaggaatgaattattttggtggcagcagaaaccagggcagcctcccaagctcctgatctatgctgcatccaaactggcctctggggtcccatcgcggttcagcggcagtggatctgggacagagttcactctcaccatcagcgacctggagtgtgccgatgctgccacttactactgtcaatgcagttatgttagtagtagtggtacttatggaaatgttttcggcggagggaccgaggtggtggtcaaa

SEQ ID NO: 293 - DNA, 327 bp, Artificial Sequence
  Description of Artificial Sequence Synthetic polynucleotide
cagtcgctggaggagtccgggggtcgcctggtaacgcctggaggatccctgacactcacctgcacagtctctggaatcgacctcagtagccatgcaatgatctgggtccgccaggctccaggggaggggctggaatggatcggaaccattgggagtcgtgatactatatattatgcgagctgggcgaaaggccgattcaccatctccaaaacctcgtcgaccacaatggatctgaaaatgaccagtctgacaatcgaggacacggccacctatttctgtgtcagaaacgccttgtggggcccaggcaccctggtcaccgtctcctca

SEQ ID NO: 294 - protein, 112 aa, Artificial Sequence
  Description of Artificial Sequence Synthetic polypeptide
DVVMTQTPASVSEPVGGTVTIKCQASQSIRNELFWWQQKPGQPPKLLIYAASKLASGVPSRFSGSGSGTEFTLTISDLECADAATYYCQCSYVSSSGTYGNVFGGGTEVVVK

SEQ ID NO: 295 - protein, 109 aa, Artificial Sequence
  Description of Artificial Sequence Synthetic polypeptide
QSLEESGGRLVTPGGSLTLTCTVSGIDLSSHAMIWVRQAPGEGLEWIGTIGSRDTIYYASWAKGRFTISKTSSTTMDLKMTSLTIEDTATYFCVRNALWGPGTLVTVSS

SEQ ID NO: 296 - DNA, 330 bp, Artificial Sequence
  Description of Artificial Sequence Synthetic polynucleotide
gcctatgatatgacccagactccagcctccgtggaggcagctgtgggaggcacagtcaccatcaattgccaggccagtgaaagcattagcaactacttatcctggtatcagcagaaaccagggcagcctcccaagctcctgatctactgggcatccactctggcatctggggtctcatcgcggttcaaaggcagtggatctgggacacagttcactctcaccatcagcggcgtggagtgtgccgatgctgccacttactactgtcaacagggttatagtagtagtaatgttgataatcttttcggcggagggaccgaggtggtggtcaaa

SEQ ID NO: 297 - DNA, 348 bp, Artificial Sequence
  Description of Artificial Sequence Synthetic polynucleotide
cagtcgctggaggagtccgggggtcgcctggtcacgcctgggacacccctgacactcacctgcaaagtctctggattctccctcagcagctacgacatgagctgggtccgccaggctccagggaaggggctggaatggatcggaattatttatgctagtggtagcacatactacgcgagctgggcgaaaggccgattcaccatctccaaaacctcgaccacggtggatctgaaaatcgccagtccgacaaccgaggacacggccacctatttctgtgccagagaccctgctggttatagcattagctttggcttgtggggcccaggcaccctggtcaccgtctcctca

SEQ ID NO: 298 - protein, 110 aa, Artificial Sequence
  Description of Artificial Sequence Synthetic polypeptide
AYDMTQTPASVEAAVGGTVTINCQASESISNYLSWYQQKPGQPPKLLIYWASTLASGVSSRFKGSGSGTQFTLTISGVECADAATYYCQQGYSSSNVDNLFGGGTEVVVK

SEQ ID NO: 299 - protein, 116 aa, Artificial Sequence
  Description of Artificial Sequence Synthetic polypeptide
QSLEESGGRLVTPGTPLTLTCKVSGFSLSSYDMSWVRQAPGKGLEWIGIIYASGSTYYASWAKGRFTISKTSTTVDLKIASPTTEDTATYFCARDPAGYSISFGLWGPGTLVTVSS

SEQ ID NO: 300 - DNA, 333 bp, Artificial Sequence
  Description of Artificial Sequence Synthetic polynucleotide
gccgccgtgctgacccagactccatctcccgtgtctgcagctgtgggaggcacagtcagcatcagttgccagtccagtcagagtgtttggcataacgactacttatcctggtatcaacagaaaccagggcagcctcccaagctcctgatctatggtgcatccactttggcatctggggtcccatcgcggttcaaaggcagtggatctgggacacagttcagtctcaccatcagcggcgtgcagtgtgacgatgctgccacttactactgtgcaggcggttatggacgtagtagtgaaaatggtttcggcggagggaccgaggtggtggtcaaa

SEQ ID NO: 301 - DNA, 351 bp, Artificial Sequence
  Description of Artificial Sequence Synthetic polynucleotide
caggagcagctgaaggagtccggaggaggcctggttgcgcctggaggaaccctgacactcacctgcgcagtctctggattctccctcagtagctacaacatgggctgggtccgccaggctccaggggaggggctggaatacatcggaatcattggtgctagtgatagcgcattgtacgcgagctgggcaaaaggccgattcaccatctccaaaacctcgaccacggtggatctgaaaatcaccagtccgacaaccgaggacacggccacctatttctgtgccagaggtggtcttggtttgagtactggttttgcgttgtggggcccaggcaccctggtcaccgtctcctca

SEQ ID NO: 302 - protein, 111 aa, Artificial Sequence
  Description of Artificial Sequence Synthetic polypeptide
AAVLTQTPSPVSAAVGGTVSISCQSSQSVWHNDYLSWYQQKPGQPPKLLIYGASTLASGVPSRFKGSGSGTQFSLTISGVQCDDAATYYCAGGYGRSSENGFGGGTEVVVK

SEQ ID NO: 303 - protein, 117 aa, Artificial Sequence
  Description of Artificial Sequence Synthetic polypeptide
QEQLKESGGGLVAPGGTLTLTCAVSGFSLSSYNMGWVRQAPGEGLEYIGIIGASDSALYASWAKGRFTISKTSTTVDLKITSPTTEDTATYFCARGGLGLSTGFALWGPGTLVTVSS

SEQ ID NO: 304 - DNA, 333 bp, Artificial Sequence
  Description of Artificial Sequence Synthetic polynucleotide
gccgccgtgctgacccagactccatctcccgtgtctgcagctgtgggaggcacagtcagcatcagttgccagtccagtaagagtgtttataataacaactggttagcctggtatcagcagaaaccagggcagcgtcccaagctcctgatctacgatgcatcgactctagattctggggtctcatcgcggttcaaaggcagtggatctgggacacagttcactctcaccatcagcgacgtgcagtgtgacgatggtgccacttactactgtgtaggcggttatagtagtcgtagtgataatggtttcggcggagggaccgaggtggtggtcaaa

SEQ ID NO: 305 - DNA, 339 bp, Artificial Sequence
  Description of Artificial Sequence Synthetic polynucleotide
cagtcgctggaggagtccgggggtcgcctggtaacgcctggaggacccctgacactcacctgcacagtctctggattctccctcagcagctacgacatgagctgggtccgccaggctccagggaaggggctggagtggatcggatatattgctactgatggtaggccatattacgcgagctgggcgaaaggccgattcaccatctccaaaccctcgtcgaccacggtggatctgaaaatcaccagtccgacaaccgaggacacggccacctatttctgtgtcagaggggggtatgctggtggcttgtggggcccaggcaccctggtcaccgtttcctca

SEQ ID NO: 306 - protein, 111 aa, Artificial Sequence
  Description of Artificial Sequence Synthetic polypeptide
AAVLTQTPSPVSAAVGGTVSISCQSSKSVYNNNWLAWYQQKPGQRPKLLIYDASTLDSGVSSRFKGSGSGTQFTLTISDVQCDDGATYYCVGGYSSRSDNGFGGGTEVVVK

SEQ ID NO: 307 - protein, 113 aa, Artificial Sequence
  Description of Artificial Sequence Synthetic polypeptide
QSLEESGGRLVTPGGPLTLTCTVSGFSLSSYDMSWVRQAPGKGLEWIGYIATDGRPYYASWAKGRFTISKPSSTTVDLKITSPTTEDTATYFCVRGGYAGGLWGPGTLVTVSS

SEQ ID NO: 308 - DNA, 336 bp, Artificial Sequence
  Description of Artificial Sequence Synthetic polynucleotide
gacattgtgatgacccagactccagcctccgtggaggcagctgtgggaggcacagtcaccatcaagtgccaggccagtcagagtattagtagttacttatcctggtatcagcagaaaccagggcagcctcccaagctcctgatctacagggcatccactctggcatctggggtcccatcgcggttcagaggcagtggatctgggacacagttcactctcaccatcagcgacctggagtgtgccgatgctgccacttattactgtcaaagctatgatgatagtagtgataataattttttttatggtttcggcggagggaccgaggtggtggtcaga

SEQ ID NO: 309 - DNA, 339 bp, Artificial Sequence
  Description of Artificial Sequence Synthetic polynucleotide
cagtcgttggaggagtccgggggaggcctggtccagcctgagggatccctgacactcacctgcacagcttttggattcaccctcaataattattatatttgctgggtccgccaggctccagggaagggactggagtggatcgcatgcattgacaatgctaatggtaggacttactacgcgagctgggcgaaaggccgattcaccatctccaaaacctcgtcgaccacggtgactctacaaatgaccagtctgacagccgcggacacggccacctatttctgtgcgaggtcattgtctactcccttgtggggcccaggcaccctggtcaccgtctcctca

SEQ ID NO: 310 - protein, 112 aa, Artificial Sequence
  Description of Artificial Sequence Synthetic polypeptide
DIVMTQTPASVEAAVGGTVTIKCQASQSISSYLSWYQQKPGQPPKLLIYRASTLASGVPSRFRGSGSGTQFTLTISDLECADAATYYCQSYDDSSDNNFFYGFGGGTEVVVR

SEQ ID NO: 311 - protein, 113 aa, Artificial Sequence
  Description of Artificial Sequence Synthetic polypeptide
QSLEESGGGLVQPEGSLTLTCTAFGFTLNNYYICWVRQAPGKGLEWIACIDNANGRTYYASWAKGRFTISKTSSTTVTLQMTSLTAADTATYFCARSLSTPLWGPGTLVTVSS

SEQ ID NO: 312 - DNA, 336 bp, Artificial Sequence
  Description of Artificial Sequence Synthetic polynucleotide
gacattgtgatgacccagactccagcctccgtggaggcagctgtgggaggcacagtcaccatcaagtgccaggccagtcagaacattgatagttacttagcctggtatcagcagaaaccagggcagcctcccaagctcctgatctacagggcatccactctggcatctggggtcccatcgcggttcaaaggcagtggatctgggacagagttcactctcaccatcagcgacctggagtgtgccgatgctgccacttactactgtcaaagctatgatgatagtaggagtagtagttttttttatggtttcggcggagggaccgaggtggtggtcaaa

SEQ ID NO: 313 - DNA, 339 bp, Artificial Sequence
  Description of Artificial Sequence Synthetic polynucleotide
cagtcgttggaggagtccgggggaggcctggtccagcctgagggatccctgacactcacctgcacagcttctggagtcaccctcagtaactattatatttgttgggtccgccaggctccagggaaggggctggagtggatcgggtgcattgacaatgttaatggtaggacctactacgcgagctgggcgaaaggccgattcaccatctccaagacctcgtcgaccacagggactctacaaatgaccagtctgacagccgcggacacggccacctatttctgtgcgaggtccttggctactcccttgtggggcccaggcaccctagtcaccgtctcctca

SEQ ID NO: 314 - protein, 112 aa, Artificial Sequence
  Description of Artificial Sequence Synthetic polypeptide
DIVMTQTPASVEAAVGGTVTIKCQASQNIDSYLAWYQQKPGQPPKLLIYRASTLASGVPSRFKGSGSGTEFTLTISDLECADAATYYCQSYDDSRSSSFFYGFGGGTEVVVK

SEQ ID NO: 315 - protein, 113 aa, Artificial Sequence
  Description of Artificial Sequence Synthetic polypeptide
QSLEESGGGLVQPEGSLTLTCTASGVTLSNYYICWVRQAPGKGLEWIGCIDNVNGRTYYASWAKGRFTISKTSSTTGTLQMTSLTAADTATYFCARSLATPLWGPGTLVTVSS

SEQ ID NO: 316 - DNA, 336 bp, Artificial Sequence
  Description of Artificial Sequence Synthetic polynucleotide
gacattgtgatgacccagactccagcctccgtggaggcagctgtgggaggcacagtcaccatcaagtgccaggccagtcagaacattaatagttacttagcctggtatcagcagaaaccagggcagcctcccaagctcctgatctacagggcatccactctggcatctggggtcccatcgcggttcaaaggcagtggatctgggacagagttcactctcaccatcagcgacctggagtgtgccgatgctgccacttactactgtcaaagctatgatgatagtaggagtattagttttttttatgctttcggcggagggaccgaggtggtggtcaaa

SEQ ID NO: 317 - DNA, 339 bp, Artificial Sequence
  Description of Artificial Sequence Synthetic polynucleotide
cagtcgttggaggagtccgggggaggcctggtccagcctgagggatccctgacactcacctgcacagcttctggagtcaccctcactaactactacatctgctgggtccgccaggctccagggaaggggctggagtggatcgggtgcattgacaatgttaatggtaggacttactacgcgagctgggcgaaaggccgattcaccatctccaaggcctcgtcgaccacagggactctacaaatgaccagtctgacagccgcggacacggccacctatttctgtgcgaggtccttggctactcccttgtggggcccaggcaccctggtcaccgtctcctca

SEQ ID NO: 318 - protein, 112 aa, Artificial Sequence
  Description of Artificial Sequence Synthetic polypeptide
DIVMTQTPASVEAAVGGTVTIKCQASQNINSYLAWYQQKPGQPPKLLIYRASTLASGVPSRFKGSGSGTEFTLTISDLECADAATYYCQSYDDSRSISFFYAFGGGTEVVVK

SEQ ID NO: 319 - protein, 113 aa, Artificial Sequence
  Description of Artificial Sequence Synthetic polypeptide
QSLEESGGGLVQPEGSLTLTCTASGVTLTNYYICWVRQAPGKGLEWIGCIDNVNGRTYYASWAKGRFTISKASSTTGTLQMTSLTAADTATYFCARSLATPLWGPGTLVTVSS

SEQ ID NO: 320 - DNA, 336 bp, Artificial Sequence
  Description of Artificial Sequence Synthetic polynucleotide
gacattgtgatgacccagactccagcctccgtggaggcagctgtgggaggcacagtcaccatcaagtgccaggccagtcagagcattagtagctacttagcctggtatcaccagaaaccagggcagcctcccaagctcctgatctatgctgcatccactctggcatctggggtcccatcgcggttcgaaggcagtggatctgggacacagttcactctcaccatcagcgacctggagtgtgccgatgctgccacttactactgtcaaagctatgatgatagtaggagtagtagttttttttatgctttcggcggagggaccgaggtggtggtcaaa

SEQ ID NO: 321 - DNA, 339 bp, Artificial Sequence
  Description of Artificial Sequence Synthetic polynucleotide
cagtcgttggaggagtccgggggaggcctggtccagcctgagggatccctgacagtcacctgcacagcttttggagtcaccctcactaactactacatctgctgggtccgccaggctccagggaaggggctggagtggatcgggtgcattgacaatattaatggtaggacttactacgcgagctgggcgaaaggccgattcaccatctccaagacctcgtcgaccacagggactctgcaaatgaccagtctgacagccgcggacacggccacctatttctgtgcgaggtccttggctactcccttgtggggcccaggcaccctggtcaccgtctcctca

SEQ ID NO: 322 - protein, 112 aa, Artificial Sequence
  Description of Artificial Sequence Synthetic polypeptide
DIVMTQTPASVEAAVGGTVTIKCQASQSISSYLAWYHQKPGQPPKLLIYAASTLASGVPSRFEGSGSGTQFTLTISDLECADAATYYCQSYDDSRSSSFFYAFGGGTEVVVK

SEQ ID NO: 323 - protein, 113 aa, Artificial Sequence
  Description of Artificial Sequence Synthetic polypeptide
QSLEESGGGLVQPEGSLTVTCTAFGVTLTNYYICWVRQAPGKGLEWIGCIDNINGRTYYASWAKGRFTISKTSSTTGTLQMTSLTAADTATYFCARSLATPLWGPGTLVTVSS

SEQ ID NO: 324 - DNA, 336 bp, Artificial Sequence
  Description of Artificial Sequence Synthetic polynucleotide
gacattgtgatgacccagactccagcctccgtggaggcagctgtgggaggcacagtcaccatcaagtgccaggccagtcagagcattagcagttggttatcctggtatcagcagaaaccagggcagcctcccaagctcctgctctacagggcaaccactctggcatctggggtcccatcgcggttcaaaggcagtggatctgggacacagttcactctcaccatcagcgacctggagtgtgccgatgctgccacttattactgtcaaagttatgatgatagtagtagtagtaattttttttatgctttcggcggagggaccgaggtggtggtcaga

SEQ ID NO: 325 - DNA, 339 bp, Artificial Sequence
  Description of Artificial Sequence Synthetic polynucleotide
cagtcgttggaggagtccgggggaggcctggtccagcctgagggatccctgacactcacctgcacagcttctggattcaccctcactaattattatatatgttgggtccgccaggctccagggaagggactggagtggatcgcatgtattgataacgctaatggtaggacttactacgcgacctgggcgaaaggccgattcaccatctccaaaacctcgtcgaccacggtgactctgcaaatgcccagtctgacagccgcggacacggccacctatttctgtgcgaggtcattgtctactaacttgtggggcccaggcaccctggtcaccgtctcctca

SEQ ID NO: 326 - protein, 112 aa, Artificial Sequence
  Description of Artificial Sequence Synthetic polypeptide
DIVMTQTPASVEAAVGGTVTIKCQASQSISSWLSWYQQKPGQPPKLLLYRATTLASGVPSRFKGSGSGTQFTLTISDLECADAATYYCQSYDDSSSSNFFYAFGGGTEVVVR

SEQ ID NO: 327 - protein, 113 aa, Artificial Sequence
  Description of Artificial Sequence Synthetic polypeptide
QSLEESGGGLVQPEGSLTLTCTASGFTLTNYYICWVRQAPGKGLEWIACIDNANGRTYYATWAKGRFTISKTSSTTVTLQMPSLTAADTATYFCARSLSTNLWGPGTLVTVSS

SEQ ID NO: 328 - DNA, 330 bp, Artificial Sequence
  Description of Artificial Sequence Synthetic polynucleotide
caagccgtggtgacccagactccatcgtccgtgtctgcagctgtgggaggcacagtcaccatcagttgccagtccagtcagagtgtttatagtaacaacctcttatcttggtatcagcagaaaccagggcagcctcccaagctcctgatctacaaggcatccactctggcatctggggtcccatcgcggttgaaaggcagtggatctgggacacagttcactctcacaatcagcgaagtacagtgtgacgatgctgccacttattactgtcaaggctattatagtggtgttatttatatgttcggcggagggaccgaggtggtggtcaaa

SEQ ID NO: 329 - DNA, 360 bp, Artificial Sequence
  Description of Artificial Sequence Synthetic polynucleotide
cagtcgttggaggagtccgggggaggcctggtcaagcctggggcatccctgacactcacctgcaaagcctctgggtcagacttcagaagctactactacatatgctgggtccgccaggctccagggaaggggctggagtgggtcgcatgtattggtggtggtaataccgatgccactgcctacgcgaggtgggcgaaaggccgattcaccatctccaaaacctcggcgaccacggtggctctccaaatgaccagtctgacagccgcggacacggccacctatttctgtgtgagaggcggtcctgataataatgtccaatttaacttgtggggcccaggcaccctggtcaccgtctcctca

SEQ ID NO: 330 - protein, 110 aa, Artificial Sequence
  Description of Artificial Sequence Synthetic polypeptide
QAVVTQTPSSVSAAVGGTVTISCQSSQSVYSNNLLSWYQQKPGQPPKLLIYKASTLASGVPSRLKGSGSGTQFTLTISEVQCDDAATYYCQGYYSGVIYMFGGGTEVVVK

SEQ ID NO: 331 - protein, 120 aa, Artificial Sequence
  Description of Artificial Sequence Synthetic polypeptide
QSLEESGGGLVKPGASLTLTCKASGSDFRSYYYICWVRQAPGKGLEWVACIGGGNTDATAYARWAKGRFTISKTSATTVALQMTSLTAADTATYFCVRGGPDNNVQFNLWGPGTLVTVSS

SEQ ID NO: 332 - DNA, 336 bp, Artificial Sequence
  Description of Artificial Sequence Synthetic polynucleotide
gacattgtgatgacccagactccattctccgtggaggcagctgtgggaggcacagtcaccatcaagtgccaggccagtcagagcattagtagttacttatcctggtatcggcagaaaccagggcagcctcccaagctcctgatctacagggcatccactctggcatctggggtcccatcgcggttcaaaggcagtggatctgggacacagttcactctcaccatcagcgacctggagtgtgccgatgctgccacttactactgtcaaagctatgatgatagtagtagtaataattttttttatggtttcggcggagggaccgaggtggtggtcaga

SEQ ID NO: 333 - DNA, 339 bp, Artificial Sequence
  Description of Artificial Sequence Synthetic polynucleotide
cagtcgttggaggagtccgggggaggcctggtccagcctgagggatccctgacactcacctgcacagcttctggattcaccctcaataattattatatttgctgggtccgccaggctccagggaagggactggagtggatcgcatgcattgacaatagtaatggtaggacttactacgcgagctgggcgaaaggccgtttcaccatctccaaaacctcgtcgaccacggtgactctgcaaatgaccagtctgacagccgcggacacggccacctatttctgtgcgaggtcattgtctactcccttgtggggcccaggcaccctggtcaccgtctcctca

SEQ ID NO: 334 - protein, 112 aa, Artificial Sequence
  Description of Artificial Sequence Synthetic polypeptide
DIVMTQTPFSVEAAVGGTVTIKCQASQSISSYLSWYRQKPGQPPKLLIYRASTLASGVPSRFKGSGSGTQFTLTISDLECADAATYYCQSYDDSSSNNFFYGFGGGTEVVVR

SEQ ID NO: 335 - protein, 113 aa, Artificial Sequence
  Description of Artificial Sequence Synthetic polypeptide
QSLEESGGGLVQPEGSLTLTCTASGFTLNNYYICWVRQAPGKGLEWIACIDNSNGRTYYASWAKGRFTISKTSSTTVTLQMTSLTAADTATYFCARSLSTPLWGPGTLVTVSS

SEQ ID NO: 336 - DNA, 336 bp, Artificial Sequence
  Description of Artificial Sequence Synthetic polynucleotide
gacattgtgatgacccagactccatcctccgtggaggcagctgtgggaggcacagtcaccatcaagtgccaggccagtcagaacattagtagctacttagcctggtatcagcagaaaccagggcagcctcccaagctcctgatctacagggcatccactctggcatctggggtcccatcgcggttcaaaggcagtggatctgggacacagttcactctcaccatcagcgacctggagtgtgccgatgctgccacttactactgtcaaagctatgatgatagtaggagtagtaattttttttatgctttcggcggagggaccgaggtggtggtcaaa

SEQ ID NO: 337 - DNA, 339 bp, Artificial Sequence
  Description of Artificial Sequence Synthetic polynucleotide
cagtcgttggaggagtccgggggaggcctggtccagcctgagggatccctgacactcacctgcacagcttttggagtcaccctcactaactactacatctgctgggtccgccaggctccaggaaaggggctggagtgggtcgggtgtattgacaatgctaatggtaggacttactacgcgagctgggcgaaaggccgattcaccatctccaagacctcgtcgaccacagggactctgcaaatgaccagtctgacagccgcggacacggccacctatttctgtgcgaggtccttggctactcccttgtggggcccaggcaccctggtcaccgtctcctca

SEQ ID NO: 338 - protein, 112 aa, Artificial Sequence
  Description of Artificial Sequence Synthetic polypeptide
DIVMTQTPSSVEAAVGGTVTIKCQASQNISSYLAWYQQKPGQPPKLLIYRASTLASGVPSRFKGSGSGTQFTLTISDLECADAATYYCQSYDDSRSSNFFYAFGGGTEVVVK

SEQ ID NO: 339 - protein, 113 aa, Artificial Sequence
  Description of Artificial Sequence Synthetic polypeptide
QSLEESGGGLVQPEGSLTLTCTAFGVTLTNYYICWVRQAPGKGLEWVGCIDNANGRTYYASWAKGRFTISKTSSTTGTLQMTSLTAADTATYFCARSLATPLWGPGTLVTVSS

SEQ ID NO: 340 - DNA, 330 bp, Artificial Sequence
  Description of Artificial Sequence Synthetic polynucleotide
gcgcaagcgctgacccagactccatcccctgtgtctgcagctgtgggaggcacagtcaccatcagttgccagtccagtgagagcgtttataatcacaactggttaggctggtatcagcagaaaccagggcagcctcccaaactcctgatctatgatgcatccactctggcatctggggtcccatcgcggttcaaaggcagtggatctgggacacagttcactctcacaatcagcgaaagtcagtgtgacgatgctgccatttactactgtcaaggctattatcaaactagtgtttgggctttcggcggagggaccgaggtggtggtcaaa

SEQ ID NO: 341 - DNA, 351 bp, Artificial Sequence
  Description of Artificial Sequence Synthetic polynucleotide
cagtcggtggaggagtccgggggtcgcctggtcacgcctgggacacccctgacactcacctgcacagtttctggagtcgacctcagtagcagtgcagtgacctgggtccgccaggctccagggatgggactggaatacatcggattcctccaagctggggatggtagcgcatactacgcgagctgggcgaaaggccgattcaccatctccaaaacctcgtcgaccacagtggatctgaaaatgaccagtctcacaaccgaggacacggccacctatttctgtgccagacataagggtaatagttacgtgcctaacttgtggggcccaggcaccctggtcaccgtctcctca

SEQ ID NO: 342 - protein, 110 aa, Artificial Sequence
  Description of Artificial Sequence Synthetic polypeptide
AQALTQTPSPVSAAVGGTVTISCQSSESVYNHNWLGWYQQKPGQPPKLLIYDASTLASGVPSRFKGSGSGTQFTLTISESQCDDAAIYYCQGYYQTSVWAFGGGTEVVVK

SEQ ID NO: 343 - protein, 117 aa, Artificial Sequence
  Description of Artificial Sequence Synthetic polypeptide
QSVEESGGRLVTPGTPLTLTCTVSGVDLSSSAVTWVRQAPGMGLEYIGFLQAGDGSAYYASWAKGRFTISKTSSTTVDLKMTSLTTEDTATYFCARHKGNSYVPNLWGPGTLVTVSS

SEQ ID NO: 344 - DNA, 336 bp, Artificial Sequence
  Description of Artificial Sequence Synthetic polynucleotide
gatgttgtgatgacccagactccagcctccgtggaggcaactgtgggaggcacagtcaccatcaagtgccagtccagtcagagtgtttatgataacaatgctttagcctggtatcagcagaatgcaggacagcgtcccagactcctgatctatggtgcatccactctggcatctggggtcccatcgcggttcagtgccagtggatctgggacagagttcactctcaccatcagcgacctggagtgtgccgatgcagccacttactactgtcaatgtacttattatgttagtagttatcaaaatgatttcggcggagggaccgaggtggtggtcaaa

SEQ ID NO: 345 - DNA, 336 bp, Artificial Sequence
  Description of Artificial Sequence Synthetic polynucleotide
cagtcggtggaggagtccgggggtcgcctggtcacgcctgggacacccctgacactcacctgcacaatctctggattctccctcagtagctatgcaatgagctgggtccgccaggctccagggaaggggctggaatggatcggaagcattggtggtggtggtagcgcagtctacgcgagctgggcgaaaggccgattcaccatctccaaaacctcgaccacggtggatctgagaatcaccagtccgacaaccgaggacacggccatgtatttctgtggcaggggattttatagtatagacttgtggggcccaggcaccctggtcaccgtctcctca

SEQ ID NO: 346 - protein, 112 aa, Artificial Sequence
  Description of Artificial Sequence Synthetic polypeptide
DVVMTQTPASVEATVGGTVTIKCQSSQSVYDNNALAWYQQNAGQRPRLLIYGASTLASGVPSRFSASGSGTEFTLTISDLECADAATYYCQCTYYVSSYQNDFGGGTEVVVK

SEQ ID NO: 347 - protein, 112 aa, Artificial Sequence
  Description of Artificial Sequence Synthetic polypeptide
QSVEESGGRLVTPGTPLTLTCTISGFSLSSYAMSWVRQAPGKGLEWIGSIGGGGSAVYASWAKGRFTISKTSTTVDLRITSPTTEDTAMYFCGRGFYSIDLWGPGTLVTVSS

SEQ ID NO: 348 - DNA, 336 bp, Artificial Sequence
  Description of Artificial Sequence Synthetic polynucleotide
gccgccgtgctgacccagactccatctcccgtgtctgcagctgtgggaggcacagtcagcgccagttgccagtccagtaagagtgtttataataagaactggttatcctggtttcagcagaaaccagggcagcctcccaagctcctgatctatggtgcatccactctggcatctggggtcccatcgcggttcaaaggcagtggatctgggacacagttcactctcaccatcagcgacgtgcagtgtgacgatgctgccacttactactgtgcaggcggttatagtagtagtagtgatacatttgctttcggcggagggaccgaggtggtggtcaag

SEQ ID NO: 349 - DNA, 375 bp, Artificial Sequence
  Description of Artificial Sequence Synthetic polynucleotide
caggagcagctggtggagtccgggggaggcctggtcaagcctggggcatccctgacactcacctgcaaagcctctggattctccttcagtagcggccaactcatgtgctgggtccgccaggctccagggaaggggctggagtggatcgcatgcattggttctggtagtaatgctattagcactttctacgcgagctgggcgcaaggccgattcaccatctccaaatcctcgtcgaccacggtgactctgcaattgaccagtctgacagccgcggacacggccacctatttctgtgcgagagtgggctccgatgactatggtgactctgatgtttttgatccctggggcccaggcaccctggtcaccgtctcctca

SEQ ID NO: 350 - protein, 112 aa, Artificial Sequence
  Description of Artificial Sequence Synthetic polypeptide
AAVLTQTPSPVSAAVGGTVSASCQSSKSVYNKNWLSWFQQKPGQPPKLLIYGASTLASGVPSRFKGSGSGTQFTLTISDVQCDDAATYYCAGGYSSSSDTFAFGGGTEVVVK

SEQ ID NO: 351 - protein, 125 aa, Artificial Sequence
  Description of Artificial Sequence Synthetic polypeptide
QEQLVESGGGLVKPGASLTLTCKASGFSFSSGQLMCWVRQAPGKGLEWIACIGSGSNAISTFYASWAQGRFTISKSSSTTVTLQLTSLTAADTATYFCARVGSDDYGDSDVFDPWGPGTLVTVSS

SEQ ID NO: 352 - DNA, 330 bp, Artificial Sequence
  Description of Artificial Sequence Synthetic polynucleotide
gcccaagtgctgacccagactccagcctccgtgtctgcagctgtgggaggcacagtcaccatcagttgccagtccagtcagagcgtttatagtagcgacctcttatcctggtatcagcagaaaccagggcagcctcccaagctcctgatctacaaggcatccactctggcatctggggtcccatcgcggttcaaaggcagtggatctgggacacagttcactctcacaatcagcgaactacagtgtgacgatgctgccacttattactgtcaaggctactatagtggtgtggtttatattttcggcggagggaccgaggtggtggtcaag

SEQ ID NO: 353 - DNA, 360 bp, Artificial Sequence
  Description of Artificial Sequence Synthetic polynucleotide
cagtcgttggaggagtccgggggaggcctggtcaagcctggggcatccctgacactcacctgcaaagcctctgggtcagacctcagtagcgcctactacatatgctggatccgccaggctccagggaaggggctggagtgggtcgcatgtattggtggtgttaatcgcgttgccactgcctacgcgacctgggcgaaaggccgattcaccatctccaaaacctcgtcgaccacggtgactctgcaaatgaccagtctgacagccgcggacacggccacttatttctgtgtgagaggcggtcctgataataatgtccaatttaacttgtggggcccaggcaccctggtcaccgtctcctca

SEQ ID NO: 354 - protein, 110 aa, Artificial Sequence
  Description of Artificial Sequence Synthetic polypeptide
AQVLTQTPASVSAAVGGTVTISCQSSQSVYSSDLLSWYQQKPGQPPKLLIYKASTLASGVPSRFKGSGSGTQFTLTISELQCDDAATYYCQGYYSGVVYIFGGGTEVVVK

SEQ ID NO: 355 - protein, 120 aa, Artificial Sequence
  Description of Artificial Sequence Synthetic polypeptide
QSLEESGGGLVKPGASLTLTCKASGSDLSSAYYICWIRQAPGKGLEWVACIGGVNRVATAYATWAKGRFTISKTSSTTVTLQMTSLTAADTATYFCVRGGPDNNVQFNLWGPGTLVTVSS

SEQ ID NO: 356 - DNA, 330 bp, Artificial Sequence
  Description of Artificial Sequence Synthetic polynucleotide
gcccaagtgctgacccagactccagcctccgtgtctgcagctgtgggaggcacagtcaccatcagttgccagtccagtcagagcgtttatagtagcgacctcttatcctggtatcagcaaaaaccagggcagcctcccaagctcctgatctacaaggcatccactctggcatctggggtcccatcgcggttcaaaggcagtggatctgggacacagttcactctcacaatcagcgaagtacagtgtgacgatgctgccacttattactgtcaaggctactatagtggtgtggtttatattttcggcggagggaccgaggtggtggtcaag

SEQ ID NO: 357 - DNA, 360 bp, Artificial Sequence
  Description of Artificial Sequence Synthetic polynucleotide
cagtcgttggaggagtccgggggaggcctggtcaagcctggggcatccctgacactcacctgcaaagcctctgggtcagacctcagtaggtactactacagttgctggatccgccaggctccagggaaggggctggagtgggtcgcatgtgttggtggtgttaatcgcgatgccactgcctacgcgacctgggcgaaaggccgattcaccatctccaaaacctcgtcgaccacggtgactctgcaaatgaccagtctgacagccgcggacacggccacttatttctgtgtgagaggcggtcctgataataatgtccaatttaacttgtggggcccaggcaccctggtcaccgtctcctca

SEQ ID NO: 358 - protein, 110 aa, Artificial Sequence
  Description of Artificial Sequence Synthetic polypeptide
AQVLTQTPASVSAAVGGTVTISCQSSQSVYSSDLLSWYQQKPGQPPKLLIYKASTLASGVPSRFKGSGSGTQFTLTISEVQCDDAATYYCQGYYSGVVYIFGGGTEVVVK

SEQ ID NO: 359 - protein, 120 aa, Artificial Sequence
  Description of Artificial Sequence Synthetic polypeptide
QSLEESGGGLVKPGASLTLTCKASGSDLSRYYYSCWIRQAPGKGLEWVACVGGVNRDATAYATWAKGRFTISKTSSTTVTLQMTSLTAADTATYFCVRGGPDNNVQFNLWGPGTLVTVSS

SEQ ID NO: 360 - DNA, 336 bp, Artificial Sequence
  Description of Artificial Sequence Synthetic polynucleotide
gacattgtgatgacccagactccattctccgtggaggcagctgtgggaggcacagtcaccatcaagtgccaggccagtcagagcattagtagttacttatcttggtatcagcagaaaccagggcagcctcccaagctcctgatctacagggcatcccctctggcatctggggtcccatcgcggttcaaaggcagtggatctgggacacagttcactctcaccatcagcgacctggagtgtgccgatgctgccacttactactgtcaaagctacgatgatagtagtagtaataattttttttatggtttcggcggagggaccgaggtggtggtcaga

SEQ ID NO: 361 - DNA, 339 bp, Artificial Sequence
  Description of Artificial Sequence Synthetic polynucleotide
cagtcgttggaggagtccgggggaggcctggtccagcctgagggatccctgacactcacctgcacagcttctggattcaccctcaataattattatatttgttgggtccgccaggctccagggaagggactggagtggatcgcatgtattgacaatgttaatggtaggacttactacgcgagctgggcgaaaggccgattcaccatctccagaacctcgtcgaccacggtgactctgcaaatgaccagtctgacagccgcggacacggccacctatttctgtgcgaggtcattgtctactcccttgtggggcccaggcaccctggtcaccgtctcctcg

SEQ ID NO: 362 - protein, 112 aa, Artificial Sequence
  Description of Artificial Sequence Synthetic polypeptide
DIVMTQTPFSVEAAVGGTVTIKCQASQSISSYLSWYQQKPGQPPKLLIYRASPLASGVPSRFKGSGSGTQFTLTISDLECADAATYYCQSYDDSSSNNFFYGFGGGTEVVVR

SEQ ID NO: 363 - protein, 113 aa, Artificial Sequence
  Description of Artificial Sequence Synthetic polypeptide
QSLEESGGGLVQPEGSLTLTCTASGFTLNNYYICWVRQAPGKGLEWIACIDNVNGRTYYASWAKGRFTISRTSSTTVTLQMTSLTAADTATYFCARSLSTPLWGPGTLVTVSS

SEQ ID NO: 364 - DNA, 336 bp, Artificial Sequence
  Description of Artificial Sequence Synthetic polynucleotide
gccgccgtgctgacccagactccatctcccgtgtctgcagctgtgggaggcacagtcaccatcaattgccagtccagtaagagtgtttataataagaactggttatcctggtttcagcagaaaccagggcagcctcccaagctcctgatctatggtgcatccactctggcatctggggtcccatcgcggttcaaaggcagtggatctgggacacagttcactctcaccatcagcgacgtgcagtgtgacgatgttgccacttactactgtgcaggcggttatagtagtagtagtgatacgtttgctttcggcggagggaccgaggtggtggtcaaa

SEQ ID NO: 365 - DNA, 372 bp, Artificial Sequence
  Description of Artificial Sequence Synthetic polynucleotide
caggagcagctggtggagtccgggggaggcctggtccagcctgagggatccctgacactcacctgcgcagcttctggattctccttcagtagcagctacttcatgtgctgggtccgccaggctccagggaaggggctggagtggatcgcatgcattgctgttggtagtagtggtagcacttactacgcgagctgggcgaaaggccgattcaccatctccaaaacctcgtcgaccacggtgactctgcaaatgaccagtctgacagccgcggacacggccacctatttctgtgcgagagtgggctacgatgactatggtgactctgatgcttttgatccctggggcccaggcaccctggtcaccgtctcctca

SEQ ID NO: 366 - protein, 112 aa, Artificial Sequence
  Description of Artificial Sequence Synthetic polypeptide
AAVLTQTPSPVSAAVGGTVTINCQSSKSVYNKNWLSWFQQKPGQPPKLLIYGASTLASGVPSRFKGSGSGTQFTLTISDVQCDDVATYYCAGGYSSSSDTFAFGGGTEVVVK

SEQ ID NO: 367 - protein, 124 aa, Artificial Sequence
  Description of Artificial Sequence Synthetic polypeptide
QEQLVESGGGLVQPEGSLTLTCAASGFSFSSSYFMCWVRQAPGKGLEWIACIAVGSSGSTYYASWAKGRFTISKTSSTTVTLQMTSLTAADTATYFCARVGYDDYGDSDAFDPWGPGTLVTVSS

SEQ ID NO: 368 - DNA, 330 bp, Artificial Sequence
  Description of Artificial Sequence Synthetic polynucleotide
gcccaagtgctgacccagactccagcctccgtgtctgcagctgtgggaggcacagtcaccatcagttgccagtccagtcagagcctttataatagcgacctcttatcctggtatcagcaaaaaccagggcagcctcccaagctcctgatctacaaggcatccactctggcatctggggtcccatcgcggttcaaaggcagtggatctgggacacagttcactctcacaatcagcgaagtacagtgtgacgatgctgccacttattactgtcaaggctactatagtggtgtggtttatattttcggcggagggaccgaggtggtggtcaag

SEQ ID NO: 369 - DNA, 360 bp, Artificial Sequence; identical to SEQ ID NO: 357
  Description of Artificial Sequence Synthetic polynucleotide

SEQ ID NO: 370 - protein, 110 aa, Artificial Sequence
  Description of Artificial Sequence Synthetic polypeptide
AQVLTQTPASVSAAVGGTVTISCQSSQSLYNSDLLSWYQQKPGQPPKLLIYKASTLASGVPSRFKGSGSGTQFTLTISEVQCDDAATYYCQGYYSGVVYIFGGGTEVVVK

SEQ ID NO: 371 - protein, 120 aa, Artificial Sequence; identical to SEQ ID NO: 359
  Description of Artificial Sequence Synthetic polypeptide

SEQ ID NO: 372 - DNA, 336 bp, Artificial Sequence
  Description of Artificial Sequence Synthetic polynucleotide
gccgccgtgctgacccagactccatctcccgtgtctgcagctgtgggaggcacagtcagcatcagttgccagtccagtaagagtgtttataataagaactggttatcctggtttcagcagaaaccagggcagcctcccaagctcctgatctatggtgcatccactctggcatctggggtcccatcgcggttcaaaggcagtggatctgggacacagttcactctcaccatcagcgacgtgcagtgtgacgatgctgccacttactactgtgcaggcggttatagtagtagtagtgatacatttgctttcggcggagggaccgaggtggtggtcaaa

SEQ ID NO: 373 - DNA, 375 bp, Artificial Sequence
  Description of Artificial Sequence Synthetic polynucleotide
caggagcagctggtggagtccgggggaggcctggtccagcctaagggatccctgacactcacctgcgcagcttctggattctccttcagtagcagctacttcatgtgctgggtccgccaggctccagggaaggggctggagtggatcgcatgcattggttctggtagtagtgctattagcactttctacgcgagctgggcgcaaggccgattcaccatctccaaaacctcgtcgaccacggtgactctgcaaatgaccagtctgacagccgcggacacggccacctatttctgtgcgagagtgggctacgatgactatggtgactctgatgcttttgatccctggggcccaggcaccctggtcaccgtctcctca

SEQ ID NO: 374 - protein, 112 aa, Artificial Sequence
  Description of Artificial Sequence Synthetic polypeptide
AAVLTQTPSPVSAAVGGTVSISCQSSKSVYNKNWLSWFQQKPGQPPKLLIYGASTLASGVPSRFKGSGSGTQFTLTISDVQCDDAATYYCAGGYSSSSDTFAFGGGTEVVVK

SEQ ID NO: 375 - protein, 125 aa, Artificial Sequence
  Description of Artificial Sequence Synthetic polypeptide
QEQLVESGGGLVQPKGSLTLTCAASGFSFSSSYFMCWVRQAPGKGLEWIACIGSGSSAISTFYASWAQGRFTISKTSSTTVTLQMTSLTAADTATYFCARVGYDDYGDSDAFDPWGPGTLVTVSS

SEQ ID NO: 376 - DNA, 336 bp, Artificial Sequence
  Description of Artificial Sequence Synthetic polynucleotide
gatgttgtgatgacccacactccagcctccctctttgaaactgtgggaggcacagtcaccatcaagtgccaggcccttcagagtgtttatgataacaatgctttatcctggtatcaacaaaatgcaggacagcgtcccatactcctgatctatggtgcatccactctggcatctggggccccatcgcggttcagtgccagtggatctgggacagatttcactctcaccatcatcgacctggagtgtgccgatgcttccacttactactgtcaatgtacttattatgttagtagttatcaaaatgatttcggcggagggaccgaggtggtggtcaaa

SEQ ID NO: 377 - DNA, 342 bp, Artificial Sequence
  Description of Artificial Sequence Synthetic polynucleotide
cagtcgctggaggagtccgggggtcgcctggtaacgcctgggacacccctgacactcacctgcacagtctctggaatcgacctcagtagctatggaatgacttgggtccgccaggctccagggaaggggctggagtggatcggatacatttggactgatgggaggacatactacgcaaactgggcgaaaggccgattcaccatctccaaaacctcgaccacagtggatctcaagatcaccagtccgacagccgaggacacggccacctatttctgtgccagaccctttgatggtaattatagggacatctggggcccaggcaccctggtcacggtctcctta

SEQ ID NO: 378 - protein, 112 aa, Artificial Sequence
  Description of Artificial Sequence Synthetic polypeptide
DVVMTHTPASLFETVGGTVTIKCQALQSVYDNNALSWYQQNAGQRPILLIYGASTLASGAPSRFSASGSGTDFTLTIIDLECADASTYYCQCTYYVSSYQNDFGGGTEVVVK

SEQ ID NO: 379 - protein, 114 aa, Artificial Sequence
  Description of Artificial Sequence Synthetic polypeptide
QSLEESGGRLVTPGTPLTLTCTVSGIDLSSYGMTWVRQAPGKGLEWIGYIWTDGRTYYANWAKGRFTISKTSTTVDLKITSPTAEDTATYFCARPFDGNYRDIWGPGTLVTVSL

SEQ ID NO: 380 - DNA, 336 bp, Artificial Sequence
  Description of Artificial Sequence Synthetic polynucleotide
gacattgtgatgacccagactccagcctccgtggaggcagctgtgggaggcacagtcaccatcaagtgccaggccagtgagagcattggcaatgcattagcctggtatcagcagaaaccagggcagcctcccagtctcctgatctacagggcatccactctggcatctggaatcccatcgcgggtcaaaggcagtggatctgggacacagttcactctcaccatcagcgacctggagtgtgccgatgctgccacttactattgtcaaagctatgatgatagtagtagtagtagttttttttatgctttcggcggagggaccgaggtggtggtcaaa

SEQ ID NO: 381 - DNA, 339 bp, Artificial Sequence
  Description of Artificial Sequence Synthetic polynucleotide
cagtcgttggaggagtccgggggaggcctggtccagcctgagggatccctgacactcacctgcacagcttctggattcaccctcaataactactacatctgctgggtccgccaggctccagggaaggggctggagtggatcgcatgtattgataatgccaatggtcggacttactacgcgaactgggcgaaaggccgattcaccatctccaagacctcgtcgaccacggtgactctgcaaatgaccagtctgacagccgcggacacggccacctatttctgtgcgaggtccttggctactcccttgtggggcccaggcaccctggtcaccgtctcctca

SEQ ID NO: 382 - protein, 112 aa, Artificial Sequence
  Description of Artificial Sequence Synthetic polypeptide
DIVMTQTPASVEAAVGGTVTIKCQASESIGNALAWYQQKPGQPPSLLIYRASTLASGIPSRVKGSGSGTQFTLTISDLECADAATYYCQSYDDSSSSSFFYAFGGGTEVVVK

SEQ ID NO: 383 - protein, 113 aa, Artificial Sequence
  Description of Artificial Sequence Synthetic polypeptide
QSLEESGGGLVQPEGSLTLTCTASGFTLNNYYICWVRQAPGKGLEWIACIDNANGRTYYANWAKGRFTISKTSSTTVTLQMTSLTAADTATYFCARSLATPLWGPGTLVTVSS

SEQ ID NO: 384 - DNA, 336 bp, Artificial Sequence
  Description of Artificial Sequence Synthetic polynucleotide
gccgccgtgctgacccagactccatctcccgtgtctgcagctgtgggaggcacagtcagcatcagttgccagtccagtaagagtgtttataataagaactggttatcctggtttcagcagaaaccagggcagcctcccaagctcctgatctatggtgcatccactctggcatctggggtcccatcgcggttcaaaggcagtggatctgggacacagttcactctcaccatcagcggcgtgcagtgtgacgatgctgccacttactactgtgcaggcggttatagtagtagtagtgatacgtttgctttcggcggagggaccgaggtggtggtcaaa

SEQ ID NO: 385 - DNA, 372 bp, Artificial Sequence
  Description of Artificial Sequence Synthetic polynucleotide
caggagcagctggtggagtccgggggaggcctggtccagcctgagggatccctgacactcacctgcgcagcttctggattctccttcagtagcagctacttcatgtgctgggtccgccaggctccagggaaggggctggagtggatcgcatgcattggtgttggtggtagtggtagcacttactacgcgaactgggcgaaaggccgattcaccatctccaaaacctcgtcgaccacggtgactctgcaaatgaccagtctgacagccgcggacacggccacctatttctgtgcgagagtggcctacgatgacgatggtgactctgatgcttttgatccctggggcccaggcaccctggtcaccgtctcctca

SEQ ID NO: 386 - protein, 112 aa, Artificial Sequence
  Description of Artificial Sequence Synthetic polypeptide
AAVLTQTPSPVSAAVGGTVSISCQSSKSVYNKNWLSWFQQKPGQPPKLLIYGASTLASGVPSRFKGSGSGTQFTLTISGVQCDDAATYYCAGGYSSSSDTFAFGGGTEVVVK

SEQ ID NO: 387 - protein, 124 aa, Artificial Sequence
  Description of Artificial Sequence Synthetic polypeptide
QEQLVESGGGLVQPEGSLTLTCAASGFSFSSSYFMCWVRQAPGKGLEWIACIGVGGSGSTYYANWAKGRFTISKTSSTTVTLQMTSLTAADTATYFCARVAYDDDGDSDAFDPWGPGTLVTVSS

SEQ ID NO: 388 - protein, 33 aa, Homo sapiens
SVSGGAASSSPAAGIPQEPQRAPTTAAAAAAGV

SEQ ID NO: 389 - protein, 32 aa, Homo sapiens
VSGGAASSSPAAGIPQEPQRAPTTAAAAAAGV

SEQ ID NO: 390 - protein, 31 aa, Homo sapiens
SGGAASSSPAAGIPQEPQRAPTTAAAAAAGV

SEQ ID NO: 391 - protein, 31 aa, Homo sapiens; identical to SEQ ID NO: 390

SEQ ID NO: 392 - protein, 30 aa, Homo sapiens
GGAASSSPAAGIPQEPQRAPTTAAAAAAGV

SEQ ID NO: 393 - protein, 29 aa, Homo sapiens
GAASSSPAAGIPQEPQRAPTTAAAAAAGV

SEQ ID NO: 394 - protein, 28 aa, Homo sapiens
AASSSPAAGIPQEPQRAPTTAAAAAAGV

SEQ ID NO: 395 - protein, 27 aa, Homo sapiens
ASSSPAAGIPQEPQRAPTTAAAAAAGV

SEQ ID NO: 396 - protein, 26 aa, Homo sapiens
SSSPAAGIPQEPQRAPTTAAAAAAGV

SEQ ID NO: 397 - protein, 25 aa, Homo sapiens
SSPAAGIPQEPQRAPTTAAAAAAGV

SEQ ID NO: 398 - protein, 24 aa, Homo sapiens
SPAAGIPQEPQRAPTTAAAAAAGV

SEQ ID NO: 399 - protein, 23 aa, Homo sapiens
PAAGIPQEPQRAPTTAAAAAAGV

SEQ ID NO: 400 - protein, 22 aa, Homo sapiens
AAGIPQEPQRAPTTAAAAAAGV

SEQ ID NO: 401 - protein, 21 aa, Homo sapiens
AGIPQEPQRAPTTAAAAAAGV

SEQ ID NO: 402 - protein, 20 aa, Homo sapiens
GIPQEPQRAPTTAAAAAAGV

SEQ ID NO: 403 - protein, 19 aa, Homo sapiens
IPQEPQRAPTTAAAAAAGV

SEQ ID NO: 404 - protein, 18 aa, Homo sapiens
PQEPQRAPTTAAAAAAGV

SEQ ID NO: 405 - protein, 17 aa, Homo sapiens
QEPQRAPTTAAAAAAGV

SEQ ID NO: 406 - protein, 16 aa, Homo sapiens
EPQRAPTTAAAAAAGV

SEQ ID NO: 407 - protein, 15 aa, Homo sapiens
PQRAPTTAAAAAAGV

SEQ ID NO: 408 - protein, 14 aa, Homo sapiens
QRAPTTAAAAAAGV

SEQ ID NO: 409 - protein, 13 aa, Homo sapiens
RAPTTAAAAAAGV

SEQ ID NO: 410 - protein, 12 aa, Homo sapiens
APTTAAAAAAGV

SEQ ID NO: 411 - protein, 11 aa, Homo sapiens
PTTAAAAAAGV

SEQ ID NO: 412 - protein, 10 aa, Homo sapiens
TTAAAAAAGV

SEQ ID NO: 413 - protein, 9 aa, Homo sapiens
TAAAAAAGV

SEQ ID NO: 414 - protein, 8 aa, Homo sapiens
AAAAAAGV

SEQ ID NO: 415 - protein, 7 aa, Homo sapiens
AAAAAGV

SEQ ID NO: 416 - protein, 6 aa, Homo sapiens
AAAAGV

SEQ ID NO: 417 - protein, 5 aa, Homo sapiens
AAAGV

SEQ ID NO: 418 - protein, 33 aa, Homo sapiens
SSVSGGAASSSPAAGIPQEPQRAPTTAAAAAAG

SEQ ID NO: 419 - protein, 32 aa, Homo sapiens
SSVSGGAASSSPAAGIPQEPQRAPTTAAAAAA

SEQ ID NO: 420 - protein, 31 aa, Homo sapiens
SSVSGGAASSSPAAGIPQEPQRAPTTAAAAA

SEQ ID NO: 421 - protein, 30 aa, Homo sapiens
SSVSGGAASSSPAAGIPQEPQRAPTTAAAA

SEQ ID NO: 422 - protein, 29 aa, Homo sapiens
SSVSGGAASSSPAAGIPQEPQRAPTTAAA

SEQ ID NO: 423 - protein, 28 aa, Homo sapiens
SSVSGGAASSSPAAGIPQEPQRAPTTAA

SEQ ID NO: 424 - protein, 27 aa, Homo sapiens
SSVSGGAASSSPAAGIPQEPQRAPTTA

SEQ ID NO: 425 - protein, 26 aa, Homo sapiens
SSVSGGAASSSPAAGIPQEPQRAPTT

SEQ ID NO: 426 - protein, 25 aa, Homo sapiens
SSVSGGAASSSPAAGIPQEPQRAPT

SEQ ID NO: 427 - protein, 24 aa, Homo sapiens
SSVSGGAASSSPAAGIPQEPQRAP

SEQ ID NO: 428 - protein, 23 aa, Homo sapiens
SSVSGGAASSSPAAGIPQEPQRA

SEQ ID NO: 429 - protein, 22 aa, Homo sapiens
SSVSGGAASSSPAAGIPQEPQR

SEQ ID NO: 430 - protein, 21 aa, Homo sapiens
SSVSGGAASSSPAAGIPQEPQ

SEQ ID NO: 431 - protein, 20 aa, Homo sapiens
SSVSGGAASSSPAAGIPQEP

SEQ ID NO: 432 - protein, 19 aa, Homo sapiens
SSVSGGAASSSPAAGIPQE

SEQ ID NO: 433 - protein, 18 aa, Homo sapiens
SSVSGGAASSSPAAGIPQ

SEQ ID NO: 434 - protein, 17 aa, Homo sapiens
SSVSGGAASSSPAAGIP

SEQ ID NO: 435 - protein, 16 aa, Homo sapiens
SSVSGGAASSSPAAGI

SEQ ID NO: 436 - protein, 15 aa, Homo sapiens
SSVSGGAASSSPAAG

SEQ ID NO: 437 - protein, 14 aa, Homo sapiens
SSVSGGAASSSPAA

SEQ ID NO: 438 - protein, 13 aa, Homo sapiens
SSVSGGAASSSPA

SEQ ID NO: 439 - protein, 12 aa, Homo sapiens
SSVSGGAASSSP

SEQ ID NO: 440 - protein, 11 aa, Homo sapiens
SSVSGGAASSS

SEQ ID NO: 441 - protein, 10 aa, Homo sapiens
SSVSGGAASS

SEQ ID NO: 442 - protein, 9 aa, Homo sapiens
SSVSGGAAS

SEQ ID NO: 443 - protein, 8 aa, Homo sapiens
SSVSGGAA

SEQ ID NO: 444 - protein, 7 aa, Homo sapiens
SSVSGGA

SEQ ID NO: 445 - protein, 6 aa, Homo sapiens
SSVSGG

SEQ ID NO: 446 - protein, 5 aa, Homo sapiens
SSVSG

SEQ ID NO: 447 - protein, 32 aa, Homo sapiens
SVSGGAASSSPAAGIPQEPQRAPTTAAAAAAG

SEQ ID NO: 448 - protein, 30 aa, Homo sapiens
VSGGAASSSPAAGIPQEPQRAPTTAAAAAA

SEQ ID NO: 449 - protein, 28 aa, Homo sapiens
SGGAASSSPAAGIPQEPQRAPTTAAAAA

SEQ ID NO: 450 - protein, 26 aa, Homo sapiens
GGAASSSPAAGIPQEPQRAPTTAAAA

SEQ ID NO: 451 - protein, 24 aa, Homo sapiens
GAASSSPAAGIPQEPQRAPTTAAA

SEQ ID NO: 452 - protein, 22 aa, Homo sapiens
AASSSPAAGIPQEPQRAPTTAA

SEQ ID NO: 453 - protein, 20 aa, Homo sapiens
ASSSPAAGIPQEPQRAPTTA

SEQ ID NO: 454 - protein, 18 aa, Homo sapiens
SSSPAAGIPQEPQRAPTT

SEQ ID NO: 455 - protein, 16 aa, Homo sapiens
SSPAAGIPQEPQRAPT

SEQ ID NO: 456 - protein, 14 aa, Homo sapiens
SPAAGIPQEPQRAP

SEQ ID NO: 457 - protein, 12 aa, Homo sapiens
PAAGIPQEPQRA

SEQ ID NO: 458 - protein, 10 aa, Homo sapiens
AAGIPQEPQR

SEQ ID NO: 459 - protein, 8 aa, Homo sapiens
AGIPQEPQ

SEQ ID NO: 460 - protein, 6 aa, Homo sapiens
GIPQEP

SEQ ID NO: 461 - protein, 4 aa, Homo sapiens
IPQE

SEQ ID NO: 462 - protein, 30 aa, Homo sapiens
SSQASTSTKSPSEDPLTRKSGSLVQFLLYK

SEQ ID NO: 463 - protein, 29 aa, Homo sapiens
SQASTSTKSPSEDPLTRKSGSLVQFLLYK

SEQ ID NO: 464 - protein, 28 aa, Homo sapiens
QASTSTKSPSEDPLTRKSGSLVQFLLYK

SEQ ID NO: 465 - protein, 27 aa, Homo sapiens
ASTSTKSPSEDPLTRKSGSLVQFLLYK

SEQ ID NO: 466 - protein, 26 aa, Homo sapiens
STSTKSPSEDPLTRKSGSLVQFLLYK

SEQ ID NO: 467 - protein, 25 aa, Homo sapiens
TSTKSPSEDPLTRKSGSLVQFLLYK

SEQ ID NO: 468 - protein, 24 aa, Homo sapiens
STKSPSEDPLTRKSGSLVQFLLYK

SEQ ID NO: 469 - protein, 23 aa, Homo sapiens
TKSPSEDPLTRKSGSLVQFLLYK

SEQ ID NO: 470 - protein, 22 aa, Homo sapiens
KSPSEDPLTRKSGSLVQFLLYK

SEQ ID NO: 471 - protein, 21 aa, Homo sapiens
SPSEDPLTRKSGSLVQFLLYK

SEQ ID NO: 472 - protein, 20 aa, Homo sapiens
PSEDPLTRKSGSLVQFLLYK

SEQ ID NO: 473 - protein, 19 aa, Homo sapiens
SEDPLTRKSGSLVQFLLYK

SEQ ID NO: 474 - protein, 18 aa, Homo sapiens
EDPLTRKSGSLVQFLLYK

SEQ ID NO: 475 - protein, 17 aa, Homo sapiens
DPLTRKSGSLVQFLLYK

SEQ ID NO: 476 - protein, 16 aa, Homo sapiens
PLTRKSGSLVQFLLYK

SEQ ID NO: 477 - protein, 15 aa, Homo sapiens
LTRKSGSLVQFLLYK

SEQ ID NO: 478 - protein, 14 aa, Homo sapiens
TRKSGSLVQFLLYK

SEQ ID NO: 479 - protein, 13 aa, Homo sapiens
RKSGSLVQFLLYK

SEQ ID NO: 480 - protein, 12 aa, Homo sapiens
KSGSLVQFLLYK

SEQ ID NO: 481 - protein, 11 aa, Homo sapiens
SGSLVQFLLYK

SEQ ID NO: 482 - protein, 10 aa, Homo sapiens
GSLVQFLLYK

SEQ ID NO: 483 - protein, 9 aa, Homo sapiens
SLVQFLLYK

SEQ ID NO: 484 - protein, 8 aa, Homo sapiens
LVQFLLYK

SEQ ID NO: 485 - protein, 7 aa, Homo sapiens
VQFLLYK

SEQ ID NO: 486 - protein, 6 aa, Homo sapiens
QFLLYK

SEQ ID NO: 487 - protein, 5 aa, Homo sapiens
FLLYK

SEQ ID NO: 488 - protein, 30 aa, Homo sapiens
SSSQASTSTKSPSEDPLTRKSGSLVQFLLY

SEQ ID NO: 489 - protein, 29 aa, Homo sapiens
SSSQASTSTKSPSEDPLTRKSGSLVQFLL

SEQ ID NO: 490 - protein, 28 aa, Homo sapiens
SSSQASTSTKSPSEDPLTRKSGSLVQFL

SEQ ID NO: 491 - protein, 27 aa, Homo sapiens
SSSQASTSTKSPSEDPLTRKSGSLVQF

SEQ ID NO: 492 - protein, 26 aa, Homo sapiens
SSSQASTSTKSPSEDPLTRKSGSLVQ

SEQ ID NO: 493 - protein, 25 aa, Homo sapiens
SSSQASTSTKSPSEDPLTRKSGSLV

SEQ ID NO: 494 - protein, 24 aa, Homo sapiens
SSSQASTSTKSPSEDPLTRKSGSL

SEQ ID NO: 495 - protein, 23 aa, Homo sapiens
SSSQASTSTKSPSEDPLTRKSGS

SEQ ID NO: 496 - protein, 22 aa, Homo sapiens
SSSQASTSTKSPSEDPLTRKSG

SEQ ID NO: 497 - protein, 21 aa, Homo sapiens
SSSQASTSTKSPSEDPLTRKS

SEQ ID NO: 498 - protein, 20 aa, Homo sapiens
SSSQASTSTKSPSEDPLTRK

SEQ ID NO: 499 - protein, 19 aa, Homo sapiens
SSSQASTSTKSPSEDPLTR

SEQ ID NO: 500 - protein, 18 aa, Homo sapiens
SSSQASTSTKSPSEDPLT

SEQ ID NO: 501 - protein, 17 aa, Homo sapiens
SSSQASTSTKSPSEDPL

SEQ ID NO: 502 - protein, 16 aa, Homo sapiens
SSSQASTSTKSPSEDP

SEQ ID NO: 503 - protein, 15 aa, Homo sapiens
SSSQASTSTKSPSED

SEQ ID NO: 504 - protein, 14 aa, Homo sapiens
SSSQASTSTKSPSE

SEQ ID NO: 505 - protein, 13 aa, Homo sapiens
SSSQASTSTKSPS

SEQ ID NO: 506 - protein, 12 aa, Homo sapiens
SSSQASTSTKSP

SEQ ID NO: 507 - protein, 11 aa, Homo sapiens
SSSQASTSTKS

SEQ ID NO: 508 - protein, 10 aa, Homo sapiens
SSSQASTSTK

SEQ ID NO: 509 - protein, 9 aa, Homo sapiens
SSSQASTST

SEQ ID NO: 510 - protein, 8 aa, Homo sapiens
SSSQASTS

SEQ ID NO: 511 - protein, 7 aa, Homo sapiens
SSSQAST

SEQ ID NO: 512 - protein, 6 aa, Homo sapiens
SSSQAS

SEQ ID NO: 513 - protein, 5 aa, Homo sapiens
SSSQA

SEQ ID NO: 514 - protein, 29 aa, Homo sapiens
SSQASTSTKSPSEDPLTRKSGSLVQFLLY

SEQ ID NO: 515 - protein, 27 aa, Homo sapiens
SQASTSTKSPSEDPLTRKSGSLVQFLL

SEQ ID NO: 516 - protein, 25 aa, Homo sapiens
QASTSTKSPSEDPLTRKSGSLVQFL

SEQ ID NO: 517 - protein, 23 aa, Homo sapiens
ASTSTKSPSEDPLTRKSGSLVQF

SEQ ID NO: 518 - protein, 21 aa, Homo sapiens
STSTKSPSEDPLTRKSGSLVQ

SEQ ID NO: 519 - protein, 19 aa, Homo sapiens
TSTKSPSEDPLTRKSGSLV

SEQ ID NO: 520 - protein, 17 aa, Homo sapiens
STKSPSEDPLTRKSGSL

SEQ ID NO: 521 - protein, 15 aa, Homo sapiens
TKSPSEDPLTRKSGS

SEQ ID NO: 522 - protein, 13 aa, Homo sapiens
KSPSEDPLTRKSG

SEQ ID NO: 523 - protein, 11 aa, Homo sapiens
SPSEDPLTRKS

SEQ ID NO: 524 - protein, 9 aa, Homo sapiens
PSEDPLTRK

SEQ ID NO: 525 - protein, 7 aa, Homo sapiens
SEDPLTR

SEQ ID NO: 526 - protein, 5 aa, Homo sapiens
EDPLT

SEQ ID NO: 527 - protein, 15 aa, Homo sapiens
LTDEESLLSSWDFPR

SEQ ID NO: 528 - protein, 14 aa, Homo sapiens
TDEESLLSSWDFPR

SEQ ID NO: 529 - protein, 13 aa, Homo sapiens
DEESLLSSWDFPR

SEQ ID NO: 530 - protein, 12 aa, Homo sapiens
EESLLSSWDFPR

SEQ ID NO: 531 - protein, 11 aa, Homo sapiens
ESLLSSWDFPR

SEQ ID NO: 532 - protein, 10 aa, Homo sapiens
SLLSSWDFPR

SEQ ID NO: 533 - protein, 9 aa, Homo sapiens
LLSSWDFPR

SEQ ID NO: 534 - protein, 8 aa, Homo sapiens
LSSWDFPR

SEQ ID NO: 535 - protein, 7 aa, Homo sapiens
SSWDFPR

SEQ ID NO: 536 - protein, 6 aa, Homo sapiens
SWDFPR

SEQ ID NO: 537 - protein, 5 aa, Homo sapiens
WDFPR

SEQ ID NO: 538 - protein, 15 aa, Homo sapiens
DLTDEESLLSSWDFP

SEQ ID NO: 539 - protein, 14 aa, Homo sapiens
DLTDEESLLSSWDF

SEQ ID NO: 540 - protein, 13 aa, Homo sapiens
DLTDEESLLSSWD

SEQ ID NO: 541 - protein, 12 aa, Homo sapiens
DLTDEESLLSSW

SEQ ID NO: 542 - protein, 11 aa, Homo sapiens
DLTDEESLLSS

SEQ ID NO: 543 - protein, 10 aa, Homo sapiens
DLTDEESLLS

SEQ ID NO: 544 - protein, 9 aa, Homo sapiens
DLTDEESLL

SEQ ID NO: 545 - protein, 8 aa, Homo sapiens
DLTDEESL

SEQ ID NO: 546 - protein, 7 aa, Homo sapiens
DLTDEES

SEQ ID NO: 547 - protein, 6 aa, Homo sapiens
DLTDEE

SEQ ID NO: 548 - protein, 5 aa, Homo sapiens
DLTDE

SEQ ID NO: 549 - protein, 14 aa, Homo sapiens
LTDEESLLSSWDFP

SEQ ID NO: 550 - protein, 12 aa, Homo sapiens
TDEESLLSSWDF

SEQ ID NO: 551 - protein, 10 aa, Homo sapiens
DEESLLSSWD

SEQ ID NO: 552 - protein, 8 aa, Homo sapiens
EESLLSSW

SEQ ID NO: 553 - protein, 6 aa, Homo sapiens
ESLLSS

SEQ ID NO: 554 - protein, 4 aa, Homo sapiens
SLLS

SEQ ID NO: 555 - protein, 238 aa, Artificial Sequence
  Description of Artificial Sequence Synthetic polypeptide
MDMRVPAQLLGLLLLWLRGARCDIVMTQTPSSVEAAVGGTVTIKCQASQNISSYLAWYQQKPGQPPKLLIYRASTLASGVPSRFKGSGSGTQFTLTISDLECADAATYYCQSYDDSRSSNFFYAFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 556 - protein, 458 aa, Artificial Sequence
  Description of Artificial Sequence Synthetic polypeptide
MDMRVPAQLLGLLLLWLRGARCQSLEESGGGLVQPEGSLTLTCTAFGVTLTNYYICWVRQAPGKGLEWVGCIDNANGRTYYASWAKGRFTISKTSSTTGTLQMTSLTAADTATYFCARSLATPLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 557 - protein, 238 aa, Artificial Sequence
  Description of Artificial Sequence Synthetic polypeptide
MDMRVPAQLLGLLLLWLRGARCDVVMTQTPASVEATVGGTVTIKCQSSQSVYDNNALAWYQQNAGQRPRLLIYGASTLASGVPSRFSASGSGTEFTLTISDLECADAATYYCQCTYYVSSYQNDFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 558 - protein, 457 aa, Artificial Sequence
  Description of Artificial Sequence Synthetic polypeptide
MDMRVPAQLLGLLLLWLRGARCQSVEESGGRLVTPGTPLTLTCTISGFSLSSYAMSWVRQAPGKGLEWIGSIGGGGSAVYASWAKGRFTISKTSTTVDLRITSPTTEDTAMYFCGRGFYSIDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 559 - protein, 238 aa, Artificial Sequence
  Description of Artificial Sequence Synthetic polypeptide
MDMRVPAQLLGLLLLWLRGARCAAVLTQTPSPVSAAVGGTVSASCQSSKSVYNKNWLSWFQQKPGQPPKLLIYGASTLASGVPSRFKGSGSGTQFTLTISDVQCDDAATYYCAGGYSSSSDTFAFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 560 - protein, 470 aa, Artificial Sequence
  Description of Artificial Sequence Synthetic polypeptide
MDMRVPAQLLGLLLLWLRGARCQEQLVESGGGLVKPGASLTLTCKASGFSFSSGQLMCWVRQAPGKGLEWIACIGSGSNAISTFYASWAQGRFTISKSSSTTVTLQLTSLTAADTATYFCARVGSDDYGDSDVFDPWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 561 - protein, 10 aa, Homo sapiens
GVYDGEEHSV

SEQ ID NO: 562 - protein, 16 aa, Homo sapiens
DLTDEESLLSSWDFPR

SEQ ID NO: 563 - protein, 12 aa, Artificial Sequence
  Description of Artificial Sequence Synthetic peptide
ERKCCVECPPCP

SEQ ID NO: 564 - protein, 16 aa, Artificial Sequence
  Description of Artificial Sequence Synthetic peptide
ELKTPLDTTHTCPRCP

SEQ ID NO: 565 - protein, 17 aa, Artificial Sequence
  Description of Artificial Sequence Synthetic peptide
ELKTPLGDTTHTCPRCP

SEQ ID NO: 566 - protein, 11 aa, Artificial Sequence
  Description of Artificial Sequence Synthetic peptide
QASQSIRNELF

SEQ ID NO: 567 - protein, 12 aa, Artificial Sequence
  Description of Artificial Sequence Synthetic peptide
ESKYGPPCPSCP

SEQ ID NO: 568 - protein, 15 aa, Artificial Sequence
  Description of Artificial Sequence Synthetic peptide
EPKSCDKTHTCPPCP

SEQ ID NO: 569 - protein, 6 aa, Artificial Sequence
  Description of Artificial Sequence Synthetic 6xHis tag
HHHHHH

SEQ ID NO: 570 - protein, 27 aa, Artificial Sequence
  Description of Artificial Sequence Synthetic peptide
SYVKVLEYVIKVSARVRFFFPSLREAA

SEQ ID NO: 571 - protein, 37 aa, Artificial Sequence
  Description of Artificial Sequence Synthetic polypeptide
SSPLVLGTLEEVPTAGSTDPPQSPQGASAFPTTINFT

SEQ ID NO: 572 - protein, 25 aa, Artificial Sequence
  Description of Artificial Sequence Synthetic peptide
PDLESEFQAAISRKMVELVHFLLLK

SEQ ID NO: 573 - protein, 37 aa, Artificial Sequence
  Description of Artificial Sequence Synthetic polypeptide
SSTLVEVTLGEVPAADSPSPPHSPQGASSFSTTINYT

SEQ ID NO: 574 - protein, 28 aa, Artificial Sequence
  Description of Artificial Sequence Synthetic peptide
STFPDLESEFQAALSRKVAELVHFLLLK

SEQ ID NO: 575 - protein, 36 aa, Artificial Sequence
  Description of Artificial Sequence Synthetic polypeptide
SSTLVEVTLGEVPAAESPDPPQSPQGASSLPTTMNY

SEQ ID NO: 576 - protein, 28 aa, Artificial Sequence
  Description of Artificial Sequence Synthetic peptide
STSPDPESVFRAALSKKVADLIHFLLLK

SEQ ID NO: 577 - protein, 37 aa, Artificial Sequence
  Description of Artificial Sequence Synthetic polypeptide
SSPLVPGTLGEVPAAGSPGPLKSPQGASAIPTAIDFT

SEQ ID NO: 578 - protein, 27 aa, Artificial Sequence
  Description of Artificial Sequence Synthetic peptide
SYEKVINYLVMLNAREPISYPSLYEEV

SEQ ID NO: 579 - protein, 31 aa, Artificial Sequence
  Description of Artificial Sequence Synthetic polypeptide
SSSVDPAQLEFMFQEALKLKVAELVHFLLHK

SEQ ID NO: 580 - protein, 31 aa, Artificial Sequence
  Description of Artificial Sequence Synthetic polypeptide
STLQVLPDSESLPRSEIDEKVTDLVQFLLFK

SEQ ID NO: 581 - protein, 31 aa, Artificial Sequence
  Description of Artificial Sequence Synthetic polypeptide
STSPDLIDPESFSQDILHDKIIDLVHLLLRK

SEQ ID NO: 582 - protein, 37 aa, Artificial Sequence
  Description of Artificial Sequence Synthetic polypeptide
SSTLNVGTLEELPAAESPSPPQSPQEESFSPTAMDAI

SEQ ID NO: 583 - protein, 28 aa, Artificial Sequence
  Description of Artificial Sequence Synthetic peptide
STFPDLETSFQVALSRKMAELVHFLLLK

SEQ ID NO: 584 - protein, 37 aa, Artificial Sequence
  Description of Artificial Sequence Synthetic polypeptide
SSTLVEVTLREVPAAESPSPPHSPQGASTLPTTINYT

SEQ ID NO: 585 - protein, 16 aa, Artificial Sequence
  Description of Artificial Sequence Synthetic peptide
NLTNDGNLSNDWDFPR

SEQ ID NO: 586 - protein, 34 aa, Artificial Sequence
  Description of Artificial Sequence Synthetic polypeptide
SSPVLGDTPTSSPAAGIPQKPQGAPPTTTAAAAV

SEQ ID NO: 587 - protein, 31 aa, Artificial Sequence
  Description of Artificial Sequence Synthetic polypeptide
SSSQASTSTERSLKDSLTRKTKMLVQFLLYK

SEQ ID NO: 588 - protein, 32 aa, Artificial Sequence
  Description of Artificial Sequence Synthetic polypeptide
SSVLRDTASSSLAFGIPQEPQREPPTTSAAAA

SEQ ID NO: 589 - protein, 30 aa, Artificial Sequence
  Description of Artificial Sequence Synthetic polypeptide
SSEVSPSTESSSSNFINIKVGLLEQFLLYK

SEQ ID NO: 590 - protein, 31 aa, Artificial Sequence
  Description of Artificial Sequence Synthetic polypeptide
NSFHGPSSSESTGRDLLNTKTGELVQFLLNK

SEQ ID NO: 591 - protein, 34 aa, Artificial Sequence
  Description of Artificial Sequence Synthetic polypeptide
SSPASQSPPQSFPNAGIPQESQRASYPSSPASAV

SEQ ID NO: 592 - protein, 31 aa, Artificial Sequence
  Description of Artificial Sequence Synthetic polypeptide
TDSESLIESEPLFTYTLDEKVDELARFLLLK

SEQ ID NO: 593 - protein, 31 aa, Artificial Sequence
  Description of Artificial Sequence Synthetic polypeptide
GTSQGLPDSESSFTYTLDEKVAELVEFLLLK

SEQ ID NO: 594 - protein, 28 aa, Artificial Sequence
  Description of Artificial Sequence Synthetic peptide
HALPESESLPRYALDEKVAELVQFLLLK

SEQ ID NO: 595 - protein, 29 aa, Artificial Sequence
  Description of Artificial Sequence Synthetic peptide
NTSRVAITLKPQDPMEQNVAELLQFLLVK

SEQ ID NO: 596 - protein, 30 aa, Artificial Sequence
  Description of Artificial Sequence Synthetic polypeptide
ALAAKALARRRAYRRLNRTVAELVQFLLVK

SEQ ID NO: 597 - protein, 16 aa, Artificial Sequence
  Description of Artificial Sequence Synthetic peptide
DLTDEESLLSSWDFPR

SEQ ID NO: 598 - protein, 31 aa, Artificial Sequence; identical to SEQ ID NO: 3
  Description of Artificial Sequence Synthetic polypeptide

SEQ ID NO: 599 - protein, 34 aa, Artificial Sequence; identical to SEQ ID NO: 2
  Description of Artificial Sequence Synthetic polypeptide

SEQ ID NO: 600 - protein, 103 aa, Artificial Sequence
  Description of Artificial Sequence Synthetic polypeptide
DGVMTQTPAPVSAAVGGTVTINCQASQSIGSDLSWYQQKPGQPPKLLIYSASKLATVPSRFNGSGSGTQFTLTISGVQCDDAATYQCGWYYNAFGGGTEVVVK

SEQ ID NO: 601 - protein, 110 aa, Artificial Sequence
  Description of Artificial Sequence Synthetic polypeptide
DVVMTQTPASVSEPVGGTVTIKCQASQSIRNELFWWQQKPGQPPKLLIYAASKLASVPSRFSGSGSGTEFTLTISDLECADAATYYQCSYVSSSGTYGNVFGGGTEVVVK

SEQ ID NO: 602 - protein, 107 aa, Artificial Sequence
  Description of Artificial Sequence Synthetic polypeptide
QAVVTQTPSSVSAAVGGTVTISCQSSQSVYSNNLLSWYQQKPGQPPKLLIYKASTLASVPSRLKGSGSGTQFTLTISEVQCDDAATYQGYYSGVIYMFGGGTEVVVK

SEQ ID NO: 603 - protein, 110 aa, Artificial Sequence
  Description of Artificial Sequence Synthetic polypeptide
DVVMTHTPASLFETVGGTVTIKCQALQSVYDNNALSWYQQNAGQRPILLIYGASTLASAPSRFSASGSGTDFTLTIIDLECADASTYYQCTYYVSSYQNDFGGGTEVVVK

SEQ ID NO: 604 - protein, 106 aa, Artificial Sequence
  Description of Artificial Sequence Synthetic polypeptide
AIDMTQTPASVEAAVGGTITINCQASESISSWLAWYQQKPGQPPKLLIYEASKLASVPSRFSGSGSGTQFTLTISGVQCDDAATYQGGYYGWYYNAFGGGTEVVVK

SEQ ID NO: 605 - protein, 107 aa, Artificial Sequence
  Description of Artificial Sequence Synthetic polypeptide
AYDMTQTPASVEAAVGGTVTINCQASESISNYLSWYQQKPGQPPKLLIYWASTLASVSSRFKGSGSGTQFTLTISGVECADAATYQQGYSSSNVDNLFGGGTEVVVK

SEQ ID NO: 606 - protein, 110 aa, Artificial Sequence
  Description of Artificial Sequence Synthetic polypeptide
DIVMTQTPASVEAAVGGTVTIKCQASQSISSYLSWYQQKPGQPPKLLIYRASTLASVPSRFRGSGSGTQFTLTISDLECADAATYYQSYDDSSDNNFFYGFGGGTEVVVR

SEQ ID NO: 607 - protein, 110 aa, Artificial Sequence
  Description of Artificial Sequence Synthetic polypeptide
DIVMTQTPASVEAAVGGTVTIKCQASQSISSYLAWYHQKPGQPPKLLIYAASTLASVPSRFEGSGSGTQFTLTISDLECADAATYYQSYDDSRSSSFFYAFGGGTEVVVK

SEQ ID NO: 608 - protein, 110 aa, Artificial Sequence
  Description of Artificial Sequence Synthetic polypeptide
DIVMTQTPASVEAAVGGTVTIKCQASQSISSWLSWYQQKPGQPPKLLLYRATTLASVPSRFKGSGSGTQFTLTISDLECADAATYYQSYDDSSSSNFFYAFGGGTEVVVR

SEQ ID NO: 609 - protein, 110 aa, Artificial Sequence
  Description of Artificial Sequence Synthetic polypeptide
DIVMTQTPFSVEAAVGGTVTIKCQASQSISSYLSWYRQKPGQPPKLLIYRASTLASVPSRFKGSGSGTQFTLTISDLECADAATYYQSYDDSSSNNFFYGFGGGTEVVVR

SEQ ID NO: 610 - protein, 110 aa, Artificial Sequence
  Description of Artificial Sequence Synthetic polypeptide
DIVMTQTPSSVEAAVGGTVTIKCQASQNISSYLAWYQQKPGQPPKLLIYRASTLASVPSRFKGSGSGTQFTLTISDLECADAATYYQSYDDSRSSNFFYAFGGGTEVVVK

SEQ ID NO: 611 - protein, 110 aa, Artificial Sequence
  Description of Artificial Sequence Synthetic polypeptide
DIVMTQTPFSVEAAVGGTVTIKCQASQSISSYLSWYQQKPGQPPKLLIYRASPLASVPSRFKGSGSGTQFTLTISDLECADAATYYQSYDDSSSNNFFYGFGGGTEVVVR

SEQ ID NO: 612 - protein, 110 aa, Artificial Sequence
  Description of Artificial Sequence Synthetic polypeptide
DIVMTQTPASVEAAVGGTVTIKCQASESIGNALAWYQQKPGQPPSLLIYRASTLASIPSRVKGSGSGTQFTLTISDLECADAATYYQSYDDSSSSSFFYAFGGGTEVVVK

SEQ ID NO: 613 - protein, 108 aa, Artificial Sequence
  Description of Artificial Sequence Synthetic polypeptide
AQVLTQTESPVSAPVGGTVTINCQASQSVYDNNYLSWYQQKPGQPPKLLIYDASKLASVPSRFSGSGSGTQFTLTISGVQCDDAATYQGSYYGWYYNAFGGGTEVVVK

SEQ ID NO: 614 - protein, 108 aa, Artificial Sequence
  Description of Artificial Sequence Synthetic polypeptide
AAVLTQTPSPVSAAVGGTVSISCQSSQSVWHNDYLSWYQQKPGQPPKLLIYGASTLASVPSRFKGSGSGTQFSLTISGVQCDDAATYAGGYGRSSENGFGGGTEVVVK

SEQ ID NO: 615 - protein, 108 aa, Artificial Sequence
  Description of Artificial Sequence Synthetic polypeptide
AQVLTQTESPVSAPVGGTVTINCQASQSVYDNNWLSWYQQKPGQPPKLLIYDASKLASVPSRFSGSGSGTQFTLTISGVQCDDAATYQGSYYGWYYNAFGGGTEVVVK

SEQ ID NO: 616 - protein, 108 aa, Artificial Sequence
  Description of Artificial Sequence Synthetic polypeptide
AAVLTQTPSPVSAAVGGTVSISCQSSKSVYNNNWLAWYQQKPGQRPKLLIYDASTLDSVSSRFKGSGSGTQFTLTISDVQCDDGATYVGGYSSRSDNGFGGGTEVVVK

SEQ ID NO: 617 - protein, 108 aa, Artificial Sequence
  Description of Artificial Sequence Synthetic polypeptide
AQALTQTPSPVSAAVGGTVTISCQSSESVYNHNWLGWYQQKPGQPPKLLIYDASTLASVPSRFKGSGSGTQFTLTISESQCDDAAIYYQGYYQTSVWAFGGGTEVVVK

SEQ ID NO: 618 - protein, 109 aa, Artificial Sequence
  Description of Artificial Sequence Synthetic polypeptide
AAVLTQTPSPVSAAVGGTVSASCQSSKSVYNKNWLSWFQQKPGQPPKLLIYGASTLASVPSRFKGSGSGTQFTLTISDVQCDDAATYAGGYSSSSDTFAFGGGTEVVVK

SEQ ID NO: 619 - protein, 109 aa, Artificial Sequence
  Description of Artificial Sequence Synthetic polypeptide
AAVLTQTPSPVSAAVGGTVTINCQSSKSVYNKNWLSWFQQKPGQPPKLLIYGASTLASVPSRFKGSGSGTQFTLTISDVQCDDVATYAGGYSSSSDTFAFGGGTEVVVK

SEQ ID NO: 620 - protein, 109 aa, Artificial Sequence
  Description of Artificial Sequence Synthetic polypeptide
AAVLTQTPSPVSAAVGGTVSISCQSSKSVYNKNWLSWFQQKPGQPPKLLIYGASTLASVPSRFKGSGSGTQFTLTISDVQCDDAATYAGGYSSSSDTFAFGGGTEVVVK

SEQ ID NO: 621 - protein, 109 aa, Artificial Sequence
  Description of Artificial Sequence Synthetic polypeptide
AAVLTQTPSPVSAAVGGTVSISCQSSKSVYNKNWLSWFQQKPGQPPKLLIYGASTLASVPSRFKGSGSGTQFTLTISGVQCDDAATYAGGYSSSSDTFAFGGGTEVVVK

SEQ ID NO: 622 - protein, 105 aa, Artificial Sequence
  Description of Artificial Sequence Synthetic polypeptide
AQVMTQTPASVSAAVGGTVTIICQASESISSYLNWYQQKLGQPPKLLIYYASTLASVPSRFKGSGSGTEYTLTISGVQCDDAATYQHGYGWYYNAFGGGTEVVVK

SEQ ID NO: 623 - protein, 110 aa, Artificial Sequence
  Description of Artificial Sequence Synthetic polypeptide
DIVMTQTPASVEAAVGGTVTIKCQASQNIDSYLAWYQQKPGQPPKLLIYRASTLASVPSRFKGSGSGTEFTLTISDLECADAATYYQSYDDSRSSSFFYGFGGGTEVVVK

SEQ ID NO: 624 - protein, 110 aa, Artificial Sequence
  Description of Artificial Sequence Synthetic polypeptide
DIVMTQTPASVEAAVGGTVTIKCQASQNINSYLAWYQQKPGQPPKLLIYRASTLASVPSRFKGSGSGTEFTLTISDLECADAATYYQSYDDSRSISFFYAFGGGTEVVVK

SEQ ID NO: 625 - protein, 110 aa, Artificial Sequence
  Description of Artificial Sequence Synthetic polypeptide
DVVMTQTPASVEATVGGTVTIKCQSSQSVYDNNALAWYQQNAGQRPRLLIYGASTLASVPSRFSASGSGTEFTLTISDLECADAATYYQCTYYVSSYQNDFGGGTEVVVK

SEQ ID NO: 626 - protein, 105 aa, Artificial Sequence
  Description of Artificial Sequence Synthetic polypeptide
AQVLTQTPASVSAAVGGTVTINCQASESISSYLNWYQQKLGQPPKLLIYYASTLASVPSRFKGSGSGTEYTLTISGVQCDDAATYQHGYGWYYNAFGGGTEVVVK

SEQ ID NO: 627 - protein, 108 aa, Artificial Sequence
  Description of Artificial Sequence Synthetic polypeptide
AQVLTQTPASVSAAVGGTVTISCQSSQSVYSSDLLSWYQQKPGQPPKLLIYKASTLASVPSRFKGSGSGTQFTLTISELQCDDAATYYQGYYSGVVYIFGGGTEVVVK

SEQ ID NO: 628 - protein, 107 aa, Artificial Sequence
  Description of Artificial Sequence Synthetic polypeptide
AQVLTQTPASVSAAVGGTVTISCQSSQSVYSSDLLSWYQQKPGQPPKLLIYKASTLASVPSRFKGSGSGTQFTLTISEVQCDDAATYQGYYSGVVYIFGGGTEVVVK

SEQ ID NO: 629 - protein, 107 aa, Artificial Sequence
  Description of Artificial Sequence Synthetic polypeptide
AQVLTQTPASVSAAVGGTVTISCQSSQSLYNSDLLSWYQQKPGQPPKLLIYKASTLASVPSRFKGSGSGTQFTLTISEVQCDDAATYQGYYSGVVYIFGGGTEVVVK

SEQ ID NO: 630 - protein, 123 aa, Artificial Sequence
  Description of Artificial Sequence Synthetic polypeptide
QSLEESGGRLVTPGGSLTLTCTVSGIDLSYAMGWVRQAPGKGLEYIGIISSSGSTYYASWAKGRFTISKTSSTTVDLKMTSLTTEDTATYFCAGAYTSSSGYYIGYYIYFNLWGPGTLVTVSS

SEQ ID NO: 631 - protein, 108 aa, Artificial Sequence
  Description of Artificial Sequence Synthetic polypeptide
QSLEESGGRLVTPGGSLTLTCTVSGIDLSHAMIWVRQAPGEGLEWIGTIGSRDTIYYASWAKGRFTISKTSSTTMDLKMTSLTIEDTATYFCVRNALWGPGTLVTVSS

SEQ ID NO: 632 - protein, 120 aa, Artificial Sequence
  Description of Artificial Sequence Synthetic polypeptide
QSVEESGGRLVTPGTPLTLTCTVSGFSLSYAMSWVRQAPGKGLEWIGIISSSGSTYYASWAKGRFTISKTSTTVDLKITSPTTEDTATYFCARSYDDYGDYGYYIYFNLWGPGTLVTVSS

SEQ ID NO: 633 - protein, 115 aa, Artificial Sequence
  Description of Artificial Sequence Synthetic polypeptide
QSLEESGGRLVTPGTPLTLTCKVSGFSLSYDMSWVRQAPGKGLEWIGIIYASGSTYYASWAKGRFTISKTSTTVDLKIASPTTEDTATYFCARDPAGYSISFGLWGPGTLVTVSS

SEQ ID NO: 634 - protein, 124 aa, Artificial Sequence
  Description of Artificial Sequence Synthetic polypeptide
EQQKESGGRLVMPGGSLTLTCTVSGFSSYNMGWVRQAPGEGLEYIGWISTGGSAYYASWVNGRFTISKTSTTMDLKMTSLTAADTATYFCARFGEGPTHHVAMVVGYYIYFNLWGPGTLVTVSS

SEQ ID NO: 635 - protein, 114 aa, Artificial Sequence
  Description of Artificial Sequence Synthetic polypeptide
EQLKESGGGLVAPGGTLTLTCAVSGFSSYNMGWVRQAPGEGLEYIGIIGASDSALYASWAKGRFTISKTSTTVDLKITSPTTEDTATYFCARGGLGLSTGFALWGPGTLVTVSS

SEQ ID NO: 636 - protein, 123 aa, Artificial Sequence
  Description of Artificial Sequence Synthetic polypeptide
QSVEESGGRLVTPGTPLTLTCTVSGFSLSYAMSWVRQAPGKGLEWIGIISSSGSTYYASWAKGRFTISKTSTTVDLKITSPTTEDTATYFCARYGVLVPMAMGYYIYYGMDLWGPGTLVTVSS

SEQ ID NO: 637 - protein, 112 aa, Artificial Sequence
  Description of Artificial Sequence Synthetic polypeptide
QSLEESGGRLVTPGGPLTLTCTVSGFSLSYDMSWVRQAPGKGLEWIGYIATDGRPYYASWAKGRFTISKPSSTTVDLKITSPTTEDTATYFCVRGGYAGGLWGPGTLVTVSS

SEQ ID NO: 638 - protein, 124 aa, Artificial Sequence
  Description of Artificial Sequence Synthetic polypeptide
QSLEESGGDLVKPGASLTLTCTASGFSFSSYYMCWVRQAPGKGLEWIACIYAGSSGSTYYASWAKGRFTISKTSSTTVTLQMTSLTAADTATYFCARSYDDYGDYGYYIYFNLWGPGTLVTVSS

SEQ ID NO: 639 - protein, 112 aa, Artificial Sequence
  Description of Artificial Sequence Synthetic polypeptide
QSLEESGGGLVQPEGSLTLTCTAFGFTLNYYICWVRQAPGKGLEWIACIDNANGRTYYASWAKGRFTISKTSSTTVTLQMTSLTAADTATYFCARSLSTPLWGPGTLVTVSS

SEQ ID NO: 640 - protein, 112 aa, Artificial Sequence
  Description of Artificial Sequence Synthetic polypeptide
QSLEESGGGLVQPEGSLTLTCTASGFTLNYYICWVRQAPGKGLEWIACIDNANGRTYYATWAKGRFTISKTSSTTVTLQMPSLTAADTATYFCARSLSTNLWGPGTLVTVSS

SEQ ID NO: 641 - protein, 119 aa, Artificial Sequence
  Description of Artificial Sequence Synthetic polypeptide
QSLEESGGGLVKPGASLTLTCKASGSDFSYYYICWVRQAPGKGLEWVACIGGGNTDATAYARWAKGRFTISKTSATTVALQMTSLTAADTATYFCVRGGPDNNVQFNLWGPGTLVTVSS

SEQ ID NO: 642 - protein, 112 aa, Artificial Sequence
  Description of Artificial Sequence Synthetic polypeptide
QSLEESGGGLVQPEGSLTLTCTASGFTLNYYICWVRQAPGKGLEWIACIDNSNGRTYYASWAKGRFTISKTSSTTVTLQMTSLTAADTATYFCARSLSTPLWGPGTLVTVSS

SEQ ID NO: 643 - protein, 119 aa, Artificial Sequence
  Description of Artificial Sequence Synthetic polypeptide
QSLEESGGGLVKPGASLTLTCKASGSDLSAYYICWIRQAPGKGLEWVACIGGVNRVATAYATWAKGRFTISKTSSTTVTLQMTSLTAADTATYFCVRGGPDNNVQFNLWGPGTLVTVSS

SEQ ID NO: 644 - protein, 119 aa, Artificial Sequence
  Description of Artificial Sequence Synthetic polypeptide
QSLEESGGGLVKPGASLTLTCKASGSDLRYYYSCWIRQAPGKGLEWVACVGGVNRDATAYATWAKGRFTISKTSSTTVTLQMTSLTAADTATYFCVRGGPDNNVQFNLWGPGTLVTVSS

SEQ ID NO: 645 - protein, 112 aa, Artificial Sequence
  Description of Artificial Sequence Synthetic polypeptide
QSLEESGGGLVQPEGSLTLTCTASGFTLNYYICWVRQAPGKGLEWIACIDNVNGRTYYASWAKGRFTISRTSSTTVTLQMTSLTAADTATYFCARSLSTPLWGPGTLVTVSS

SEQ ID NO: 646 - protein, 124 aa, Artificial Sequence
  Description of Artificial Sequence Synthetic polypeptide
QSLEESGGDLVKPGASLTLTCTASGFSFSSYYMCWVRQAPGKGLEWIACIYAGSSGSTYYASWAKGRFTISKTSSTTVTLQMTSLTAADTATYFCARSYDDYGDYYYIDAFDPWGPGTLVTVSS

SEQ ID NO: 647 - protein, 112 aa, Artificial Sequence
  Description of Artificial Sequence Synthetic polypeptide
QSLEESGGGLVQPEGSLTLTCTASGVTLNYYICWVRQAPGKGLEWIGCIDNVNGRTYYASWAKGRFTISKTSSTTGTLQMTSLTAADTATYFCARSLATPLWGPGTLVTVSS

SEQ ID NO: 648 - protein, 112 aa, Artificial Sequence
  Description of Artificial Sequence Synthetic polypeptide
QSLEESGGGLVQPEGSLTLTCTASGVTLNYYICWVRQAPGKGLEWIGCIDNVNGRTYYASWAKGRFTISKASSTTGTLQMTSLTAADTATYFCARSLATPLWGPGTLVTVSS

SEQ ID NO: 649 - protein, 112 aa, Artificial Sequence
  Description of Artificial Sequence Synthetic polypeptide
QSLEESGGGLVQPEGSLTVTCTAFGVTLNYYICWVRQAPGKGLEWIGCIDNINGRTYYASWAKGRFTISKTSSTTGTLQMTSLTAADTATYFCARSLATPLWGPGTLVTVSS

SEQ ID NO: 650 - protein, 112 aa, Artificial Sequence
  Description of Artificial Sequence Synthetic polypeptide
QSLEESGGGLVQPEGSLTLTCTAFGVTLNYYICWVRQAPGKGLEWVGCIDNANGRTYYASWAKGRFTISKTSSTTGTLQMTSLTAADTATYFCARSLATPLWGPGTLVTVSS

SEQ ID NO: 651 - protein, 112 aa, Artificial Sequence
  Description of Artificial Sequence Synthetic polypeptide
QSLEESGGGLVQPEGSLTLTCTASGFTLNYYICWVRQAPGKGLEWIACIDNANGRTYYANWAKGRFTISKTSSTTVTLQMTSLTAADTATYFCARSLATPLWGPGTLVTVSS

SEQ ID NO: 652 - protein, 120 aa, Artificial Sequence
  Description of Artificial Sequence Synthetic polypeptide
QSLEESGGRLVTPGGSLTLTCTVSGIDLSYAMGWVRQAPGKGLEYIGIISSSGSTYYASWAKGRFTISKTSSTTVDLKMTSLTTEDTATYFCAGYAGSSWDGYYIYFNLWGPGTLVTVSS

SEQ ID NO: 653 - protein, 116 aa, Artificial Sequence
  Description of Artificial Sequence Synthetic polypeptide
QSVEESGGRLVTPGTPLTLTCTVSGVDLSSAVTWVRQAPGMGLEYIGFLQAGDGSAYYASWAKGRFTISKTSSTTVDLKMTSLTTEDTATYFCARHKGNSYVPNLWGPGTLVTVSS

SEQ ID NO: 654 - protein, 123 aa, Artificial Sequence
  Description of Artificial Sequence Synthetic polypeptide
QSVEESGGRLVTPGTPLTLTCTVSGFSLSYAMSWVRQAPGKGLEWIGIISSSGSTYYASWAKGRFTISKTSTTVDLKITSPTTEDTATYFCARAYTSSSGYYIYYIYYGMDLWGPGTLVTVSS

SEQ ID NO: 655 - protein, 111 aa, Artificial Sequence
  Description of Artificial Sequence Synthetic polypeptide
QSVEESGGRLVTPGTPLTLTCTISGFSLSYAMSWVRQAPGKGLEWIGSIGGGGSAVYASWAKGRFTISKTSTTVDLRITSPTTEDTAMYFCGRGFYSIDLWGPGTLVTVSS

SEQ ID NO: 656 - protein, 125 aa, Artificial Sequence
  Description of Artificial Sequence Synthetic polypeptide
EQLEESGGDLVKPEGSLTLTCTASGFSSSYWICWVRQAPGKGLEWIACIYAGSSGSTYYASWAKGRFTISKTSSTTVTLQMTSLTAADTATYFCARYYSYGYAYATYYIDAFDPWGPGTLVTVSS

SEQ ID NO: 657 - protein, 122 aa, Artificial Sequence
  Description of Artificial Sequence Synthetic polypeptide
EQLVESGGGLVKPGASLTLTCKASGFSSGQLMCWVRQAPGKGLEWIACIGSGSNAISTFYASWAQGRFTISKSSSTTVTLQLTSLTAADTATYFCARVGSDDYGDSDVFDPWGPGTLVTVSS

SEQ ID NO: 658 - protein, 123 aa, Artificial Sequence
  Description of Artificial Sequence Synthetic polypeptide
EQLEESGGDLVKPEGSLTLTCTASGFSSSYWICWVRQAPGKGLEWIACIYAGSSGSTYYASWAKGRFTISKTSSTTVTLQMTSLTAADTATYFCARSYDDYGDYYYIDAFDPWGPGTLVTVSS

SEQ ID NO: 659 - protein, 121 aa, Artificial Sequence
  Description of Artificial Sequence Synthetic polypeptide
EQLVESGGGLVQPEGSLTLTCAASGFSSSYFMCWVRQAPGKGLEWIACIAVGSSGSTYYASWAKGRFTISKTSSTTVTLQMTSLTAADTATYFCARVGYDDYGDSDAFDPWGPGTLVTVSS

SEQ ID NO: 660 - protein, 122 aa, Artificial Sequence
  Description of Artificial Sequence Synthetic polypeptide
EQLVESGGGLVQPKGSLTLTCAASGFSSSYFMCWVRQAPGKGLEWIACIGSGSSAISTFYASWAQGRFTISKTSSTTVTLQMTSLTAADTATYFCARVGYDDYGDSDAFDPWGPGTLVTVSS

SEQ ID NO: 661 - protein, 120 aa, Artificial Sequence
  Description of Artificial Sequence Synthetic polypeptide
QSVEESGGRLVTPGTPLTLTCTVSGFSLSYAMSWVRQAPGKGLEWIGIISSSGSTYYASWAKGRFTISKTSTTVDLKITSPTTEDTATYFCARSYDDYGDYGYYIYFNIWGPGTLVTVSS

SEQ ID NO: 662 - protein, 113 aa, Artificial Sequence
  Description of Artificial Sequence Synthetic polypeptide
QSLEESGGRLVTPGTPLTLTCTVSGIDLSYGMTWVRQAPGKGLEWIGYIWTDGRTYYANWAKGRFTISKTSTTVDLKITSPTAEDTATYFCARPFDGNYRDIWGPGTLVTVSL

SEQ ID NO: 663 - protein, 122 aa, Artificial Sequence
  Description of Artificial Sequence Synthetic polypeptide
EQLEESGGDLVKPEGSLTLTCTASGFSSSYWICWVRQAPGKGLEWIACIYAGSSGSTYYASWAKGRFTISKTSSTTVTLQMTSLTAADTATYFCARYAGSSWDYYIDAFDPWGPGTLVTVSS

SEQ ID NO: 664 - protein, 121 aa, Artificial Sequence
  Description of Artificial Sequence Synthetic polypeptide
EQLVESGGGLVQPEGSLTLTCAASGFSSSYFMCWVRQAPGKGLEWIACIGVGGSGSTYYANWAKGRFTISKTSSTTVTLQMTSLTAADTATYFCARVAYDDDGDSDAFDPWGPGTLVTVSS